(19) United States
(12) Patent Application Publication
(10) Pub. No.: US 2025/0000910 A1
(43) Pub. Date: Jan. 2, 2025

(54) Methods of Improving Therapeutic Efficacy of Mesenchymal Stromal Cells for Treatment of Osteoarthritis

(71) Applicants: Georgia Tech Research Corporation, Atlanta, GA (US); Emory University, Atlanta, GA (US)

(72) Inventors: Nick Willett, Atlanta, GA (US); Levi B. Wood, Atlanta, GA (US); Jay M. McKinney, Atlanta, GA (US)

(21) Appl. No.: 18/292,483

(22) PCT Filed: Jul. 29, 2022
(86) PCT No.: PCT/US2022/074305

§ 371 Date: Jan. 26, 2024

Related U.S. Application Data
(60) Provisional application No. 63/227,009, filed on Jul. 29, 2021.

(51) Int. Cl. A61K 35/28; A61P 19/02; C12N 5/0775

(52) U.S. Cl. CPC A61K 35/28; A61P 19/02; C12N 5/0663; C12N 2501/2301; C12N 2501/727

(57) ABSTRACT

The invention is directed to methods of improving the therapeutic efficacy of mesenchymal stromal cells (MSCs) for use in treating osteoarthritis (OA), and more specifically to methods of altering levels of certain proteins, phospho-proteins, genes, and/or MSC-secreted cytokines, chemokines, and/or growth factors in order to improve therapeutic efficacy, as well as methods of distinguishing and classifying MSCs as being highly therapeutic or less therapeutic based on levels of certain phospho-proteins and/or MSC-secreted cytokines, chemokines, and/or growth factors and methods of treating a subject having OA with MSCs having improved therapeutic efficacy.

### CROSS-REFERENCE TO RELATED APPLICATIONS

This application claims priority to U.S. Provisional Application No. 63/227,009, filed on Jul. 29, 2021, the disclosure of which is herein incorporated by reference in its entirety.

### GOVERNMENT SPONSORSHIP

This invention was made with government support under Grant No. W81XWH1810505 awarded by the Department of Defense. The government has certain rights in the invention.

### BACKGROUND OF THE INVENTION

### 1. Field of the Invention

Embodiments of the present invention relate generally to methods of improving the therapeutic efficacy of mesenchymal stromal cells (MSCs) for use in treating osteoarthritis (OA), and more specifically to methods of altering levels of certain proteins, phospho-proteins, genes, and/or MSC-secreted cytokines, chemokines, and/or growth factors in order to improve therapeutic efficacy, as well as methods of distinguishing and classifying MSCs as being highly therapeutic or less therapeutic based on levels of certain phospho-proteins and/or MSC-secreted cytokines, chemokines, and/or growth factors and methods of treating a subject having OA with MSCs having improved therapeutic efficacy.

### 2. Background

Osteoarthritis (OA) is the most prevalent chronic disease of the joints and leads to degeneration of articular cartilage surfaces. While physical therapy and weight loss have demonstrated improved functionality in patients with OA, current drugs are limited to providing symptomatic relief. Thus, there is a notable need for the development of a disease-modifying therapeutic for OA. Mesenchymal stromal cells (MSC) offer a promising treatment strategy for OA due to the regenerative and immunomodulatory capacity these cells possess. MSC therapeutics for cartilage regeneration have been widely studied, in both the pre-clinical and clinical environment. While pre-clinical studies have shown improved cartilage repair with MSC treatment, effective translation into the clinic has been limited by numerous factors ranging from high variability and heterogeneity of MSCs to poor understanding of critical quality and potency attributes. There is therefore a need to identify certain cellular attributes that relate to the therapeutic efficacy of MSCs, including human MSCs (hMSCs), for treatment of OA. Such attributes may include MSC-secreted cytokines, ribonucleic acid (RNA) transcripts, and levels of intracellular signaling phospho-proteins.

What is needed, therefore, is a method of distinguishing and classifying which MSCs are more therapeutically effective or less therapeutically effective by determining the levels of certain proteins, phospho-proteins, genes, and/or MSC-secreted cytokines, chemokines, and/or growth factors and comparing such levels to standard levels of these proteins, phospho-proteins, genes, and/or MSC-secreted cytokines, chemokines, and/or growth factors, as well as methods of improving the therapeutic efficacy of MSCs that have been identified as being less therapeutic. The MSCs obtained from such methods can have consistently higher therapeutic efficacies and can be used to treat a subject having OA. Further, systems and devices for performing such distinction, classification, and/or optimization of therapeutic efficacy are contemplated herein. It is to such methods, systems, and devices that embodiments of the present invention are primarily directed.

### BRIEF SUMMARY OF THE INVENTION

As specified in the Background Section, there is a great need in the art to identify technologies for characterizing the therapeutic efficacies of MSCs and using this understanding to develop novel methods of distinguishing and classifying MSCs as being highly therapeutic or less therapeutic based on the levels of certain proteins, phospho-proteins, genes, and/or MSC-secreted cytokines, chemokines, and/or growth factors, as well as methods of improving therapeutic efficacies of MSCs by altering the levels of such proteins, phospho-proteins, genes, and/or MSC-secreted cytokines, chemokines, and/or growth factors, and treating a subject with OA using highly therapeutically effective MSCs obtained from these methods. Systems and devices for performing such distinction, classification, and/or optimization of therapeutic efficacy are contemplated herein. The present invention satisfies this and other needs.

Embodiments of the present invention relate generally to methods of improving the therapeutic efficacy of MSCs for use in treating OA, and more specifically to methods of altering levels of certain proteins, phospho-proteins, genes, and/or MSC-secreted cytokines, chemokines, and/or growth factors in order to improve therapeutic efficacy, as well as methods of distinguishing and classifying MSCs as being highly therapeutic or less therapeutic based on levels of certain phospho-proteins and/or MSC-secreted cytokines, chemokines, and/or growth factors and methods of treating a subject having OA with MSCs having improved therapeutic efficacy.

In one aspect, the invention provides a method of distinguishing highly therapeutic mesenchymal stromal cells (MSCs) from less therapeutic MSCs for use in treating osteoarthritis in a subject in need thereof, the method comprising: (a) isolating MSCs from a biological sample; (b) incubating the MSCs with interleukin 1 beta (IL-1B), IL-6, interferon gamma (IFN-γ), tumor necrosis factor alpha (TNF-alpha), and combinations thereof; (c) measuring (i) levels of cytokines, chemokines, and/or growth factors secreted by the MSCs and/or (ii) measuring proteomic, phospho-proteomic, or transcription profiles of genes in mitogen-activated protein kinase (MAPK) and phosphatidylinositol 3-kinase/protein kinase B (PI3K/Akt) pathways; and (d) distinguishing highly therapeutic MSCs from less therapeutic MSCs by one or more steps of: (i) classifying the MSCs as being highly therapeutic if there is greater secretion of cytokines, chemokines, and/or growth factors associated with an increased phospho-c-Jun N-terminal kinase (p-JNK) level relative to secretion from a standard control MSC, or if the secretion of the cytokines, chemokines, and/or growth factors associated with the increased p-JNK level is substantially similar to secretion from a standard control MSC that has been previously identified as being highly therapeutic; (ii) classifying the MSCs as being less therapeutic if there is equal or lower secretion of cytokines, chemokines, and/or growth factors associated with the increased p-JNK level relative to secretion from a standard control MSC, or if the secretion of the cytokines, chemokines, and/or growth factors associated with the increased p-JNK level is substantially similar to secretion from a standard control MSC that has been previously identified as being less therapeutic; and/or (iii) classifying the MSCs as being highly therapeutic if levels of proteins, phospho-proteins, and/or expression of genes in the MAPK pathway are increased relative to levels of the same proteins, phospho-proteins, and/or genes in the MAPK pathway from a standard control MSC or substantially similar to levels of the same proteins, phospho-proteins, and/or genes in the MAPK pathway from a standard control MSC that has been previously identified as being highly therapeutic, and/or classifying the MSCs as being less therapeutic if levels of proteins, phospho-proteins, or expression of genes in the PI3K/Akt pathway are increased relative to levels of proteins, phospho-proteins, and/or genes in the PI3K/Akt pathway from a standard control MSC that has been previously identified as being highly therapeutic or are substantially similar to levels of proteins, phospho-proteins, and/or genes in the PI3K/Akt pathway from a standard control MSC that has been previously identified as being less therapeutic.

In another aspect, the invention provides a method of identifying and/or producing mesenchymal stromal cells as being therapeutically effective for treating osteoarthritis in a subject in need thereof, the method comprising: (a) incubating the MSCs with interleukin 1 beta (IL-1B), IL-6, interferon gamma (IFN-γ), tumor necrosis factor alpha (TNF-alpha), and combinations thereof; (b) measuring levels of cytokines, chemokines, and/or growth factors secreted by the MSCs; (c) optionally measuring transcription profiles of genes in mitogen-activated protein kinase (MAPK) and phosphatidylinositol 3-kinase/protein kinase B (PI3K/Akt) pathways; and (d) identifying the MSCs as being therapeutic if: (i) there is greater secretion of cytokines, chemokines, and/or growth factors associated with an increased phospho-c-Jun N-terminal kinase (p-JNK) level relative to secretion from a standard control MSC, or if the secretion of the cytokines, chemokines, and/or growth factors associated with the increased p-JNK level is substantially similar to secretion from a standard control MSC that has been previously identified as being therapeutic; and/or (ii) the levels of proteins, phospho-proteins, and/or expression of genes in the MAPK pathway are increased relative to levels of the same proteins, phospho-proteins, and/or genes in the MAPK pathway from a standard control MSC or substantially similar to levels of the same proteins, phospho-proteins, and/or genes in the MAPK pathway from a standard control MSC that has been previously identified as being highly therapeutic; and (e) optionally propagating therapeutic MSCs.

In another aspect, the invention provides a method of altering the cytokine, chemokine, and/or growth factor expression profile of a plurality of mesenchymal stromal cells (MSCs) for use in treating osteoarthritis in a subject in need thereof, the method comprising: (a) isolating MSCs from a biological sample; (b) incubating the MSCs with interleukin 1 beta (IL-1B), IL-6, interferon gamma (IFN-γ), tumor necrosis factor alpha (TNF-alpha), and combinations thereof; (c) measuring (i) levels of cytokines, chemokines, and/or growth factors secreted by the MSCs and/or (ii) measuring proteomic, phospho-proteomic, or transcription profiles of genes in mitogen-activated protein kinase (MAPK) and phosphatidylinositol 3-kinase/protein kinase B (PI3K/Akt) pathways; and (d) classifying the MSCs as being highly therapeutic or less therapeutic by one or more steps of: (i) classifying the MSCs as being highly therapeutic if there is greater secretion of cytokines, chemokines, and/or growth factors associated with an increased phospho-c-Jun N-terminal kinase (p-JNK) level relative to secretion from a standard control MSC, or if the secretion of the cytokines, chemokines, and/or growth factors associated with the increased p-JNK level is substantially similar to secretion from a standard control MSC that has been previously identified as being highly therapeutic; and/or (ii) classifying the MSCs as being less therapeutic if there is equal or lower secretion of cytokines, chemokines, and/or growth factors associated with the increased p-JNK level relative to secretion from a standard control MSC, or if the secretion of the cytokines, chemokines, and/or growth factors associated with the increased p-JNK level is substantially similar to secretion from a standard control MSC that has been previously identified as being less therapeutic; and/or (iii) classifying the MSCs as being highly therapeutic if levels of proteins, phospho-proteins, and/or expression of genes in the MAPK pathway are increased relative to levels of the same proteins, phospho-proteins, and/or genes in the MAPK pathway from a standard control MSC or substantially similar to levels of the same proteins, phospho-proteins, and/or genes in the MAPK pathway from a standard control MSC that has been previously identified as being highly therapeutic, and/or classifying the MSCs as being less therapeutic if levels of proteins, phospho-proteins, and/or expression of genes in the PI3K/Akt pathway are increased relative to levels of the same proteins, phospho-proteins, and/or genes in the PI3K/Akt pathway from a standard control MSC or substantially similar to levels of the same proteins, phospho-proteins, and/or genes in the PI3K/Akt pathway from a standard control MSC that has been previously identified as being less therapeutic; and (e) treating the less therapeutic MSCs with an activator of the MAPK pathway and/or an inhibitor of the PI3K/Akt pathway, optionally prior to or simultaneously with administration of the less therapeutic MSCs to the subject in need thereof; and/or (f) treating the highly therapeutic MSCs with an activator of the MAPK pathway and/or an inhibitor of the PI3K/Akt pathway, optionally prior to or simultaneously with administration of the less therapeutic MSCs to the subject in need thereof.

In another aspect, the invention provides a method of treating osteoarthritis in a subject in need thereof, the method comprising: (a) isolating MSCs from a biological sample; (b) incubating the MSCs with interleukin 1 beta (IL-1B), IL-6, interferon gamma (IFN-γ), tumor necrosis factor alpha (TNF-alpha), and combinations thereof; (c) measuring (i) levels of cytokines, chemokines, and/or growth factors secreted by the MSCs and/or (ii) measuring proteomic, phospho-proteomic, or transcription profiles of genes in mitogen-activated protein kinase (MAPK) and phosphatidylinositol 3-kinase/protein kinase B (PI3K/Akt) pathways; and (d) distinguishing highly therapeutic MSCs from less therapeutic MSCs by one or more steps of: (i) classifying the MSCs as being highly therapeutic if there is greater secretion of cytokines, chemokines, and/or growth factors associated with an increased phospho-c-Jun N-terminal kinase (p-JNK) level relative to secretion from a standard control MSC, or if the secretion of the cytokines, chemokines, and/or growth factors associated with the increased p-JNK level is substantially similar to secretion from a standard control MSC that has been previously identified as being highly therapeutic; and/or (ii) classifying the MSCs as being less therapeutic if there is equal or lower secretion of cytokines, chemokines, and/or growth factors associated with the increased p-JNK level relative to secretion from a standard control MSC, or if the secretion of the cytokines, chemokines, and/or growth factors associated with the increased p-JNK level is substantially similar to secretion from a standard control MSC that has been previously identified as being less therapeutic; and/or (iii) classifying the MSCs as being highly therapeutic if levels of proteins, phospho-proteins, and/or expression of genes in the MAPK pathway are increased relative to levels of the same proteins, phospho-proteins, and/or genes in the MAPK pathway from a standard control MSC or substantially similar to levels of the same proteins, phospho-proteins, and/or genes in the MAPK pathway from a standard control MSC that has been previously identified as being highly therapeutic, and/or classifying the MSCs as being less therapeutic if levels of proteins, phospho-proteins, and/or expression of genes in the PI3K/Akt pathway are increased relative to levels of the same proteins, phospho-proteins, and/or genes in the PI3K/Akt pathway from a standard control MSC or substantially similar to levels of the same proteins, phospho-proteins, and/or genes in the PI3K/Akt pathway from a standard control MSC that has been previously identified as being less therapeutic; and wherein: (i) the highly therapeutic MSCs are administered to the subject in need thereof or are optionally treated with an activator of the MAPK pathway and/or an inhibitor of the PI3K/Akt pathway prior to or simultaneously with administration of the highly therapeutic MSCs to the subject in need thereof; (ii) the less therapeutic MSCs are treated with an activator of the MAPK pathway and/or an inhibitor of the PI3K/Akt pathway prior to or simultaneously with administration of the less therapeutic MSCs to the subject in need thereof.

These and other objects, features and advantages of the present invention will become more apparent upon reading the following specification in conjunction with the accompanying description, claims and drawings.

### BRIEF DESCRIPTION OF THE DRAWINGS

The accompanying Figures, which are incorporated in and constitute a part of this specification, illustrate several aspects described below.

FIG. 1 illustrates characteristic pathological phenotypes of OA. Primary phenotypic outcomes of OA include articular cartilage lesion formation and exposed bone with associated phenotypes of osteophyte formation (bone spur formation on the marginal edges of the joint), synovial capsule inflammation, and subchondral bone sclerosis (thickening and hardening).

FIG. 2 illustrates a comparison of OA progression in pre-clinical MMT rat model and clinical human disease using human OA research society international (OARSI) scoring system (grades 1-6). Damage to articular cartilage is similar for the rat MMT model and early changes in human disease pathology. The prominence of osteophytes and changes to subchondral bone occurred earlier in the rat MMT model compared to findings for human OA.

FIGS. 3a-3i illustrate representative microCT images demonstrating articular cartilage, osteophyte, and subchondral bone volume of interest (VOI). (3a) Rat tibial articular cartilage thickness map overlays on bone indicating articular cartilage VOI. Color heat map indicates cartilage thickness according to the scale bar. (3b) Representative images of coronal sections of rat tibial joint showing outline of total medial articular cartilage, (3c) medial 1/3 (indicated in white) of medial articular cartilage, outline of (3d) medial tibial subchondral bone, and (3e) cortical subchondral bone (indicated in white) of medial tibia. Representative coronal sections of medial tibial joint illustrating (3f) no osteophyte in sham joint, (3g) contour of osteophyte in MMT joint, (3h) cartilaginous osteophyte volume (indicated in white) in MMT joint, and (3i) mineralized osteophyte volume (indicated in white) in MMT joint. Scale bar in (3a) is universal for all representative images of coronal sections.

FIGS. 4a-4o illustrate an analysis of surface roughness, lesion volume and full thickness lesion area of articular cartilage. (4a, 4b) Representative microCT images of a 2D coronal-sectioned sham sample demonstrates no surface fibrillations or lesion formation in the articular cartilage surface. (4c) This smooth surface is further represented in a 3D reconstruction of the cartilage surface (for only the region highlighted in the figure). (4e, 4f) White circles on the articular cartilage surface depict the true cartilage boundary determined by the MATLAB script. (4g) These actual surface values are compared to the modeled surface (root mean square) produced by the code to determine surface roughness, which can be readily visualized in the surface rendering. (4i, 4j) Lesion volume was determined using a similar approach, but volume was calculated by comparing the actual surface (white) to modeled surface (gray). (4k) The lesion volumes modeled in the figure are clearly represented in the surface renderings of the cartilage surface. (4m-4o) Exposed bone was quantified by calculating the area where the pixel spacing between the cartilage surface and underlying subchondral bone were <3 pixels. (4d, 4h, 4l) The formulas for surface roughness, lesion volume, and full thickness lesion area (exposed bone) are also provided.

FIGS. 5a-5c illustrate a PLSR analysis methods overview. (5a) PCA identifies axes of maximum variation among samples in the data when measurement variables (X1 and X2) are plotted against one another. Through incorporation of a response variable Y, PLSR enables identification of maximum co-variation between the X variables and different Y responses. PLSR outputs new linear combinations of X variables, referred to as LVs. (5b) Each latent variable is comprised of weights, which ranks the importance of each input variable Xi, in determining the final composite values for each sample data point. (5c) To obtain the PLSR scores plot, the raw data is multiplied by the calculated weights for each LV (LV1 and LV2). The new axes defined by these LVs (LV1 and LV2) better separates the data with respect to the identity of the Y response variables.

FIGS. 6a-6f illustrate an encapsulated hMSC characterization. Multipotency of hMSCs confirmed prior to encapsulation, as cultured hMSCs were differentiated into (6a) chondrogenic, (6b) adipogenic, and (6c) osteogenic phenotypes as demonstrated by collagen type II, oil red O, and alizarin red staining. (6d) Fluorescence activated cell sorting (FACS) analysis demonstrated that hMSCs expressed typical MSC surface markers: CD73, CD90 and CD105; but not hematopoietic markers: CD45, CD34, CD11b, CD79A, and HLA-DR. (6e) Bright field images of encapsulated hMSCs immediately following sodium alginate encapsulation showed capsule diameters of 170±27 μm. (6f) Fluorescent viability assay showed 96±2.4% of cells were viable immediately following encapsulation in sodium alginate. Scale bars=100 μm.

FIGS. 7a-7d illustrate an encapsulated hMSC in vitro viability and in vivo tracking following intra-articular injection into naïve joints. (7a) hMSCs in alginate capsules, remained 70-80% viable at early time points (days 1-7), with viability slowly declining to approximately 30% at day 28 and 35. (7b) Representative maximum projection images of capsules from key time points qualitatively demonstrated encapsulated hMSC viability over time in vitro. (7c) In vivo bioluminescent imaging demonstrated an overall increase in quantified bioluminescence of encapsulated hMSCs versus nonencapsulated hMSCs. Initial time points showed similar bioluminescent signals, while later time points (day 5 and 7) demonstrated differences in bioluminescent signal. Complete clearance of nonencapsulated hMSCs (<1%) was observed at day 7 and complete clearance of encapsulated hMSCs was observed at day 9. (7d) Representative images, from key study time points, of the rat knee joint qualitatively illustrate bioluminescent signal for each of the study groups. Relative luminescence units (RLU) with a color heat map according to the scale bar. Data presented as mean±SD. n=5/group for in vivo study. p<0.05. Scale bars=50 μm.

FIGS. 8a-8f illustrate representative histological images of full joint histology of rat hind limb knee joints at 3 days and 9 days post injection of encapsulated hMSCs. (8a, 8b) At day 3, Saf-O-stained joints showed the presence of sodium alginate capsules in the infrapatellar fat pad. (8c) H&E staining demonstrated the presence of encapsulated cells in the sodium alginate capsules at this time point. (8d, 8e) Saf-O staining at day 9 displayed intact sodium alginate capsules in the synovial lining of the knee joint space surrounded by remnants of broken-down capsules. (8f) No encapsulated cells were identifiable at day 9, as demonstrated by H&E staining, but lacunae were identifiable appearing either empty or containing cell debris. In each image, the anterior hindlimb is located on the right and the posterior hindlimb on the left. Scale bars for 8a, 8d=1 mm; 8b, 8e=200 μm; 8e, 8f=60 μm.

FIGS. 9a-9o illustrate representative histology and microCT coronal images of medial tibial condyle following encapsulated hMSC treatment in developing OA. (9a-9e) H&E and (9f-9j) Saf-O stained MMT joints showed no cartilage damage or osteophyte development in (9a, 9f) sham control; (9b-9e, 9g-9j) proteoglycan loss, degeneration of cartilaginous surfaces, and osteophyte development in all MMT groups. (9k-9o) Corresponding microCT images showed similar disease progression as shown by histology. (9k) No cartilage damage was observed in sham control; (91-90) increased areas of cartilage attenuation, specifically in the medial 1/3, was observed in all MMT groups in addition to osteophyte volumes. Darker shading may indicate higher cartilage attenuation, corresponding to lower proteoglycan content. In all images, the medial tibial condyle is located on the left and the lateral tibial condyle on the right. Scale bar (bottom right corner) is universal for all histology and microCT representative images.

FIGS. 10a-10c illustrate microCT quantification of articular cartilage structure and composition in the medial 1/3 of the medial articular cartilage in MMT joints treated with encapsulated hMSCs. (10a) Cartilage attenuation was significantly increased in all MMT groups as compared to sham control. (10b) Cartilage thickness was significantly increased in all MMT groups as compared to sham control; MMT/Encap hMSC reduced increases in cartilage thickness as compared to all other MMT groups. (10c) Cartilage surface roughness was significantly increased in all MMT groups as compared to sham control; MMT/Saline potentiated cartilage surface roughness as compared to all other MMT groups; MMT/Encap hMSC reduced increases in cartilage surface roughness as compared to all other MMT groups. Data presented as mean±SD. n=7/group for MMT/Empty Caps; n=8/group for all other groups. * represents significant differences (p<0.05) between individual MMT groups and sham. Horizontal black bars indicate significance (p<0.05) between individual MMT groups.

FIGS. 11a-11c illustrate microCT quantification of osteophyte formation and subchondral bone morphology of medial side of MMT joints treated with encapsulated hMSCs. (11a) Cartilaginous osteophyte volume was significantly increased in all MMT groups as compared to sham control; MMT Encap hMSC potentiated osteophyte volumes as compared to MMT/Saline and MMT/Empty Caps. (11b) Mineralized osteophyte volume was significantly increased in all MMT groups as compared to sham control; MMT/Encap hMSC potentiated mineralized osteophyte volumes as compared to all other MMT groups. (11c) Subchondral bone thickness was significantly increased in all MMT groups as compared to sham control. Data presented as mean+SD. n=7/group MMT/empty caps; n=8/group for all other groups. * represents significant differences (p<0.05) between individual MMT groups and sham. Horizontal black bars indicate significance (p<0.05) between individual MMT groups. FIGS. 12a-12o illustrate representative histology and microCT coronal images of medial tibial condyle following encapsulated hMSC treatment in established OA. Serial H&E (12a-12e) and Saf-O (12f-12j) coronal sections of rat medial tibial plateaus at 6 weeks after sham or MMT operation of rat hindlimbs. (12b-12e, 12g-12j) For MMT-induced OA there is presence of increased articular cartilage degeneration [increased proteoglycan loss (loss of dark gray coloration in Saf-O images), loss of articular chondrocytes (lack of hematoxylin stain), surface fibrillations, formation of erosions and lesions], and the presence of osteophyte formations on the marginal edges for all MMT groups. sham operated hindlimbs (12a, 12f) did not show any damage to articular cartilage or the presence of osteophyte formations. The MMT/Encap hMSC group (12e, 12j) showed less overall cartilage damage (smoother cartilage surface with less erosion and lesion formation) with respect to all other MMT groups. (12k-12o) microCT representative images were matched with representative histology. All images are oriented with the medial aspect of the tibia on the left. Scale bar (bottom right corner) is universal for all histology representative images. Scale bar for the microCT images (bottom center) is also included.

FIGS. 13a-13l illustrate microCT quantification of articular cartilage structure and composition in the total and medial 1/3 of the medial articular cartilage in 6-week MMT joints treated with encapsulated hMSCs. (13a) Total articular cartilage volume for all MMT groups was significantly greater than sham animals; total articular cartilage volume for MMT/Encap hMSC group is significantly lower than MMT/Saline and MMT/Empty Cap groups. (13b) Medial 1/3 articular cartilage volumes for MMT/Saline, MMT/Empty Caps, and MMT/hMSC groups were significantly greater than sham; hMSC/Encap hMSC group was not significantly different from sham. (13c, 13d) Total and medial 1/3 articular cartilage thickness values for MMT/Saline, MMT/Empty Caps, and MMT/hMSC groups were significantly greater than sham; no differences were found for either parameter between MMT/Encap hMSC and sham; medial 1/3 articular cartilage thickness for MMT/Encap hMSC group was significantly lower than MMT/Saline and MMT/Empty Caps groups. (13e) Total articular cartilage attenuation for the MMT/hMSC group was significantly greater than the sham group. (13f) Medial 1/3 cartilage attenuation values for all MMT groups were significantly greater than the sham group and no differences were found among MMT groups. (13g) Total cartilage surface roughness showed significantly higher values for all MMT groups relative to sham; the MMT/Encap hMSC group did show significantly less surface roughness than all other MMT groups. (13h) Medial 1/3 analysis of surface roughness yielded identical findings to total analysis except no difference was found between sham and MMT/Encap hMSC groups. (13i, 13j) A difference in total exposed bone was found only for the MMT/hMSC group compared to all other groups; for medial 1/3 analysis of exposed bone, MMT/Saline and MMT/hMSC groups were significantly increased from sham group. (13k) PLSDA assessment of the overall effect of the therapeutics applied on articular cartilage damage showed distinct separation with sham and MMT Encap hMSC separating to the left and all other MMT groups separating to the right along LV1. (13I) Quantification of the scores obtained from PLSDA analysis demonstrated that all MMT groups had significantly more cartilage damage than sham; in addition, MMT/Encap hMSC had significantly less damage than all other MMT groups. Data presented as mean+/−SD. n=6 for sham, n=7 for MMT/Saline, n=7 for MMT/Empty Caps, n=8 for MMT/hMSC and n=7 for MMT/Encap hMSC. * represents significant differences (p<0.05) between individual MMT groups and sham. Horizontal black bars indicate significance (p<0.05) between individual MMT groups.

FIGS. 14a-14e illustrate microCT quantification of osteophyte volumes of the medial side of 6-week MMT joints treated with encapsulated hMSCs. (14a) Mineralized osteophyte volumes for all MMT groups were significantly greater than the sham group; MMT/Encap hMSC and MMT/hMSC groups yielded significant increases in mineralized osteophyte volume compared to MMT/Saline and MMT/Empty Caps groups. (14b) Cartilaginous osteophyte volumes for all MMT groups were significantly greater than the sham group; MMT/Encap hMSC demonstrated significant increases in cartilaginous volume relative to MMT/hMSC and MMT/Empty Caps. (14c) Total osteophyte volumes (mineralized+cartilaginous) for all MMT groups were again significantly greater than the sham group; MMT/Encap hMSC yielded significantly greater total osteophyte volumes than all MMT groups. (14d) PLSDA analysis of overall osteophyte volumes depicted distinct separation of all groups based on osteophyte size, with sham to the left and MMT/Encap hMSC to the right along LV1. (14e) Statistical analysis of LV1 scores demonstrated significantly higher values for all MMT groups, compared to sham, with MMT/Encap hMSC showing increased volumes relative to all MMT groups. Data presented as mean+/−SD. n=6 for sham, n=7 for MMT/Saline, n=7 for MMT/Empty Caps, n=8 for MMT/hMSC and n=7 for MMT/Encap hMSC. * represents significant differences (p<0.05) between individual MMT groups and sham. Horizontal black bars indicate significance (p<0.05) between individual MMT groups.

FIGS. 15a-15h illustrate microCT quantification of subchondral bone morphology of the medial side of 6-week MMT joints treated with encapsulated hMSCs. (15a, 15b) Total and medial 1/3 subchondral bone volumes for all MMT groups, except MMT/Encap hMSC, were significantly greater than the sham group. (15c, 15d) Total and medial 1/3 subchondral bone thickness analysis yielded significant increases in all MMT groups relative to sham. (15e) MMT/Empty Caps and MMT/Saline total subchondral bone attenuation was significantly greater than the sham while showing no differences with MMT/hMSC and MMT/Encap hMSC groups. (15f) In the medial 1/3 region, all MMT groups had significantly greater attenuation values compared to shams; the only difference found between MMT groups was between MMT/Empty Caps and MMT/Encap hMSC. (15g) PLSDA analysis of total and medial 1/3 subchondral bone parameters depicted significant separation between sham, to the left, from all MMT groups to the right, along LV1 based on the level of subchondral bone remodeling. (15h) Statistical analysis of these scores demonstrated a significant difference between the sham group and all the MMT groups, with no differences between the respective MMT groups. Data presented as mean+/−SD. n=6 for sham, n=7 for MMT/Saline, n=7 for MMT/Empty Caps, n=8 for MMT/hMSC and n=7 for MMT/Encap hMSC. * represents significant differences (p<0.05) between individual MMT groups and sham. Horizontal black bars indicate significance (p<0.05) between individual MMT groups.

FIGS. 16a-16f illustrate a GEKO histomorphometry analysis of 6-week MMT samples with encapsulated hMSC treatment. (16a) For total cartilage degeneration width all MMT groups yielded an increase degeneration width relative to sham; MMT/Encap demonstrated less degeneration width than both MMT/Saline and MMT/Empty Caps. (16b) Surface cartilage matrix loss width yielded similar outcomes to total cartilage degeneration, with exception to no difference being yielded between MMT/Saline and MMT/Encap hMSC. (16c) For middle depth cartilage matrix loss width all MMT groups, except MMT/Encap hMSC demonstrated increased loss width relative to sham. (16d) Deep cartilage matrix loss width showed no significant differences between any of the groups assessed. (16e) For osteophyte area, all MMT groups showed significantly higher volumes than the sham control; however, no differences were noted between respective MMT groups. (16f) Growth plate thickness showed no significant differences between any groups assessed. Data presented as mean+/−SD. n=3 for all groups. * represents significant differences (p<0.05) between individual MMT groups and sham. Horizontal black bars indicate significance (p<0.05) between individual MMT groups.

FIGS. 17a-17d illustrate quantification of paracrine signaling profiles of encapsulated and nonencapsulated hMSCs. (17a) Multiplexed immunoassay analysis of 41 cytokines (columns; z-scored) secreted from hMSCs with and without encapsulation in unconditioned (+CTRL) and conditioned environments (+IL-1β; each row represents a single sample). +IL-1β conditioning demonstrated increased paracrine cytokine secretion relative to +CTRL conditioning. Encapsulation, with +IL-1β conditioning, yielded a more targeted cytokine profile relative to nonencapsulated hMSCs. (17b) PLSDA analysis identified two profiles of cytokines, LV1 and LV2, that identified a distinct separation between treatment groups for both encapsulated and nonencapsulated hMSCs. (17c, 17d) Independent analysis of scores on each of the respective latent variables demonstrated significant differences between unconditioned and conditioned environments for both encapsulated and nonencapsulated hMSCs. Data presented as mean+/−SD. n=6 for all groups. Horizontal black bars indicate significant differences between unconditioned and conditioned groups.

FIGS. 18a-18j illustrate identification of cytokines contributing to therapeutic efficacy of encapsulated hMSC therapeutics. (18a) PLSDA analysis of encapsulated hMSCs identified a single latent variable, LV1, that distinguished between Encap hMSC+CTRL on the left and Encap hMSC+IL-1β to the right. (18b) The weighted profiles of cytokines showed relative expression of cytokines in CTRL conditions (left) and IL-1β conditions (right). Error bars on each cytokine were computed by PLSDA model regeneration using iterative (1000 iterations) LOOCV. (18c-18j) All measured cytokines that showed significant increased expression with IL-1β conditioning were assessed independently, using t-test with Bonferroni correction, for significance between CTRL and IL-1β conditions, with all significant findings presented. Encap hMSCs+IL-1β yielded increased expression in pro-inflammatory (IL-1β, IL-6, IL-7, IL-8), anti-inflammatory (IL-1RA), and chemotactic (G-CSF, MDC, IP10) cytokines. Data presented as mean+/−SD. n=6 for all groups. Horizontal black bars indicate significant differences between unconditioned (+CTRL) and conditioned (+IL-1β) groups.

FIG. 19 illustrates an overview of statistical approaches used herein. Statistical approaches were implemented to identify cellular attributes of therapeutic hMSCs. More specifically, correlation analysis, PLSR, and PCA were used to relate cytokine secretion, RNA transcripts, and intracellular signaling phospho-proteins to therapeutic outcomes in the MMT model of OA.

FIGS. 20a-20j illustrate the effect of hMSC donor heterogeneity on articular cartilage therapeutic outcomes in the total and medial 1/3 of the medial articular cartilage in 6-week MMT joints. (20a) For total articular cartilage volume, all MMT groups except MMT/hMSC (2.3) demonstrated significant increases relative to sham; between MMT groups both MMT/hMSC(2.3) and MMT/hMSC(2.4) demonstrated reduced increases in volume. (20b) In the medial 1/3 region MMT/Saline, MMT/hMSC(2.1), and MMT/hMSC(2.2) showed significant increases in articular cartilage volume; all MMT/hMSC donor groups yielded reduced articular cartilage volume in the medial 1/3 region. (20c) For articular cartilage thickness, all MMT groups showed significant increases in thickness relative to sham, however no differences were noted between respective MMT groups. (20d) The medial 1/3 region showed similar outcomes to total thickness assessment except both the MMT/hMSC (2.3) and MMT hMSC(2.4) groups showed reduced thickness increases. (20e, 20f) For articular cartilage attenuation, both total and medial 1/3 analyses demonstrated increased attenuation for all MMT groups relative to sham; no notable differences between MMT groups were found. (20g) For articular cartilage surface roughness all MMT groups showed significantly higher values than sham control for the total tibia; MMT/hMSC(2.2) showed potentiated surface roughness increases and MMT/hMSC(2.3) showed reduced surface roughness increases; furthermore, both MMT/hMSC(2.3) and MMT/hMSC(2.4) groups showed reduced articular cartilage surface roughness relative to the other two MMT/hMSC groups. (20h) In the medial 1/3 region again all MMT groups showed increased surface roughness relative to the sham; both MMT/hMSC(2.3) and MMT/hMSC(2.4) groups showed reduced articular cartilage surface roughness relative to the other two MMT/hMSC groups. (20i) For articular cartilage lesion volume of the total tibia all MMT groups, except MMT/hMSC(2.3) and MMT/hMSC(2.4) groups, demonstrated significantly higher lesion volumes; the MMT/hMSC(2.3) and MMT/hMSC(2.4) groups also demonstrated significantly less lesion volumes than the MMT/Saline and MMT/hMSC(2.2) group. (20j) No significant differences were found between any groups for exposed bone of the total region. Data presented as mean+/−SD. n=7 for MMT/hMSC(2.3) and n=8 for all other groups. * represents significant differences (p<0.05) between individual MMT groups and sham. Horizontal black bars indicate significance (p<0.05) between individual MMT groups.

FIGS. 21a-21h illustrate the effect of hMSC donor heterogeneity on osteophyte and subchondral bone therapeutic outcomes in the total and medial 1/3 of the medial articular cartilage in 6-week MMT joints. (21a) Mineralized osteophyte volumes were significantly increased in all MMT groups relative to sham; MMT/hMSC(2.1) showed significantly greater osteophyte volumes than MMT/Saline; MMT/hMSC(2.2), MMT/hMSC(2.3), and MMT/hMSC(2.4) showed reduced osteophyte volumes relative to MMT/Saline and MMT/hMSC(2.1). (21b) For cartilaginous osteophyte volumes, all MMT groups showed significantly greater volumes than sham; no differences were noted between MMT groups. (21c) For total subchondral bone volume MMT/Saline, MMT/hMSC(2.2), and MMT/hMSC (2.4) groups showed significantly higher volume than sham; all MMT/hMSC donor groups showed significantly reduced volume relative to sham. (21d) In the medial 1/3 region the MMT/Saline group was the only group to show significantly reduced volume relative to sham; all hMSC groups except MMT/hMSC(2.2) showed significantly reduced volume relative to MMT/Saline. (21e) For subchondral bone thickness the MMT/Saline, MMT/hMSC(2.2), and MMT/hMSC(2.4) showed significant increases in thickness relative to sham; MMT/hMSC(2.1) and MMT/hMSC(2.3) groups showed reduced thickness relative to MMT/Saline. (21f) In the medial 1/3 region results were similar to the total region analysis except no significant difference was noted between MMT/Saline and MMT/hMSC(2.3). (21g) For subchondral bone attenuation of the total tibial plateau no significant differences were noted. (21h) In the medial 1.3 region, the MMT/Saline, MMT/hMSC(2.2), and MMT/hMSC(2.4) showed significantly higher attenuation values than sham. Data presented as mean+/−SD. n=7 for MMT/hMSC(2.3) and n=8 for all other groups. * represents significant differences (p<0.05) between individual MMT groups and sham. Horizontal black bars indicate significance (p<0.05) between individual MMT groups.

FIG. 22 illustrates characterization of SF collected from patients with and without clinically diagnosed OA. OA SF samples yielded increase overall cytokine content relative to non-OA (SF CTRL) SF samples. Among donors, for either OA SF or SF CTRL, there were distinct differences between human donors. The pooled samples for both CTRL SF and OA SF, samples were combined in equal parts.

FIG. 23 illustrates the effect of hMSC donor heterogeneity on hMSC quantification of paracrine signaling profiles. Multiplexed immunoassay analysis of 44 cytokines (columns; z-scored) secreted from hMSCs in unconditioned media (+CTRL), IL-1β conditioned media (+IL-1β), SF (collected from patients without an OA clinical diagnosis) conditioned media (+SF CTRL), and OA SF (collected from patients with clinically diagnosed OA) conditioned media (+OA SF; each row represents a single sample) for four unique hMSC donors. +IL-1β conditioning demonstrated increased paracrine cytokine secretion relative to all other conditions with donors L1 and L2 demonstrating potentiated cytokine secretion levels relative to donors M1 and M2.

FIG. 24 illustrates the paracrine signaling response of hMSCs with variable OA therapeutic efficacy under different in vitro conditioning strategies. PLSDA analysis identified two profiles of cytokines, LV1 and LV2, that identified a distinct separation between less therapeutic (Donors L1+CTRL, L2+CTRL, L1+IL1B, L2+IL1B, L1+SF CTRL, L2+SF CTRL, L1+OA SF, L2+OA SF) and more therapeutic hMSCs (Donors M1+CTRL, M2+CTRL, M1+IL1B, M2+IL1B, M1+SF CTRL, M2+SF CTRL, M1+OA SF, M2+OA SF), as determined by microCT, on LV2. Furthermore, on LV1 there is clear separation between hMSCs conditioned with IL-1β and hMSCs conditioned with media only (CTRL), SF CTRL, or OA SF. In this redefined plane, two distinct clusters formed with IL-1β (within larger circle on left) clustering to the left and hMSCs conditioned with media only (CTRL), SF CTRL, or OA SF (within smaller circle on right) clustering to the right. Variability accounted for in each LV is included on respective axes labels.

FIG. 25 illustrates identification of hMSC cytokines that correlate with hMSC therapeutic efficacy in vivo, according to example implementations of the disclosed technology. Pearson's correlation coefficients for in vivo MMT therapeutic data and in vitro multiplexed immunoassay cytokine analysis (+IL-1β). The cytokines GM-CSF, GRO, IL-4, PDGF-AA, and TGFβ3 showed significant inverse correlations with microCT therapeutic outcomes (increased cytokine secretion led to therapeutic outcomes; negative correlation coefficients represented by dark grey circles). Black X's indicate coefficients that are not significant (significance level set at p<0.05).

FIGS. 26a-26c illustrate paracrine signaling response of hMSCs in an IL-1β OA simulated microenvironment. (26a) Multiplexed immunoassay analysis of 44 cytokines (columns; z-scored) secreted from hMSCs in IL-1β conditioned media. donors L1 and L2 demonstrated potentiated overall cytokine secretion levels relative to donors M1 and M2 which yielded a more targeted paracrine profile. (26b) PLSR analysis (input: in vitro cytokine data; output: microCT data) identified a profile of cytokines along LV1 that identified a distinct separation between less therapeutic (left side of figure) and more therapeutic hMSCs (right side of figure), as determined by microCT. (26c) Loadings plot demonstrating relative contribution of cytokines to PLSR scores obtained show that the cytokines GM-CSF, GRO, IL-4, PDGF-AA, and TGF-β3 contribute to separating out more therapeutic hMSC donors while all other cytokines assessed contribute more to less therapeutic hMSCs. Variability accounted for in each LV is included on respective axes labels.

FIGS. 27a-27b illustrate gene expression profiles of more and less therapeutic hMSCs following IL-1β conditioning in OA simulated microenvironment. (27a) Quantification of 24,475 genes using RNA-Seq yielded two clusters with cluster 1 demonstrating unique gene expression of less therapeutic hMSCs and cluster 2 demonstrating the gene expression of more therapeutic hMSCs. (27b) These unique gene expression profiles were further demonstrated quantitatively using PCA which clearly showed that less therapeutic hMSCs (to the left) and more therapeutic hMSCs (to the right) separate along the PC1 axis.

FIG. 28 illustrates GSVA gene expression pathway profiles of more and less therapeutic hMSCs. Quantification of 6,287 gene expression pathways screened using GSVA yielded three distinct clusters with cluster 1 demonstrating unique gene expression of more therapeutic hMSCs, cluster 2 categorizing the gene expression pathways which had no relation to therapy as they were conserved between more and less therapeutic hMSCs, and cluster 3 demonstrating the gene expression of less therapeutic hMSCs.

FIGS. 29a-29c illustrate differences in gene expression pathway enrichment scores between more therapeutic and less therapeutic hMSCs. (29a) More therapeutic hMSCs demonstrated significant increased enrichment of GM-CSF and GCSF, IL-4, and TGFβ pathway signaling. (29b) No significant differences were identified in assessing differences in enrichment scores between more therapeutic and less therapeutic hMSCs for GAG and proteoglycan OA associated pathways. (29c) Less therapeutic hMSCs yielded increased enrichment of the Akt and NF-κB signaling pathways while more therapeutic hMSCs yielded increased enrichment of the MAPK signaling pathway. Horizontal dark grey bars indicate significance (p<0.25) between more and less therapeutic hMSCs enrichment scores.

FIG. 30 illustrates identification of hMSC MAPK and Akt phospho-proteins that correlate with hMSC therapeutic efficacy in vivo, according to example implementations of the disclosed technology. Pearson's correlation coefficients for in vivo MMT therapeutic data and in vitro intracellular signaling analysis (+IL-1β). The phospho-proteins p-Atf-2 and p-JNK from the MAPK signaling cascade and p-mTOR and p-PTEN from the Akt signaling cascade showed significant inverse correlations with microCT therapeutic outcomes (increased phospho-protein expression led to therapeutic outcomes; negative correlation coefficients represented by dark gray circles). The phospho-protein p-Akt from the Akt signaling cascade showed a significant correlation with microCT therapeutic outcomes (decreased phospho-protein expression led to therapeutic outcomes; positive correlation coefficients represented by dark gray circles). Correlations that demonstrated >50% significant correlations with microCT therapeutic outcome metrics were selected as several other phospho-proteins showed single and multiple respective correlations with individual microCT therapeutic outcome metrics. Black X's indicate coefficients that are not significant (significance level set at p<0.05).

FIGS. 31a-31f illustrate a temporal analysis of target phospho-protein signals in the MAPK and Akt signaling pathways. (31a-31c) p-JNK, p-p 38, and p-Atf-2 phospho-protein signaling in the MAPK pathway yield signaling spikes in 15 mins, relative to the 5 and 60 min time points. (31d-31f) Furthermore, p-Akt, p-mTOR, and P-IGF1R in the Akt pathway also yield spikes in signaling at 15 mins, relative to the 5 and 60 min time points. Time demonstrated a significant effect (p<0.0001) for all MAPK and Akt phospho-protein signaling assessed.

FIGS. 32a-32b illustrate MAPK phospho-protein signaling levels in more therapeutic (donors M1 and M2) and less therapeutic (donors L1 and L2) hMSCs. (32a) Signaling schematic of MAPK pathway (ERK pathway is not included) in hMSCs with inhibitor sites highlighted for the p-JNK and p-p38 phospho-protein signaling levels. (32b) Quantification of MAPK phospho-protein signaling levels in all cell lines demonstrated increased overall phospho-protein signaling levels in the more therapeutic hMSCs relative to the less therapeutic hMSCs; more specifically, more therapeutic hMSCs demonstrated increased p-JNK and p-p38 phospho-protein signaling levels relative to less therapeutic hMSCs.

FIGS. 33a-33b illustrate paracrine signaling profiles of more therapeutic hMSCs (donors M1 and M2) treated with p-p38 (SB203580), p-JNK inhibitor (SP600125), and inhibitors in combination (p-p38+p-JNK). (33a) Quantification of immunomodulatory cytokines for more therapeutic hMSCs, with different inhibitor conditions applied, show distinct shifts in overall cytokine signaling with addition of single and combinatorial inhibitor conditions. More specifically, treatment of more therapeutic hMSCs with all inhibitor conditions yielded decreased secretion of GM-CSF, GRO, IL-4, and PDGF-AA. (33b) PLSDA analysis identified a profile of cytokines along LV1 that identified a distinct separation between less therapeutic hMSCs to the left (Donors M1 CTRL and M2 CTRL) and more therapeutic hMSCs treated with inhibitors to the right (Donors M1+Jnkl, M1+p381, M1+JNKI+p381, M2+Jnkl, M2+p381, M2+JNKI+p381), relative to more therapeutic hMSCs (Donors L1 CTRL and L2 CTRL). Variability accounted for in each LV is included on respective axes labels.

FIGS. 34a-34b illustrate Akt phospho-protein signaling levels in more therapeutic (donors M1 and M2) and less therapeutic (donors L1 and L2) hMSCs. (34a) Signaling schematic of Akt pathway in hMSCs with inhibitor (stops) and activator (arrows) sites highlighted for the p-IGF1R, p-Akt, and p-mTOR phospho-proteins. (34b) Quantification of Akt phospho-protein signaling levels in all cell lines demonstrated increased signaling levels for all phospho-proteins, except Akt, in the more therapeutic hMSCs relative to the less therapeutic hMSCs; more specifically, more therapeutic hMSCs demonstrated increased p-IGF1R and p-mTOR phospho-protein signaling levels relative to less therapeutic hMSCs and less therapeutic hMSCs demonstrated increased p-Akt levels relative to more therapeutic hMSCs.

FIG. 35 illustrates paracrine signaling profiles of more therapeutic hMSCs (donors M1 and M2) treated with an Akt activator (SC79), p-IGF1R inhibitor (BMS-536924), p-mTOR inhibitor (Rapamycin). Quantification of immunomodulatory cytokines for more therapeutic hMSCs, with different inhibitor conditions applied, show potentiated overall cytokine signaling with addition of an Akt activator and mTOR inhibitor. More specifically, treatment of more therapeutic hMSCs with an Akt activator potentiated overall cytokine signaling further than treatment with an mTOR or IGF1R inhibitor.

FIG. 36 illustrates paracrine signaling profiles of less therapeutic hMSCs (donors L1 and L2) treated with an Akt inhibitor (MK-2206). Quantification of immunomodulatory cytokines for more therapeutic hMSCs, with different inhibitor conditions applied, show potentiated overall cytokine signaling with addition of all inhibitors implemented. More specifically, treatment of more therapeutic hMSCs with an Akt activator potentiated overall cytokine signaling further than treatment with an mTOR or IGF1R inhibitor.

FIGS. 37a-37b illustrate p-JNK and p-Akt phospho-protein signaling levels in more therapeutic hMSC donor M2 treated with combination therapy of SP600125 (p-JNK inhibitor) and SC79 (p-Akt activator). (37a) p-JNK phospho-protein signaling in the MAPK pathway for hMSCs (donor M2) treated with SP600125 yielded a significant (p<0.05) decrease in overall p-JNK phospho-protein levels, relative to CTRL; significant decreases in p-JNK, with SP600125 treatment, were identified at 15 mins and 24 hours post conditioning. (37b) p-Akt phospho-protein signaling in the Akt pathway for hMSCs (donor M2) treated with SC79 yielded a significant (p<0.001) increase in overall p-Akt phospho-protein levels, relative to CTRL; significant increases in p-Akt, with SC79 treatment, were identified at 60 mins and 24 hours post conditioning. * represents significant differences (p<0.05) between inhibitor treated and CTRL groups at individual time points.

FIGS. 38a-38b illustrate p-JNK and p-Akt phospho-protein signaling levels in less therapeutic hMSC donor L2 treated with combination therapy of Metformin (p-JNK activator) and MK-2206 (p-Akt inhibitor). (38a) p-JNK phospho-protein signaling in the MAPK pathway for hMSCs (donor L2) treated with Metformin yielded a significant (p<0.01) increase in overall p-JNK phospho-protein levels, relative to CTRL; significant increases in p-JNK, with Metformin treatment, were identified at 15 and 60 mins post conditioning. (38b) p-Akt phospho-protein signaling in the Akt pathway for hMSCs (donor L2) treated with MK-2206 yielded a significant (p<0.01) decrease in overall p-Akt phospho-protein levels, relative to CTRL; significant decreases in p-Akt, with MK-2206 treatment, were identified at 15 and 60 mins post conditioning. * represents significant differences (p<0.05) between inhibitor treated and CTRL groups at individual time points.

FIGS. 39a-39c illustrate paracrine signaling profiles of more therapeutic hMSCs (donors M1 and M2) treated with combination therapy of an Akt activator (SC79) and JNK inhibitor (SP600125). (39a) Quantification of immunomodulatory cytokines for more therapeutic hMSC donor M1, with combination therapy applied, showed a less targeted (increase in overall cytokine secretion) paracrine profile relative to the donor M1 CTRL. More specifically, hMSCs (donor M1) treated with the Akt activator and JNK inhibitor showed reduced cytokine secretion of GM-CSF, GRO, IL-4, and PDGF-AA relative to the donor M1 CTRL. (39b) hMSC donor M2 yielded similar outcomes to donor M1 with less targeted cytokine secretion and reduced secretion of GM-CSF, GRO, IL-4, and PDGF-AA with treatment of an Akt activator and JNK inhibitor. (39c) PLSDA analysis identified a profile of cytokines along LV1 that identified a distinct separation between more therapeutic hMSCs to the left and less therapeutic hMSCs to the right. Addition of the combination therapy shifted the more therapeutic hMSCs towards a less therapeutic paracrine profile (rightward shift on LV1; Donors M1+JNKI+AktA, M2+JNKI+AktA). Furthermore, differences in donor paracrine signaling were maintained with interventions as indicated by separation along LV2. Variability accounted for in each LV is included on respective axes labels.

FIGS. 40a-40c illustrate paracrine signaling profiles of less therapeutic hMSCs (donors L1 and L2) treated with combination therapy of an Akt inhibitor (MK-2206) and JNK activator (Metformin). (40a) Quantification of immunomodulatory cytokines for less therapeutic hMSC donor L1, with combination therapy applied, showed a more targeted (decrease in overall cytokine secretion) paracrine profile relative to the donor L1 CTRL. More specifically, hMSCs (donor L1) treated with the Akt inhibitor and JNK activator showed potentiated cytokine secretion of GM-CSF, GRO, IL-4, and PDGF-AA relative to the donor L1 CTRL. (40b) hMSC donor L2 yielded similar outcomes to donor L1 with targeted cytokine secretion and reduced secretion of GM-CSF, GRO, and IL-4 (but not PDGF-AA) with treatment of an Akt inhibitor and JNK activator. (40c) PLSDA analysis identified a profile of cytokines along LV1 that identified a distinct separation between less therapeutic hMSCs to the left and more therapeutic hMSCs to the right. Addition of the combination therapy shifted the less therapeutic hMSCs towards a more therapeutic paracrine profile (rightward shift on LV1; Donors L1+JNKA+Akt1 and L2+JNKA+Akt1). Furthermore, differences in donor paracrine signaling were maintained with interventions as indicated by separation along LV2. Variability accounted for in each LV is included on respective axes labels.

FIGS. 41a-41p illustrate the effect of hMSC donor heterogeneity on articular cartilage therapeutic outcomes in the total and medial 1/3 of the medial articular cartilage in 6-week MMT joints. (41a) For total articular cartilage volume, all MMT groups except MMT/hMSC (2.3) demonstrated significant increases relative to sham; between MMT groups both MMT/hMSC(2.3) and MMT/hMSC(2.4) demonstrated reduced increases in volume. (41b) In the medial 1/3 region MMT/Saline, MMT/hMSC(2.1), and MMT/hMSC(2.2) showed significant increases in articular cartilage volume; all MMT/hMSC donor groups yielded reduced articular cartilage volume in the medial 1/3 region. (41c) For articular cartilage thickness, all MMT groups showed significant increases in thickness relative to sham, however no differences were noted between respective MMT groups. (41d) The medial 1/3 region showed similar outcomes to total thickness assessment except both the MMT/hMSC (2.3) and MMT hMSC(2.4) groups showed reduced thickness increases. (41e, 41f) For articular cartilage attenuation, both total and medial 1/3 analyses demonstrated increased attenuation for all MMT groups relative to sham; no notable differences between MMT groups were found. (41g) For articular cartilage surface roughness all MMT groups showed significantly higher values than sham control for the total tibia; MMT/hMSC(2.2) showed potentiated surface roughness increases and MMT/hMSC(2.3) showed reduced surface roughness increases; furthermore, both MMT/hMSC(2.3) and MMT/hMSC(2.4) groups showed reduced articular cartilage surface roughness relative to the other two MMT/hMSC groups. (41h) In the medial 1/3 region again all MMT groups showed increased surface roughness relative to the sham; both MMT/hMSC(2.3) and MMT/hMSC(2.4) groups showed reduced articular cartilage surface roughness relative to the other two MMT/hMSC groups. (41i) For articular cartilage lesion volume of the total tibia all MMT groups, except MMT/hMSC(2.3) and MMT/hMSC(2.4) groups, demonstrated significantly higher lesion volumes; the MMT/hMSC(2.3) and MMT/hMSC(2.4) groups also demonstrated significantly less lesion volumes than the MMT/Saline and MMT/hMSC(2.2) group. (41j) No significant differences were found between any groups for exposed bone of the total region. (41k-p) Representative microCT and histological images are provided for all groups assessed. Data presented as mean+/−SD. n=7 for MMT/hMSC(2.3) and n=8 for all other groups. * represents significant differences (p<0.05) between individual MMT groups and sham. Horizontal black bars indicate significance (p<0.05) between individual MMT groups.

FIG. 42a-42h illustrate the effect of hMSC donor heterogeneity on osteophyte and subchondral bone therapeutic outcomes in the total and medial 1/3 of the medial articular cartilage in 6-week MMT joints.

FIGS. 43a-43g illustrate paracrine signaling response of hMSCs in an IL-1β OA simulated microenvironment. (43a) Multiplexed immunoassay analysis of 41 cytokines (columns; z-scored) secreted from hMSCs in IL-1β conditioned media. Donors L1 and L2 demonstrated potentiated overall cytokine secretion levels relative to donors M1 and M2, which yielded a more targeted paracrine profile. (43b) PLSR analysis (input: in vitro cytokine data; output: microCT data) identified a profile of cytokines along LV1 that identified a distinct separation between less therapeutic and more therapeutic hMSCs, as determined by microCT. Variability accounted for in each LV is included on respective axes labels. (43c) Loadings plot demonstrating relative contribution of cytokines to PLSR scores obtained show that the cytokines GM-CSF, GRO, IL-4, and PDGF-AA contribute to separating out more therapeutic hMSC donors while all other cytokines assessed contribute more to less therapeutic hMSCs.

(43d-43g) GM-CSF, GRO, IL-4, and PDGF-AA were assessed independently and demonstrated significantly higher secretion levels in more therapeutic hMSC donors (M1 and M2) relative to less therapeutic donors (L1 and L2).

FIGS. 44a-44c illustrate unique gene set expression profiles of more therapeutic hMSCs and less therapeutic hMSCs following IL-1β conditioning in OA simulated microenvironment.

FIGS. 45a-45i illustrate paracrine signaling profiles of more therapeutic hMSCs (donors M1 and M2) treated with a p-JNK inhibitor (SP600125).

FIGS. 46a-46f illustrate temporal analysis of target phospho-protein signals in the MAPK and Akt signaling pathways.

FIGS. 47a-47i illustrate paracrine signaling profiles of more therapeutic hMSCs (donors M1 and M2) treated with an Akt activator (SC79).

FIGS. 48a-48d illustrate paracrine signaling profiles of more therapeutic hMSCs (donors M1 and M2) treated with a JNK inhibitor (SP600125) and p-Akt activator (SC79).

FIGS. 49a-49d illustrate paracrine signaling profiles of more therapeutic hMSCs (donors M1 and M2) treated with combination therapy of an Akt activator (SC79) and JNK inhibitor (SP600125).

### DETAILED DESCRIPTION OF THE INVENTION

As specified in the Background Section, there is a great need in the art to identify technologies for characterizing the therapeutic efficacies of MSCs and using this understanding to develop novel methods of distinguishing and classifying MSCs as being highly therapeutic or less therapeutic based on the levels of certain proteins, phospho-proteins, genes, and/or MSC-secreted cytokines, chemokines, and/or growth factors, as well as methods of improving therapeutic efficacies of MSCs by altering the levels of such proteins, phospho-proteins, genes, and/or MSC-secreted cytokines, chemokines, and/or growth factors, and treating a subject with OA using highly therapeutically effective MSCs obtained from these methods. Systems and devices for performing such distinction, classification, and/or optimization of therapeutic efficacy are contemplated herein. The present invention satisfies this and other needs.

Embodiments of the present invention relate generally to methods of improving the therapeutic efficacy of MSCs for use in treating OA, and more specifically to methods of altering levels of certain proteins, phospho-proteins, genes, and/or MSC-secreted cytokines, chemokines, and/or growth factors in order to improve therapeutic efficacy, as well as methods of distinguishing and classifying MSCs as being highly therapeutic or less therapeutic based on levels of certain phospho-proteins and/or MSC-secreted cytokines, chemokines, and/or growth factors and methods of treating a subject having OA with MSCs having improved therapeutic efficacy.

### Definitions

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or examples. The invention is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

As used herein, the term “and/or” may mean “and,” it may mean “or,” it may mean “exclusive-or,” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

As used herein, the term “about” should be construed to refer to both of the numbers specified as the endpoint(s) of any range. Any reference to a range should be considered as providing support for any subset within that range. Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to +20%, preferably up to +10%, more preferably up to +5%, and more preferably still up to #1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present invention as many comparable parameters, sizes, ranges, and/or values may be implemented. The terms “first,” “second,” and the like, “primary,” “secondary,” and the like, do not denote any order, quantity, or importance, but rather are used to distinguish one element from another.

It is noted that terms like “specifically,” “preferably,” “typically,” “generally,” and “often” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention. It is also noted that terms like “substantially” and “about” are utilized herein to represent the inherent degree of uncertainty that may be attributed to any quantitative comparison, value, measurement, or other representation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “50 mm” is intended to mean “about 50 mm.”

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

As used herein, the term “subject” or “patient” refers to mammals and includes, without limitation, humans and animals, e.g., horses, cats, and dogs. In a preferred embodiment, the subject is human.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “therapeutic” as used herein means a treatment and/or prophylaxis. A therapeutic effect is obtained by suppression, diminution, remission, or eradication of a disease state.

As used herein the term “therapeutically effective” applied to a dose or amount refers to that quantity of a compound or pharmaceutical composition that when administered to a subject for treating (e.g., preventing or ameliorating) a state, disorder or condition, is sufficient to effect such treatment. The “therapeutically effective amount” will vary depending on the compound administered as well as the disease and its severity and the age, weight, physical condition and responsiveness of the subject to be treated.

In the context of the field of medicine, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985); Transcription and Translation (B. D. Hames & S. J. Higgins, eds. (1984); Animal Cell Culture (R. I. Freshney, ed. (1986); Immobilized Cells and Enzymes (IRL Press, (1986); B. Perbal, A Practical Guide To Molecular Cloning (1984); F. M. Ausubel et al. (eds.), Current Protocols in Molecular Biology, John Wiley & Sons, Inc. (1994); among others.

### Methods of the Invention

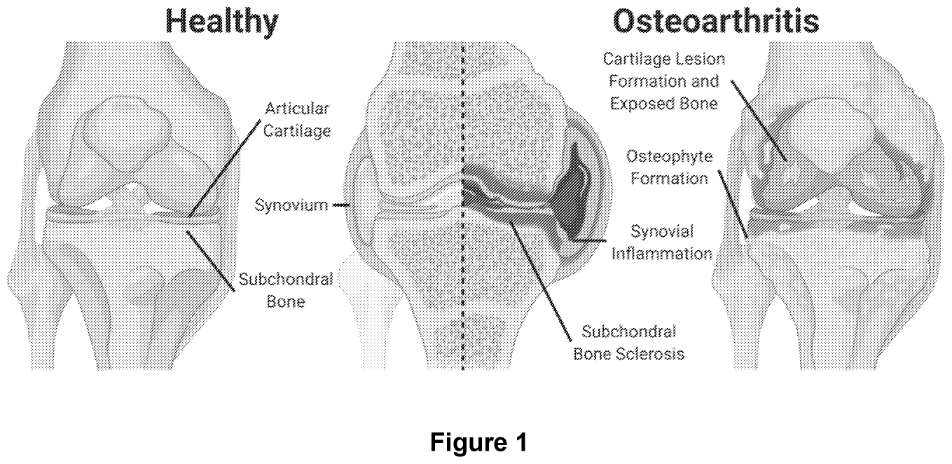

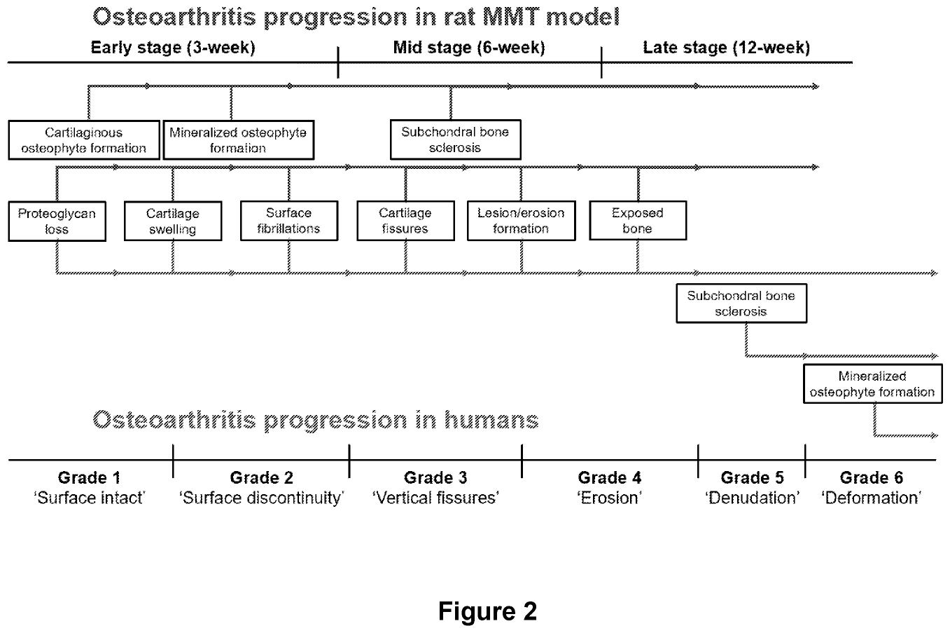

Generally, the invention provides methods for identifying, classifying, and/or distinguishing MSCs that are likely to be highly therapeutic for treating OA (FIGS. 1 and 2), i.e., are able to promote healing, tissue remodeling, recruitment of stem and progenitor cells, and mediation of the immune response, cartilage repair; improve cartilage smoothness; decrease changes in cartilage thickness and/or volume; decrease fibrillation development; reduce development of cartilage lesions; decrease proteoglycan loss; decrease subchondral bone sclerosis; decrease synovitis or synovial hypertrophy; decrease pain; and/or decrease loss of function of the joint or limb. The methods can also identify MSCs that are likely to be less therapeutic for treating OA, i.e., are less able or unable to promote healing, tissue remodeling, recruitment of stem and progenitor cells, mediation of the immune response, cartilage repair, or cartilage smoothness, and can result in increased cartilage swelling, thickness and/or volume, increased fibrillation development, increased development of cartilage lesions, increased proteoglycan loss, increased subchondral bone sclerosis, increased synovitis or synovial hypertrophy, increased pain, and/or increased loss of function of the joint or limb. The invention also provides methods of modifying the less therapeutic MSCs to become more therapeutic, as well as methods of treating OA in a subject by administering highly therapeutic MSCs to a joint of the subject.

The identification, classification, and/or distinction of highly therapeutic MSCs from less therapeutic MSCs can be performed by analyzing secretion of cytokines, chemokines, and/or growth factors, and/or by analyzing levels (e.g., amounts and/or expression levels) of proteins, phospho-proteins, and/or genes in certain kinase signaling cascades related to cell proliferation, growth, and the cell cycle. The cytokine, chemokine, and/or growth factor secretion profile and/or the levels (e.g., amounts and/or expression levels) of the proteins, phospho-proteins, and/or genes are compared to a standard in order to classify them as highly therapeutic or less therapeutic. Non-limiting examples of such standards, usable in any of the methods described herein, can be profiles or levels from a standard control MSC or profiles or levels from a MSC that has previously been identified as highly therapeutic or less therapeutic.

The invention also provides methods of modifying the less therapeutic MSCs to become highly therapeutic. As discussed in more detail herein, the cytokine, chemokine, and/or growth factor secretion profile and/or the levels (e.g., amounts and/or expression levels) of the proteins, phospho-proteins, and/or genes in the signaling pathways of the less therapeutic MSCs can be modified to be more similar to the secretion profile and/or the levels (e.g., amounts and/or expression levels) of the proteins, phospho-proteins, and/or genes in the signaling pathways of the highly therapeutic MSCs. Optionally, highly therapeutic MSCs can undergo a similar modification to increase their therapeutic efficacy.

The invention also provides methods of treating OA using the highly therapeutic MSCs described herein in a joint in a patient in need thereof. Preferably, the joint is osteoarthritic, i.e., has at least early-stage OA or is at risk for the formation of OA, e.g., after a joint injury or a ligament, tendon, or meniscal tear. The MSCs may be administered directly to the joint, for example by intra-articular injection or intra-synovial injection, and/or can be introduced into the joint during a surgical procedure on the joint.

In any of the methods described herein, the cytokines, chemokines, and/or growth factors secreted by the MSCs can be associated with the level of phospho-c-Jun N-terminal kinase (p-JNK) in the MSCs. For example, if the level of phospho-c-Jun N-terminal kinase (p-JNK) is increased compared to a standard, this increased level can be associated with increased levels of the following non-limiting exemplary cytokines, chemokines, and/or growth factors: granulocyte macrophage colony stimulating factor (GM-CSF), chemokine ligand 1 (GRO), interleukin-4 (IL-4), and/or platelet derived growth factor (PDGF)-AA. Increased levels of these cytokines, chemokines, and/or growth factors are secreted from highly therapeutic MSCs, meaning that increased levels of GM-CSF, GRO, IL-4, and/or PDGF-AA can be indicative of, and associated with, highly therapeutic MSCs. Alternatively, if the level of phospho-c-Jun N-terminal kinase (p-JNK) is decreased compared to a standard, this decreased level can be associated with decreased levels of the following non-limiting exemplary cytokines, chemokines, and/or growth factors: GM-CSF, GRO, IL-4, and/or PDGF-AA. Decreased levels of these cytokines, chemokines, and/or growth factors are secreted from less therapeutic MSCs, meaning that decreased levels of GM-CSF, GRO, IL-4, and/or PDGF-AA can be indicative of, and associated with, less therapeutic MSCs.

In any of the methods described herein, the proteomic, phospho-proteomic, or transcription profiles of proteins and/or genes in certain signaling pathways can be indicative of, and associated with, highly therapeutic MSCs or less therapeutic MSCs. Thus, determining the proteomic, phospho-proteomic, or transcription profiles of genes in these pathways can distinguish highly therapeutic MSCs from less therapeutic MSCs. Non-limiting examples of such signaling pathways include the mitogen-activated protein kinase (MAPK) and phosphatidylinositol 3-kinase/protein kinase B (PI3K/Akt) pathways.

For example, increased levels of proteins, phospho-proteins, and/or expression of genes in the MAPK pathway can be associated with, and indicative of, highly therapeutic MSCs. Non-limiting exemplary proteins, phospho-proteins, and/or genes in the MAPK pathway that have increased levels compared to a standard include cJun, JNK, heat shock protein (HSP)-27, p38 MAP kinase, extracellular signal-regulated kinase (ERK), MAPK/ERK kinase (MEK), and/or activating transcription factor (Atf)-2. Increased levels of these proteins, phospho-proteins, and/or genes are found in highly therapeutic MSCs, meaning that increased levels of cJun, JNK, heat shock protein (HSP)-27, p38 MAP kinase, extracellular signal-regulated kinase (ERK), MAPK/ERK kinase (MEK), and/or activating transcription factor (Atf)-2 can be indicative of, and associated with, highly therapeutic MSCs. Alternatively, decreased levels of proteins, phospho-proteins, and/or expression of genes in the MAPK pathway can be associated with, and indicative of, less therapeutic MSCs. Thus, decreased levels or expression levels of cJun, JNK, heat shock protein (HSP)-27, p38 MAP kinase, extracellular signal-regulated kinase (ERK), MAPK/ERK kinase (MEK), and/or activating transcription factor (Atf)-2 relative to a standard can be indicative of, and associated with, less therapeutic MSCs.

For example, increased levels of proteins, phospho-proteins, and/or expression of genes in the PI3K/Akt pathway can be associated with, and indicative of, less therapeutic MSCs. Non-limiting exemplary proteins, phospho-proteins, and/or genes in the PI3K/Akt pathway that have increased levels compared to a standard include phosphorylated Akt, (p-Akt), glycogen synthase kinase 3 (GSK3)-alpha, GSK3-beta, insulin like growth factor 1 receptor (IGF1R), insulin receptor (IR), insulin receptor substrate 1 (IRS1), mammalian target of rapamycin (mTor), ribosomal protein S6 kinase (p70S6k), phosphatase and tensin homologue (PTEN), ribosomal protein S6 (RPS6), and/or tuberous sclerosis complex 2 (TSC2). Increased levels of these proteins, phospho-proteins, and/or genes are found in less therapeutic MSCs, meaning that increased levels of phosphorylated Akt, (p-Akt), glycogen synthase kinase 3 (GSK3)-alpha, GSK3-beta, insulin like growth factor 1 receptor (IGF1R), insulin receptor (IR), insulin receptor substrate 1 (IRS1), mammalian target of rapamycin (mTor), ribosomal protein S6 kinase (p70S6k), phosphatase and tensin homologue (PTEN), ribosomal protein S6 (RPS6), and/or tuberous sclerosis complex 2 (TSC2) can be indicative of, and associated with, less therapeutic MSCs.

In order to modify the less therapeutic MSCs to become highly therapeutic MSCs for use in any of the methods described herein, the proteomic, phospho-proteomic, or transcription profiles of proteins and/or genes, and/or the cytokine, chemokine, and/or growth factor secretion profiles of the less therapeutic MSCs can be modified to more closely match those of highly therapeutic MSCs. For example, the levels or expression levels of proteins, phospho-proteins, and/or genes in the MAPK pathway can be increased in the less therapeutic MSCs by treating the less therapeutic MSCs with compounds to activate the MAPK pathway. Non-limiting examples of compounds that can activate the MAPK pathway include JNK activators such as metformin, Sodium phenylbutyrate, AEBSF hydrocholoride, Azaspiracid-1, Scriptaid, MT-21, Anisomycin, Angiotensin II, and combinations thereof. Alternatively, the levels or expression levels of proteins, phospho-proteins, and/or genes in the PI3K/Akt pathway can be decreased in the less therapeutic MSCs by treating the less therapeutic MSCs with compounds to inhibit the PI3K/Akt pathway. Non-limiting examples of compounds that can inhibit the PI3K/Akt pathway include phosphorylated Akt (p-Akt) inhibitors such as MK-2206, Miltefosine, magnolia extract NSC 293100, NSC 154020, KRX-0401, and combinations thereof.

In any of the methods described herein, the highly therapeutic MSCs can be propagated in order to increase the number of such MSCs using standard tissue culture techniques. For example, the highly therapeutic MSCs can be propagated before administration to a joint of a subject.

In any of the methods described herein, the modification of the less therapeutic MSCs can occur prior to or simultaneously with administration of the less therapeutic MSCs to the subject in need thereof. Specifically, a JNK activator and/or a p-Akt inhibitor can be used to treat the less therapeutic MSCs prior to or simultaneously with the administration of the less therapeutic MSCs to the subject. Optionally, highly therapeutic MSCs can also be treated with a JNK activator and/or a p-Akt inhibitor prior to or simultaneously with the administration of the highly therapeutic MSCs to the subject.

In any of the methods described herein, the MSCs can be obtained from a biological source, specifically, from a donor. Non-limiting examples of such biological sources include bone marrow aspirate or bone marrow aspirate concentrate (BMAC); a lipoaspirate; a micronized lipoaspirate stromal vascular fraction; or tissue isolated from a placenta or umbilical cord.

In any of the methods described herein, the isolated MSCs can be incubated in certain cytokines, chemokines, and/or growth factors. Non-limiting examples of such cytokines, chemokines, and/or growth factors include interleukin 1 beta (IL-1β), IL-6, interferon gamma (IFN-γ), tumor necrosis factor alpha (TNF-alpha), and combinations thereof.

In any of the methods described herein, the subject receiving the highly therapeutic MSCs is a mammal. Preferably, the subject is a horse, cat, dog, or human.

In an exemplary embodiment, the invention provides a method of distinguishing highly therapeutic mesenchymal stromal cells (MSCs) from less therapeutic MSCs for use in treating osteoarthritis in a subject in need thereof, the method comprising: (a) isolating MSCs from a biological sample; (b) incubating the MSCs with interleukin 1 beta (IL-1β), IL-6, interferon gamma (IFN-γ), tumor necrosis factor alpha (TNF-alpha), and combinations thereof; (c) measuring (i) levels of cytokines, chemokines, and/or growth factors secreted by the MSCs and/or (ii) measuring proteomic, phospho-proteomic, or transcription profiles of genes in mitogen-activated protein kinase (MAPK) and phosphatidylinositol 3-kinase/protein kinase B (PI3K/Akt) pathways; and (d) distinguishing highly therapeutic MSCs from less therapeutic MSCs by one or more steps of: (i) classifying the MSCs as being highly therapeutic if there is greater secretion of cytokines, chemokines, and/or growth factors associated with an increased phospho-c-Jun N-terminal kinase (p-JNK) level relative to secretion from a standard control MSC, or if the secretion of the cytokines, chemokines, and/or growth factors associated with the increased p-JNK level is substantially similar to secretion from a standard control MSC that has been previously identified as being highly therapeutic; (ii) classifying the MSCs as being less therapeutic if there is equal or lower secretion of cytokines, chemokines, and/or growth factors associated with the increased p-JNK level relative to secretion from a standard control MSC, or if the secretion of the cytokines, chemokines, and/or growth factors associated with the increased p-JNK level is substantially similar to secretion from a standard control MSC that has been previously identified as being less therapeutic; and/or (iii) classifying the MSCs as being highly therapeutic if levels of proteins, phospho-proteins, and/or expression of genes in the MAPK pathway are increased relative to levels of the same proteins, phospho-proteins, and/or genes in the MAPK pathway from a standard control MSC or substantially similar to levels of the same proteins, phospho-proteins, and/or genes in the MAPK pathway from a standard control MSC that has been previously identified as being highly therapeutic, and/or classifying the MSCs as being less therapeutic if levels of proteins, phospho-proteins, or expression of genes in the PI3K/Akt pathway are increased relative to levels of proteins, phospho-proteins, and/or genes in the PI3K/Akt pathway from a standard control MSC that has been previously identified as being highly therapeutic or are substantially similar to levels of proteins, phospho-proteins, and/or genes in the PI3K/Akt pathway from a standard control MSC that has been previously identified as being less therapeutic.

In another exemplary embodiment, the invention provides a method of identifying and/or producing mesenchymal stromal cells as being therapeutically effective for treating osteoarthritis in a subject in need thereof, the method comprising: (a) incubating the MSCs with interleukin 1 beta (IL-1β), IL-6, interferon gamma (IFN-γ), tumor necrosis factor alpha (TNF-alpha), and combinations thereof; (b) measuring levels of cytokines, chemokines, and/or growth factors secreted by the MSCs; (c) optionally measuring transcription profiles of genes in mitogen-activated protein kinase (MAPK) and phosphatidylinositol 3-kinase/protein kinase B (PI3K/Akt) pathways; and (d) identifying the MSCs as being therapeutic if: (i) there is greater secretion of cytokines, chemokines, and/or growth factors associated with an increased phospho-c-Jun N-terminal kinase (p-JNK) level relative to secretion from a standard control MSC, or if the secretion of the cytokines, chemokines, and/or growth factors associated with the increased p-JNK level is substantially similar to secretion from a standard control MSC that has been previously identified as being therapeutic; and/or (ii) the levels of proteins, phospho-proteins, and/or expression of genes in the MAPK pathway are increased relative to levels of the same proteins, phospho-proteins, and/or genes in the MAPK pathway from a standard control MSC or substantially similar to levels of the same proteins, phospho-proteins, and/or genes in the MAPK pathway from a standard control MSC that has been previously identified as being highly therapeutic; and (e) optionally propagating therapeutic MSCs.

In another exemplary embodiment, the invention provides a method of altering the cytokine, chemokine, and/or growth factor expression profile of a plurality of mesenchymal stromal cells (MSCs) for use in treating osteoarthritis in a subject in need thereof, the method comprising: (a) isolating MSCs from a biological sample; (b) incubating the MSCs with interleukin 1 beta (IL-1β), IL-6, interferon gamma (IFN-γ), tumor necrosis factor alpha (TNF-alpha), and combinations thereof; (c) measuring (i) levels of cytokines, chemokines, and/or growth factors secreted by the MSCs and/or (ii) measuring proteomic, phospho-proteomic, or transcription profiles of genes in mitogen-activated protein kinase (MAPK) and phosphatidylinositol 3-kinase/protein kinase B (PI3K/Akt) pathways; and (d) classifying the MSCs as being highly therapeutic or less therapeutic by one or more steps of: (i) classifying the MSCs as being highly therapeutic if there is greater secretion of cytokines, chemokines, and/or growth factors associated with an increased phospho-c-Jun N-terminal kinase (p-JNK) level relative to secretion from a standard control MSC, or if the secretion of the cytokines, chemokines, and/or growth factors associated with the increased p-JNK level is substantially similar to secretion from a standard control MSC that has been previously identified as being highly therapeutic; and/or (ii) classifying the MSCs as being less therapeutic if there is equal or lower secretion of cytokines, chemokines, and/or growth factors associated with the increased p-JNK level relative to secretion from a standard control MSC, or if the secretion of the cytokines, chemokines, and/or growth factors associated with the increased p-JNK level is substantially similar to secretion from a standard control MSC that has been previously identified as being less therapeutic; and/or (iii) classifying the MSCs as being highly therapeutic if levels of proteins, phospho-proteins, and/or expression of genes in the MAPK pathway are increased relative to levels of the same proteins, phospho-proteins, and/or genes in the MAPK pathway from a standard control MSC or substantially similar to levels of the same proteins, phospho-proteins, and/or genes in the MAPK pathway from a standard control MSC that has been previously identified as being highly therapeutic, and/or classifying the MSCs as being less therapeutic if levels of proteins, phospho-proteins, and/or expression of genes in the PI3K/Akt pathway are increased relative to levels of the same proteins, phospho-proteins, and/or genes in the PI3K/Akt pathway from a standard control MSC or substantially similar to levels of the same proteins, phospho-proteins, and/or genes in the PI3K/Akt pathway from a standard control MSC that has been previously identified as being less therapeutic; and (e) treating the less therapeutic MSCs with an activator of the MAPK pathway and/or an inhibitor of the PI3K/Akt pathway, optionally prior to or simultaneously with administration of the less therapeutic MSCs to the subject in need thereof; and/or (f) treating the highly therapeutic MSCs with an activator of the MAPK pathway and/or an inhibitor of the PI3K/Akt pathway, optionally prior to or simultaneously with administration of the less therapeutic MSCs to the subject in need thereof.

In another exemplary embodiment, the invention provides a method of treating osteoarthritis in a subject in need thereof, the method comprising: (a) isolating MSCs from a biological sample; (b) incubating the MSCs with interleukin 1 beta (IL-1β), IL-6, interferon gamma (IFN-γ), tumor necrosis factor alpha (TNF-alpha), and combinations thereof; (c) measuring (i) levels of cytokines, chemokines, and/or growth factors secreted by the MSCs and/or (ii) measuring proteomic, phospho-proteomic, or transcription profiles of genes in mitogen-activated protein kinase (MAPK) and phosphatidylinositol 3-kinase/protein kinase B (PI3K/Akt) pathways; and (d) distinguishing highly therapeutic MSCs from less therapeutic MSCs by one or more steps of: (i) classifying the MSCs as being highly therapeutic if there is greater secretion of cytokines, chemokines, and/or growth factors associated with an increased phospho-c-Jun N-terminal kinase (p-JNK) level relative to secretion from a standard control MSC, or if the secretion of the cytokines, chemokines, and/or growth factors associated with the increased p-JNK level is substantially similar to secretion from a standard control MSC that has been previously identified as being highly therapeutic; and/or (ii) classifying the MSCs as being less therapeutic if there is equal or lower secretion of cytokines, chemokines, and/or growth factors associated with the increased p-JNK level relative to secretion from a standard control MSC, or if the secretion of the cytokines, chemokines, and/or growth factors associated with the increased p-JNK level is substantially similar to secretion from a standard control MSC that has been previously identified as being less therapeutic; and/or (iii) classifying the MSCs as being highly therapeutic if levels of proteins, phospho-proteins, and/or expression of genes in the MAPK pathway are increased relative to levels of the same proteins, phospho-proteins, and/or genes in the MAPK pathway from a standard control MSC or substantially similar to levels of the same proteins, phospho-proteins, and/or genes in the MAPK pathway from a standard control MSC that has been previously identified as being highly therapeutic, and/or classifying the MSCs as being less therapeutic if levels of proteins, phospho-proteins, and/or expression of genes in the PI3K/Akt pathway are increased relative to levels of the same proteins, phospho-proteins, and/or genes in the PI3K/Akt pathway from a standard control MSC or substantially similar to levels of the same proteins, phospho-proteins, and/or genes in the PI3K/Akt pathway from a standard control MSC that has been previously identified as being less therapeutic; and wherein: (i) the highly therapeutic MSCs are administered to the subject in need thereof or are optionally treated with an activator of the MAPK pathway and/or an inhibitor of the PI3K/Akt pathway prior to or simultaneously with administration of the highly therapeutic MSCs to the subject in need thereof; (ii) the less therapeutic MSCs are treated with an activator of the MAPK pathway and/or an inhibitor of the PI3K/Akt pathway prior to or simultaneously with administration of the less therapeutic MSCs to the subject in need thereof.

In any of the methods of the invention described herein, the MSCs can be formulated for administration to a subject having OA in need thereof. For example, the MSCs can be formulated for intra-articular or intra-synovial administration directly to the joints of a subject having OA. The MSCs can be formulated to be introduced directly into an arthritic joint of a subject undergoing surgery to repair or treat the arthritis. The MSCs can be formulated to be injected into an arthritic joint of a subject.

Formulations suitable for intra-articular or intra-synovial administration include aqueous and nonaqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. The formulations can further include hyaluronic acid. Suitable excipients include, by way of example and without limitation, water, saline, dextrose, glycerol or ethanol. In addition, if desired, the pharmaceutical compositions to be administered may also contain minor amounts of non-toxic auxiliary substances, such as wetting or emulsifying agents, pH buffering agents, stabilizers, solubility enhancers, and other such agents, such as, for example, sodium acetate, sorbitan monolaurate, triethanolamine oleate and cyclodextrins.

### Systems and Devices of the Invention

The invention also provides systems and devices for carrying out any of the methods described herein. For example, the invention provides a system comprising an incubator for incubating isolated MSCs with cytokines, chemokines, and/or growth factors such as interleukin 1 beta (IL-1β), IL-6, interferon gamma (IFN-γ), tumor necrosis factor alpha (TNF-alpha), and combinations thereof under appropriate conditions and a device configured to measure levels of cytokines, chemokines, and growth factors secreted by the MSCs and/or the proteomic, phospho-proteomic, or transcription profiles of proteins and/or genes in the mitogen-activated protein kinase (MAPK) and phosphatidylinositol 3-kinase/protein kinase B (PI3K/Akt) pathways described herein, and compare such levels to standard values (as described herein) in order to classify the MSCs as being highly therapeutic or less therapeutic, and/or to identify highly therapeutic MSCs.

Measurement of the levels of such molecules can occur by any method known in the art, such as for example and not limitation, immunosorbent colorimetric assays, enzyme-linked immunosorbent assays, dot blots, microarrays, fluorescent assays, quantitative PCR, RNA sequencing, Western blotting, or mass spectrometry.

The system can be configured to directly compare the levels of the cytokines, chemokines, and growth factors secreted by the MSCs and/or the proteomic, phospho-proteomic, or transcription profiles of proteins and/or genes in the mitogen-activated protein kinase (MAPK) and phosphatidylinositol 3-kinase/protein kinase B (PI3K/Akt) pathways to standards, and then to indicate whether the MSCs are highly therapeutic or less therapeutic, and/or to identify highly therapeutic MSCs. Such standards can include a standard MSC, or MSCs that have been previously identified as being highly therapeutic or less therapeutic.

Additionally, the system can be configured to perform statistical analyses in order to compare the measured levels of the cytokines, chemokines, and growth factors secreted by the MSCs and/or the proteomic, phospho-proteomic, or transcription profiles of proteins and/or genes in the mitogen-activated protein kinase (MAPK) and phosphatidylinositol 3-kinase/protein kinase B (PI3K/Akt) pathways to standards, and then to indicate whether the MSCs are highly therapeutic or less therapeutic, and/or to identify highly therapeutic MSCs. For example, the system can perform partial least squares discriminant analysis (PLSDA) to distinguish the MSCs as being highly therapeutic or less therapeutic, and/or to identify highly therapeutic MSCs.

The system can be configured to treat the MSCs with compounds to activate JNK and/or inhibit p-Akt as discussed herein.

The system can also be configured to propagate the selected highly therapeutic MSCs using tissue culture.

A computer can be connected to the system to perform any of the comparisons, statistical analyses, and/or indications of the MSCs discussed herein, as well as to control the device regarding incubation, treatment with the compounds to activate JNK and/or inhibit p-Akt, and/or propagation of the selected highly therapeutic MSCs.

The system can be encapsulated into a single point of use device, such as a microfluidic chamber, meaning that device(s) for incubating, analyzing/measuring, treating, and/or propagating the MSCs can be miniaturized and/or these steps can be performed on a chip.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### EXAMPLE 1. Evaluation of Human Mesenchymal Stromal Cell Paracrine Action in Osteoarthritis

It has been proposed that MSCs may be acting through direct engraftment in addition to paracrine signaling in concert with the local environment. However, MSC viability studies, following intra-articular injection, have shown short term survival (˜7 days) and low numbers of engrafted cells, indicating that a majority of newly regenerated tissue comes from host cells. These findings suggest that recruitment of endogenous cells may be a critical component to MSC therapies, such as via paracrine communication. hMSCs possess the capacity to secrete a wide range of paracrine factors to facilitate tissue remodeling, recruit stem and progenitor cells and mediate the immune response. In response to cartilage degeneration, hMSCs possess the ability to induce tissue remodeling through secretion of factors such as IL-6 and stromal cell derived factor (SDF)-1. hMSCs also possess the capacity to mediate the host immune response with factors like TNF-α stimulated gene 6 (TSG-6), TGF-β1 and adenosine. Encapsulating the MSCs allows an independent assessment of the paracrine signaling properties of these cells, by preventing their direct engraftment into the native tissue, and enables experiments focusing on the secretion of different factors and their effects on OA.

This Example demonstrates that encapsulated hMSCs have a therapeutic effect, through paracrine-mediated action, in both delaying OA onset and in preventing further development of established OA.

### Materials and Methods

hMSC Culture and Characterization

hMSCs derived from bone marrow were obtained from Emory Personalized Cell Therapy (EPIC) core facility at Emory University. hMSCs were cultured in complete minimum essential medium Eagle-a modification (α-MEM; 12561; Gibco) supplemented with 10% heat-inactivated fetal bovine serum (FBS; S11110H; Atlanta Biologicals), 2 mM L-glutamine (SH300340; HyClone), and 100 μg/mL penicillin/streptomycin (P/S) (B21110; Atlanta Biologicals) and sub-cultured at 70% confluency. hMSC phenotype was confirmed by adipogenic, chondrogenic, and osteogenic differentiation (kit protocols A1007001, A1007101, A1007201; StemPro Differentiation Kits; ThermoFisher Scientific). Flow cytometry was also used to characterize the hMSCs. A hMSC Verification Flow Kit (FMC020; R&D Systems) was used to confirm that MSCs expressed characteristic MSC surface markers (CD73, CD90, CD105) and lacked hematopoietic markers (CD45, CD34, CD11b, CD79A, HLA-DR).

Encapsulation of hMSCs

1×106 cells/mL passage 4 hMSCs were suspended in 1% ultrapure low viscosity sodium alginate LVG (UP-LVG; 4200006; NovaMatrix). An electrostatic encapsulator (VARV1; Nisco Engineering AG) with a 0.2 μm nozzle, 2.5 mL/h flow rate, and 7 kV voltage was used. Capsules were gelled in 50 mM BaCl2. hMSC capsules were washed six times with 0.9% saline (NaCl), re-suspended to the appropriate dose, and stored at 4° C. in saline until injection. hMSC viability was confirmed with Live/Dead Viability/Cytotoxicity kit (L3224; Invitrogen) following encapsulation. Cell viability was quantified using ImageJ software. All rats were injected within 2 hours of injection.

In vitro Encapsulated hMSC Viability

Immediately following encapsulation and washing, encapsulated hMSCs were placed in complete α-MEM medium in 12-well plates and cultured at 37° C., 5% CO₂ until different time points were reached (1, 3, 5, 7, 9, 14, 21, 28, and 35 days post plating), with medium changed every 3 days. At the specified time point, hMSC viability in capsules was determined with Live/Dead Viability/Cytotoxicity kit (L3224; Invitrogen). Percentage viability was quantified by comparing the relative number of live cells stained with calcein-AM to ethidium-homodimer-stained dead cells counted through ImageJ software on serial z-stacked images, each containing 3-17 capsules, obtained by a confocal microscope, at 3.99 μm z-thickness. Image collection and quantification was completed for every 14 sections, ensuring encapsulated cells were counted only once.

In vivo Encapsulated hMSC Viability

Bioluminescence imaging (BLI) Naïve Lewis rats were injected with an intra-articular injection of 5×10⁵ cells/knee of either encapsulated or nonencapsulated luciferase-expressing hMSCs (n=5 for each group). Following cellular injections at day 0, animals received an intra-articular injection of 40 mg/mL luciferin (Promega Beetle Luciferin Potassium Salts; ThermoFisher Scientific) diluted in α-MEM (12561; Gibco). Incubation times for initial and subsequent luciferin injections were optimized in a prior pilot study, in which incubation time points that yielded maximum signal were selected, using the Bruker In-Vivo Xtreme (Bruker). At day 0, a 30 min incubation time was allotted before BLI was conducted using the Bruker In-Vivo Xtreme imaging system. Additional BLI readings were performed at 1, 3, 5, 7, and 9 days post hMSC injections, with subsequent luciferin injections administered 20 min (incubation time) before readings. The minimum detection limit for luciferase-expressing hMSCs, in vitro, using the In-Vivo Xtreme imaging system was determined to be 10,000 cells. Bioluminescence intensity values were quantified using ImageJ software and plotted as percentage of maximum intensity. Background (naïve animals with luciferin alone) images (n=4) were also collected, and the averaged intensity value was subtracted from intensity values collected for all study samples.

In vivo MMT Model of OA

Animal care and experiments were conducted in accordance with the institutional guidelines of the Atlanta Veteran Affairs Medical Center (VAMC) and experimental procedures were approved by the Atlanta VAMC Institutional Animal Care and Use Committee (IACUC; Protocol: V004-15). Weight-matched wild type male Lewis rats (strain code: 004; Charles River), weighing 300-350 g, were acclimatized for 1 week after they were received. A surgical instability animal model, MMT, was used to induce OA. Animals were anesthetized using isoflurane and injected subcutaneously with 1 mg/kg sustained-release buprenorphine (ZooPharm). Skin over the medial aspect of the left femoro-tibial joint was shaved and sterilized. Blunt dissection was used to expose the medial collateral ligament (MCL), which was next transected to expose the meniscus. Then, a full-thickness cut was made through the meniscus at its narrowest point. Following transection of the meniscus, soft tissues were re-approximated and closed using 4.0 Vicryl sutures and the skin was closed using wound clips. Sham surgery was also performed in rats. For shams, the MCL was transected followed by closure of the skin without transection of the meniscus.

Two separate time courses were implemented to assess the effects of therapeutics to delay onset of OA (3-week time course) and prevent further development of established OA (6-week time course). For the 3-week time course MMT study, injections were administered the day after surgery with the study endpoint coming at 3 weeks, which is the time point in the MMT model that corresponds with the presentation of OA phenotypes. Furthermore, for the 6-week time course study therapeutics were injected at 3 weeks, corresponding to OA phenotype presentation followed by animal takedown 3 weeks later at the 6-week end point. At the time of injection for both MMT time course studies, MMT animals received 50 μL intra-articular injections using a 25-gauge needle. Animals were injected with 1) Hanks balanced salt solution [HBSS; MMT/Saline; n=8 (3-week); n=7 (6-week)], 2) empty sodium alginate capsules [MMT/Empty Caps; n=7 (3-week); n=7 (6-week)], 3) 5×10⁵ hMSC/knee in HBSS [MMT/hMSC; n=8 (3-week); n=8 (6-week)], and 4) 5×10⁵ encapsulated hMSC/knee [MMT/Encap hMSC; n=8 (3-week); n=7 (6-week)]. Sample sizes and specific treatment groups varied based on the respective studies being run and are further specified below. The cell dose (5×10⁵ cells/knee) used for injection was the maximum concentration that could be encapsulated and delivered in a 50 μL volume. Sham animals were not injected post-surgery [n=8 (3-week); n=6 (6-week)].

Tissue Preparation for microCT and Histology

Animals were euthanized at different timepoints post-surgery (3- and 6-weeks) via CO₂ asphyxiation. Cervical dislocation was used as a secondary euthanasia method after asphyxiation. Left hindlimbs were dissected and fixed in 10% neutral buffered formalin. Muscle and connective tissues were removed from the hindlimbs. The femur was disarticulated from the tibia. Meniscus and residual soft tissue surrounding the medial tibial condyle were dissected and discarded.

microCT Quantitative Analysis of Articular Joint Parameters

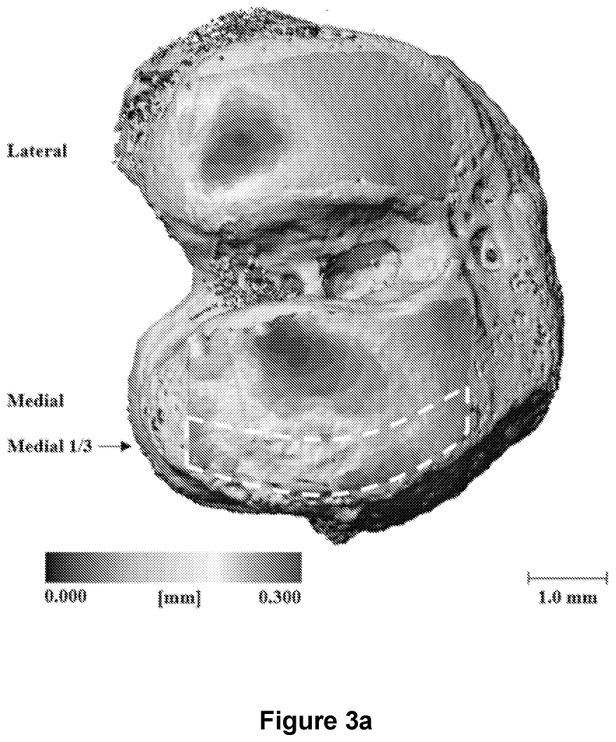

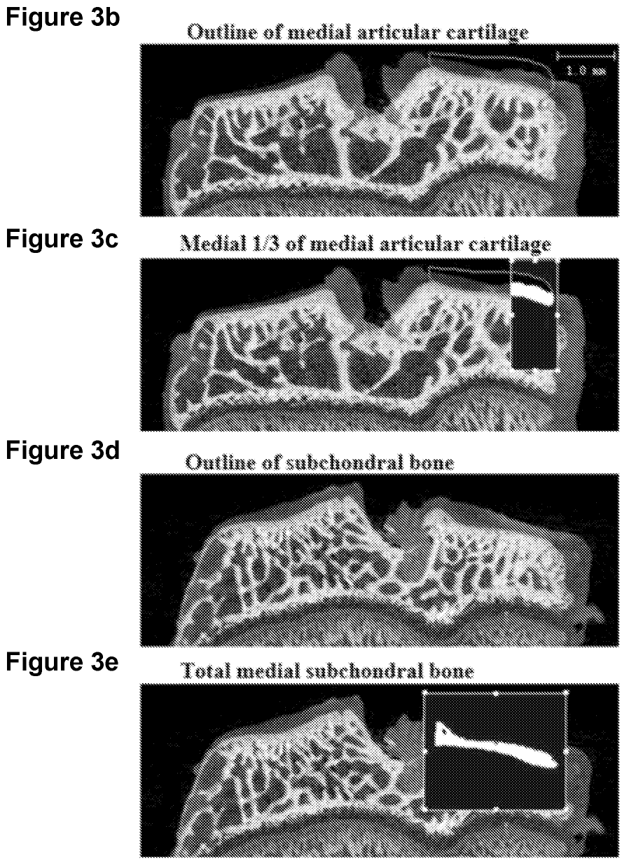

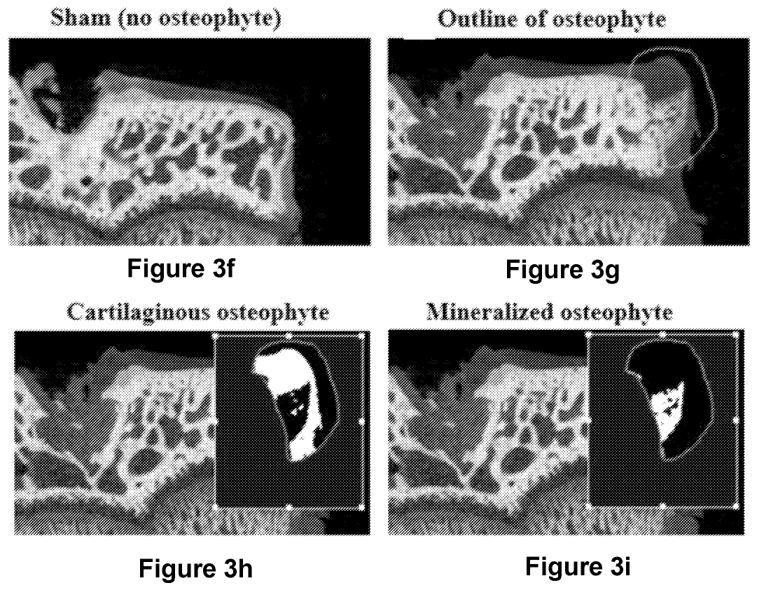

Prior to scanning, all muscle and connective tissue from collected fixed hind limbs was removed, the femur was disarticulated from the tibia, and all peripheral connective tissue surrounding the joint was removed to expose the articular cartilage of the medial tibial plateau. Exclusion criteria were employed to study tissue samples and included nicks of the medial articular cartilage surface (incurred either in the MMT surgical procedure or in dissection of the tissue samples), dissection error in the intercondylar area (due to dissection error), or loss of osteophyte structures on the medial edge of the joint (as a result of dissection error). All damage to the tissue samples was noted during the dissection stage and verified with microCT. Tibiae were immersed in 30% [diluted in phosphate buffered saline (PBS)] hexabrix 320 contrast reagent (NDC 67684-5505-5; Guerbet) at 37° C. for 30 mins before being scanned. All samples were scanned using microCT through the use of a Scanco μCT 40 (Scanco Medical) using the following parameters: 45 kVp, 177 μA, 200 ms integration time, isotropic 16 μm voxel size, and about a 27 min scan time. Scans were read out as 2D tomograms which were subsequently orthogonally transposed to yield 3D reconstructions for all scanned samples. All microCT parameters (articular cartilage, osteophyte, and subchondral bone) were evaluated as previously described (FIGS. 3a-3i). For cartilage parameters, thresholding of 110-435 mg hydroxyapatite per cubic cm (mg HA/cm³) was used to isolate the cartilage from the surrounding air and bone. Furthermore, for bone parameters, thresholds of 435-1200 mg HA/cm³ were implemented to isolate bone from the overlying cartilage.

Representative microCT images were taken demonstrating articular cartilage, osteophyte, and subchondral bone volume of interest (VOI) (FIGS. 3a-3i). As shown in FIG. 3a, rat tibial articular cartilage thickness map overlays on bone indicating articular cartilage VOI. Heat map indicates cartilage thickness according to the scale bar. FIG. 3b shows representative images of coronal sections of rat tibial joint showing outline of total medial articular cartilage. FIGS. 3c, 3d, and 3e respectively show the medial 1/3 (indicated in white) of the medial articular cartilage, an outline of the medial tibial subchondral bone, and the cortical subchondral bone (indicated in white) of the medial tibia. Representative coronal sections of medial tibial joint are also shown illustrating a lack of osteophyte in sham joint (FIG. 3f), contour of osteophyte in MMT joint (FIG. 3g), cartilaginous osteophyte volume (indicated in white) in MMT joint (FIG. 3h), and mineralized osteophyte volume (indicated in white) in MMT joint (FIG. 3i). The scale bar in FIG. 3a is universal for all representative images of coronal sections.

Coronal sections were both evaluated along the full length of the cartilage surface (total) and in the medial region of the medial tibial condyle. The medial 1/3 region of the articular cartilage was analyzed as this region has been shown to demonstrate high damage incidence in the MMT model. For articular cartilage, volume, thickness, and attenuation parameters were quantified. Attenuation is inversely related to sulfated glycosaminoglycans (sGAG) content. In OA, sGAG concentration in articular cartilage decreases due to degradation, creating a gradient which leads to an increased hexabrix concentration in the cartilage. High hexabrix and low sGAG levels (increased sGAG loss) correspond to a higher attenuation value. In addition to microCT analysis of articular cartilage, osteophyte volumes found on the most medial aspect of the medial tibial plateau were evaluated for their cartilaginous and mineralized portions. Osteophytes are a thickening and partial mineralization of cartilage tissue at the marginal edge of the medial tibial plateau and are a staple of OA development. Osteophytes consist of cartilaginous and mineralized portions, as they undergo an endochondral-like ossification process in formation. Additionally, subchondral bone was evaluated for volume, thickness, and attenuation (indirect measure of bone mineral density) along the total and medial regions, similar to the approach used for articular cartilage analysis.

### MATLAB Articular Cartilage Surface Roughness Analysis

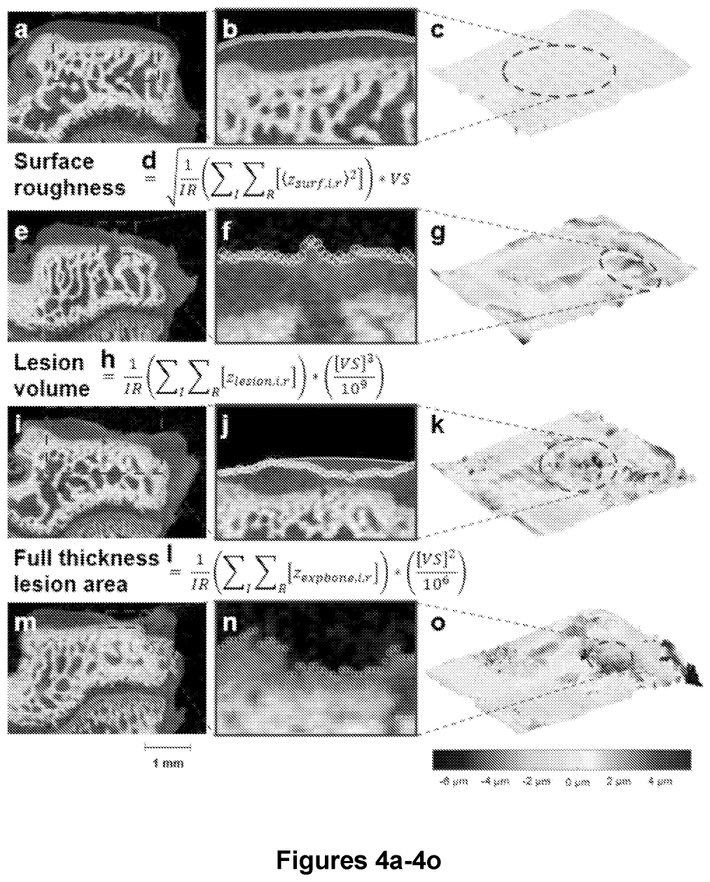

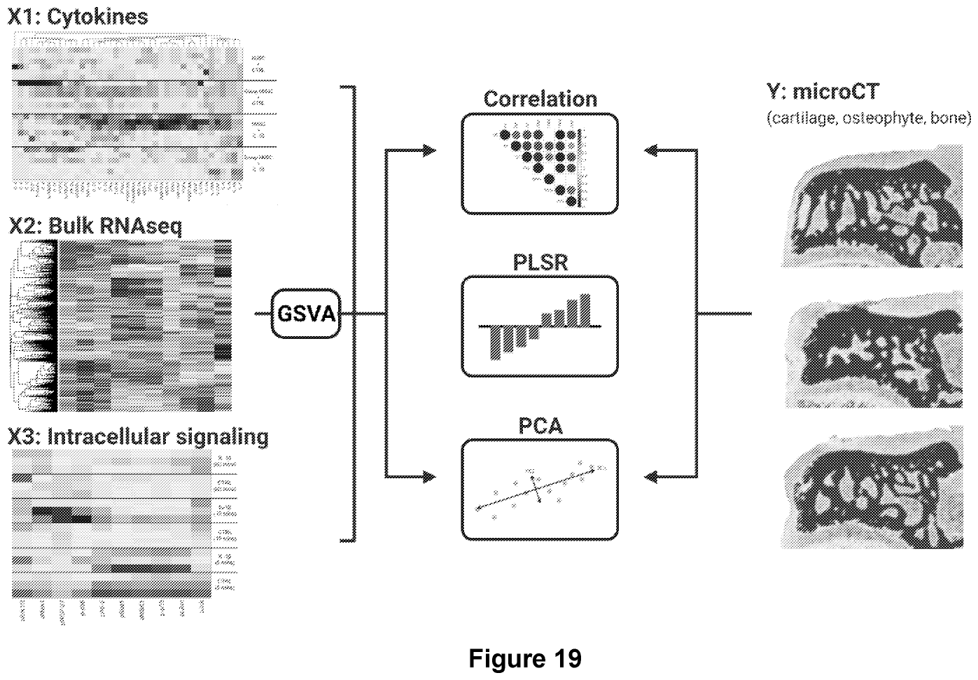

Serial 2D images of the proximal tibiae were analyzed using a customized algorithm in MATLAB (Math Works) to quantify surface roughness, lesion volume, and full-thickness lesion area. Images were processed to generate a 3D surface of the articular cartilage surface, as shown in FIGS. 4a-40. This 3D rendering was fitted along a 3D polynomial surface: fourth order along the ventral-dorsal axis and second order along the medial-lateral axis.

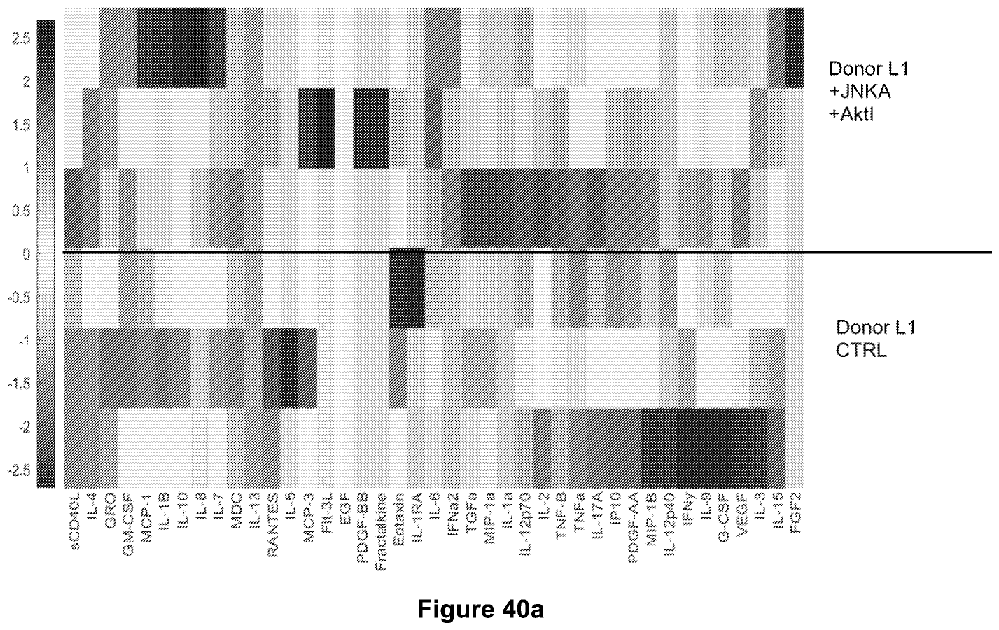

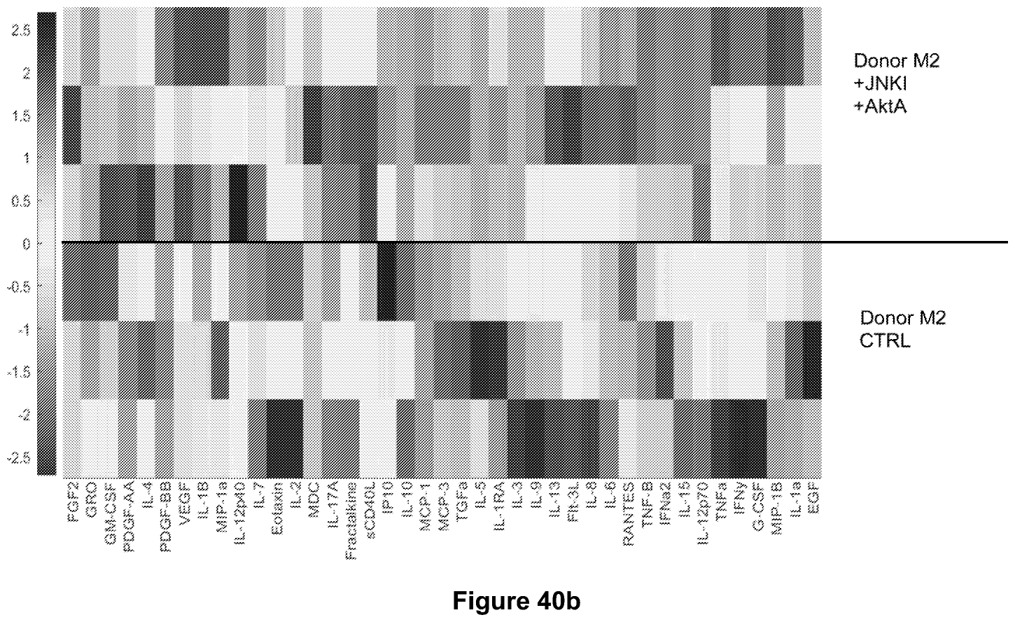

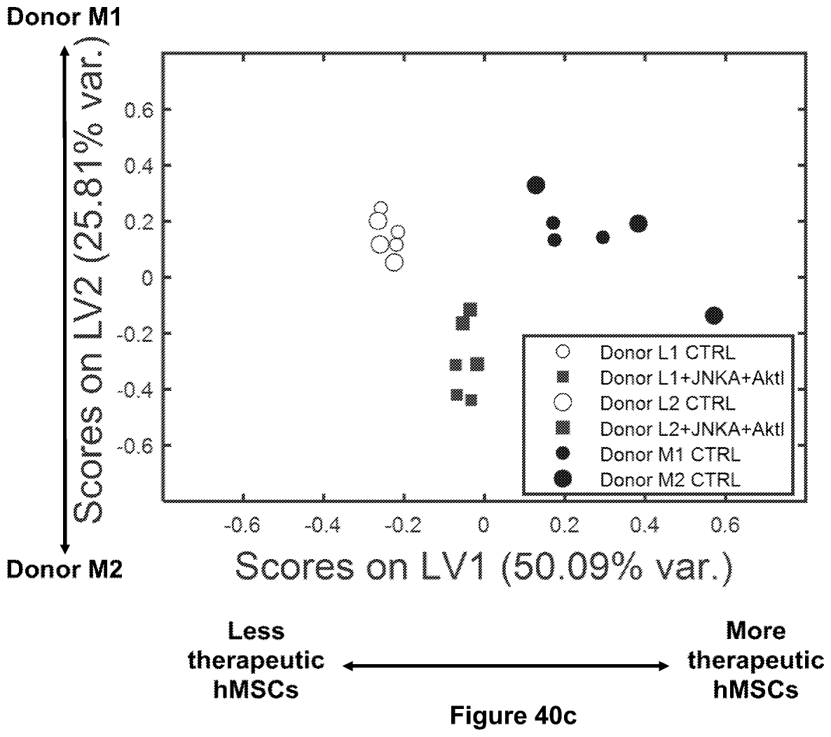

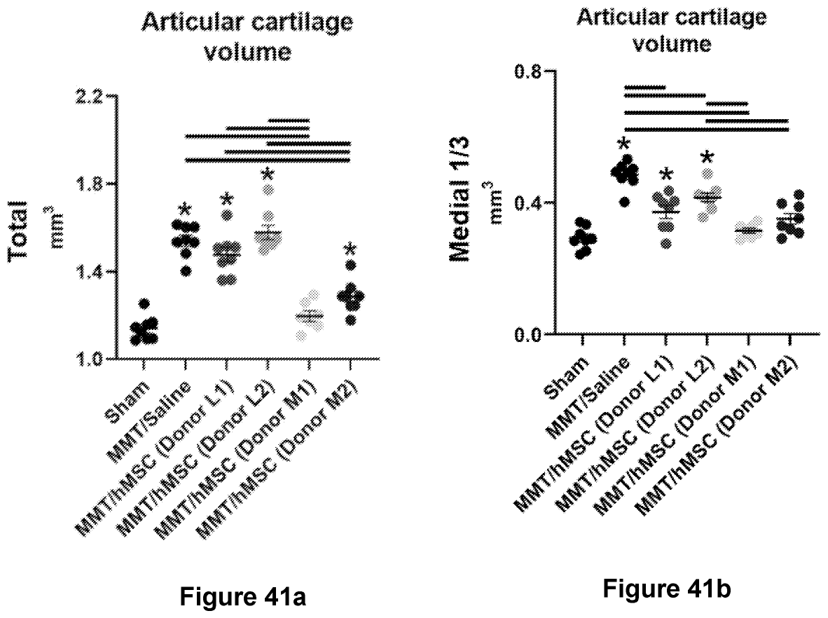

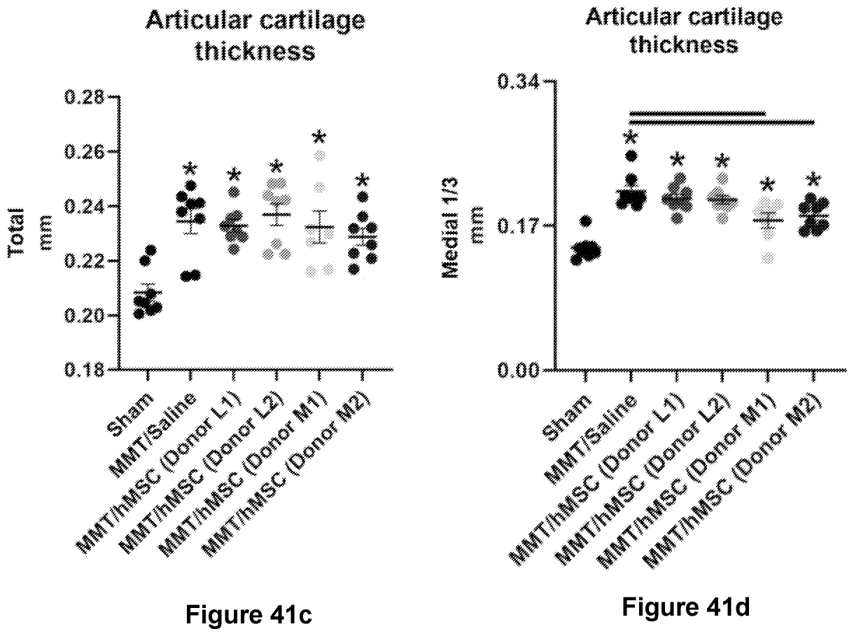

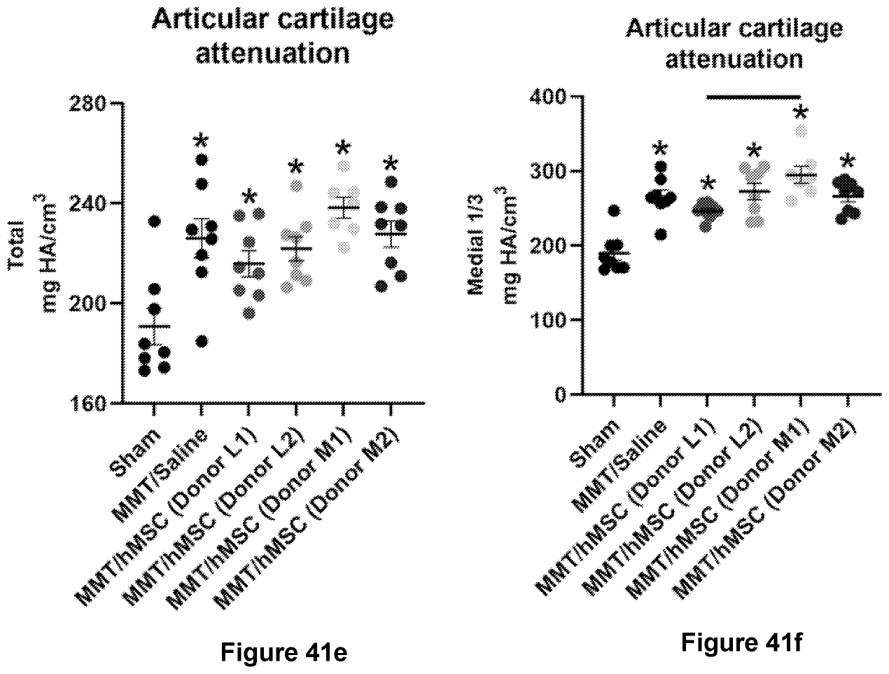

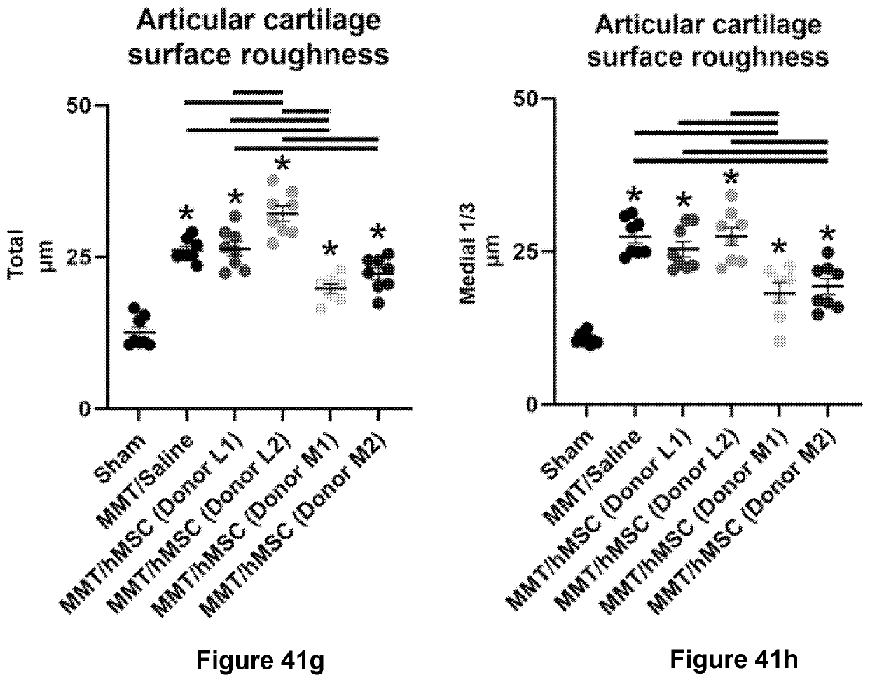

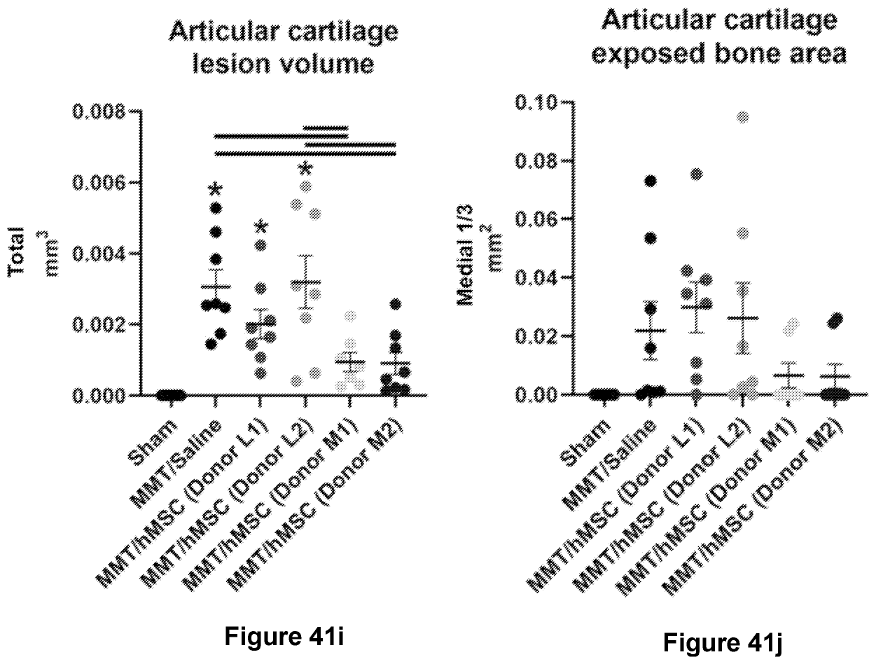

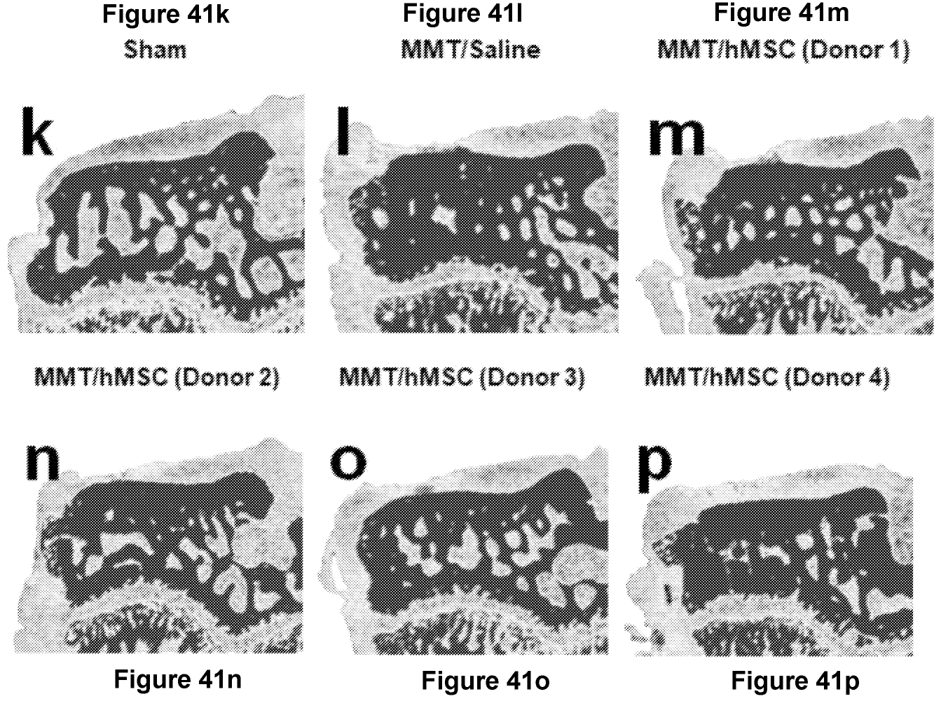

The root mean square difference between the generated (actual) and polynomial fitted (predicted) surfaces was the measure of cartilage layer surface roughness (FIGS. 4d-4g). Lesion volume (FIGS. 4h-4k) was calculated as the volume of root mean square difference between the generated fitted surface and the polynomial surface where >25% of total (predicted) cartilage thickness was exceeded. Full-thickness lesion area (FIGS. 41-40), also called exposed bone, was the sum of the area on the tibial condyle where no cartilage layer was present. Surface roughness, lesion volume, and full-thickness lesion area were calculated for full and medial 1/3 region of the articular cartilage.

As shown in FIGS. 4a-4b, representative microCT images of a 2D coronal-sectioned sham sample demonstrates a lack of surface fibrillations or lesion formation in the articular cartilage surface. This smooth surface is further represented in a 3D reconstruction of the cartilage surface (for only the outlined/zoomed region in the figure) (FIG. 4c). White circles on the articular cartilage surface depict the true cartilage boundary determined by the MATLAB script (FIGS. 4e-4f). These actual surface values are compared to the modeled surface (root mean square) produced by the code to determine surface roughness, which can be readily visualized in the surface rendering (FIG. 4g). Shown in FIGS. 4i-4j, lesion volume was determined using a similar approach, but volume was calculated by comparing the actual surface to modeled surface. The lesion volumes modeled in the figure are clearly represented in the surface renderings of the cartilage surface (FIG. 4k). Exposed bone was quantified by calculating the area where the pixel spacing between the cartilage surface and underlying subchondral bone were <3 pixels (FIGS. 4m-4o). The formulas for surface roughness, lesion volume, and full thickness lesion area (exposed bone) are also provided (FIGS. 4d, 4h, 4l).

### Histological Analysis

Tibiae were decalcified with Immunocal (SKU-1414-32; StatLab) for 7-10 days. Dehydrated samples were processed into paraffin-embedded blocks, 5 μm-thick sectioned, and stained with haematoxylin and eosin (H&E; 517-28-2; Fisherbrand) or safranin O and fast green (Saf-O; 20800; Electron Microscopy Sciences), following manufacturer protocols. For all samples, a single representative image was selected for H&E and Saf-O (serial sections). For the 6-week MMT study histological sections (n=3/treatment group; matched samples with microCT) were graded using a graphic user interface for the evaluation of knee OA (GEKO). GEKO is a quantitative histological grading tool based on the OARSI histopathology recommendations.

In vitro hMSC Cytokine Analysis Model

Passage 4 hMSCs, matching donor with in vivo MMT model, were utilized in vitro. Nonencapsulated hMSCs were sub-cultured to 80% confluency in complete α-MEM medium in 12-well plates and cultured at 37° C., 5% CO₂. For encapsulated hMSCs, immediately following encapsulation and washing, cells were placed in monolayer culture in treatment medium (unconditioned or conditioned) in 12-well plates at 37° C., 5% CO₂. Unconditioned media (+CTRL) contained complete α-MEM medium only and conditioned media (+IL-1β) contained 20 ng/ml IL-1β (FHC05510; Promega) in complete α-MEM medium. IL-1β was used to model the OA inflammatory environment in the current study as it is a major pro-inflammatory modulator in OA. IL-1β concentration (and group sample size) were selected based on prior experiments and preliminary data. Conditioned and unconditioned media were added to nonencapsulated and encapsulated hMSCs at day 0 (n=6; biological replicates from a single human donor) followed by a 24 hr conditioning period in monolayer culture with media collection at the end point for conditioning for the four study groups. Additional filtering steps were implemented in order to remove encapsulated hMSCs by passing collected media through a 9 μm filter. Samples were stored at −80° C. until Luminex analysis was performed. Loaded samples (2.03 μL) were determined to be within the linear range of detection of the MAGPIX (MAGPIX-XPON4.1-CEIVD; EMD Millipore Corporation) system. Cytokines were quantified using a bead based multiplex immunoassay, Luminex Cytokine/Chemokine 41 Plex Immunomodulatory Kit (HCYTMAG-60K-PX41; EMD Millipore Corporation). Median fluorescent intensity values were read out using Luminex xPONENT software V4.3 in the MAGPIX system. Background subtraction was performed on unconditioned and conditioned conditions using read out values from media only and 20 ng/mL IL-1β supplemented media, respectively.

### Partial Least Squares Discriminant Analysis (PLSDA)

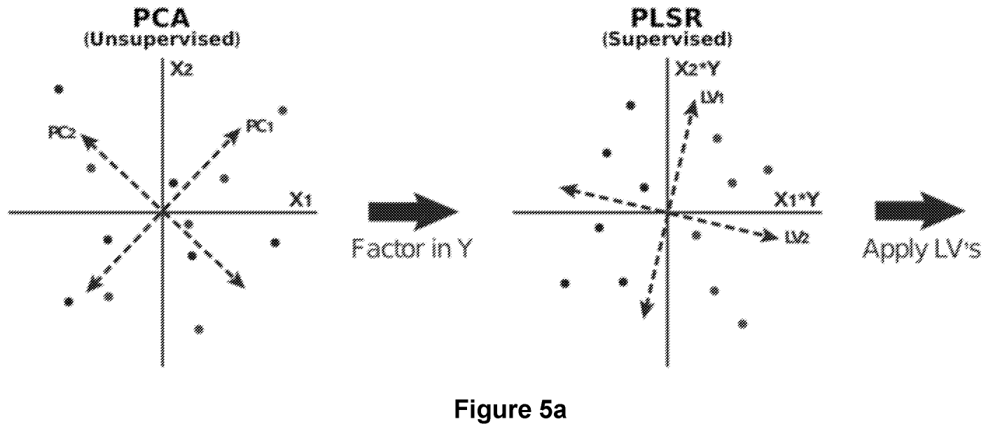

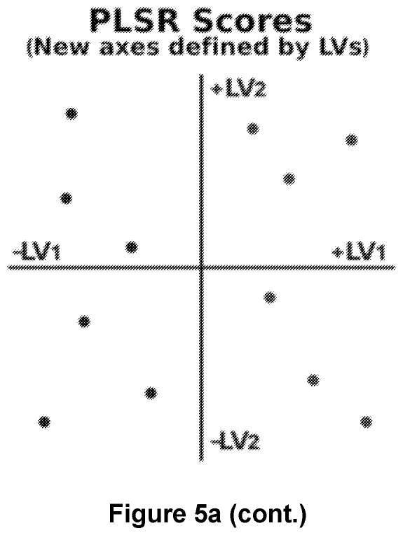

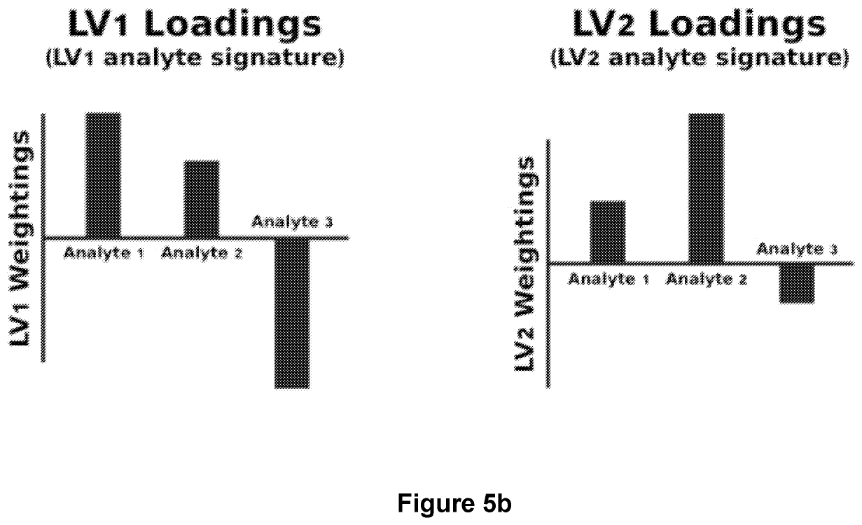

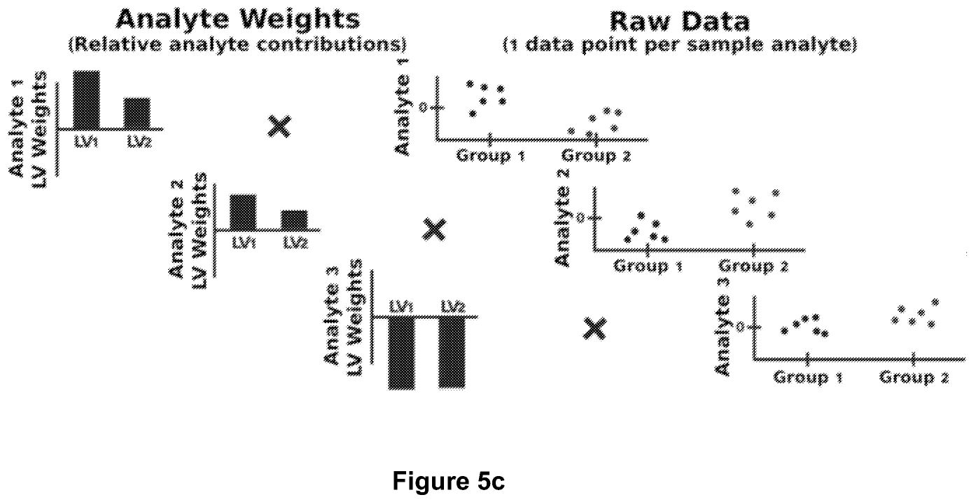

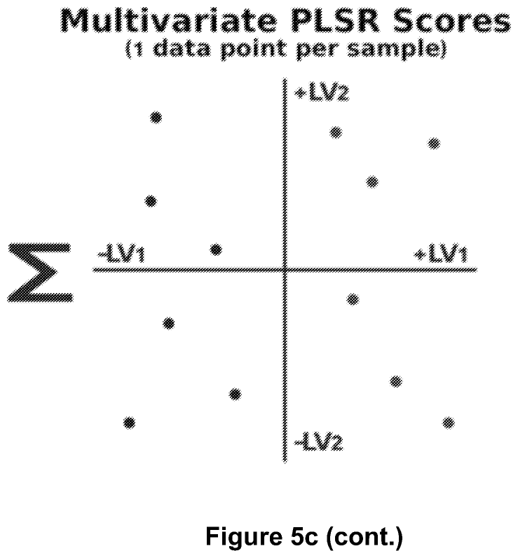

PLSDA was performed in MATLAB (Mathworks) using a function written by Cleiton Nunes (Mathworks File Exchange), as illustrated in FIGS. 5a-5c. This approach accounts for the multivariate nature of the data without overfitting. Prior to inputting the data into the algorithm, all cytokines were z-scored [(observed-mean)/standard deviation (SD)]. For articular cartilage, osteophyte, and subchondral bone in vivo analyses, total and medial microCT parameters were used as the independent variables and the five separate treatment groups were used as the outcome variables. For cytokine analysis of the in vitro cell culture model, cytokine measurements were used as the independent variables and the four individual treatment groups were used as the outcome variable. Latent variables (LV) in a multidimensional space (dimensionality varied by number of independent input variables) were defined and the two primary LVs were used for orthogonal rotation to best separate groups in the new plane defined by LV1 and LV2 (FIG. 5a). In the in vitro cell culture model orthogonality between encapsulated hMSCs (LV1 horizontal axis) and nonencapsulated hMSCs (LV2 vertical access) was confirmed via dot product (LV1 LV2=−1.059×10-16˜0). Loadings plots were generated from this analysis and display the relative importance of input variables (microCT parameters or cytokines) in contributing to the final composite values (scores) for each sample (FIGS. 5b-5c). Error bars on each cytokine (in the loadings plots) were computed by PLSR model regeneration using iterative (1000 iterations) leave one out cross validation (LOOCV).

As shown in FIG. 5a, PCA identifies axes of maximum variation among samples in the data when measurement variables (X1 and X2) are plotted against one another. Through incorporation of a response variable Y, PLSR enables identification of maximum co-variation between the X variables and different Y responses. PLSR outputs new linear combinations of X variables, referred to as LVs. As shown in FIG. 5b, each latent variable is comprised of weights, which ranks the importance of each input variable Xi, in determining the final composite values for each sample data point. To obtain the PLSR scores plot, the raw data is multiplied by the calculated weights for each LV (LV1 and LV2), as shown in FIG. 5c. The new axes defined by these LVs (LV1 and LV2) better separates the data with respect to the identity of the Y response variables.

### Statistical Analysis

A priori power analysis was run using α=0.05 and β=0.2, giving a power level of 0.8. From the power analysis, using medial 1/3 cartilage thickness as the primary outcome measure, it was determined that a sample size of at least 8 animals per group for both 3- and 6-week studies was necessary to find statistical differences between treatment groups. All data is presented as mean+SD. Significance for all microCT parameters was determined with one-way analysis of variance (ANOVA) with post hoc Tukey honest test for articular cartilage and subchondral bone parameters. Bonferroni correction was used for post hoc analysis for the exposed bone and osteophyte parameters due to their nonparametric nature. For all PLSDA scores plots, significance was determined with one-way ANOVA and post hoc Tukey honest test. To determine significant differences between encapsulated hMSCs in unconditioned (+CTRL) and conditioned (+IL-1β) conditions, two tailed t-tests were used with Bonferroni correction to account for the independent analysis of multiple groups. Statistical significance was set at p<0.05. All data were analyzed using the R stats, ggsignif, and ggpubr packages in R (The R Foundation).

Characterization of Encapsulated hMSCs

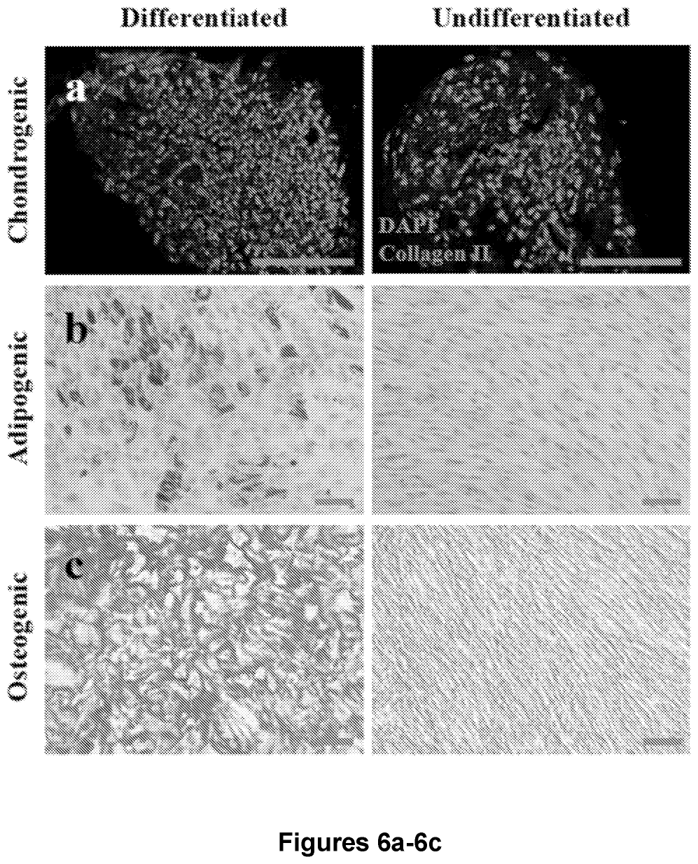

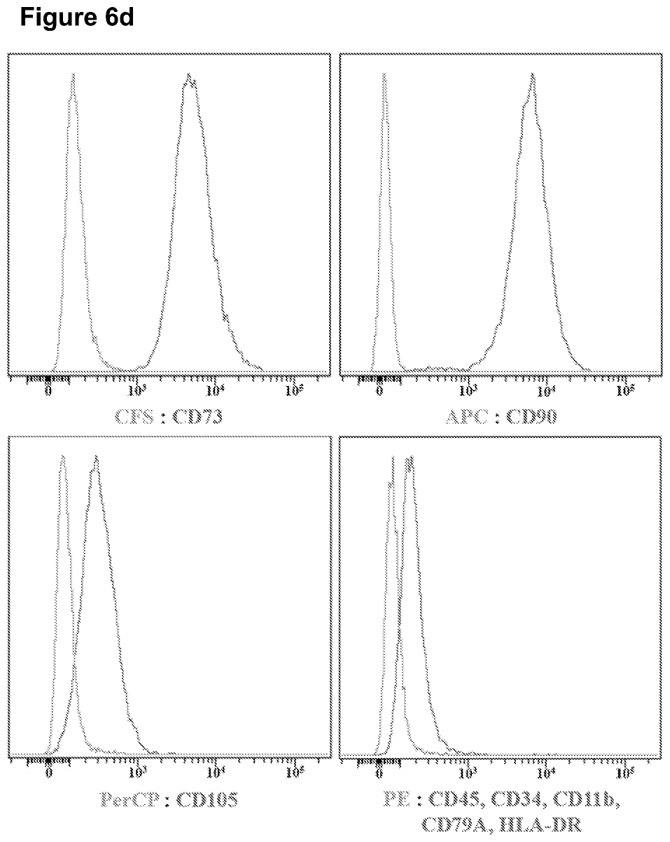

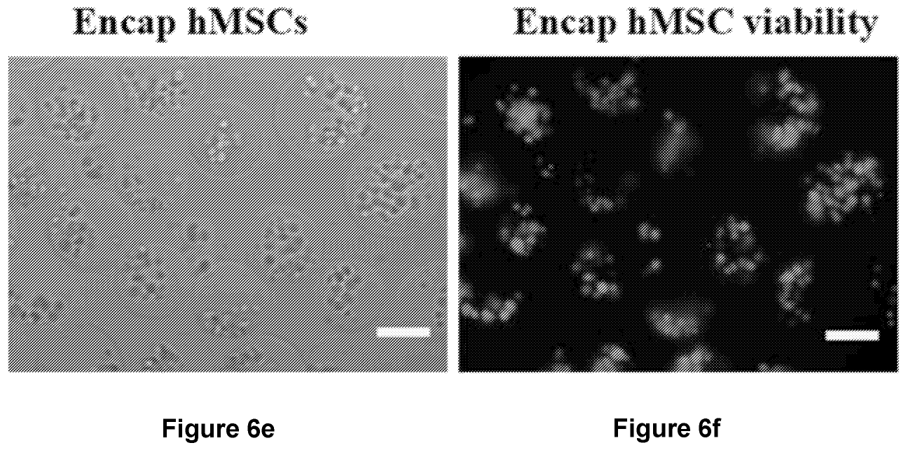

hMSC differentiation was confirmed with immunofluorescent staining for type II collagen of paraffin-sectioned pellets, oil red O staining, and alizarin red S staining for chondrogenesis, adipogenesis, and osteogenesis, respectively, as illustrated in FIGS. 6a-6c. Additionally, hMSCs were confirmed to be positive for typical MSC markers, including CD73, CD90, and CD105, and negative for hematopoietic markers, including CD45, CD34, CD11b, CD79A, and HLA-DR (FIG. 6d). The average diameter of encapsulated hMSC microspheres was 144±16 μm. Encapsulated hMSC viability immediately following encapsulation was 96±2.4%.

Multipotency of hMSCs was confirmed prior to encapsulation, as cultured hMSCs were differentiated into chondrogenic (FIG. 6a), adipogenic (FIG. 6b), and osteogenic phenotypes (FIG. 6c) as demonstrated by collagen type II, oil red O, and alizarin red staining. As shown in FIG. 6d, fluorescence activated cell sorting (FACS) analysis demonstrated that hMSCs expressed typical MSC surface markers: CD73, CD90 and CD105; but not hematopoietic markers: CD45, CD34, CD11b, CD79A, and HLA-DR. Bright field images of encapsulated hMSCs immediately following sodium alginate encapsulation showed capsule diameters of 170±27 μm, as shown in FIG. 6e. Fluorescent viability assay showed 96±2.4% of cells were viable immediately following encapsulation in sodium alginate, as shown in FIG. 6f. The scale bars shown equate to 100 μm.

Microencapsulation Moderately Potentiates hMSC Viability Following Intra-articular Injection

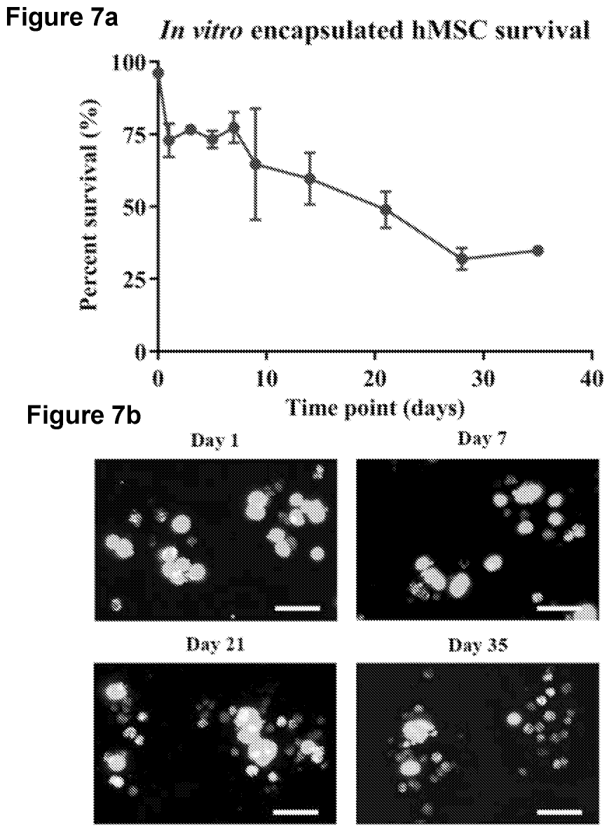

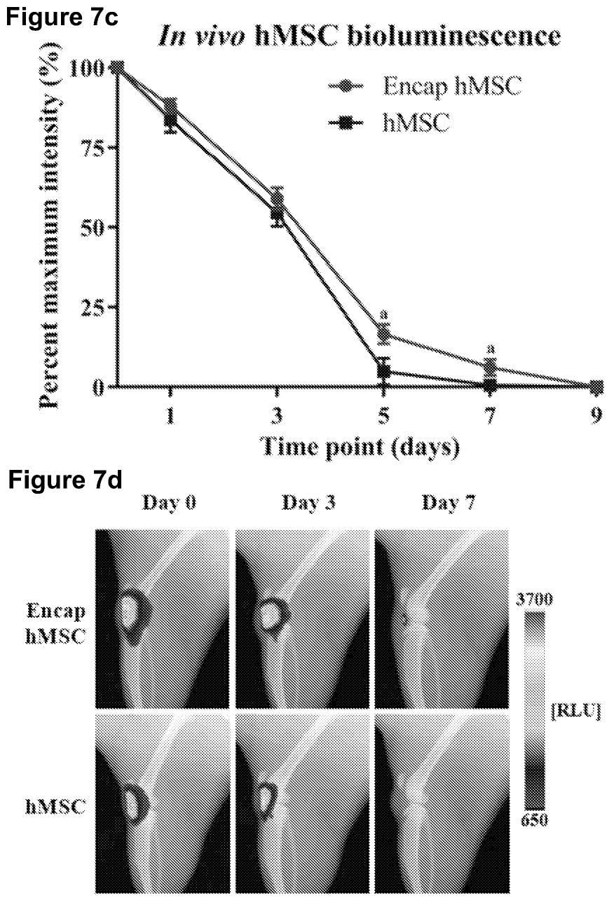

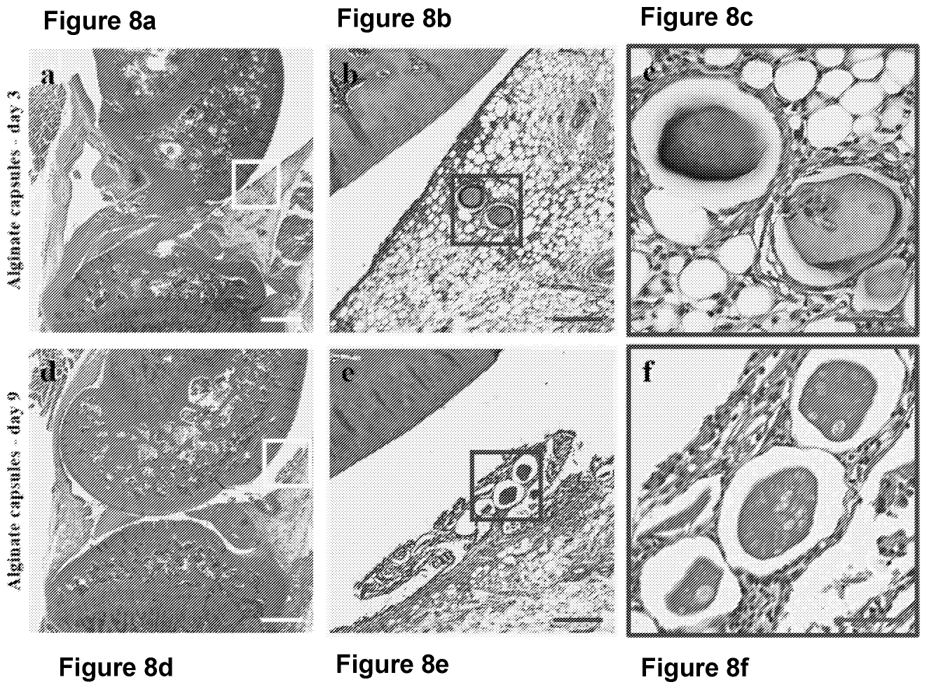

Luciferase-expressing hMSCs were used to assess the effect of encapsulation on cellular viability, retention, proliferation, and metabolic state both in culture and following intra-articular injections in rat knees, as illustrated in FIGS. 7a-7d and 8a-8f. In vitro viability studies demonstrated that encapsulated hMSCs were approximately 75% viable for the first 7 days following encapsulation and remained approximately 30% viable for at least 35 days under standard culture conditions (FIG. 7a). Representative maximum projection images are included displaying encapsulated cells at four key study time points (FIG. 7b).

As shown in FIG. 7a, hMSCs in alginate capsules, remained 70-80% viable at early time points (days 1-7), with viability slowly declining to approximately 30% at day 28 and 35. As shown in FIG. 7b, representative maximum projection images of capsules from key time points qualitatively demonstrated encapsulated hMSC viability over time in vitro. As shown in FIG. 7c, in vivo bioluminescent imaging demonstrated an overall increase in quantified bioluminescence of encapsulated hMSCs versus nonencapsulated hMSCs. Initial time points showed similar bioluminescent signals, while later time points (day 5 and 7) demonstrated differences in bioluminescent signal. Complete clearance of nonencapsulated hMSCs (<1%) was observed at day 7 and complete clearance of encapsulated hMSCs was observed at day 9. FIG. 7d shows representative images, from key study time points, of the rat knee joint that qualitatively illustrate bioluminescent signal for each of the study groups. Relative luminescence units (RLU) are provided with a heat map according to the scale bar. This data is presented as mean±SD, where n=5/group for in vivo study, p<0.05, and the scale bars equate to 50 μm.

In vivo bioluminescence was plotted as percentage of maximum intensity, with maximum intensity (100%) being expressed at day 0 for all animals. Encapsulated and nonencapsulated hMSCs showed similar initial decreases in bioluminescence, as no differences in signal were detectable for the first three study time points (day 0, 1, and 3). However, at later time points (day 5 and 7), nonencapsulated hMSCs had a small but statistically significant decrease in bioluminescence when compared to encapsulated hMSCs. While complete loss of hMSC bioluminescent signal (<1% of original intensity) was observed at day 7, encapsulated hMSCs yielded only about 6% cellular bioluminescence at this time point, with complete clearance at day 9 (FIG. 7c). Qualitative data, in the form of representative images selected from key time points for both groups, are included (FIG. 7d).

Full joint histology was performed on the hind limbs of animals (n=2/time point) injected with encapsulated hMSCs, at 3 and 9 days post-injection, to qualitatively assess cell and capsule retention following intra-articular injection. Capsules can be readily visualized with Saf-O, a cationic stain that binds to negatively charged alginate. Intact alginate capsules containing hMSCs were visible within the infrapatellar fat pad of the knee at day 3 (FIGS. 8a-8c). Alginate capsules were also visible in the joint space at day 9, surrounded by capsule remnants (FIGS. 8d-8f). While no hMSCs could be identified within the capsules at day 9, lacunae empty or potentially containing cell debris were present in the capsules. Identification of encapsulated hMSCs at day 3 and the absence of hMSCs in alginate capsules at day 9 was consistent with the in vivo bioluminescent analysis (FIG. 7c).

FIGS. 8a-8f illustrate representative histological images of full joint histology of rat hind limb knee joints at 3 days and 9 days post injection of encapsulated hMSCs. As shown in FIGS. 8a-8b, at day 3, Saf-O-stained joints showed the presence of sodium alginate capsules in the infrapatellar fat pad. As shown in FIG. 8c, H&E staining demonstrated the presence of encapsulated cells in the sodium alginate capsules at this time point. As shown in FIGS. 8d-8e, Saf-O staining at day 9 displayed intact sodium alginate capsules in the synovial lining of the knee joint space surrounded by remnants of broken-down capsules. As shown in FIG. 8f, no encapsulated cells were identifiable at day 9, as demonstrated by H&E staining, but lacunae were identifiable appearing either empty or containing cell debris. In each of FIGS. 8a-8f, the anterior hindlimb is located on the right and the posterior hindlimb on the left. The scale bars in 8a, 8d equate to 1 mm, 200 μm (8b, 8e), and 60 μm (8c, 8f).

Encapsulated hMSC Treatment Qualitatively Decreased OA Onset

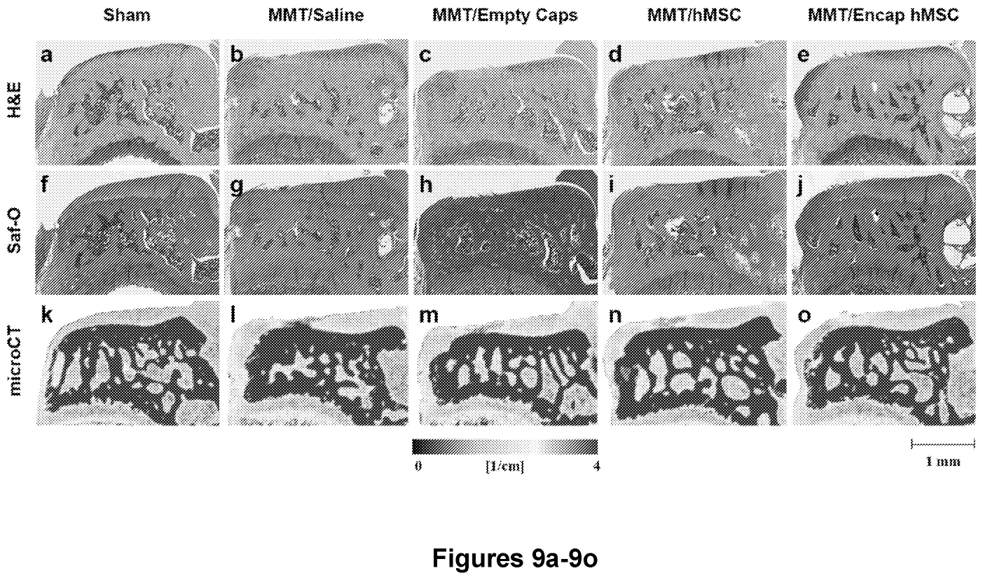

Histology was performed on collected tibiae to qualitatively analyze effects of encapsulated hMSC therapeutics on developing OA (3-week MMT study), as illustrated in FIGS. 9a-9o. Representative histological images of coronal tibial sections showed proteoglycan loss, degeneration of the cartilage surfaces and development of cartilaginous osteophytes in all MMT conditions (FIGS. 9b-9e, 9g-9j). The sham group showed good proteoglycan staining and smooth cartilage surfaces with no osteophyte development (FIGS. 9a, 9f). The MMT/Encap hMSC group also demonstrated decreased proteoglycan loss (decreased loss of haematoxylin staining in H&E; decreased loss of Saf-O staining in Saf-O; and decreased attenuation in microCT) relative to other MMT conditions (FIGS. 9e, 9j, 9o). However, no further analysis was performed on H&E and Saf-O images as qualitative changes were not readily evident. Representative coronal slices from both histology and microCT showed qualitatively similar disease progression over the 3-week study time course (FIGS. 9a-9o).

FIGS. 9a-9e provide images with H&E; FIGS. 9f-9j illustrate Saf-O stained MMT joints showing a lack of cartilage damage or osteophyte development in sham control (FIGS. 9a and 9f). FIGS. 9b-9e, 9g-9j show proteoglycan loss, degeneration of cartilaginous surfaces, and osteophyte development in all MMT groups. FIGS. 9k-9o illustrate corresponding microCT images showing similar disease progression as shown by histology. No cartilage damage was observed in sham control (FIG. 9k); and increased areas of cartilage attenuation, specifically in the medial 1/3, was observed in all MMT groups in addition to osteophyte volumes (FIGS. 9l-9o). Darker shading may indicate higher cartilage attenuation, corresponding to lower proteoglycan content. In all images, the medial tibial condyle is located on the left and the lateral tibial condyle on the right. The scale bar (bottom right corner) is universal for all histology and microCT representative images.

Encapsulated hMSCs Reduced the Degeneration of Articular Cartilage in Developing OA

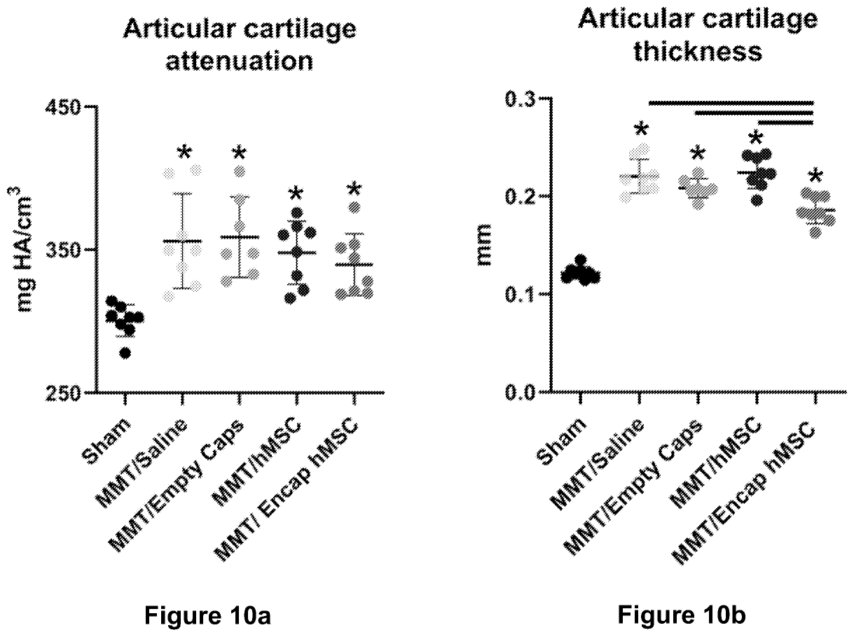

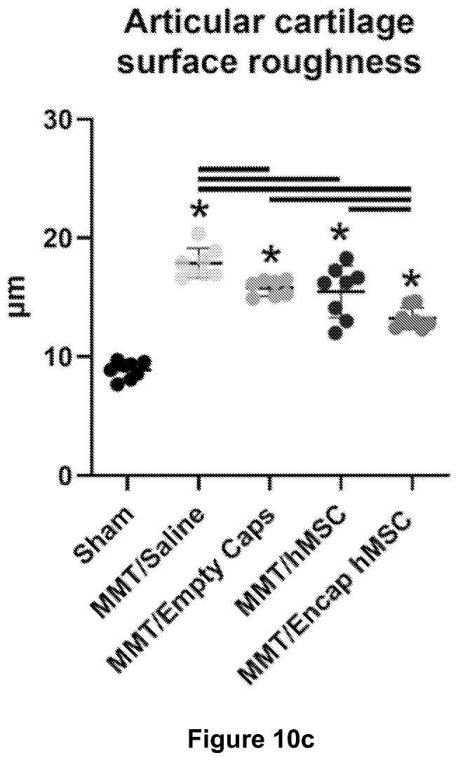

microCT was implemented to quantitatively analyze the effects of encapsulated hMSCs in articular cartilage structure and composition of the medial 1/3 of the medial tibial condyle in 3D, as illustrated in FIGS. 10a-10c. Analysis within the medial 1/3 of the medial tibial plateau showed a significant increase in attenuation, representative of a decrease in sGAG content, for all MMT groups as compared to the sham control (FIG. 10a). However, no significant differences in attenuation were noted between any of the MMT groups. Cartilage thickness of the medial 1/3 of the medial tibial condyle showed significant increases in thickness values for all MMT groups in comparison to the sham control (FIG. 10b). Additionally, the MMT/Encap hMSC group yielded reduced cartilage thickness increases in comparison to all other MMT groups. MATLAB surface roughness analysis of the medial 1/3 of the medial tibial plateau showed an increased articular cartilage surface roughness for all MMT groups as compared to sham animals (FIG. 10c). Furthermore, the MMT/Encap hMSC group yielded reduced surface roughness values in comparison to all other MMT groups. Additionally, the MMT/Saline group yielded increased surface roughness in comparison to all MMT groups.

As shown in FIG. 10a, cartilage attenuation was significantly increased in all MMT groups as compared to sham control. As shown in FIG. 10b, cartilage thickness was significantly increased in all MMT groups as compared to sham control; and MMT/Encap hMSC reduced increases in cartilage thickness as compared to all other MMT groups. As shown in FIG. 10c, cartilage surface roughness was significantly increased in all MMT groups as compared to sham control; MMT/Saline potentiated cartilage surface roughness as compared to all other MMT groups; and MMT/Encap hMSC reduced increases in cartilage surface roughness as compared to all other MMT groups. This data is presented as mean+SD; n=7/group for MMT/Empty Caps; n=8/group for all other groups. * represents significant differences (p<0.05) between individual MMT groups and sham. Horizontal black bars indicate significance (p<0.05) between individual MMT groups.

Encapsulated hMSCs Potentiated Osteophyte Development in Developing OA

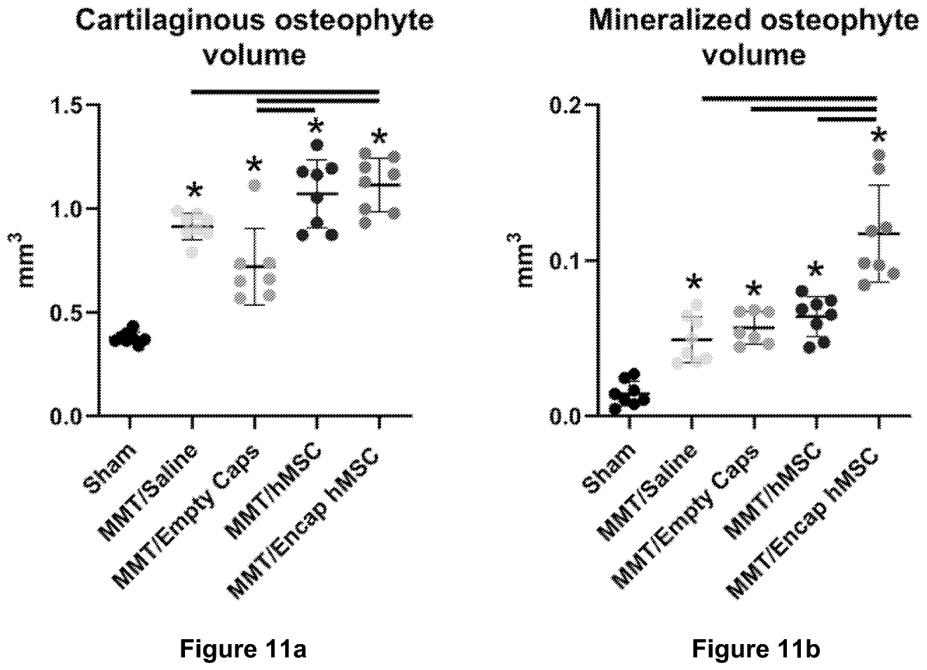

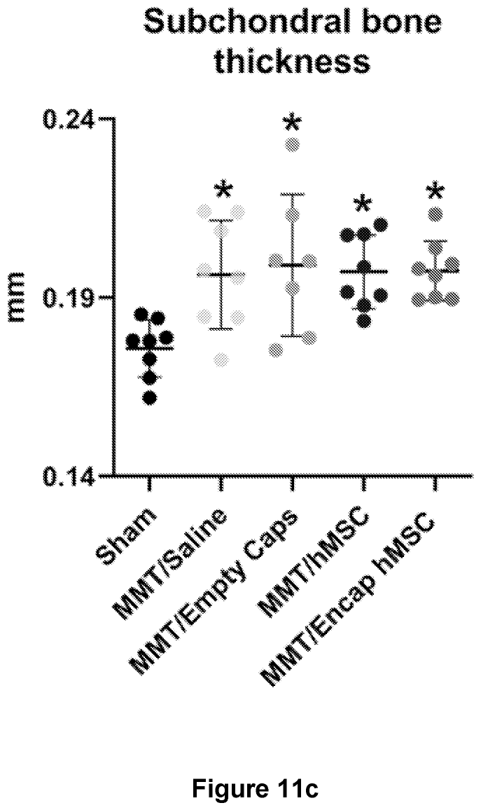

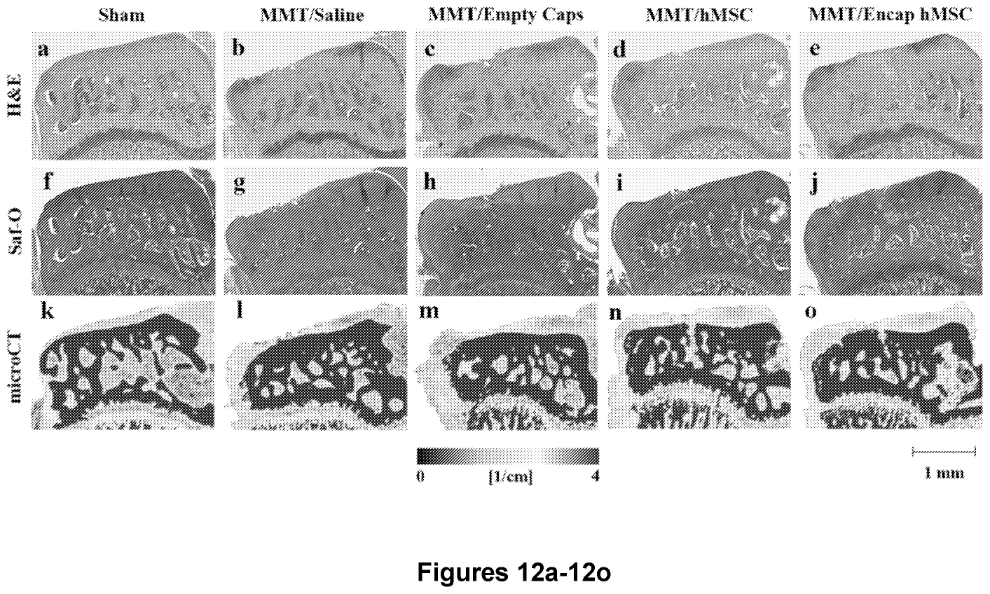

Cartilaginous and mineralized tissue volumes on the most medial aspect of the medial tibial condyle were quantified in 3D by microCT, as illustrated in FIGS. 11a-11c. Cartilaginous osteophyte volumes were significantly larger for all MMT groups in comparison to the sham control (FIG. 11a). Furthermore, MMT/Encap hMSC showed increased cartilaginous osteophyte volumes in comparison to all other groups, except MMT/hMSC. Histological representative images qualitatively confirmed the results of the cartilaginous osteophyte volume analysis (FIGS. 9f-9j). Mineralized osteophyte volumes showed an increase for all MMT groups as compared to the sham control (FIG. 11b). Additionally, MMT/Encap hMSC yielded increased mineralized osteophyte volumes in comparison to all other experimental groups. microCT representative images qualitatively confirmed the results of the mineralized osteophyte volume analysis (FIGS. 9k-9o). Subchondral bone, the layer of bone just below the articular cartilage in load-bearing joints, showed an increased thickness in all MMT groups as compared to the sham control (FIG. 11c). No significant differences were found between any of the MMT groups for this parameter.

As shown in FIG. 11a, cartilaginous osteophyte volume was significantly increased in all MMT groups as compared to sham control; MMT Encap hMSC potentiated osteophyte volumes as compared to MMT/Saline and MMT/Empty Caps. As shown in FIG. 11b, mineralized osteophyte volume was significantly increased in all MMT groups as compared to sham control; MMT/Encap hMSC potentiated mineralized osteophyte volumes as compared to all other MMT groups. As shown in FIG. 11c, subchondral bone thickness was significantly increased in all MMT groups as compared to sham control. This data is presented as mean+SD; n=7/group MMT/empty caps; n=8/group for all other groups. * represents significant differences (p<0.05) between individual MMT groups and sham. Horizontal black bars indicate significance (p<0.05) between individual MMT groups.

Encapsulated hMSC Treatment Qualitatively Reduced OA Progression in Established OA

Histology was performed on collected tibiae to qualitatively analyze effects of encapsulated hMSC therapeutics on established OA (6-week MMT study), as illustrated in FIGS. 12-12o. Representative histological images of the sham group showed consistent proteoglycan staining and a smoothness along the entire medial articular cartilaginous surface, and no presence of osteophyte development on the most medial aspect of the joint (FIGS. 12a, 12f). All MMT conditions showed proteoglycan loss (loss of Saf-O staining), loss of chondrocytes (lack of haematoxylin staining in certain regions of the articular cartilage layer), presence of fibrillations in the articular cartilage layer, and the development of osteophytes (FIGS. 12b-12e, 12g-12j). While all MMT conditions did show variable levels of cartilage damage, the MMT/Encap group showed qualitatively less cartilage degeneration and surface roughness, relative to all other MMT conditions (FIGS. 12e, 12j). Furthermore, while all MMT conditions showed osteophytes developing on the marginal edges of the joint, the MMT/hMSC and MMT/Encap hMSC group showed qualitatively larger areas relative to the MMT/Saline and MMT/Empty Caps group (FIGS. 12d-12e, 12i-12j). Together, these qualitative metrics demonstrated that with encapsulated hMSC treatment there was qualitatively less cartilage degeneration when compared to the other MMT groups.

Serial H&E (FIGS. 12a-12e) and Saf-O (FIGS. 12f-12j) coronal sections of rat medial tibial plateaus are shown at 6 weeks after sham or MMT operation of rat hindlimbs. As shown in FIGS. 12b-12e and 12g-12j, for MMT-induced OA, there is presence of increased articular cartilage degeneration [increased proteoglycan loss, loss of articular chondrocytes (lack of hematoxylin stain), surface fibrillations, formation of erosions and lesions], and the presence of osteophyte formations on the marginal edges for all MMT groups. Sham operated hindlimbs (FIGS. 12a, 12f) did not show any damage to articular cartilage or the presence of osteophyte formations. The MMT/Encap hMSC group (FIGS. 12e, 12j) showed less overall cartilage damage (smoother cartilage surface with less erosion and lesion formation) with respect to all other MMT groups. As shown in FIGS. 12k-12o, microCT representative images were matched with representative histology. All images are oriented with the medial aspect of the tibia on the left. The scale bar (bottom right corner) is universal for all histology representative images. A scale bar for the microCT images (bottom center) is also included.

Encapsulated hMSCs Reduced the Degeneration of Articular Cartilage in Established OA

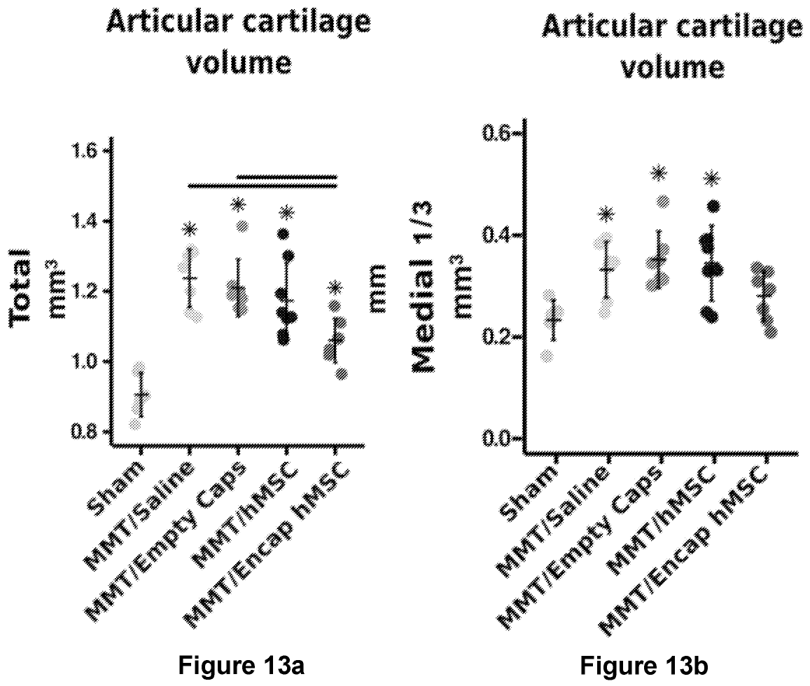

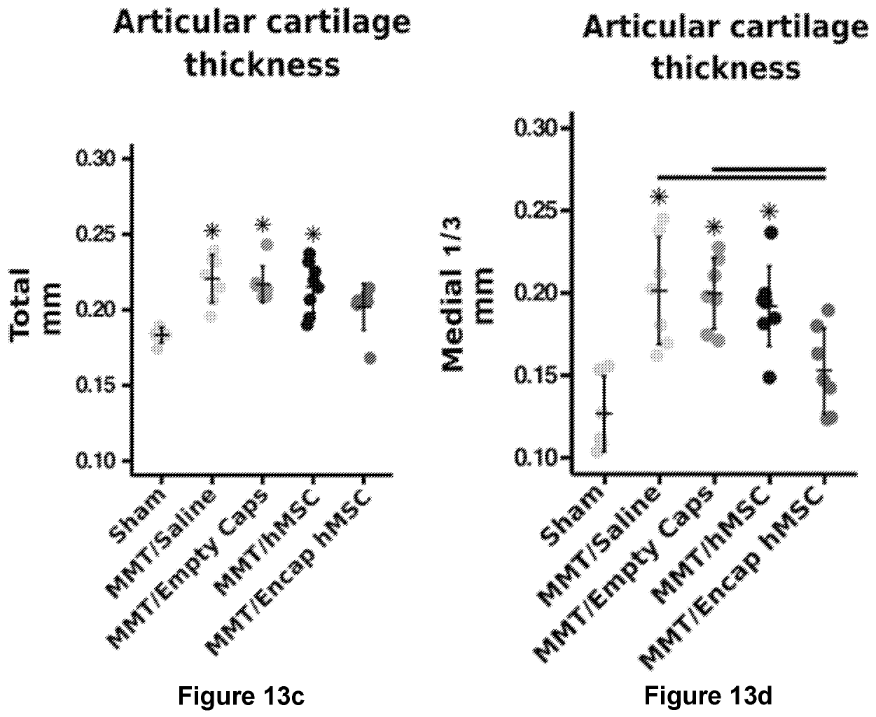

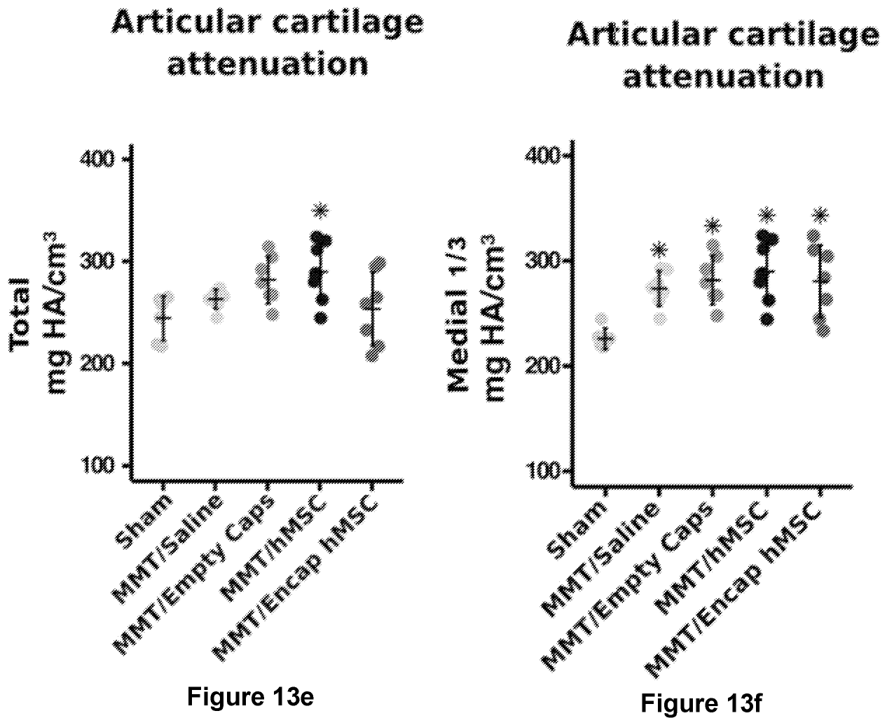

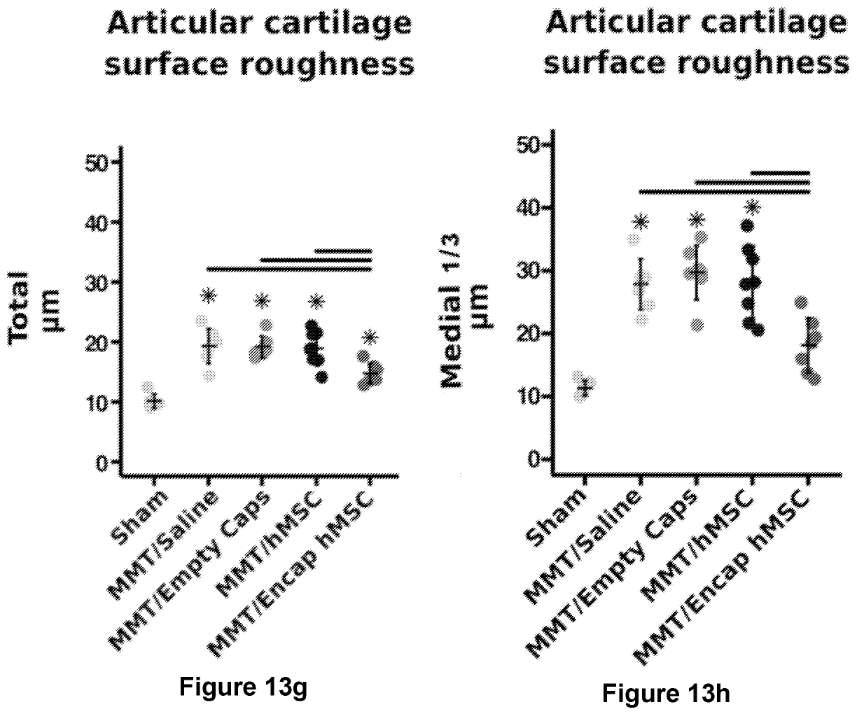

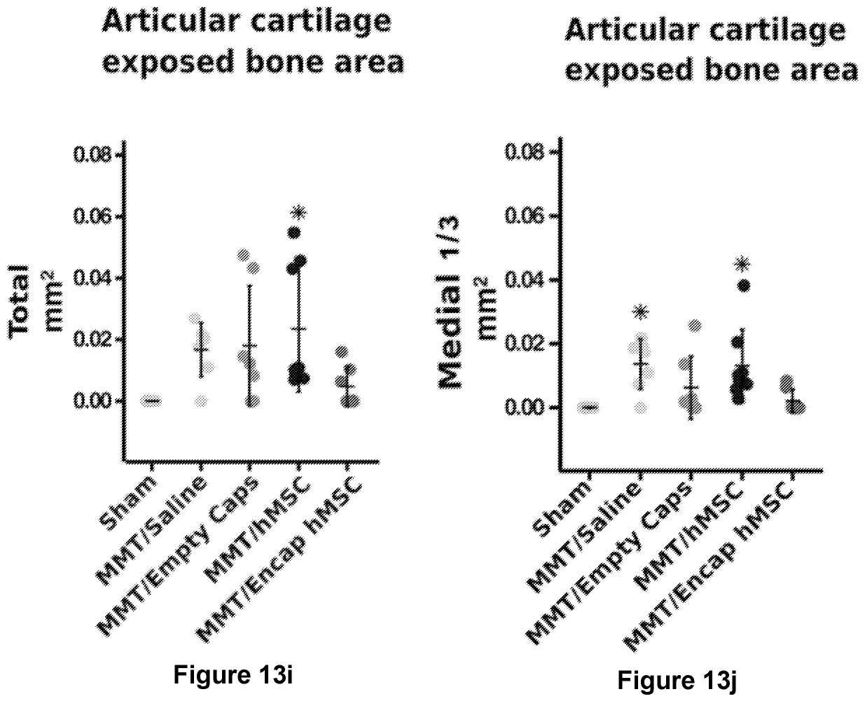

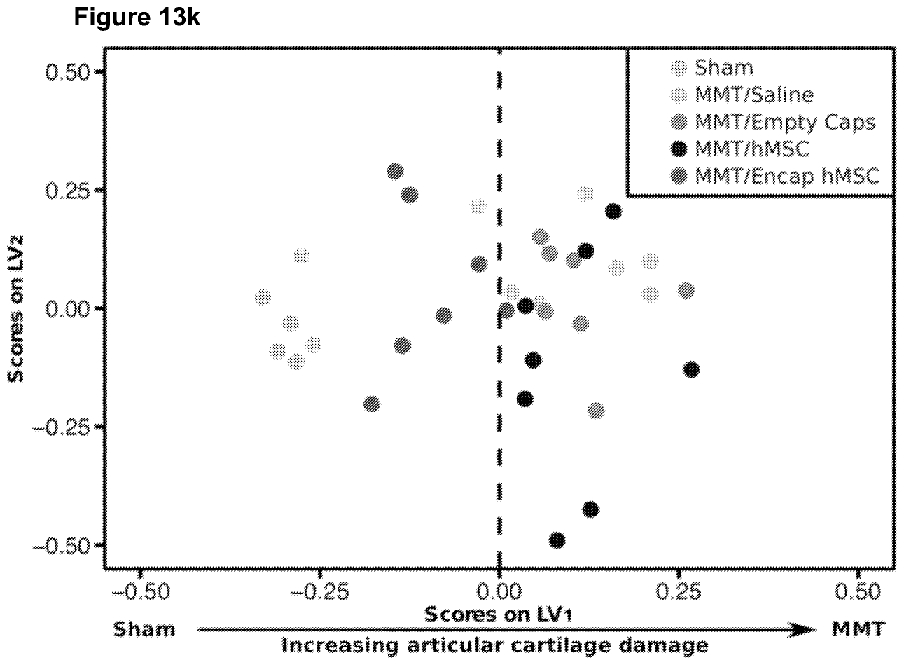

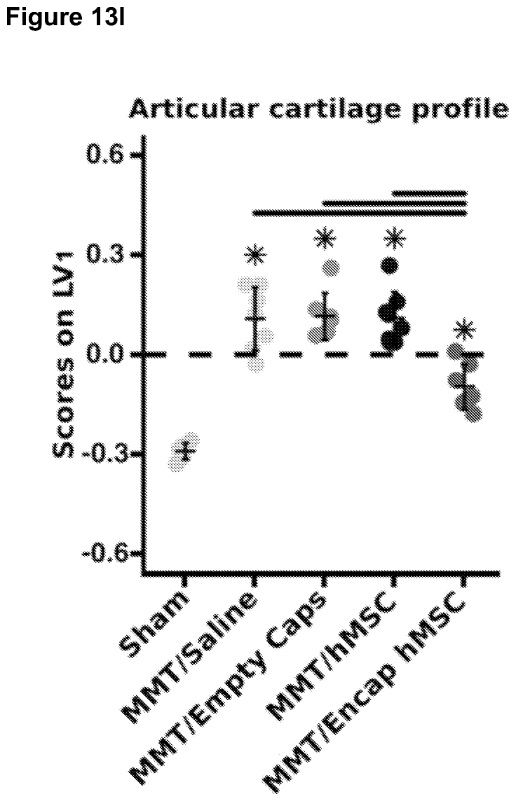

Detailed quantitative analysis of the articular cartilage changes in established OA were performed on various morphological parameters for the total and medial 1/3 of the medial tibial condyle, as illustrated in FIGS. 13a-13l. For total cartilage volume, all MMT groups showed elevated cartilage volume relative to sham (FIG. 13a). Treatment with MMT/Encap hMSCs reduced the MMT induced increase in cartilage volume that was found in the MMT/Saline and MMT/Empty Caps groups. However, no significant difference was found between MMT/Encap hMSC and MMT/hMSC alone, suggesting a milder effect of nonencapsulated cells relative to the encapsulated hMSCs. For medial cartilage volume analysis, significant increases in volume were yielded for the MMT/Saline, MMT/Empty Caps, and MMT hMSC groups. The MMT/Encap hMSC group did not show significant increases in cartilage volume relative to sham (FIG. 13b). Cartilage thickness yielded similar outcomes to the volume parameter as no significant difference for cartilage thickness were found between the MMT/Encap hMSC group relative to the sham group for both total and medial analysis (FIGS. 13c, 13d). Furthermore, for the medial thickness parameter there were no significant differences noted between MMT/Encap hMSC and MMT/hMSC, further demonstrating the mild therapeutic effect of the nonencapsulated hMSCs. Cartilage attenuation, which permits the indirect quantification of proteoglycan content, yielded a single significant difference between sham and MMT/hMSC for total analysis (FIG. 13e). While the medial analysis did discern differences between the sham and all MMT groups, there were no differences found between the MMT groups as they all demonstrated increased attenuation (decreased proteoglycan content) relative to the sham group (FIG. 13f). Additional analysis of the articular cartilage surface was performed using a custom MATLAB script to quantify surface roughness and exposed bone surface area (full thickness lesion surface area).

As shown in FIG. 13a, total articular cartilage volume for all MMT groups was significantly greater than sham animals; total articular cartilage volume for MMT/Encap hMSC group is significantly lower than MMT/Saline and MMT/Empty Cap groups. As shown in FIG. 13b, medial 1/3 articular cartilage volumes for MMT/Saline, MMT/Empty Caps, and MMT/hMSC groups were significantly greater than sham; hMSC/Encap hMSC group was not significantly different from sham. As shown in FIGS. 13c and 13d, total and medial 1/3 articular cartilage thickness values for MMT/Saline, MMT/Empty Caps, and MMT/hMSC groups were significantly greater than sham; no differences were found for either parameter between MMT/Encap hMSC and sham; medial 1/3 articular cartilage thickness for MMT/Encap hMSC group was significantly lower than MMT/Saline and MMT/Empty Caps groups. As shown in FIG. 13e, total articular cartilage attenuation for the MMT/hMSC group was significantly greater than the sham group. As shown in FIG. 13f, medial 1/3 cartilage attenuation values for all MMT groups were significantly greater than the sham group and no differences were found among MMT groups. As shown in FIG. 13g, total cartilage surface roughness showed significantly higher values for all MMT groups relative to sham; the MMT/Encap hMSC group did show significantly less surface roughness than all other MMT groups. As shown in FIG. 13h, medial 1/3 analysis of surface roughness yielded identical findings to total analysis except no difference was found between sham and MMT/Encap hMSC groups. As shown in FIGS. 13i and 13j, a difference in total exposed bone was found only for the MMT/hMSC group compared to all other groups; for medial 1/3 analysis of exposed bone, MMT/Saline and MMT/hMSC groups were significantly increased from sham group. As shown in FIG. 13k, PLSDA assessment of the overall effect of the therapeutics applied on articular cartilage damage showed distinct separation with sham and MMT Encap hMSC separating to the left and all other MMT groups separating to the right along LV1. As shown in FIG. 13l, quantification of the scores obtained from PLSDA analysis demonstrated that all MMT groups had significantly more cartilage damage than sham; in addition, MMT/Encap hMSC had significantly less damage than all other MMT groups. This data is presented as mean+/−SD; n=6 for sham, n=7 for MMT/Saline, n=7 for MMT/Empty Caps, n=8 for MMT/hMSC, and n=7 for MMT/Encap hMSC. * represents significant differences (p<0.05) between individual MMT groups and sham. Horizontal black bars indicate significance (p<0.05) between individual MMT groups.

Surface roughness analysis of the articular cartilage surface provides a quantitative measure of changes that may arise from matrix fibrillation, erosion and lesion formation, and full-thickness cartilage loss (exposed bone surface area). All MMT groups showed significantly increased surface roughness relative to sham for analysis of the total tibial plateau (FIG. 13g). For medial surface roughness analysis, the MMT/Saline, MMT/Empty Caps, and MMT/hMSC groups showed significantly increased surface roughness compared to sham but no difference was detected between MMT/Encap hMSC and sham (FIG. 13h). Additionally, treatment with MMT/Encap reduced the surface roughness to a level significantly lower than the other MMT groups for both total and medial analyses (FIGS. 13g, 13h). To further characterize changes to articular cartilage, exposed bone surface area was quantified. The total surface area of exposed bone was significantly different between the MMT/hMSC group and the sham group (FIG. 13i). For medial analysis of exposed bone, both MMT/Saline and MMT/hMSC showed a significant increase relative to sham (FIG. 13j).

To assess the overall effect of encapsulated hMSCs on MMT induction, factoring in all articular cartilage parameters (total and medial) as model inputs, PLSDA was implemented to identify new axes which better separate the data with respect to the identity of the treatment group (sham and MMT groups). LV1 separated out groups by severity of cartilage damage with the sham and MMT/Encap hMSC groups separating to the left from the MMT/Saline, MMT/Empty Caps, and MMT/hMSC groups, on the right (FIG. 13k). A one-way ANOVA of the LV1 scores demonstrated that all MMT groups had significantly higher scores (increased damage) than the sham; while the MMT/Encap hMSC group was significantly lower than the other MMT groups (FIG. 13l). However, MMT/Encap hMSC also demonstrated a higher LV1 score compared to the sham, suggesting increased cartilage damage (FIG. 13l). Overall, these metrics reveal that encapsulated hMSCs provided a positive therapeutic protective effect on articular cartilage in established OA.

Encapsulated hMSCs Potentiated Osteophyte Development in OA

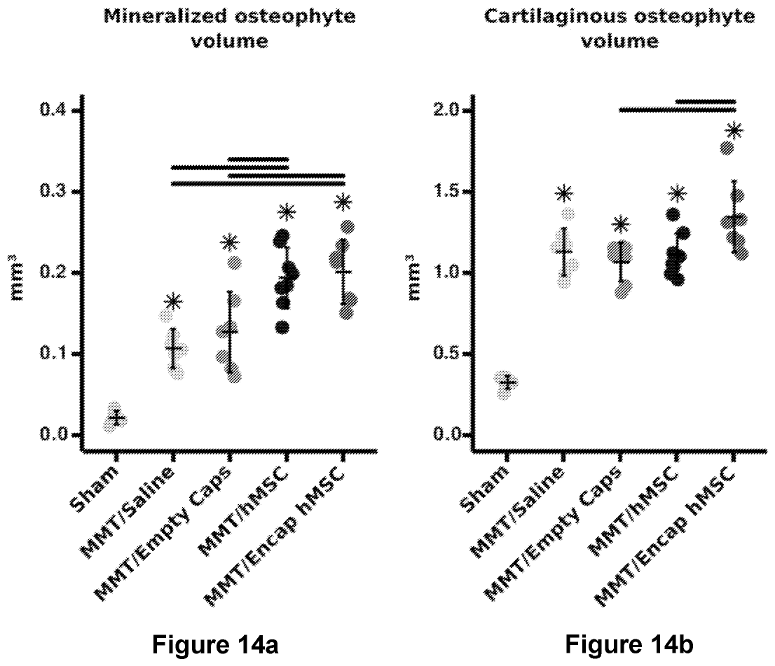

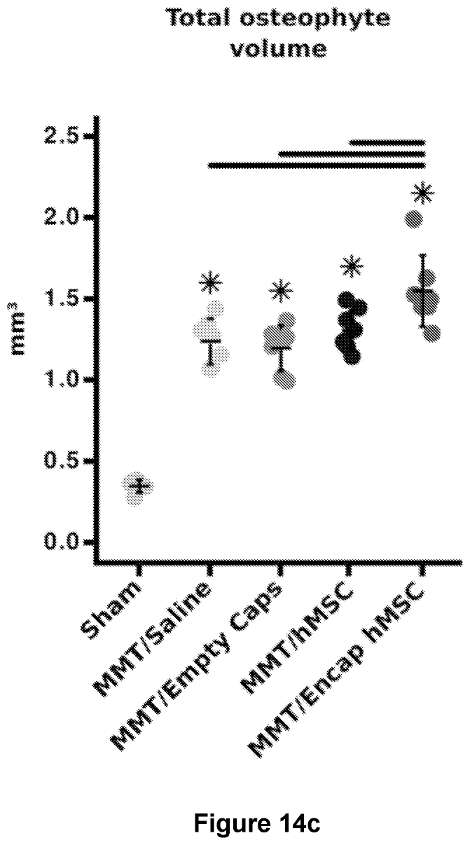

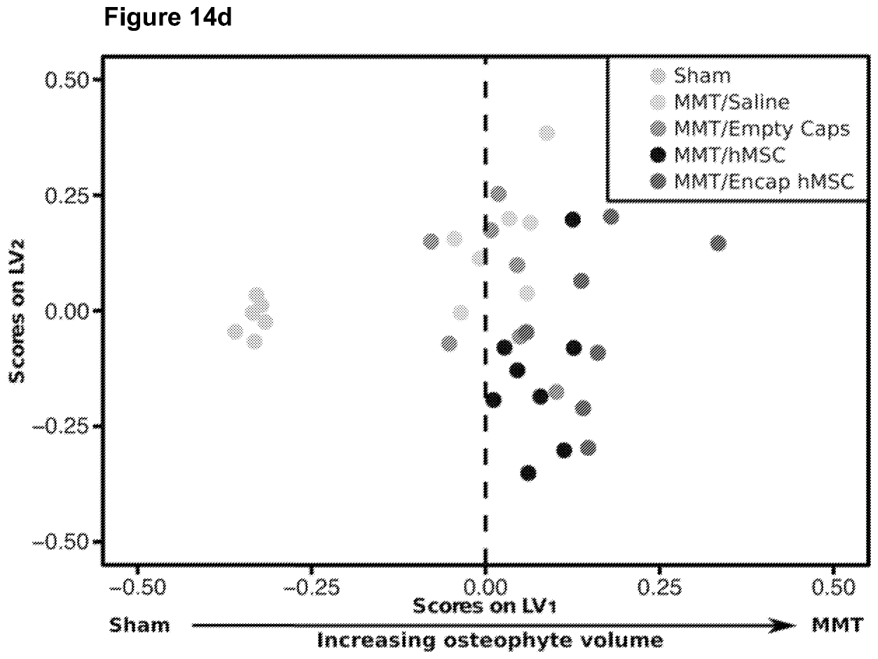

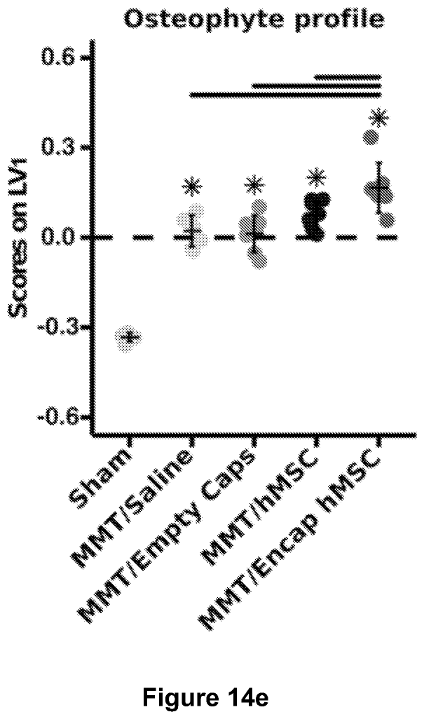

MicroCT analysis was implemented to quantitatively assess osteophyte volumes, as illustrated in FIGS. 14a-14e. Mineralized osteophyte volume was significantly greater in all MMT groups relative to the sham group (FIG. 14a). Furthermore, MMT/Encap hMSC and MMT/hMSC, which were not found to be different from one another, had significantly higher osteophyte volumes than the other two MMT groups (FIG. 14a). Analysis of the other major osteophyte component (cartilaginous osteophytes) showed significantly higher volumes for all MMT groups relative to sham (FIG. 14b). The MMT/Encap hMSC group also demonstrated increased cartilaginous osteophyte volumes relative to the MMT/hMSC and MMT/Empty Caps groups (FIG. 14b). Qualitative representation of these cartilaginous osteophytes can be viewed in the Saf-O histological images (FIGS. 12f-12j). Total osteophytes, a summation of mineralized and cartilaginous osteophytes, demonstrated similar findings to those for the individual parameters (FIG. 14c). A key difference identified was between MMT/Encap hMSC and MMT/hMSC, with the encapsulated group demonstrating a significantly higher total osteophyte volume.

To assess the overall effect of encapsulated hMSCs on both cartilaginous and mineralized osteophytes, PLSDA established an LV1 that separated sham to the left, and all MMT groups to the right based on increasing osteophyte volumes (FIG. 14d). ANOVA of LV1 scores displayed that all MMT groups were significantly higher (increased volume) than sham and that MMT/Encap hMSC was significantly higher than all other MMT groups (FIG. 14e). Importantly, these results indicate that hMSCs, particularly the encapsulated hMSCs, potentiated osteophyte volumes relative to other MMT groups that did not receive treatment.

As shown in FIG. 14a, mineralized osteophyte volumes for all MMT groups were significantly greater than the sham group; MMT/Encap hMSC and MMT/hMSC groups yielded significant increases in mineralized osteophyte volume compared to MMT/Saline and MMT/Empty Caps groups. As shown in FIG. 14b, cartilaginous osteophyte volumes for all MMT groups were significantly greater than the sham group; MMT/Encap hMSC demonstrated significant increases in cartilaginous volume relative to MMT/hMSC and MMT/Empty Caps. As shown in FIG. 14c, total osteophyte volumes (mineralized+cartilaginous) for all MMT groups were again significantly greater than the sham group; MMT/Encap hMSC yielded significantly greater total osteophyte volumes than all MMT groups. As shown in FIG. 14d, PLSDA analysis of overall osteophyte volumes depicted distinct separation of all groups based on osteophyte size, with sham to the left and MMT/Encap hMSC to the right along LV1. As shown in FIG. 14e, statistical analysis of LV1 scores demonstrated significantly higher values for all MMT groups, compared to sham, with MMT/Encap hMSC showing increased volumes relative to all MMT groups. This data is presented as mean+/−SD; n=6 for sham, n=7 for MMT/Saline, n=7 for MMT/Empty Caps, n=8 for MMT/hMSC and n=7 for MMT/Encap hMSC. * represents significant differences (p<0.05) between individual MMT groups and sham. Horizontal black bars indicate significance (p<0.05) between individual MMT groups.

Encapsulated hMSCs Had a Minimal Therapeutic Effect on Subchondral Bone Remodeling in Established OA

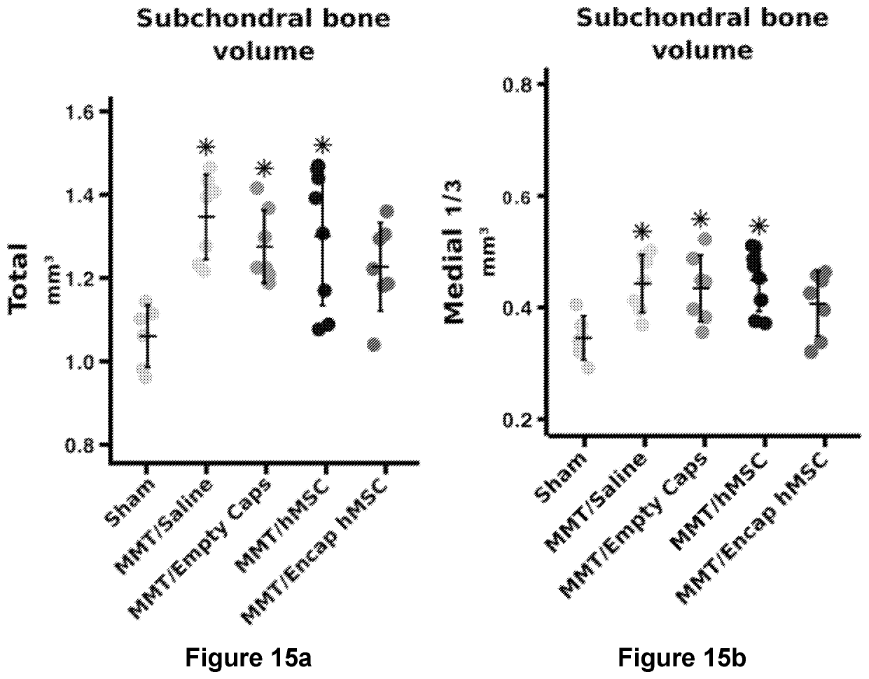

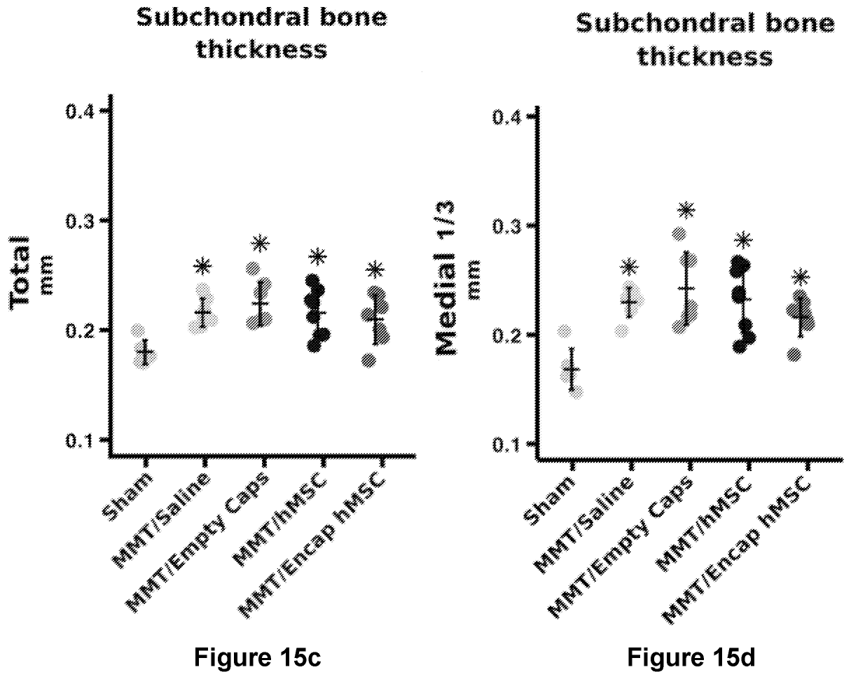

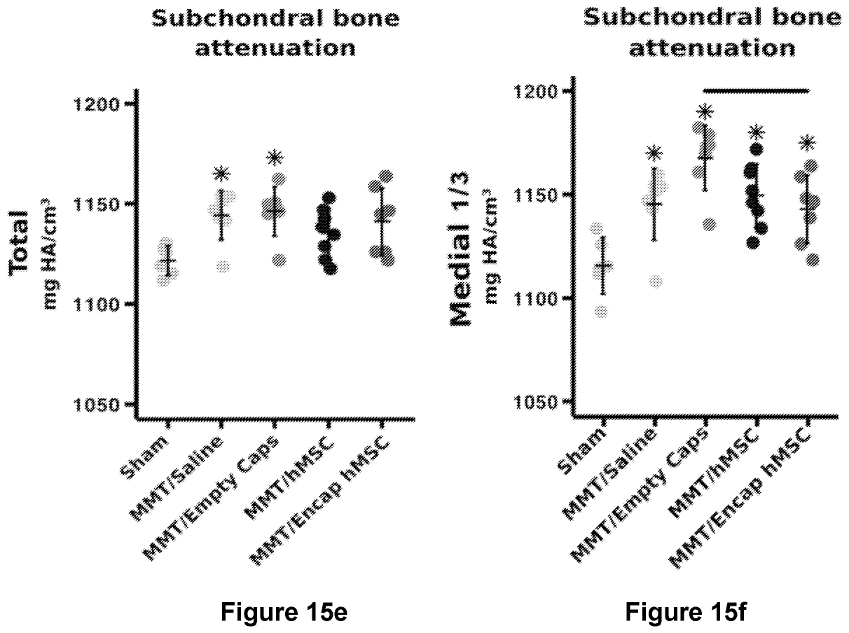

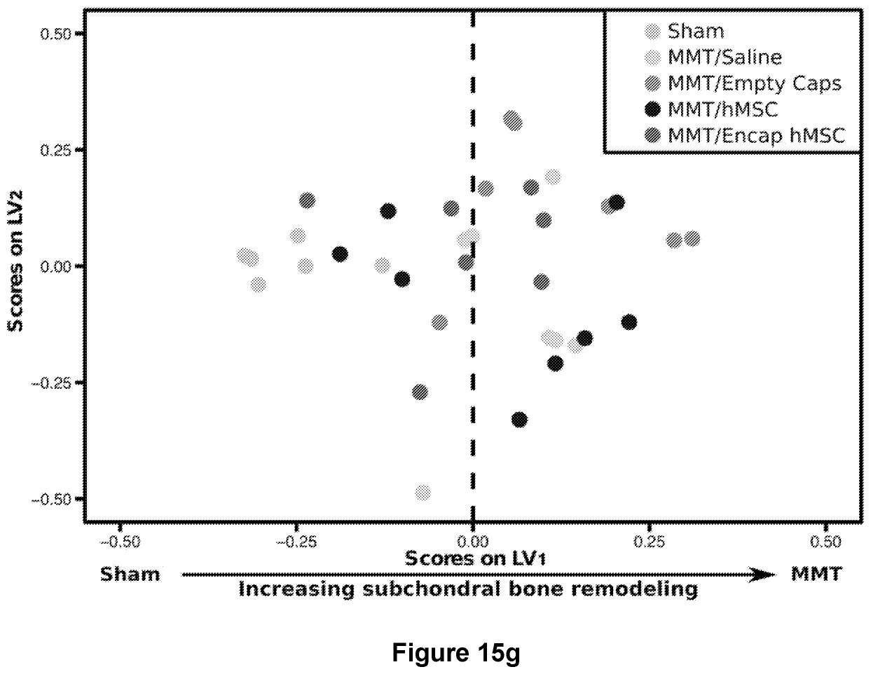

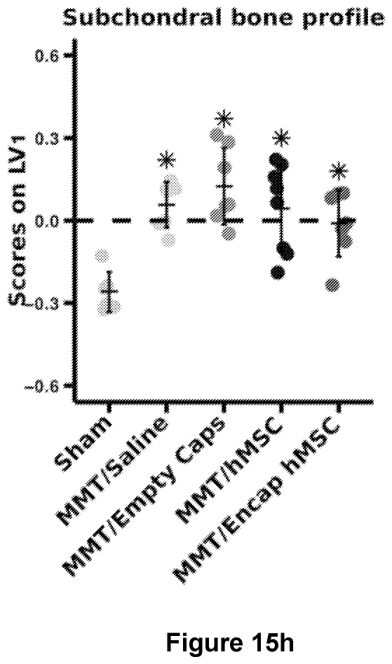

Detailed analysis of subchondral bone was performed on various morphological parameters for both the total and medial regions of the medial tibial condyle, as illustrated in FIGS. 15a-15h. Subchondral bone volume, for both total and medial regions, showed MMT/Saline, MMT/Empty Caps, and MMT/hMSC groups were significantly elevated relative to sham (FIGS. 15a, 15b). For the subchondral bone thickness parameter, all MMT groups demonstrated significantly increased values relative to sham (FIGS. 15c, 15d). Total attenuation was not significantly different between MMT/Encap hMSC and sham or between MMT/hMSC and sham (FIG. 15e). The other two MMT groups (MMT/Saline and MMT/Empty Caps) had a significant increase in attenuation values relative to the sham group, indicating increased bone mineral density (hardening) of the subchondral bone (FIG. 15e). However, the analysis of attenuation for the medial 1/3 plateau showed significantly higher values for all MMT groups compared to sham (FIG. 15f).

Cumulative analysis of the efficacy of encapsulated hMSCs on the subchondral bone layer in OA (accounting for volume, thickness, and attenuation parameters) was analyzed with PLSDA. LV1 separated out all study groups by levels of subchondral bone remodeling with sham separating out on the left from all MMT groups on the right (FIG. 15g). This finding was further confirmed with ANOVA on LV1 scores which demonstrated that there were no cumulative significant differences between respective MMT groups (FIG. 15h). Consideration of all these findings suggests encapsulated hMSCs yield a minimal therapeutic effect on subchondral bone in OA as this therapeutic yielded less bone thickening (volume) and hardening (attenuation). These disease modifying effects were similar to those found for articular cartilage analyses as further disease development from the time point of intervention was reduced. Furthermore, encapsulated hMSCs again did not provide a restorative effect as there was still significant subchondral bone thickening (thickness) and bone hardening (medial 1/3 attenuation).

As shown in FIGS. 15a-15b, total and medial 1/3 subchondral bone volumes for all MMT groups, except MMT/Encap hMSC, were significantly greater than the sham group. As shown in FIGS. 15c-15d, total and medial 1/3 subchondral bone thickness analysis yielded significant increases in all MMT groups relative to sham. As shown in FIG. 15e, MMT/Empty Caps and MMT/Saline total subchondral bone attenuation was significantly greater than the sham while showing no differences with MMT/hMSC and MMT/Encap hMSC groups. As shown in FIG. 15f, in the medial 1/3 region, all MMT groups had significantly greater attenuation values compared to shams; the only difference found between MMT groups was between MMT/Empty Caps and MMT/Encap hMSC. As shown in FIG. 15g, PLSDA analysis of total and medial 1/3 subchondral bone parameters depicted significant separation between sham, to the left, from all MMT groups to the right, along LV1 based on the level of subchondral bone remodeling. As shown in FIG. 15h, statistical analysis of these scores demonstrated a significant difference between the sham group and all the MMT groups, with no differences between the respective MMT groups. This data is presented as mean+/−SD; n=6 for sham, n=7 for MMT/Saline, n=7 for MMT/Empty Caps, n=8 for MMT/hMSC and n=7 for MMT/Encap hMSC. * represents significant differences (p<0.05) between individual MMT groups and sham. Horizontal black bars indicate significance (p<0.05) between individual MMT groups.

Histomorphometric Analysis of OA Tissues Demonstrate Reduced Disease Progression in Established OA with Encapsulated hMSC Treatment

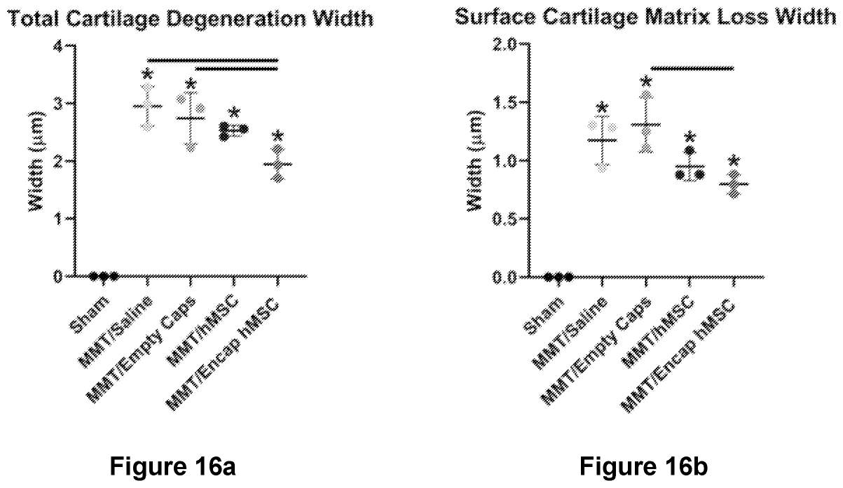

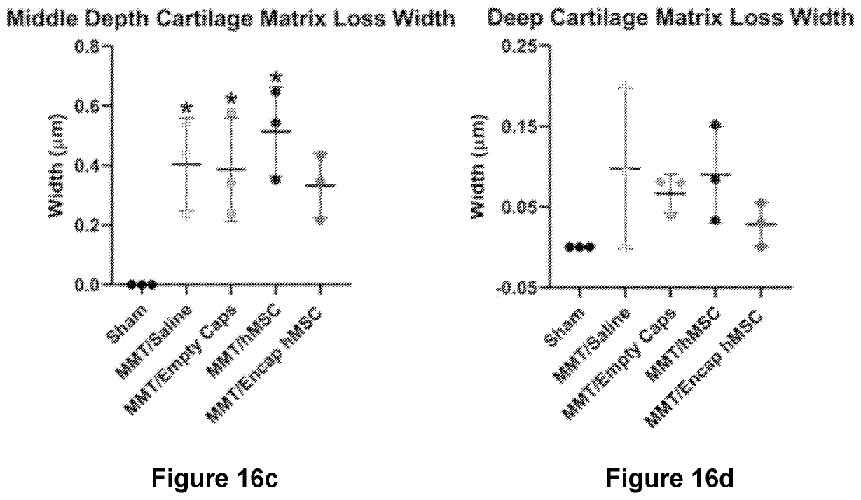

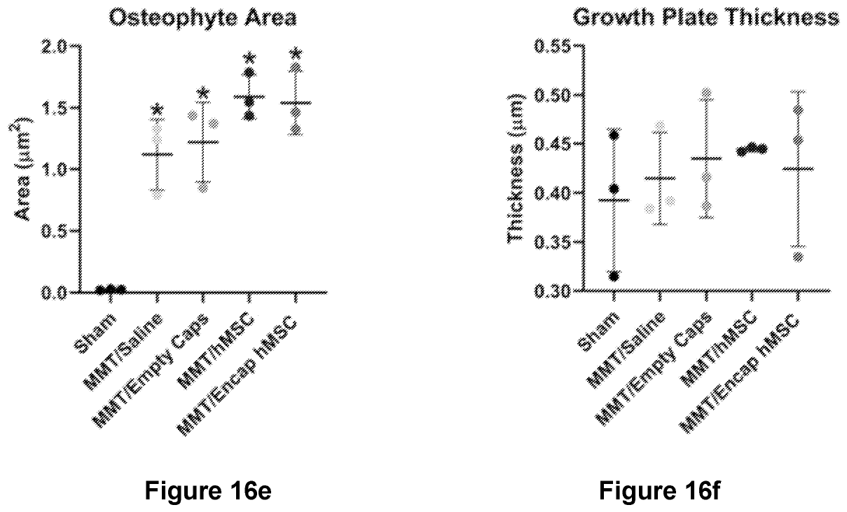

To confirm the findings of microCT analysis, histological scoring using GEKO was performed on rat tibiae, as illustrated in FIGS. 16a-16f. For total cartilage degeneration width, representing the total width showing any cartilage damage, the MMT/Encap hMSC group did not yield a significantly higher width in damage compared to sham but was significantly lower than that of the MMT/Saline and MMT/Empty Caps groups (negative controls; FIG. 16a). Cartilage surface matrix loss width, the width of surface cartilage lost due to a lesion, showed similar outcomes with all MMT groups showing significantly higher widths than the sham, with the MMT/Encap hMSC group yielding significantly lower width relative to MMT/Empty Caps (FIG. 16b). Furthermore, for middle depth cartilage matrix loss width, representing the width of the cartilage lost within a lesion at 50% the cartilage depth, the MMT/Encap hMSC group was the only MMT group to not show a significantly greater width than the sham, as all other MMT groups showed significant increases relative to sham (FIG. 16c). However, for deep cartilage matrix loss width, the width of cartilage lost within a lesion at full cartilage depth, no significant differences were found between any groups (FIG. 16d). These histological findings further support the microCT outcome measures as the MMT/Encap hMSC group was shown to demonstrate decreased overall articular cartilage degeneration.

As shown in FIG. 16a, for total cartilage degeneration width, all MMT groups yielded an increase degeneration width relative to sham; MMT/Encap demonstrated less degeneration width than both MMT/Saline and MMT/Empty Caps. As shown in FIG. 16b, surface cartilage matrix loss width yielded similar outcomes to total cartilage degeneration, with exception to no difference being yielded between MMT/Saline and MMT/Encap hMSC. As shown in FIG. 16c, for middle depth cartilage matrix loss width, all MMT groups, except MMT/Encap hMSC demonstrated increased loss width relative to sham. As shown in FIG. 16d, deep cartilage matrix loss width showed no significant differences between any of the groups assessed. As shown in FIG. 16e, for osteophyte area, all MMT groups showed significantly higher volumes than the sham control; however, no differences were noted between respective MMT groups. As shown in FIG. 16f, growth plate thickness showed no significant differences between any groups assessed. This data is presented as mean+/−SD; n=3 for all groups. * represents significant differences (p<0.05) between individual MMT groups and sham. Horizontal black bars indicate significance (p<0.05) between individual MMT groups.

The findings from the microCT analysis of osteophytes were also confirmed using GEKO histological scoring. While all MMT groups showed significantly higher osteophyte (mineralized+cartilaginous) volumes than sham no differences were detectable between the MMT groups (FIG. 16e).

Biomaterial Encapsulation of hMSCs Induced a Targeted Paracrine Response

Numerous studies have shown that biomaterials can alter MSC function, survival, and mechanotransduction; however, there is limited understanding of the effects encapsulation has on MSC cytokine secretion. To assess the effects of biomaterial encapsulation on the secreted cytokines from hMSCs in a simulated OA microenvironment, an in vitro cell culture model was used where the media was supplemented with the primary OA cytokine IL-1β. Cell viability immediately following encapsulation was 97.1±3.1%, after which cells were plated. Cells were either conditioned in media alone (+CTRL) or conditioned with IL-1β in media (+IL-1β).

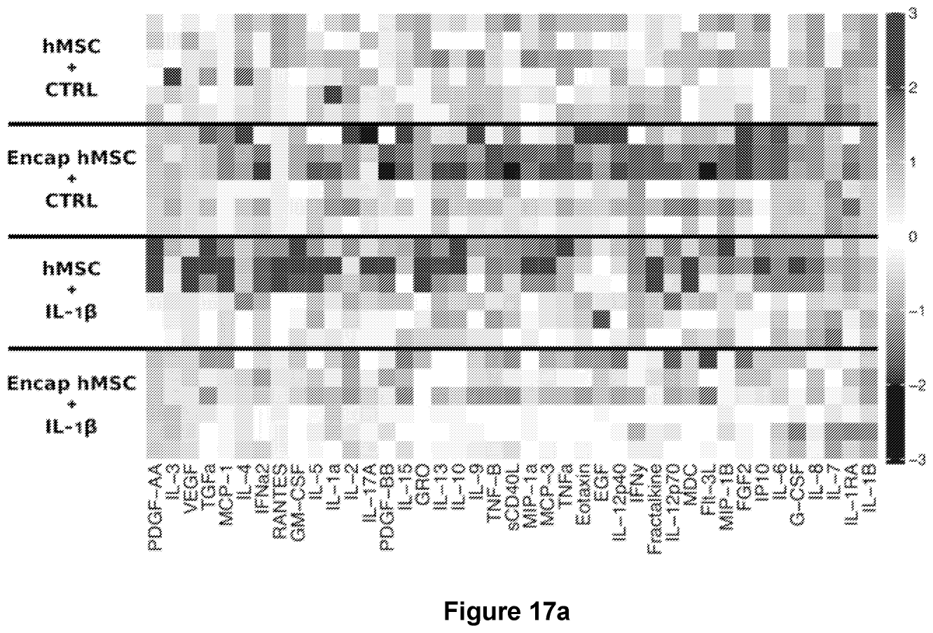

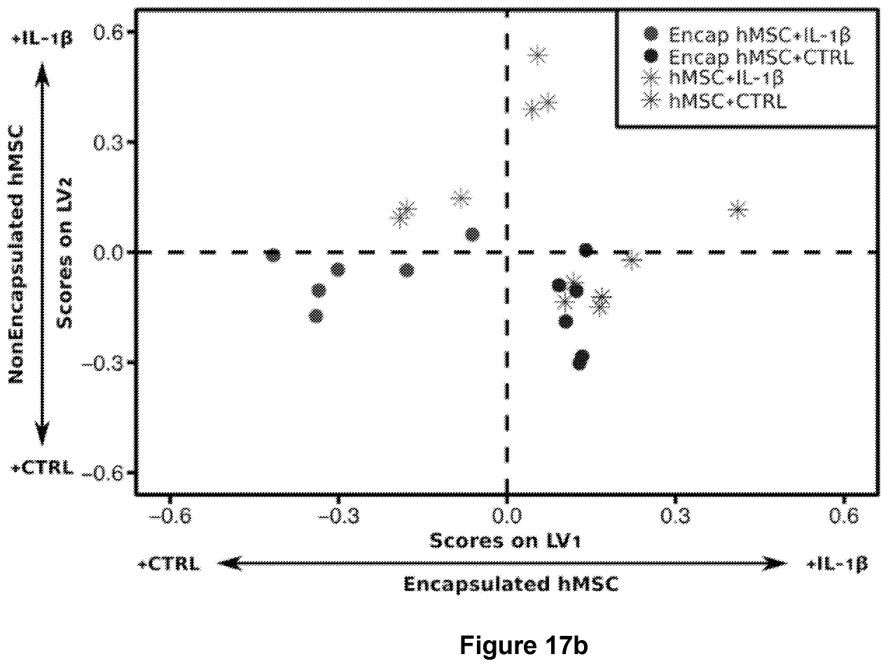

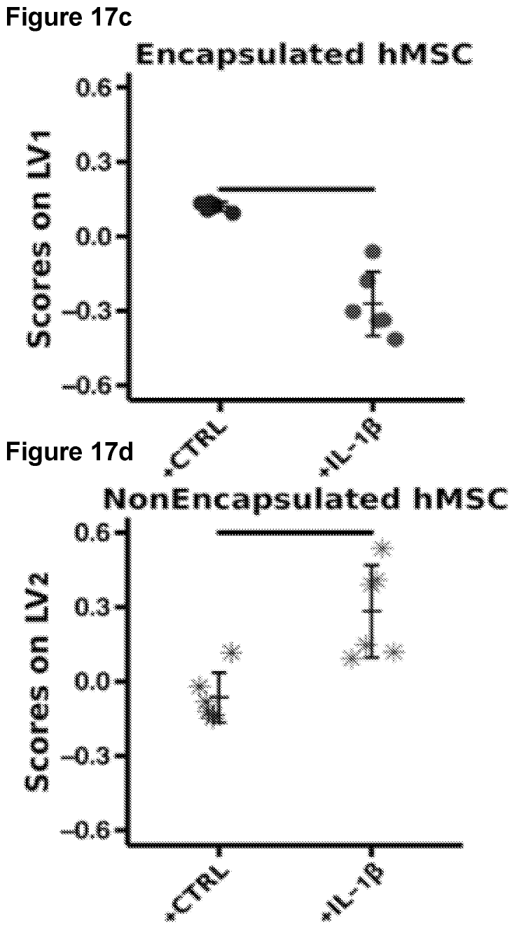

FIGS. 17a-17d illustrate quantification of paracrine signaling profiles of encapsulated and nonencapsulated hMSCs. Following treatment with or without IL-1β for 24 hours, media was collected and assessed for 41 immunomodulatory cytokines and chemokines (FIG. 17a). Background subtraction was performed for both unconditioned and conditioned conditions; all cytokine values only show the cytokine levels resulting from hMSC paracrine expression. Both nonencapsulated and encapsulated hMSCs were responsive to IL-1β conditioning when compared to CTRL conditions. For nonencapsulated hMSCs, IL-1β conditioning yielded indiscriminate upregulation of all measured cytokines, compared to the nonencapsulated hMSC control (+CTRL). In contrast, IL-1β conditioning of encapsulated hMSCs yielded a more targeted response with distinct qualitative increases in six cytokines: IL-1β, IL-1RA, IL-7, IL-8, Granulocyte Colony Stimulating Factor (G-CSF), and IL-6, relative to the encapsulated hMSC control (+CTRL). PLSDA revealed LV1 and LV2 axes that separated differentially mediated cellular paracrine responses with encapsulated hMSC conditions separating on LV1 (+IL-1β separated left and +CTRL separated right) and nonencapsulated hMSCs separating along LV2 (+CTRL at the bottom and +IL-1β at the top of the axis; FIG. 17b). Significant separation of latent variable scores was confirmed with t-tests of both LVs, with Bonferroni correction applied, for encapsulated and nonencapsulated hMSCs on the LV1 and LV2 axes, respectively (FIGS. 17c, 17d).

FIG. 17a shows a multiplexed immunoassay analysis of 41 cytokines (columns; z-scored) secreted from hMSCs with and without encapsulation in unconditioned (+CTRL) and conditioned environments (+IL-1β; each row represents a single sample). +IL-1β conditioning demonstrated increased paracrine cytokine secretion relative to +CTRL conditioning. Encapsulation, with +IL-1β conditioning, yielded a more targeted cytokine profile relative to nonencapsulated hMSCs. FIG. 17b illustrates PLSDA analysis identified two profiles of cytokines, LV1 and LV2, that identified a distinct separation between treatment groups for both encapsulated and nonencapsulated hMSCs. As shown in FIGS. 17c-17d, independent analysis of scores on each of the respective latent variables demonstrated significant differences between unconditioned and conditioned environments for both encapsulated and nonencapsulated hMSCs. This data is presented as mean+/−SD; n=6 for all groups. Horizontal black bars indicate significant differences between unconditioned and conditioned groups.

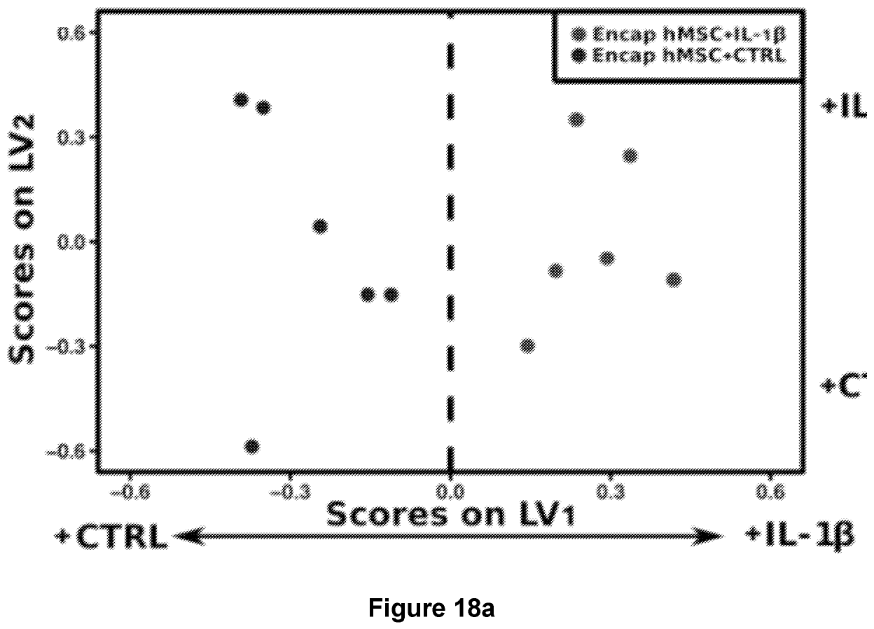

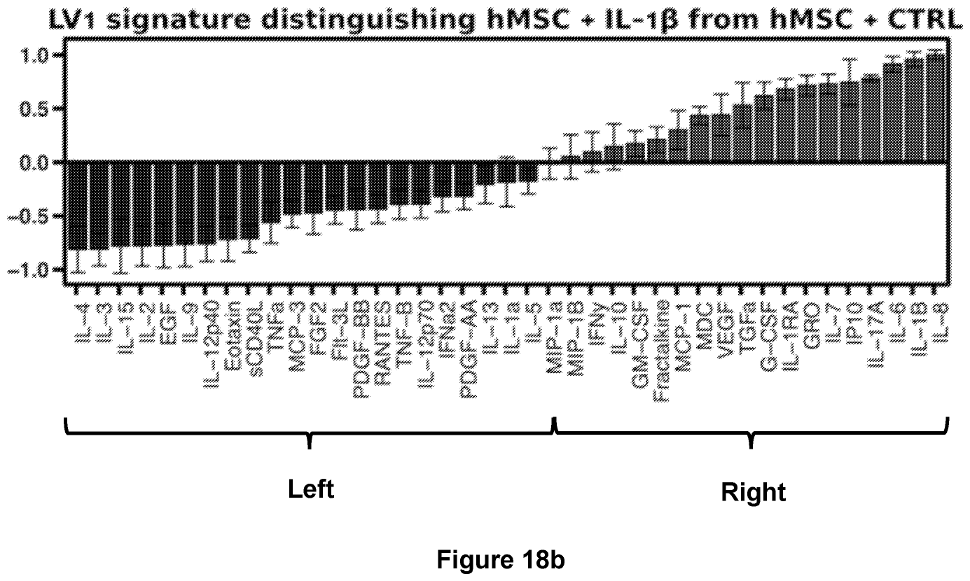

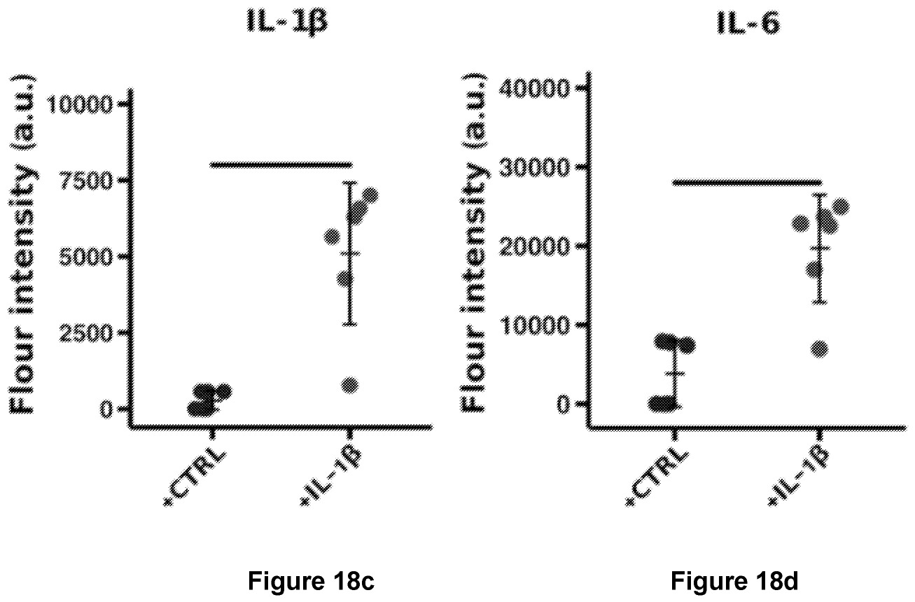

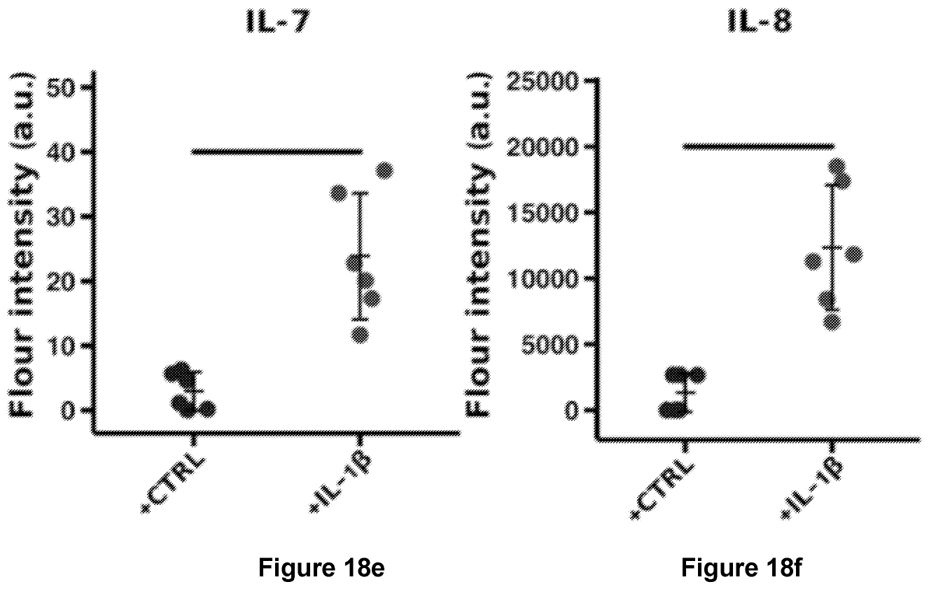

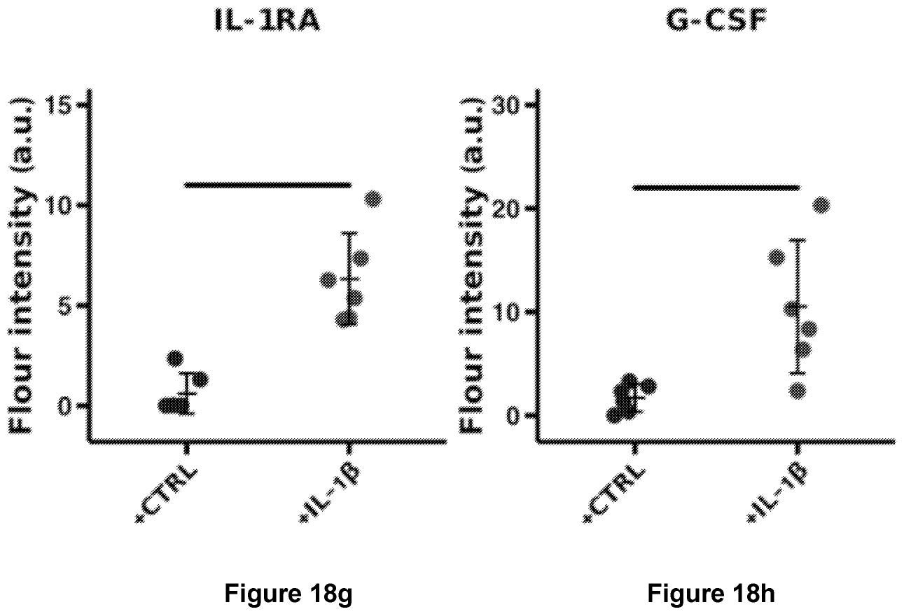

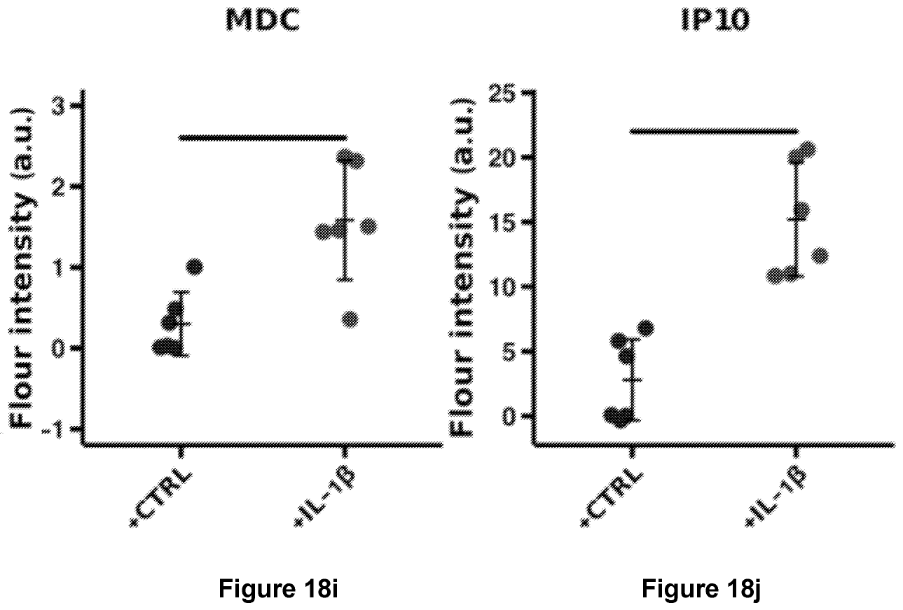

FIGS. 18a-18j illustrate identification of cytokines contributing to therapeutic efficacy of encapsulated hMSC therapeutics. To assess the effects of IL-1β conditioning on encapsulated cells, PLSDA was conducted on the encapsulated data alone (FIG. 18a). From LV1, the separation between CTRL to the left and IL-1β to the right can be clearly observed. LV1 consisted of a profile of cytokines that correlated with the CTRL group left) or IL-1β treated cells (right; FIG. 18b). To determine which cytokines yielded differences in cytokine expression (Encap hMSC+CTRL vs. Encap hMSC+IL-1β), univariate analysis was performed on all cytokines that were upregulated with IL-1β conditioning (right; FIG. 18b). Of the 18 cytokines that showed upregulation with IL-1β conditioning, eight were found to be significantly elevated, including the pro-inflammatory cytokines IL-1β, IL-6, IL-7, and IL-8, the anti-inflammatory cytokine IL-1RA and the chemokines G-CSF, macrophage derived chemokine (MDC; CCL-12), and interferon gamma-induced protein (IP) 10 (CXCL-10; FIGS. 18c-18j). These in vitro findings demonstrate a more mediated, and targeted response of encapsulated hMSCs when compared to the expression profile of nonencapsulated hMSCs, which yielded increased expression of all cytokines.

As shown in FIG. 18a, PLSDA analysis of encapsulated hMSCs identified a single latent variable, LV1, that distinguished between Encap hMSC+CTRL on the left and Encap hMSC+IL-1β to the right. As shown in FIG. 18b, the weighted profiles of cytokines showed relative expression of cytokines in CTRL conditions (left) and IL-1β conditions (right). Error bars on each cytokine were computed by PLSDA model regeneration using iterative (1000 iterations) LOOCV. As shown in FIGS. 18c-18j, all measured cytokines that showed significant increased expression with IL-1β conditioning were assessed independently, using t-test with Bonferroni correction, for significance between CTRL and IL-1β conditions, with all significant findings presented. Encap hMSCs+IL-1β yielded increased expression in pro-inflammatory (IL-1β, IL-6, IL-7, IL-8), anti-inflammatory (IL-1RA), and chemotactic (G-CSF, MDC, IP10) cytokines. This data is presented as mean+/−SD; n=6 for all groups. Horizontal black bars indicate significant differences between unconditioned (+CTRL) and conditioned (+IL-1β) groups.

### DISCUSSION

While encapsulated hMSCs elicited a pro-inflammatory response in the current Example, they also secreted anti-inflammatory cytokines and chemokines which may have therapeutic potential. Specifically, the anti-inflammatory cytokine IL-1RA has been studied extensively in the context of OA with pre-clinical studies demonstrating a protective capacity on articular cartilage [162,163]. Furthermore, a number of chemokines were increased (G-CSF, MDC, IP10) when conditioned with IL-1β, which would suggest that hMSCs could induce a response to recruit native stem and immune cells to the injury site. The cytokine IP10 (CXCL-10) was also upregulated by encapsulated hMSCs.

The data in this Example showed that hMSCs can exert a chondroprotective therapeutic effect through paracrine signaling, independent of direct engraftment, as encapsulated hMSCs yielded an early protective role on articular cartilage in OA. Furthermore, data in this Example showed the effects hMSCs, through their paracrine signaling properties, can have on osteophyte formation as encapsulated hMSCs increased osteophyte volumes. These increased tissue volumes are especially relevant in clinical applications as many clinical trials are currently ongoing but have not been investigating the effects of osteophyte development.

Though the encapsulated hMSCs provided a disease modifying protective effect shown in this Example, the treatment did not regenerate or restore the cartilage back to levels comparable to sham operated controls in either model of disease assessed. These data suggest that the timing of hMSC treatment in the OA disease progression can be critical, as this treatment protected the integrity of the remaining tissue and thus suggests that treatment during earlier disease stages (when there is still tissue to protect) may have longer and more potent therapeutic effects. Though protective effects were observed on the cartilage, encapsulated hMSCs yielded increased osteophyte volumes in both developing OA and established OA, which have been identified as an unwanted phenotype for restoring joint function. In addition to assessing in vivo therapeutic efficacy of these encapsulated hMSCs, this Example describes a novel in vitro OA simulated microenvironment to study the therapeutic profile of these hMSC therapeutics. Without wishing to be bound by theory, these paracrine signaling properties may be of particular interest for hMSC therapeutics due to their implications as a major mechanism of action for OA treatment. The immunomodulatory potential of biomaterial encapsulation on hMSC function demonstrated a targeted paracrine response to a simulated OA microenvironment while nonencapsulated hMSCs showed an indiscriminate upregulation of all cytokines in the cytokine panel. While expression of numerous anti-inflammatory and regenerative cytokines were increased with hMSC encapsulation, there were also a number of pro-inflammatory cytokines that showed increased expression. In considering these latter findings, it is important to consider that this hMSC paracrine response is an acute response and that the secretion of these pro-inflammatory cytokines may be critical in resolving the chronic OA inflammatory environment. Characterization of these cytokine profiles can provide valuable insight linking specific factors to therapeutic efficacy in vivo and provide an innovative approach for future assessment of MSC therapeutics for OA. Together, the data in this Example demonstrated that biomaterial encapsulation of hMSCs mediated the paracrine response to a simulated OA microenvironment and enhanced the in vivo therapeutic efficacy of the hMSCs in preventing further disease progression in treating established OA.

### EXAMPLE 2: Identification of Cellular Attributes of Therapeutic Human Mesenchymal Stromal Cells in Osteoarthritis

MSCs have been and continue to be studied extensively for their therapeutic efficacy as OA therapeutics in the clinic, as over 950 clinical trials have been registered with the FDA. Taken together, these studies have reported high variability in clinical outcomes and thus no consensus has been made on best practices in administering these therapeutics clinically. A major gap in knowledge that currently exists is the role that donor heterogeneity has on the efficacy of these therapeutics in OA. Prior research has demonstrated that MSCs isolated from different human donors exhibit large differences in proliferation, senescence, differentiation potential, and paracrine signaling activity. While prior research has demonstrated that therapeutic potency varies in MSCs isolated from different human donors for treatment of other disease states, this donor heterogeneity remains understudied for MSCs as OA therapeutics.

In current clinical trials for MSC treatment in OA, the SOCs typically involve isolating MSCs from the bone marrow of the iliac crest of clinical patients, expansion of these cells in vitro to appropriate cell dosage numbers followed by reinjection of these MSCs autologously back into the patients that they were isolated from. However, no screening metrics are implemented to assess for potency of these cells, as the screening criterion typically used to identify and isolate MSCs include: the cells ability to adhere to plastic and express standard MSC phenotypic cell surface markers. However, it has been demonstrated that this standard criterion employed to characterize MSCs (phenotypic characterization, differentiation potential, and adherence to plastic surfaces) defines a largely heterogenous population of cells. This absence of screening for additional cellular attributes is potentially a major factor contributing to the lack of success of these therapies in ongoing clinical trials as donor heterogeneity remains a critical factor for MSC efficacy.

In this Example, cellular attributes of therapeutic MSCs were studied given the data regarding paracrine signaling shown in Example 1. In MSCs, as with all mammalian cells, paracrine signaling is regulated by intracellular phospho-protein signaling which permit external stimuli (cytokines, growth factors, etc.) to be sensed by local cells using cell surface receptors. These signals are then internalized through intracellular phospho-protein signaling networks yielding transcription processes which can lead to the secretion of cytokines from these cells.

Cellular attributes from each of these critical steps (MSCs sensing of an external stimulus to the subsequent cytokine secretion by MSCs) were quantified to identify cellular attributes of hMSCs that relate to therapeutic hMSC paracrine signaling properties in OA. The primary objective of this Example was to assess hMSC donor heterogeneity to identify secreted cytokines, RNA transcripts, and intracellular signaling phospho-proteins (see Example 3 herein) of hMSCs that relate to the therapeutic efficacy of hMSCs in OA. More specifically, a multiplexed immunoassay was implemented to quantify cytokine secretion and RNA-Seq was used to quantify gene expression in four unique hMSC donors in vitro in an OA simulated microenvironment. An in vivo preclinical established model of OA (6-week MMT) was used to assess therapeutic efficacy of the four unique hMSC donors (matching those used in the in vivo analysis) as it serves as a more clinically relevant model, as patients present to the clinic following the presentation of disease manifestations after OA has developed. Correlation analysis, PLSR, and PCA were used to identify hMSC cellular attributes that relate to the therapeutic efficacy of hMSCs in OA (in vivo). It is suggested that a profile of hMSC cellular attributes could be identified in an OA in vitro microenvironment that relate to the therapeutic efficacy of hMSCs in OA in vivo.

Materials and Methods hMSC Culture and Characterization hMSCs derived from bone marrow were obtained from EPIC core facility at Emory University and RoosterBio (MSC-004; RoosterBio, Inc). EPIC hMSCs (2.1 and 2.2) were cultured in Mesenchymal Stem Cell Basal Medium (PT-3238; Lonza) supplemented with 10% heat-inactivated FBS (S11110H; Atlanta Biologicals), 1 mM L-glutamine (SH3003401; HyClone), and 100 μg/mL P/S (B21110; Atlanta Biologicals) and sub-cultured at 80% confluency. Rooster hMSCs were cultured in RoosterNourish-MSC (KT-001; RoosterBio, Inc) media supplemented with 2% RoosterBooster (SU-003; RoosterBio, Inc) and sub-cultured at 80% confluency. hMSC phenotypes for all donor hMSCs were confirmed by adipogenic, chondrogenic, and osteogenic differentiation. Flow cytometry was also used to characterize the hMSCs to confirm that all donor cells expressed characteristic MSC surface markers (CD73, CD90, CD105) and lacked hematopoietic markers (CD45, CD34, CD11b, CD79A, HLA-DR). Metadata is also included for all hMSC donors, as provided below in Table 1.

TABLE 1
Metadata for hMSC donors used.

| donor # | Source | Sex | Age | Tissue | Passage |
|---|---|---|---|---|---|
| 1.1 | EPIC | — | — | Bone Marrow | 4 |
| 2.1 (L1) | EPIC | F | 5 | Bone Marrow | 4 |
| 2.2 (L2) | EPIC | M | 4-20 | Bone Marrow | 4 |
| 2.3 (M1) | RoosterBio | M | 18-30 | Bone Marrow | 4 |
| 2.4 (M2) | RoosterBio | F | 26 | Bone Marrow | 4 |

In vivo MMT Model of OA

Animal care and experiments were conducted in accordance with the institutional guidelines of the VAMC and experimental procedures were approved by the Atlanta VAMC IACUC (Protocol: V004-15). Weight-matched wild type male Lewis rats (strain code: 004; Charles River), weighing 300-350 g, were acclimatized for 1 week after they were received. A surgical instability animal model, MMT, was used to induce OA. Animals were anesthetized using isoflurane and injected subcutaneously with 1 mg/kg sustained-release buprenorphine (ZooPharm). Skin over the medial aspect of the left femoro-tibial joint was shaved and sterilized. Blunt dissection was used to expose the MCL, which was next transected to expose the meniscus. Then, a full-thickness cut was made through the meniscus at its narrowest point. Following transection of the meniscus, soft tissues were re-approximated and closed using 4.0 Vicryl sutures and the skin was closed using wound clips. sham surgery was also performed in rats. For shams, the MCL was transected followed by closure of the skin without transection of the meniscus.

To assess the effects of hMSC therapeutics for preventing further development of establish OA a 6-week time course study was implemented, with therapeutics injected at 3 weeks corresponding to OA phenotype presentation followed by animal takedown 3 weeks later at the 6-week end point. All MMT animals received 50 μL intra-articular injections using a 25-gauge needle. Animals were injected with 1) HBSS (MMT/Saline; n=8), 2) 5×10⁵ hMSC/knee of donor 2.1 [MMT/hMSC(2.1); n=8], 3) 5×10⁵ hMSC/knee of donor 2.2 [MMT/hMSC(2.2), 4) 5×10⁵ hMSC/knee; n=8] of donor 2.3 [MMT/hMSC(2.3); n=7], 5) 5×10⁵ hMSC/knee of donor 2.4 [MMT/hMSC(2.4); n=8]. The cell dose used for injection was 5×10⁵ cells/knee. Sham animals were not injected post-surgery (n=8).

Tissue Preparation for microCT and Histology

Animals were euthanized at 6-weeks post-surgery via CO₂ asphyxiation. Cervical dislocation was used as a secondary euthanasia method after asphyxiation. Left hindlimbs were dissected and fixed in 10% neutral buffered formalin. Muscle and connective tissues were removed from the hindlimbs. The femur was disarticulated from the tibia. Meniscus and residual soft tissue surrounding the medial tibial condyle were dissected and discarded. microCT Quantitative Analysis of Articular Joint Parameters

Prior to scanning, all muscle and connective tissue from collected fixed hind limbs was removed, the femur was disarticulated from the tibia, and all peripheral connective tissue surrounding the joint was removed to expose the articular cartilage of the medial tibial plateau. Exclusion criteria for study tissue samples included nicks of the medial articular cartilage surface (incurred either in the MMT surgical procedure or in dissection of the tissue samples), dissection error in the intercondylar area (due to dissection error), or loss of osteophyte structures on the medial edge of the joint (as a result of dissection error). All damage to the tissue samples was noted during the dissection stage and verified with microCT. Tibiae were immersed in 30% (diluted in PBS) hexabrix 320 contrast reagent (NDC 67684-5505-5; Guerbet) at 37° C. for 30 mins before being scanned. All samples were scanned using microCT through the use of a Scanco μCT 40 (Scanco Medical) using the following parameters: 45 kVp, 177 μA, 200 ms integration time, isotropic 16 μm voxel size, and an about 27 min scan time. Scans were read out as 2D tomograms which were subsequently orthogonally transposed to yield 3D reconstructions for all scanned samples. All microCT parameters (articular cartilage, osteophyte, and subchondral bone) were evaluated as previously described (FIGS. 3a-3i). For cartilage parameters, thresholding of 110-435 mg hydroxyapatite per cubic cm (mg HA/cm³) was used to isolate the cartilage from the surrounding air and bone. Furthermore, for bone parameters, thresholds of 435-1200 mg HA/cm³ were implemented to isolate bone from the overlying cartilage.

Coronal sections were both evaluated along the full length of the cartilage surface (total) and in the medial region of the medial tibial condyle. The medial 1/3 region of the articular cartilage was analyzed as this region has been shown to demonstrate high damage incidence in the MMT model. For articular cartilage, volume, thickness, and attenuation parameters were quantified. Attenuation is inversely related to sGAG content. In OA, sGAG concentration in articular cartilage decreases due to degradation, creating a gradient which leads to an increased hexabrix concentration in the cartilage. High hexabrix and low sGAG levels (increased sGAG loss) correspond to a higher attenuation value. In addition to microCT analysis of articular cartilage, osteophyte volumes found on the most medial aspect of the medial tibial plateau were evaluated for their cartilaginous and mineralized portions. Osteophytes are a thickening and partial mineralization of cartilage tissue at the marginal edge of the medial tibial plateau and are a staple of OA development. Osteophytes consist of cartilaginous and mineralized portions, as they undergo an endochondral-like ossification process in formation. Additionally, subchondral bone was evaluated for volume, thickness, and attenuation (indirect measure of bone mineral density) along the total and medial regions, similar to the approach used for articular cartilage analysis.

### MATLAB Articular Cartilage Surface Roughness Analysis

Serial 2D images of the proximal tibiae were analyzed using a customized algorithm in MATLAB (Math Works) to quantify surface roughness, lesion volume, and full-thickness lesion area. Images were processed to generate a 3D surface of the articular cartilage surface, as illustrated in FIGS. 4a-40. This 3D rendering was fitted along a 3D polynomial surface: fourth order along the ventral-dorsal axis and second order along the medial-lateral axis.

The root mean square difference between the generated (actual) and polynomial fitted (predicted) surfaces was the measure of cartilage layer surface roughness (FIGS. 4d-4g). Lesion volume (FIGS. 4h-4k) was calculated as the volume of root mean square difference between the generated fitted surface and the polynomial surface where >25% of total (predicted) cartilage thickness was exceeded. Full-thickness lesion area (FIGS. 4l-4o), also called exposed bone, was the sum of the area on the tibial condyle where no cartilage layer was present. Surface roughness, lesion volume and full-thickness lesion area were calculated for full and medial 1/3 region of the articular cartilage.

In vitro OA Simulated Microenvironment hMSCs, matching donor with in vivo MMT model, were utilized in vitro. hMSCs were sub-cultured to 80% confluency in complete Lonza for hMSC donors 2.1 and 2.2 and Rooster medium for hMSC donors 2.3 and 2.4 in 12-well plates and cultured at 37° C., 5% CO₂. For encapsulated hMSCs, immediately following encapsulation and washing, cells were placed in monolayer culture in treatment medium (unconditioned or conditioned) in 12-well plates at 37° C., 5% CO₂.

TABLE 1
Metadata for human synovial fluid (SF)
control samples collected from cadavers.
Synovial fluid control

| ID | Cause of death | Sex | Age | Race |
|---|---|---|---|---|
| SF-1435 | Respiratory failure | M | 63 | Caucasian |
| SF-1458 | Cardio-pulmonary failure | M | 77 | Caucasian |
| SF-1466 | Alcoholic liver failure | M | 48 | Caucasian |

Each of the four hMSC donors were independently conditioned with 1) unconditioned media (+CTRL), 2) 20 ng/ml IL-1β conditioned media (FHC05510; Promega; +IL-1β), 3) 10% SF control conditioned media (+SF CTRL), and 4) 10% OA SF conditioned media (+OA SF), respectively. IL-1β was used to model the OA inflammatory environment in the current study as it is a major pro-inflammatory modulator in OA. IL-1β and SF concentrations were selected based on prior experiments and preliminary data. Control SF (Articular Engineering LLC) was pooled (3×donors; Table 2) from collections from cadaveric donors within 48 hours of death with no prior clinical diagnosis of OA. OA SF was pooled (6×donors; Table 3) from collections from patients in the clinic receiving knee aspirate procedures with a prior clinical diagnosis of OA. Patients were recruited from Emory University Sports Medicine under an Institutional Review Board (IRB) protocol (IRB00090172); all patients gave informed consent. OA SF was directly removed from OA patients by an orthopaedic physician. The immunomodulatory cytokine content of all SF samples was characterized using a bead based multiplex immunoassay, Luminex Cytokine/Chemokine 41 Plex Immunomodulatory Kit (HCYTMAG-60K-PX41; EMD Millipore Corporation). Median fluorescent intensity values were read out using Luminex xPONENT software V4.3 in the MAGPIX system.

TABLE 2
Metadata for human OA synovial fluid
samples collected from clinical patients.
OA synovial fluid

| ID | Date collected | Sex | Age | KL grade |
|---|---|---|---|---|
| SF-1 | Feb. 7, 2018 | M | 67 | 3 |
| SF-3 | Oct. 12, 2018 | M | 67 | 2 |
| SF-5 | Feb. 9, 2018 | F | 70 | 2 |
| SF-6 | Nov. 2, 2018 | M | 80 | 4 |
| SF-7 | Nov. 2, 2018 | M | 53 | 2 |
| SF-9 | May 29, 2018 | F | 54 | 1 |

SF samples were kept frozen in 1 mL aliquots at −80° C. until use. Conditioned media was added to hMSCs at day 0 followed by a 24-hr conditioning period in monolayer culture with media collection (Luminex cytokine analysis) and cell lysate collection (RNA-Seq analysis) at the study end point. Samples were stored at −80° C. until analysis was performed.

hMSC Cytokine Analysis

Cytokines were quantified for all donors with +CTRL (n=3), +IL-1β (n=3), +CTRL SF (n=3), and +OA SF (n=3) conditioned media, respectively. Loaded samples (2.03 μL) were determined to be within the linear range of detection of the MAGPIX (MAGPIX-XPON4.1-CEIVD; EMD Millipore Corporation) system. Cytokines were quantified using a bead based multiplex immunoassay, Luminex Cytokine/Chemokine 41 Plex Immunomodulatory Kit (HCYTMAG-60K-PX41; EMD Millipore Corporation) and 3 Plex TGF-Beta Kit (TGFBMAG-64K-03; EMD Millipore Corporation). Median fluorescent intensity values were read out using Luminex xPONENT software V4.3 in the MAGPIX system. Background subtraction was performed on CTRL, IL-1β, CTRL SF, and OA SF conditions using read out values from media only, 20 ng/ml IL-1β conditioned media, SF control conditioned media, and 10% OA SF conditioned media, respectively.

hMSC RNA-Seq Analysis

RNA transcripts were quantified for all hMSC donors with +IL-1β conditioned media only (due to outcomes from the cytokine analysis study). RNA was isolated from hMSCs using the Qiagen® RNeasy kit (217804; Qiagen®) according to the manufacturer's protocols. RNA samples were submitted to the Molecular Evolution Core at the Georgia Institute of Technology for sequencing. Quality Control was run on all samples using a bioanalyzer to determine that the RNA Integrity Number (RIN) of the samples was greater than 7. A NEBNext Poly(A) mRNA Magnetic Isolation Module (E7490S; New England Biolab) and NEBNext Ultra II Directional RNA Library Prep Kit (E7760; New England Biolab) were used to generate libraries for sequencing. Quality control was run on all generated libraries using the bioanalyzer and the library was quantified using fluorometric methods. Sequencing was performed using the NovaSeq 6000 Sequencing System to obtain a sequencing depth of 30-40 million reads per sample. Transcripts obtained were aligned using the Homo sapiens (human) genome assembly GRCh37 (hg19) from the genome reference database and all duplicate reads were eliminated. RNA levels were calculated in reads per kilobase per million mapped reads (RPKM).

### Gene Set Variation Analysis (GSVA)

To establish differences in each gene set, GSVA was used to identify enrichment of gene sets across all donors. GSVA is an improved gene set enrichment method that detects subtle variations of pathway activity over a sample population in an unsupervised manner. The GSVA was conducted using the Molecular Signatures Database C2 and C7 gene sets (MSigDB). Statistical differences in enrichment scores for each gene set between groups were computed by comparing the true differences in means against the differences computed by a random distribution obtained by permuting the gene labels 1000 times. False discovery rate (FDR) adjusted p-values were computed for detection of differences between donors statistical significance was set at FDR<0.25. GSVA was performed using the GSVA v1.36.1 in R (The R Foundation).
Correlation Analysis

To determine the association of hMSC cellular attributes (cytokines, RNA transcripts, and intracellular signaling phospho-proteins) with in vivo therapeutic outcomes correlation analysis was conducted. To quantify the relationship between therapeutic outcomes and hMSC cellular attributes, a least squares linear regression model was fitted and Pearson's correlation coefficient, R, was reported along with the p-values calculated from an F test based, with the null hypothesis that the overall slope is zero. Correlation matrices were generated using the cor function in R (The R Foundation).

### Principal Component Analysis (PCA)

PCA was conducted in R using the stats package v.3.6.2. Prior to inputting the data into the algorithm, all data was z-scored [(observed-mean)/SD]. An orthogonal rotation in the principal component (PC) 1-PC 2 plane was used to obtain new PC's that better separated treatment groups based on maximizing variance of the data set (covariance=0). RNA transcript read outs were used as the independent variable, while no dependent variable was set as PCA is an unsupervised approach.

### Partial Least Squares Discriminant Analysis

PLSDA was performed in MATLAB (Mathworks) using a function written by Cleiton Nunes (Mathworks File Exchange). This approach accounts for the multivariate nature of the data without overfitting. Prior to inputting the data into the algorithm, all data was z-scored [(observed-mean)/SD]. Secreted cytokines level read outs were used as the independent variables and the different hMSC donor and OA simulated conditions combinations were used as the outcome variables. LVs in a multidimensional space (dimensionality varied by number of independent input variables) were defined and the two primary LVs were used for orthogonal rotation to best separate groups in the new plane defined by LV1 and LV2 (FIGS. 5a-5c).

### Partial Least Squares Regression (PLSR)

PLSR was performed in MATLAB (Mathworks) using the built in plsregress function. This approach accounts for the multivariate nature of the data without overfitting. Prior to inputting the data into the algorithm, all cytokines were z-scored [(observed-mean)/SD]. For determination of cellular attributes of hMSCs four separate sets of inputs were used, independently, 1) cytokines, 2) RNA transcripts, 3) intracellular signaling phospho-proteins, and 4) all three in combination with the outputs for all these models being microCT outcomes (articular cartilage, osteophyte, and subchondral bone analyses) from all four donors used. LVs in a multidimensional space (dimensionality varied by number of independent input variables) were defined and the two primary LVs were used for orthogonal rotation to best separate groups in the new plane defined by LV1 and LV2 (FIG. 5a). Loadings plots were generated from this analysis and display the relative importance of input variables (microCT parameters or cytokines) in contributing to the final composite values (scores) for each sample (FIGS. 5b, 5c). Error bars on each cytokine (in the loadings plots) were computed by PLSR model regeneration using iterative (1000 iterations) LOOCV. To further confirm significant differences between groups assessed in PLSDA, the true differences in centroids (center of mass) of all groups were compared against the differences computed by a random distribution obtained by permuting the group labels 100 times. For each test, true group assignment showed Pₚₑᵣₘᵤₜₑ<0.05 compared to the randomly permuted distribution, further confirming the validity of the data. PLSDA was implemented for data obtained from the OA simulated microenvironment cytokine secretion data serving as the independent variable and each of the four donors in each condition (CTRL, IL-1β, CTRL SF, and OA SF) as the categorical outcome variable. PLSDA was performed in MATLAB (Mathworks) using a function written by Cleiton Nunes (Mathworks File Exchange).

### Statistical Analysis

A priori power analysis was run using α=0.05 and β=0.2, giving a power level of 0.8. From the power analysis, using medial 1/3 cartilage thickness as the primary outcome measure, it was determined that a sample size of at least 8 animals per group, for the MMT study, was necessary to find statistical differences between treatment groups. A post-hoc power analysis using α=0.05 and β=0.2, power=0.8, using average cytokine levels (average cytokine levels for all cytokines quantified), determined that a sample size of at least 3 was necessary to find statistical differences between treatment groups for the cytokine and RNA-Seq analyses, respectively. All data is presented as mean+SD. Significance for all microCT parameters was determined with a linear mixed model. This approach was employed as the to account for the non-independent nature of the microCT data (multiple hMSC donors) to assess both 1) therapeutic efficacy of each hMSC donor, relative to controls and 2) differences between hMSC donors. Post hoc analysis of Tukey honest test for articular cartilage and subchondral bone parameters and Bonferroni correction for exposed bone and osteophyte parameters (nonparametric) were used to determine differences between all treatment groups. Statistical significance was set at p<0.05. All data were analyzed using the R stats, ggsignif, and ggpubr packages in R (The R Foundation).
Characterization of hMSCs

hMSC differentiation was confirmed for all four hMSC donors with immunofluorescent staining for type II collagen of paraffin-sectioned pellets, oil red O staining, and alizarin red S staining for chondrogenesis, adipogenesis, and osteogenesis, respectively. Additionally, all hMSC donors were confirmed to be positive for typical MSC markers, including CD73, CD90, and CD105, and negative for hematopoietic markers, including CD45, CD34, CD11b, CD79A, and HLA-DR.
hMSCs From Different Donors Yield Variable Therapeutic Outcomes on Articular Cartilage

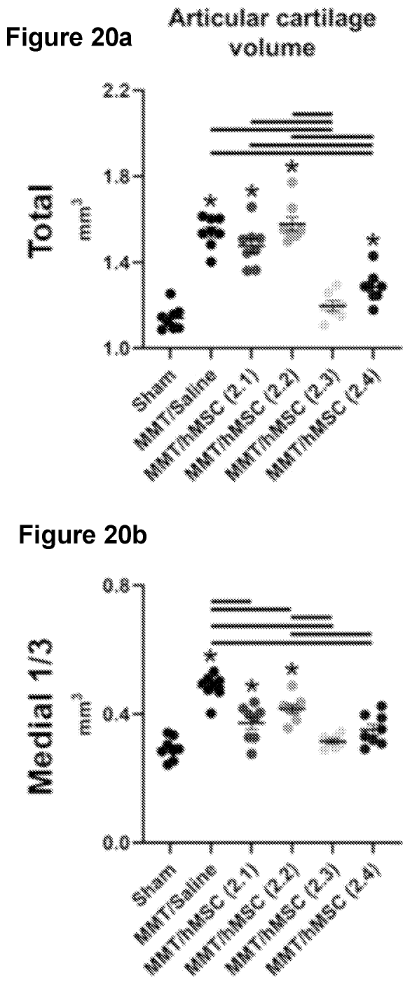

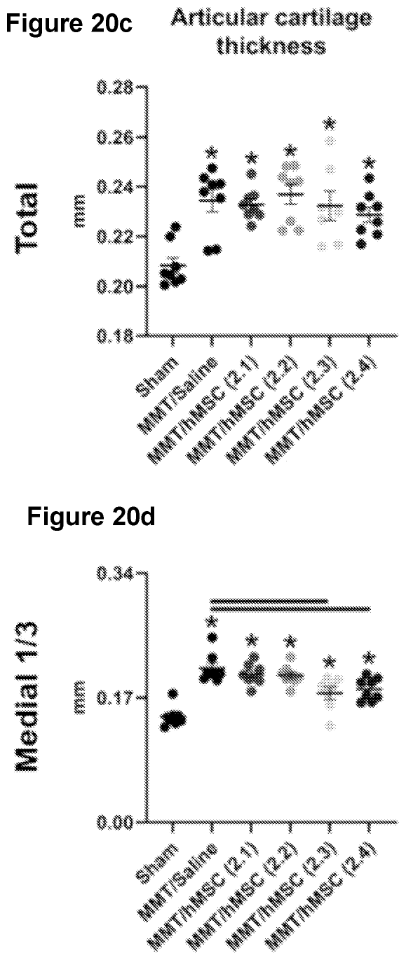

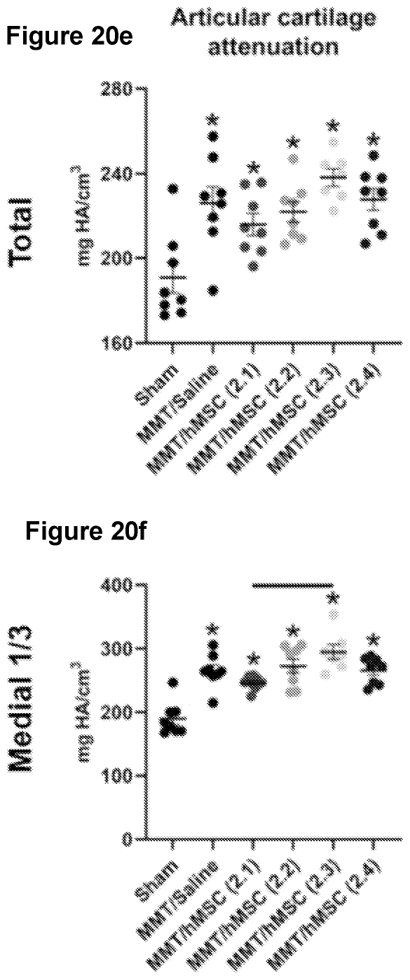

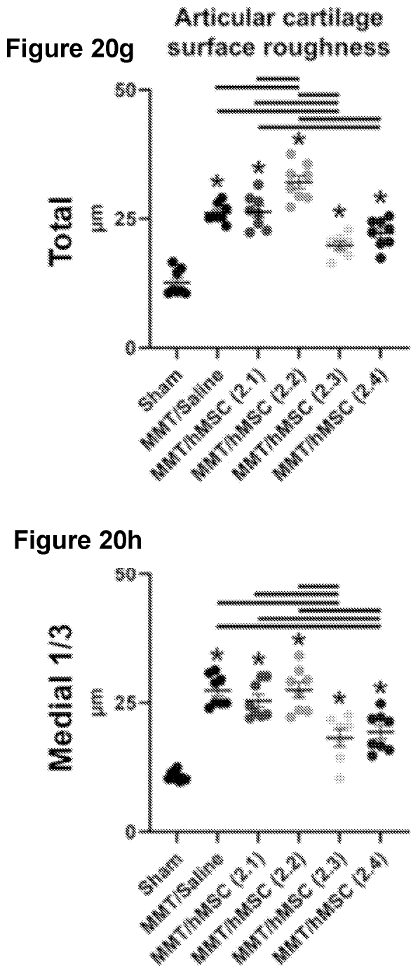

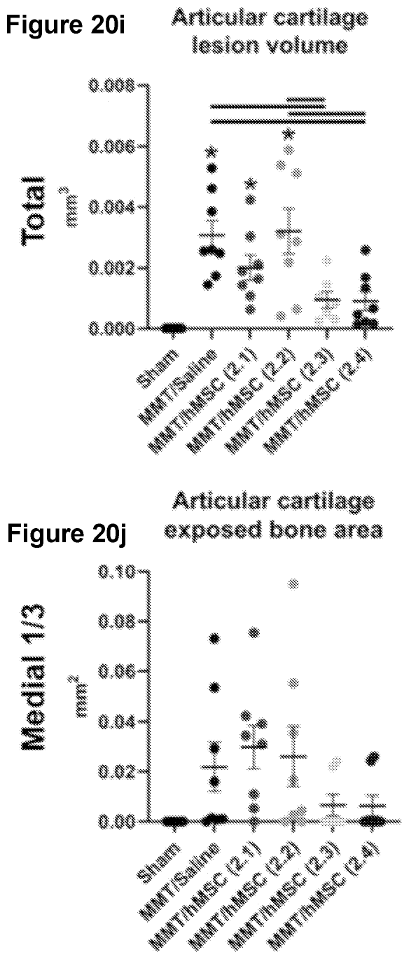

To assess donor heterogeneity of hMSCs as therapeutics for OA detailed quantitative analysis of the articular cartilage changes in established OA were performed on various morphological parameters for the total and medial 1/3 of the medial tibial condyle, as illustrated in FIGS. 20a-20j. For total articular cartilage volume, donors MMT/hMSC(2.3) and MMT/hMSC(2.4) showed reduced increases in articular cartilage volume relative to the MMT/Saline disease control; however, donors MMT/hMSC(2.1) and MMT/hMSC(2.2) showed no difference in volume with the MMT/Saline disease control (FIG. 20a). Similar outcomes were observed in the medial 1/3 region for articular cartilage volume (FIG. 20b). For articular cartilage thickness volume, while no differences were observed between MMT conditions for the total analyses in the medial 1/3 region donors MMT/hMSC(2.3) and MMT/hMSC(2.4) showed reduced articular cartilage thickness relative to the MMT/Saline disease control (FIGS. 20c, 20d). For articular cartilage attenuation, which is inversely related to articular cartilage proteoglycan content, no notable differences were observed between hMSC donors as all MMT conditions showed significantly higher attenuation in both the total and medial 1/3 analyses (FIGS. 20e, 20f). For total articular cartilage surface roughness, donors MMT/hMSC(2.3) and MMT/hMSC(2.4) showed reduced increases in articular cartilage surface roughness relative to the MMT/Saline disease control; however, donors MMT/hMSC(2.1) and MMT/hMSC(2.2) showed no difference in surface roughness with the MMT/Saline disease control (FIG. 20g). Similar outcomes were observed in the medial 1/3 region for articular cartilage surface roughness (FIG. 20h). For articular cartilage lesion volume, donors MMT/hMSC(2.3) and MMT/hMSC(2.4) showed reduced lesion volume relative to the other donors as they showed no significant increase relative to the sham, while MMT/hMSC(2.1) and MMT/hMSC(2.2) did; furthermore, donors MMT/hMSC(2.3) and MMT/hMSC(2.4) showed significantly less lesion volume relative to donors MMT/hMSC(2.1) and MMT/hMSC(2.2; FIG. 20i). No differences were observed between any MMT conditions for the exposed bone area (FIG. 20j). Overall, these metrics reveal that hMSC donors 2.3 and 2.4 yield more therapeutic outcomes, relative to hMSC donors 2.1 and 2.2, as they yielded reduced increases in articular cartilage swelling (volume and thickness), fibrillation development (surface roughness), and development of lesions (lesion volume). Based on the outcomes of this articular cartilage analysis, the more therapeutic hMSC donors (2.3 and 2.4) were relabelled M1 and M2 while the less therapeutic donors (2.1 and 2.2) were relabelled L1 and L2.

As shown in FIG. 20a, for total articular cartilage volume, all MMT groups except MMT/hMSC(2.3) demonstrated significant increases relative to sham; between MMT groups both MMT/hMSC(2.3) and MMT/hMSC(2.4) demonstrated reduced increases in volume. As shown in FIG. 20b, in the medial 1/3 region MMT/Saline, MMT/hMSC(2.1), and MMT/hMSC(2.2) showed significant increases in articular cartilage volume; all MMT/hMSC donor groups yielded reduced articular cartilage volume in the medial 1/3 region. As shown in FIG. 20c, for articular cartilage thickness, all MMT groups showed significant increases in thickness relative to sham, however no differences were noted between respective MMT groups. As shown in FIG. 20d, the medial 1/3 region showed similar outcomes to total thickness assessment except both the MMT/hMSC(2.3) and MMT hMSC(2.4) groups showed reduced thickness increases. As shown in FIGS. 20e-20f, for articular cartilage attenuation, both total and medial 1/3 analyses demonstrated increased attenuation for all MMT groups relative to sham; no notable differences between MMT groups were found. As shown in FIG. 20g, for articular cartilage surface roughness, all MMT groups showed significantly higher values than sham control for the total tibia; MMT/hMSC(2.2) showed potentiated surface roughness increases and MMT/hMSC(2.3) showed reduced surface roughness increases; furthermore, both MMT/hMSC(2.3) and MMT/hMSC(2.4) groups showed reduced articular cartilage surface roughness relative to the other two MMT/hMSC groups. As shown in FIG. 20h, in the medial 1/3 region again all MMT groups showed increased surface roughness relative to the sham; both MMT/hMSC (2.3) and MMT/hMSC(2.4) groups showed reduced articular cartilage surface roughness relative to the other two MMT/hMSC groups. As shown in FIG. 20i, for articular cartilage lesion volume of the total tibia all MMT groups, except MMT/hMSC(2.3) and MMT/hMSC (2.4) groups, demonstrated significantly higher lesion volumes; the MMT/hMSC(2.3) and MMT/hMSC(2.4) groups also demonstrated significantly less lesion volumes than the MMT/Saline and MMT/hMSC(2.2) group. As shown in FIG. 20j, no significant differences were found between any groups for exposed bone of the total region. This data is presented as mean+/−SD; n=7 for MMT/hMSC(2.3) and n=8 for all other groups. * represents significant differences (p<0.05) between individual MMT groups and sham. Horizontal black bars indicate significance (p<0.05) between individual MMT groups. hMSCs From Different Donors Yield Variable Therapeutic Outcomes on Osteophyte

### Development

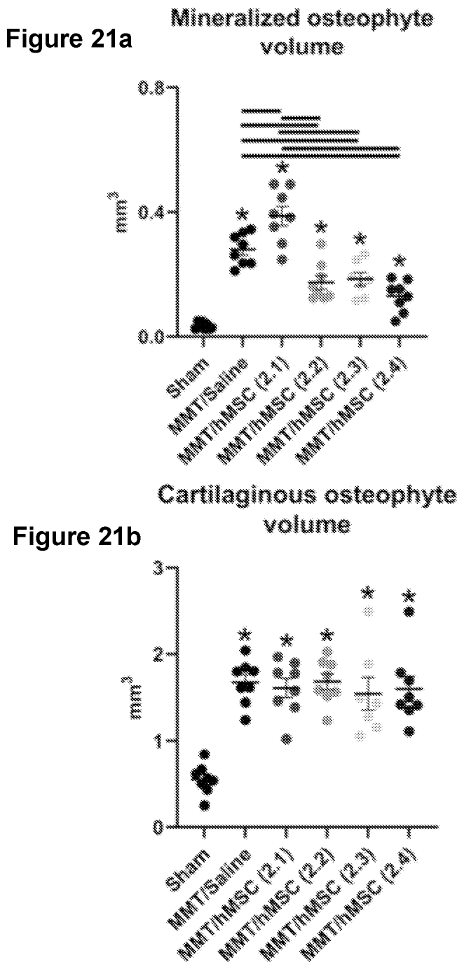

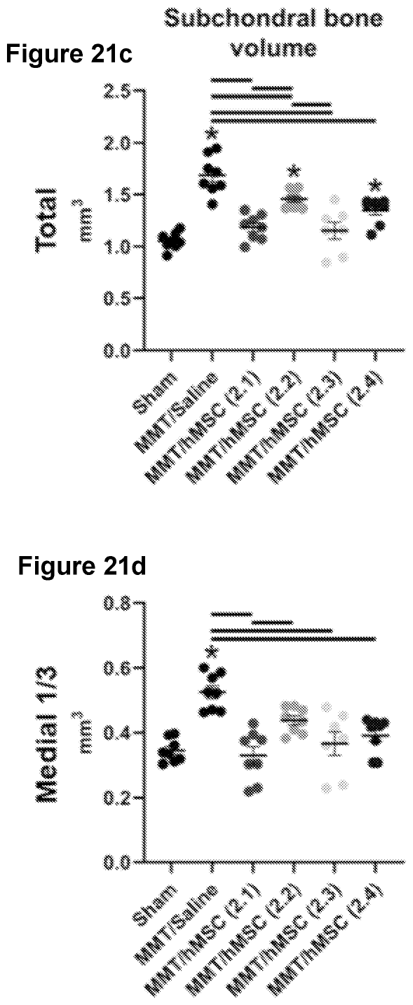

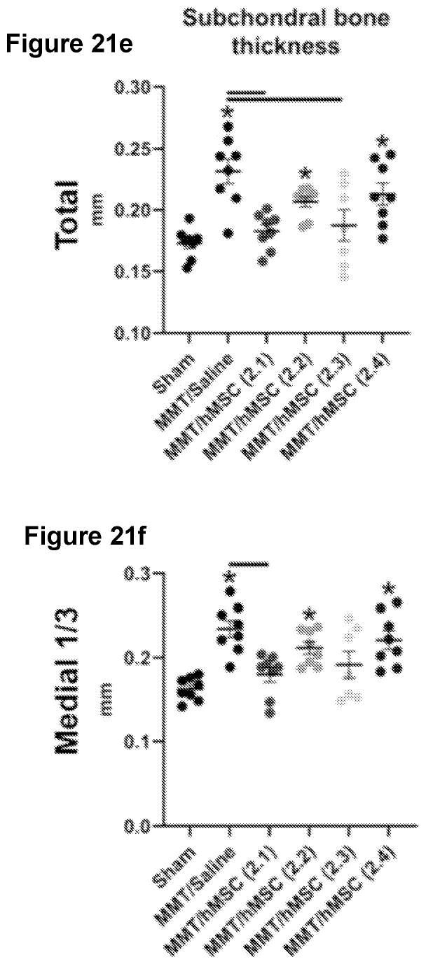

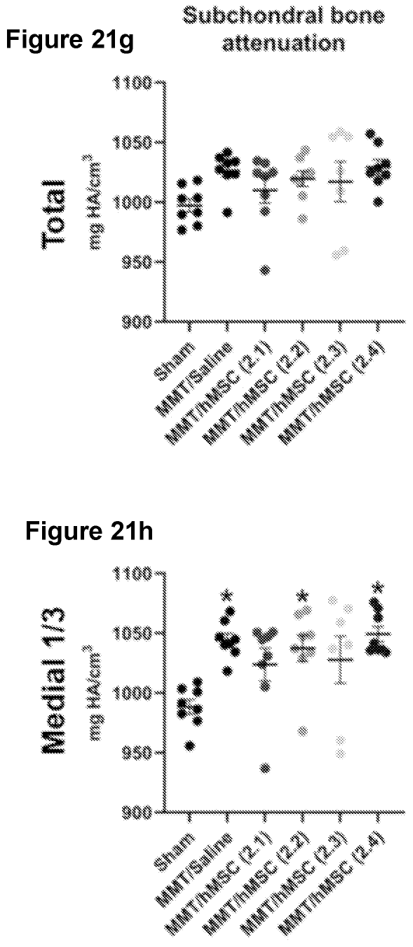

To further assess the donor heterogeneity of hMSCs for OA treatment microCT was implemented to quantitatively assess the associated phenotypes of OA, including osteophyte development, as illustrated in FIGS. 21a-21f. For mineralized osteophyte volume, donor MMT/hMSC(2.1) yielded a significant increase in volume relative to the MMT/Saline disease control while all three other donors yielded significantly decreased mineralized osteophyte volumes relative to sham (FIG. 21a). For cartilaginous osteophyte volume, while all MMT conditions yielded significant increases in volume relative to the sham, no differences were detected between any of the MMT conditions (FIG. 21b). Overall, these metrics reveal that hMSC donors 2.2, 2.3 and 2.4 yield more therapeutic outcomes, relative to hMSC donors 2.1, for the osteophyte data alone as they yielded reduced osteophyte development relative to the disease control.

As shown in FIG. 21a, mineralized osteophyte volumes were significantly increased in all MMT groups relative to sham; MMT/hMSC(2.1) showed significantly greater osteophyte volumes than MMT/Saline; MMT/hMSC(2.2), MMT/hMSC(2.3), and MMT/hMSC(2.4) showed reduced osteophyte volumes relative to MMT/Saline and MMT/hMSC(2.1). As shown in FIG. 21b, for cartilaginous osteophyte volumes, all MMT groups showed significantly greater volumes than sham; no differences were noted between MMT groups. As shown in FIG. 21c, for total subchondral bone volume MMT/Saline, MMT/hMSC(2.2), and MMT/hMSC(2.4) groups showed significantly higher volume than sham; all MMT/hMSC donor groups showed significantly reduced volume relative to sham. As shown in FIG. 21d, in the medial 1/3 region the MMT/Saline group was the only group to show significantly reduced volume relative to sham; all hMSC groups except MMT/hMSC (2.2) showed significantly reduced volume relative to MMT/Saline. As shown in FIG. 21e, for subchondral bone thickness the MMT/Saline, MMT/hMSC(2.2), and MMT/hMSC(2.4) showed significant increases in thickness relative to sham; MMT/hMSC(2.1) and MMT/hMSC(2.3) groups showed reduced thickness relative to MMT/Saline. As shown in FIG. 21f, in the medial 1/3 region results were similar to the total region analysis except no significant difference was noted between MMT/Saline and MMT/hMSC(2.3). As shown in FIG. 21g, for subchondral bone attenuation of the total tibial plateau no significant differences were noted. As shown in FIG. 21h, in the medial 1.3 region, the MMT/Saline, MMT/hMSC(2.2), and MMT/hMSC(2.4) showed significantly higher attenuation values than sham. This data is presented as mean+/−SD; n=7 for MMT/hMSC(2.3) and n=8 for all other groups. * represents significant differences (p<0.05) between individual MMT groups and sham. Horizontal black bars indicate significance (p<0.05) between individual MMT groups.
hMSCs From Different Donors Yield Variable Therapeutic Outcomes on Subchondral Bone Remodeling

In addition to osteophyte developments, subchondral bone composition and morphology were quantified to further assess associated phenotypes of OA. For total subchondral bone volume, while all hMSC donors yield significantly less volume than the MMT/Saline control only donors MMT/hMSC(2.2) and MMT/hMSC(2.4) showed significant increases relative to sham (FIG. 21c). For the medial 1/3 region, no significant differences were noted between the sham control and any of the hMSC donors, while the MMT/Saline control showed significantly higher volume (FIG. 21d). For subchondral bone thickness, donors MMT/hMSC(2.2) and MMT/hMSC(2.4) showed significant increases in thickness relative to sham while the other two donors showed no difference; furthermore, MMT/hMSC(2.1) and MMT/hMSC(2.3) showed significantly less thickness than the MMT/Saline disease control (FIG. 21e). The medial 1/3 region showed nearly identical results (FIG. 21f). For subchondral bone attenuation (which serves as a quantitative measure of the mineralized content of the bone), while no differences were noted in the total region the medial 1/3 region showed significant increases for donors MMT/hMSC(2.2) and MMT/hMSC(2.4) while the other two donors showed no difference. Overall, these metrics reveal that hMSC donors 2.1 and 2.3 yield more therapeutic outcomes, relative to hMSC donors 2.2 and 2.4, as they yielded reduced increases in subchondral bone thickening (volume and thickness) and hardening (attenuation).
Synovial Fluid From Different Human Donors Yields Unique Cytokine Profiles

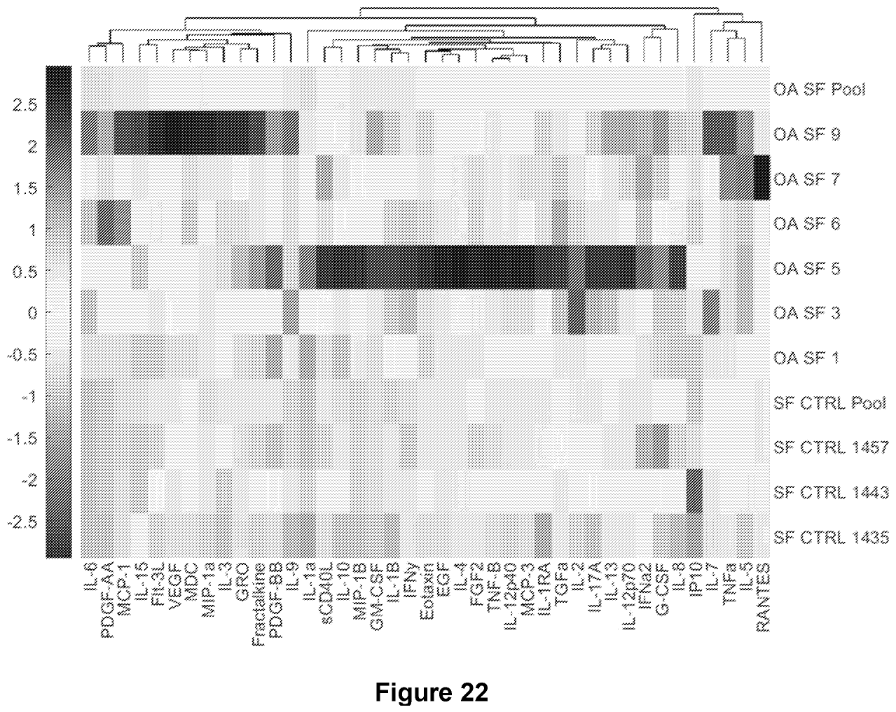

To develop an OA simulated microenvironment to assess the paracrine signaling properties SF was collected from human patients. Qualitative comparison of these SF samples demonstrated that OA SF samples yielded increased immunomodulatory cytokine content relative to the SF CTRL samples, as illustrated in FIG. 22. This included increased concentrations of major pro-inflammatory mediators (IL-1β, TGFα, IFNγ, IL-6) in OA, relative to the SF CTRL samples. Correlation analysis was performed between OA SF cytokine content and Kellgren and Lawrence (KL) grade, for respective donors, to see if this clinical phenotype could account for the differences in SF content between donors; however, no relationship existed between these two variables (Table 3). For treatment in the OA simulated microenvironment, all hMSCs were treated with pooled CTRL SF samples (3 donors) and OA SF samples (6 donors) to assess the overall effect of SF on these cells, rather than donor specific SF samples on hMSCs.

OA SF samples yielded increased overall cytokine content relative to non-OA (SF CTRL) SF samples. Among donors, for either OA SF or SF CTRL, there were distinct differences between human donors. The pooled samples for both CTRL SF and OA SF, samples were combined in equal parts.
hMSC Conditioning With IL-1β Yields the Most Effective OA Simulated Microenvironment In Vitro

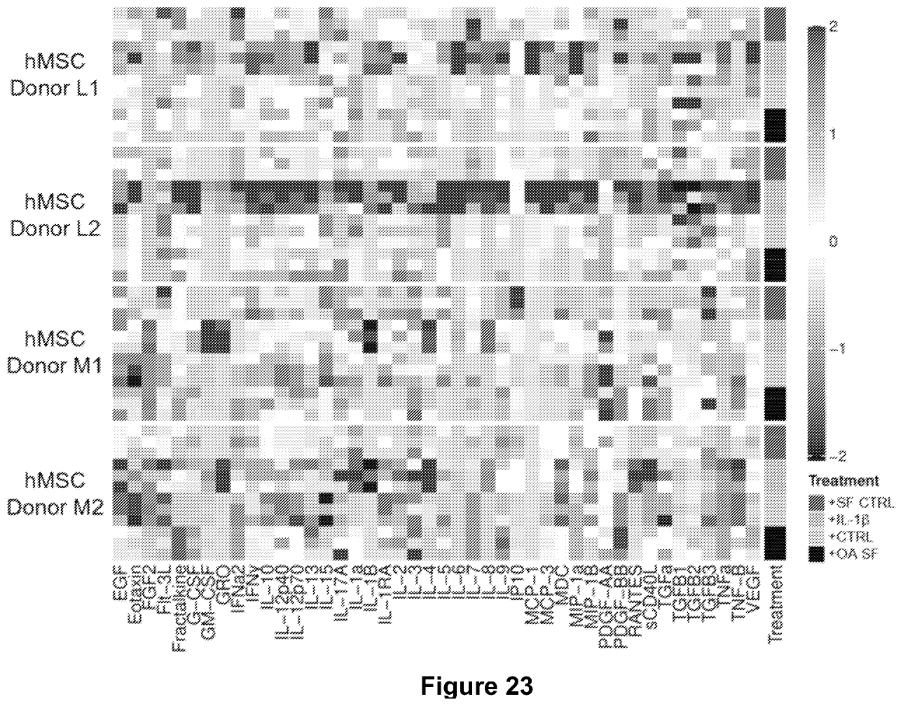

To further study the effects of the paracrine signaling properties of hMSCs on OA an OA simulated microenvironment was constructed to recapitulate the in vivo microenvironment (knee joint space following intra-articular injection) in vitro. Thus, hMSCs were independently conditioned with 1) unconditioned media (+CTRL), 2) 20 ng/ml IL-1β conditioned media (+IL-1β), 3) 10% SF control conditioned media (+SF CTRL), and 4) 10% OA SF conditioned media (+OA SF), respectively. For all hMSC donors, conditioning with IL-1β demonstrated increased cytokine secretion relative to all other conditioning strategies, as illustrated in FIG. 23. However, no distinct differences were notable in terms of overall cytokine secretion of hMSCs between the CTRL, SF CTRL, and OA SF conditioning groups.

Multiplexed immunoassay analysis of 44 cytokines (columns; z-scored) secreted from hMSCs in unconditioned media (+CTRL), IL-1β conditioned media (+IL-1β), SF (collected from patients without an OA clinical diagnosis) conditioned media (+SF CTRL), and OA SF (collected from patients with clinically diagnosed OA) conditioned media (+OA SF; each row represents a single sample) for four unique hMSC donors. +IL-1β conditioning demonstrated increased paracrine cytokine secretion relative to all other conditions with donors L1 and L2 demonstrating potentiated cytokine secretion levels relative to donors M1 and M2.

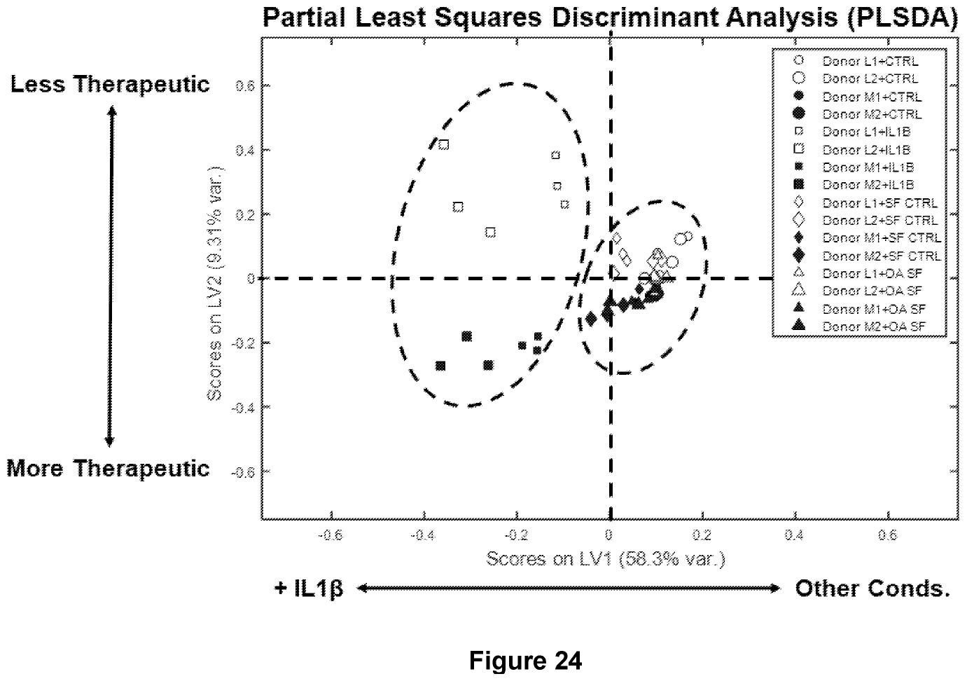

Based on the qualitative analysis of the cytokine secretion data for all OA simulated microenvironment conditioning strategies, a PLSDA analysis was implemented to determine which conditioning strategy would be most viable to assess differences in paracrine signaling between more therapeutic and less therapeutic hMSCs (as determined with microCT). From LV1, the separation between IL-1β to the left and all other conditioning strategies (CTRL, SF CTRL, OA SF) to the right can be clearly observed, as illustrated in FIG. 24. This separation was clearly identified through establishing clusters for IL-1β (within larger circle on left) and other conditions (within smaller circle on right; FIG. 24). Furthermore, from LV2, the separation between more therapeutic (Donors M1+CTRL, M2+CTRL, M1+IL1B, M2+IL1B, M1+SF CTRL, M2+SF CTRL, M1+OA SF, M2+OA SF) and less therapeutic (Donors L1+CTRL, L2+CTRL, L1+IL1B, L2+IL1B, L1+SF CTRL, L2+SF CTRL, L1+OA SF, L2+OA SF) was established (FIG. 24). Another important observation to make from this dataset is that IL-1β conditioning yielded better separation between more therapeutic and less therapeutic hMSCs, relative to the other conditioning strategies. Thus, IL-1β was used for conditioning in the OA simulated microenvironment for RNA-Seq analysis and intracellular signaling analysis.

PLSDA analysis identified two profiles of cytokines, LV1 and LV2, that identified a distinct separation between less therapeutic (Donors L1+CTRL, L2+CTRL, L1+IL1B, L2+IL1B, L1+SF CTRL, L2+SF CTRL, L1+OA SF, L2+OA SF) and more therapeutic hMSCs (Donors M1+CTRL, M2+CTRL, M1+IL1B, M2+IL1B, M1+SF CTRL, M2+SF CTRL, M1+OA SF, M2+OA SF), as determined by microCT, on LV2. Furthermore, on LV1 there is clear separation between hMSCs conditioned with IL-1β and hMSCs conditioned with media only (CTRL), SF CTRL, or OA SF. In this redefined plane, two distinct clusters formed with IL-1β (within larger circle on left) clustering to the left and hMSCs conditioned with media only (CTRL), SF CTRL, or OA SF (within smaller circle on right) clustering to the right. Variability accounted for in each LV is included on respective axes labels.
hMSC Secretion of GM-CSF, GRO, IL-4, PDGF-AA, and TGF-β3 Relate to OA Therapeutic Outcomes In Vivo

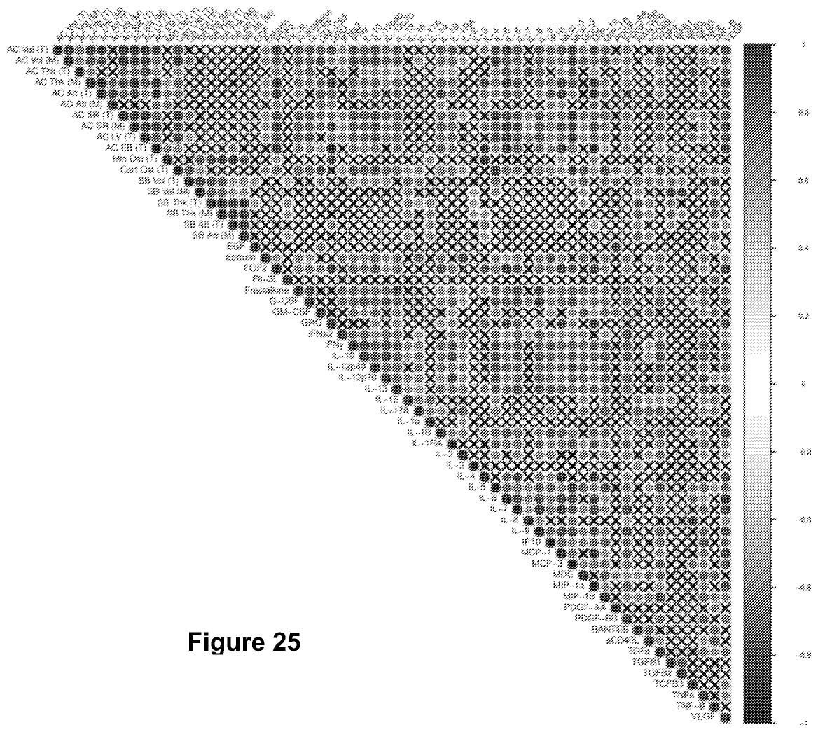

To determine what profile of hMSC cytokines related to therapeutic outcomes in vivo a correlation analysis was run. The hMSC cytokine data, for all four donors, from the in vitro OA simulated microenvironment (using the IL-1β stimulus only) and the in vivo data from the microCT study were used to identify correlative relationships. This analysis demonstrated that significant inverse correlations existed (negative Pearson correlation coefficients) between microCT therapeutic outcomes and granulocyte macrophage colony stimulating factor (GM-CSF), chemokine ligand 1 (GRO), IL-4, platelet derived growth factor (PDGF)-AA, and TGFβ3, as illustrated in FIG. 25. Thus, increased secretion of these five factors yielded more therapeutic outcomes in OA. There were many significant positive correlations (positive Pearson's correlation coefficient) in the assessment run. However, these are not of interest as the objective was to identify factors that show increased expression in therapeutic hMSCs and could be targeted to identify underlying signaling pathways mediating these secreted cytokines.

Pearson's correlation coefficients for in vivo MMT therapeutic data and in vitro multiplexed immunoassay cytokine analysis (+IL-1β). The cytokines GM-CSF, GRO, IL-4, PDGF-AA, and TGFβ3 showed significant inverse correlations with microCT therapeutic outcomes (increased cytokine secretion led to therapeutic outcomes). Black X's indicate coefficients that are not significant (significance level set at p<0.05).

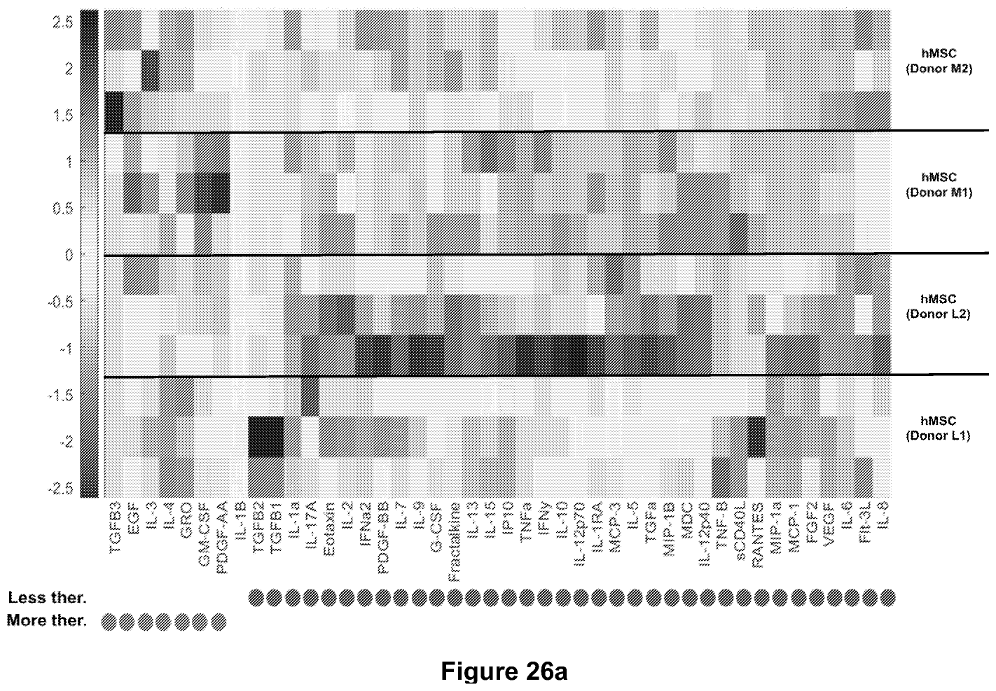

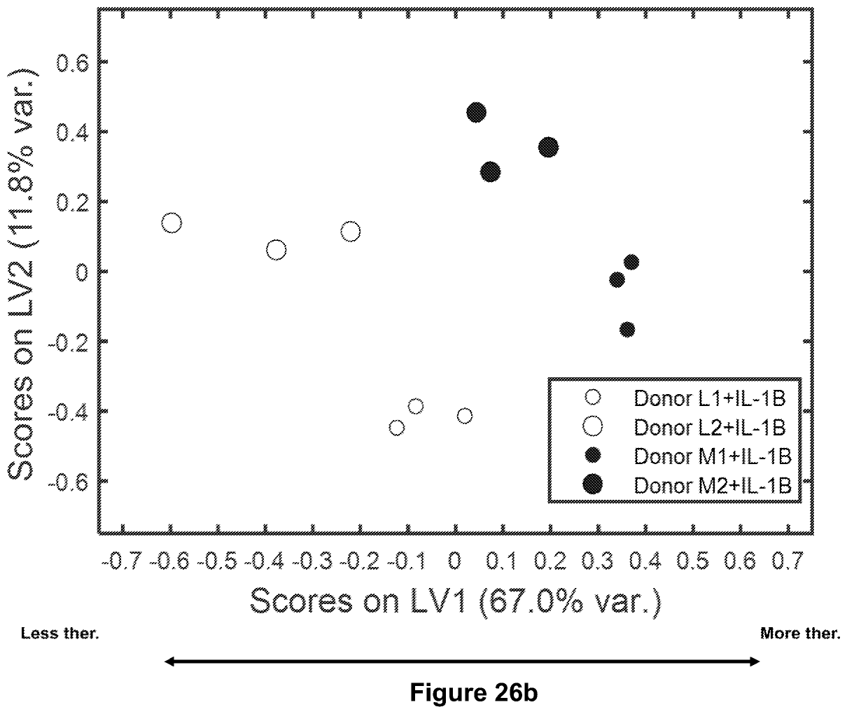

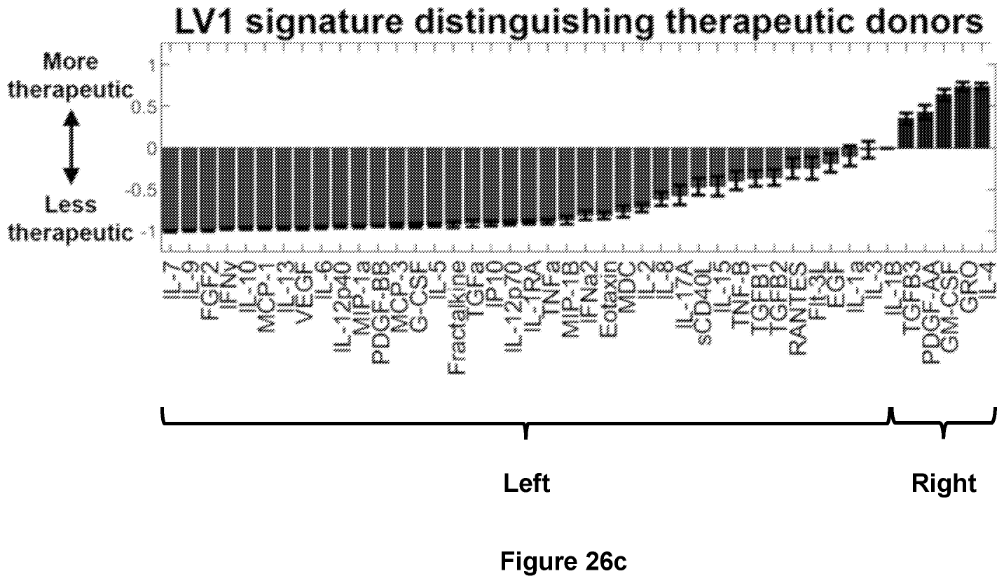

FIGS. 26a-26c illustrate paracrine signaling response of hMSCs in an IL-1β OA simulated microenvironment. To assess differences in hMSC cytokine signaling between more therapeutic and less therapeutic donors (as identified in the microCT study), the cytokine profiles of all cells were observed qualitatively, for the IL-1β conditioning only based on its enhanced utility (FIG. 26a). In comparing more therapeutic and less therapeutic hMSCs, the more therapeutic hMSCs yielded a targeted response relative to the indiscriminate response (increase in majority of cytokines assessed) of less therapeutic hMSCs (FIG. 26a). To further quantify these differences between less therapeutic and more therapeutic hMSCs a PLSR approach was implemented. From LV1, the separation between less therapeutic hMSC donors (L1 and L2) to the left from more therapeutic donors (M1 and M2) to the right can be clearly observed (FIG. 26b). While no clear overall trend was established along LV2 the separation between both more therapeutic and less therapeutic hMSC donors along this axis demonstrate the inherent differences that exist between hMSCs from different donors (FIG. 26b). In addition to these findings, the inventors generated loadings plots from PLSR that display the relative importance of specific cytokines in contributing to the final LV1 (FIG. 26c). In this loadings plot, the factors that contribute to less therapeutic hMSCs are shown on the left and those that contribute to more therapeutic donors are shown on the right; thus, the more therapeutic hMSCs yielded increased levels of GM-CSF, GRO, IL-4, PDGF-AA, and TGF-β3.

FIG. 26a provides a multiplexed immunoassay analysis of 44 cytokines (columns; z-scored) secreted from hMSCs in IL-1 conditioned media. donors L1 and L2 demonstrated potentiated overall cytokine secretion levels relative to donors M1 and M2 which yielded a more targeted paracrine profile. FIG. 26b provides PLSR analysis (input: in vitro cytokine data; output: microCT data) identified a profile of cytokines along LV1 that identified a distinct separation between less therapeutic and more therapeutic hMSCs, as determined by microCT. As shown in FIG. 26c, loadings plot demonstrating relative contribution of cytokines to PLSR scores obtained show that the cytokines GM-CSF, GRO, IL-4, PDGF-AA, and TGF-β3 contribute to separating out more therapeutic hMSC donors while all other cytokines assessed contribute more to less therapeutic hMSCs. Variability accounted for in each LV is included on respective axes labels.
More Therapeutic hMSCs Demonstrate Unique Gene Expression Pathway Profiles Relative to Less Therapeutic hMSCs

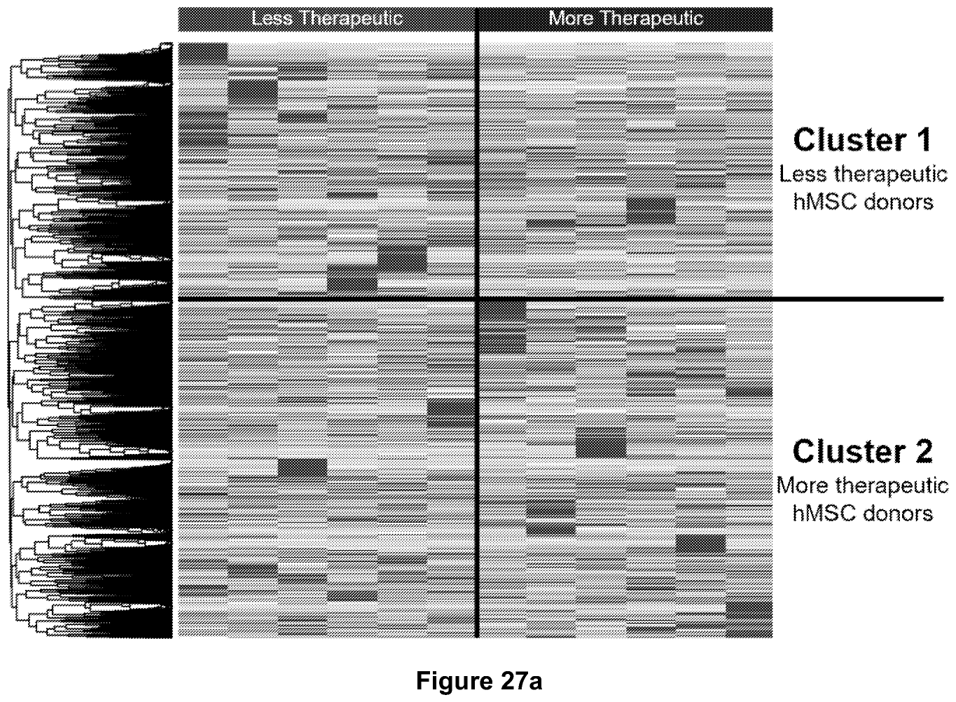

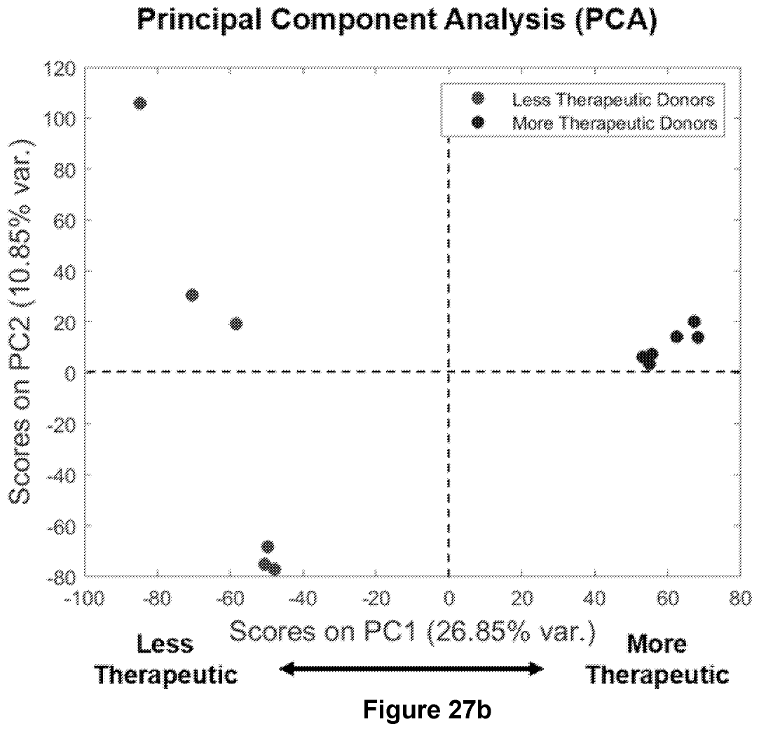

To take a more holistic approach to identifying cellular attributes that relate to therapeutic efficacy of hMSCs in OA, RNA-Seq was utilized to quantify gene expression of all hMSC donors, as illustrated in FIGS. 27a-27b. To do this all hMSCs were again conditioned for 1 day in IL-1β in our OA simulated microenvironment after which 24,475 genes were quantified for each of the respective donors, which were grouped together into their more and less therapeutic cell groupings based on the outcomes from our microCT analysis (FIG. 27a). In comparing at this data overall the inventors can roughly approximate the formation of two clusters, with cluster 1 representing the gene expression profile of less therapeutic hMSCs and cluster 2 representing the profile of more therapeutic hMSC donors (FIG. 27a). The differences yielded between more and less therapeutic cells was quantified using PCA which demonstrated clear separation of less therapeutic hMSCs to the left and more therapeutic hMSCs to the right along the PC1 axis (FIG. 27b). While looking at individual genes can provide insights into the differences between these groups (less therapeutic and more therapeutic hMSCs), the sheer magnitude of this data set and absence of biological context motivate additional methods to be employed.

FIG. 27a illustrates quantification of 24,475 genes using RNA-Seq yielded two clusters with cluster 1 demonstrating unique gene expression of less therapeutic hMSCs and cluster 2 demonstrating the gene expression of more therapeutic hMSCs. As shown in FIG. 27b, these unique gene expression profiles were further demonstrated quantitatively using PCA which clearly showed that less therapeutic hMSCs (to the left) and more therapeutic hMSCs (to the right) separate along the PC1 axis.

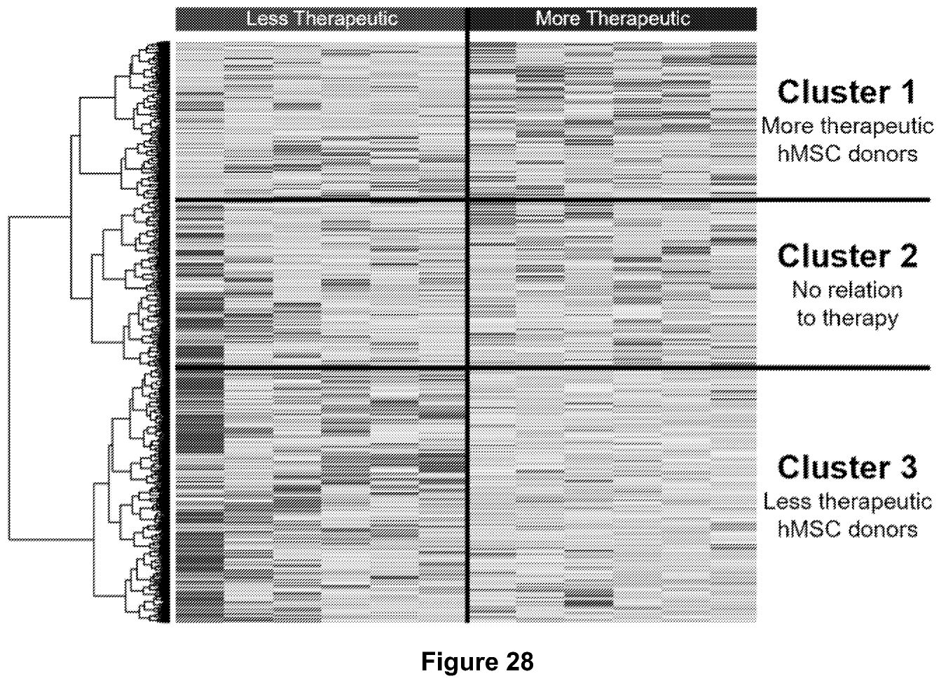

GSVA is a generalized gene set enrichment method that quantifies gene pathway enrichment scores. This provides added biological context as differences in gene expression pathways are able to be identified between more therapeutic hMSCs and less therapeutic hMSCs. Furthermore, it reduces the magnitude of the gene expression dataset into a smaller number of gene expression pathways. More specifically, in this study 6,287 gene pathways were screened for differences in these cell groups. This analysis yielded the formation of three distinct clusters, with a profile of gene expression for more therapeutic hMSCs identified in cluster 1 and those for less therapeutic hMSCs in cluster 3, as illustrated in FIG. 28. Furthermore, for the pathways conserved for both more and less therapeutic hMSCs, in cluster 2, they were identified as having no relation to therapy based on the lack of differences between the two respective hMSC groups (FIG. 28).

Quantification of 6,287 gene expression pathways screened using GSVA yielded three distinct clusters with cluster 1 demonstrating unique gene expression of more therapeutic hMSCs, cluster 2 categorizing the gene expression pathways which had no relation to therapy as they were conserved between more and les therapeutic hMSCs, and cluster 3 demonstrating the gene expression of less therapeutic hMSCs.

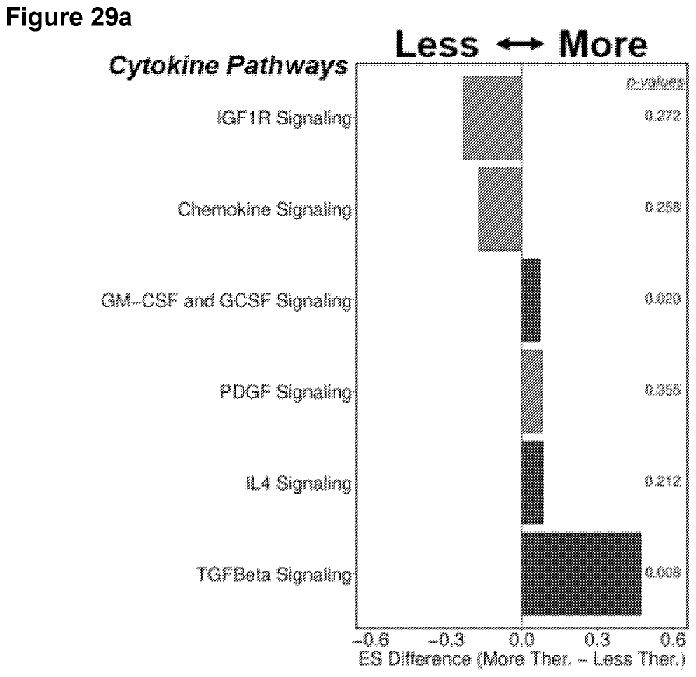

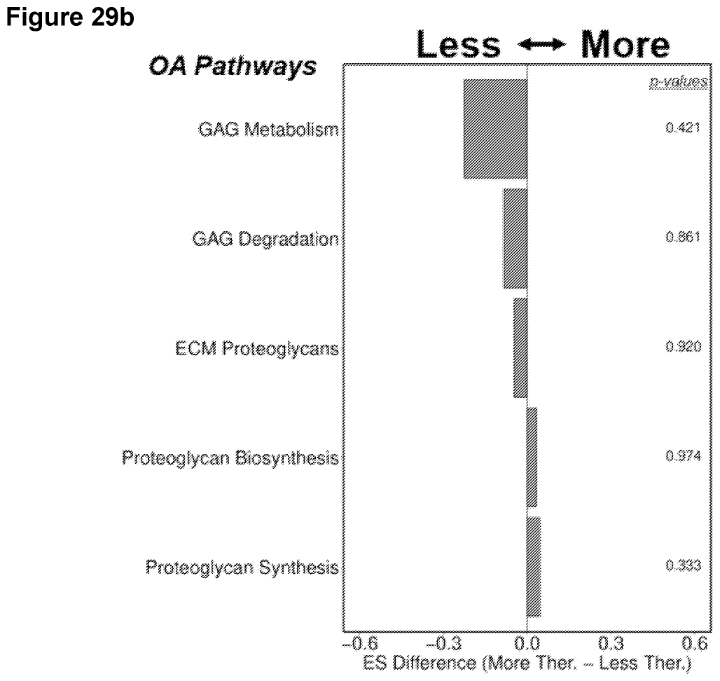

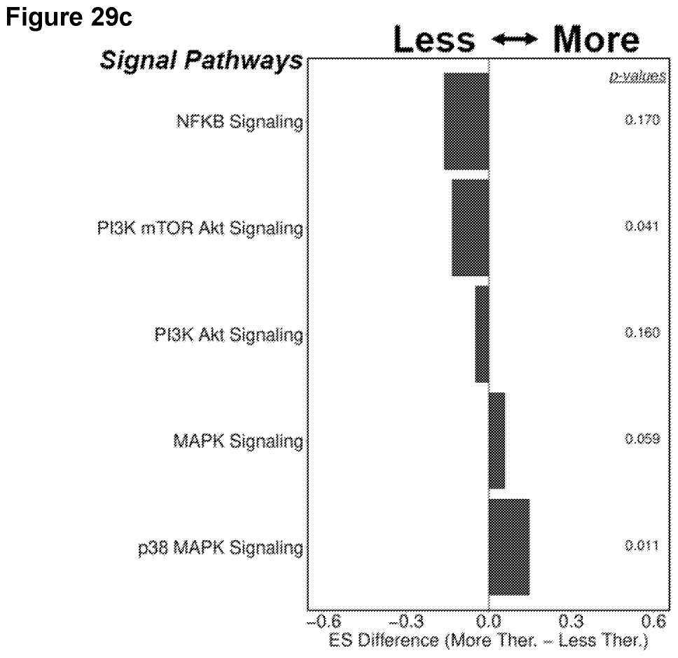

To look more quantitatively at the outcomes of the GSVA analysis the inventors can compare the relative enrichment scores of less therapeutic hMSCs and more therapeutic hMSCs, as illustrated in FIGS. 29a-29c. To further substantiate the outcomes of the inventors' cytokine analysis they can see that more therapeutic hMSCs demonstrate increased enrichment scores for most of the therapeutic cytokines identified (FIG. 29a). However, PDGF-AA did not yield a significant difference in enrichment scores between the two groups of cells. The database used for gene pathways also contained no specific pathway for GRO and therefore a generalized chemokine pathway was used. Furthermore, the inventors did some additional exploratory studies and pulled out OA related pathways, including three proteoglycan and GAG pathways, both of which serve as the major biochemical components of articular cartilage. While there were differences in enrichment scores for these pathways in more and less therapeutic hMSCs, none of these were significant (FIG. 29b). In addition to these cytokine and OA related gene expression pathways, further exploratory studies identified a large number of intracellular signaling pathways that showed differences in enrichment scores between more and less therapeutic hMSCs, including the MAPK, AKT, and NFKB signaling pathways (FIG. 29c).

As shown in FIG. 29a, more therapeutic hMSCs demonstrated significant increased enrichment of GM-CSF and GCSF, IL-4, and TGFβ pathway signaling. As shown in FIG. 29b, no significant differences were identified in assessing differences in enrichment scores between more therapeutic and less therapeutic hMSCs for GAG and proteoglycan OA associated pathways. As shown in FIG. 29c, less therapeutic hMSCs yielded increased enrichment of the Akt and NF-κB signaling pathways while more therapeutic hMSCs yielded increased enrichment of the MAPK signaling pathway. Horizontal bars labeled GM-CSF and GCSF Signaling, IL4 Signaling, and TGFBeta Signaling (FIG. 29a), and those bars shown in FIG. 29c, indicate significance (p<0.25) between more and less therapeutic hMSCs enrichment scores.
More Therapeutic hMSCs Demonstrate Increased p-Atf-2, p-JNK, p-mTOR, and p-PTEN and Decreased p-Akt phospho-protein Expression

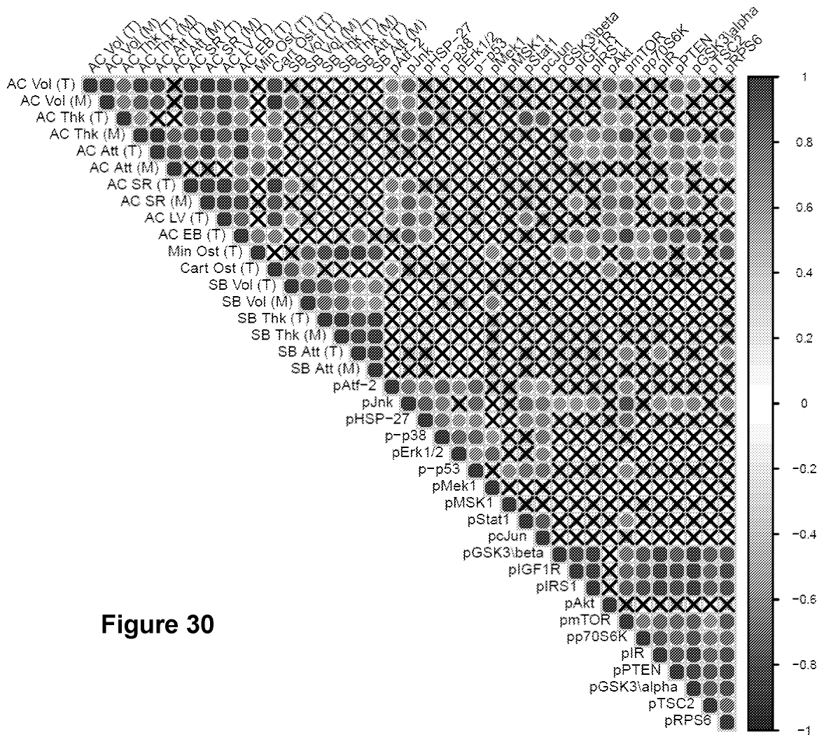

To determine what profile of hMSC phospho-proteins related to therapeutic outcomes in vivo a correlation analysis was run. The hMSC phospho-protein, for all four donors, from the in vitro OA simulated microenvironment (using the IL-1β stimulus only; in the MAPK and Akt intracellular signaling pathways) and the in vivo data from the microCT study were used to identify correlative relationships. This analysis demonstrated that significant inverse correlations existed (negative Pearson correlation coefficients) between microCT therapeutic outcomes and p-activating transcription factor (Atf)-2 and p-JNK in the MAPK signaling pathway and p-mechanistic target of rapamycin (mTOR), p-phosphatase and tensin homolog (PTEN), and p-glycogen synthase kinase 3 (GSK3) a in the Akt signaling pathway, as illustrated in FIG. 30. Thus, increased secretion of these five phospho-proteins yielded more therapeutic outcomes in OA. There was also a single positive correlation (positive Pearson's correlation coefficient) in the p-Akt phospho-protein of the Akt signaling pathway (FIG. 30). While there were several other phospho-proteins in both intracellular signaling pathways that yielded both inverse and positive correlations with individual therapeutic outcomes the intent of the study was to identify phospho-proteins that related to overall therapeutic outcomes so a threshold of >50% of inverse/positive correlations was set in identifying the defining phospho-proteins.

Pearson's correlation coefficients for in vivo MMT therapeutic data and in vitro intracellular signaling analysis (+IL-1β). The phospho-proteins p-Atf-2 and p-JNK from the MAPK signaling cascade and p-mTOR and p-PTEN from the Akt signaling cascade showed significant inverse correlations with microCT therapeutic outcomes (increased phospho-protein expression led to therapeutic outcomes). The phospho-protein p-Akt from the Akt signaling cascade showed a significant correlation with microCT therapeutic outcomes (decreased phospho-protein expression led to therapeutic outcomes). Correlations that demonstrated >50% significant correlations with microCT therapeutic outcome metrics were selected as several other phospho-proteins showed single and multiple respective correlations with individual microCT therapeutic outcome metrics. Black X's indicate coefficients that are not significant (significance level set at p<0.05).

### DISCUSSION

To identify hMSC cellular attributes in vitro that relate to in vivo OA therapeutic outcomes, the microenvironment these cells are exposed to following their delivery into the arthritic joint should be simulated. Inflammation has been well characterized as playing a key role in OA pathogenesis and thus in this aim an OA simulated microenvironment was engineered to quantify cellular attributes of hMSCs. To recapitulate the in vivo microenvironment (knee joint space following intra-articular injection) in vitro hMSCs were independently conditioned with OA SF, collected from patients with clinically diagnosed OA, and IL-1β as it is a major pro-inflammatory modulator in OA. While all conditioning strategies (including+CTRL and +SF CTRL) demonstrated an ability to differentiate between more therapeutic and less therapeutic hMSCs, as demonstrated using a PLSR model with cytokine data as the input, IL-1β conditioning demonstrated an ability to better differentiate between these two therapeutic groups of hMSCs. Thus, the +IL-1β conditioning strategy was selected as the best system to simulate the OA microenvironment.

Relating in vitro hMSC cytokine secretion with in vivo therapeutic outcomes, with correlation analysis and PLSR, demonstrated that more therapeutic hMSCs yielded increased secretion of the chemokines (GM-CSF and GRO), cytokines (IL-4) and growth factors (PDGF-AA and TGFβ3). For all these factors identified, there is a large body prior research studying the role of these cytokines in MSCs in the context of OA, permitting this cytokine secretion to be contextualized. GM-CSF has been shown to enhance the mobilization of MSCs from the bone marrow and while this has not been directly linked to enhanced therapeutic efficacy in OA potentiation of the MSC response with GM-CSF has demonstrated potentiated therapeutic efficacy of articular cartilage repair induced by microfracture. Furthermore, while the chemokine GRO (CXCL1) has been shown to initiate cartilage degradation and potentiate inflammation in the joints, these resulting cellular actions may make intuitive sense with the well-defined role GRO plays in monocyte and neutrophil trafficking to the site of injury. However, this proinflammatory event induced by hMSC secretion of GRO does not necessarily point to increased catabolismin the joint space as there may be important distinctions between the chronic nature of the OA inflammatory environment and the acute response induced by the hMSC secretome. Briefly, to resolve chronic inflammation, an acute event is needed to bring in immune cells and activate different inflammatory cascades to resolve and induce a pro-regenerative response. Thus, GRO induced trafficking of monocytes and neutrophils may be contributing to this acute event (short duration inflammatory event) rather than potentiating the chronic inflammatory state of OA with more sustained inflammation. In addition to secretion of chemotactic factors, more therapeutic hMSCs demonstrated potentiated secretion of the potent anti-inflammatory cytokine IL-4. IL-4 has demonstrated chondroprotective effects in pre-clinical models of OA, where MSC spheroids transduced with IL-4 yielded better cartilage protection and pain relief, relative to naïve MSCs in vivo. These outcomes may be partly explained by further studies that have demonstrated IL-4 can protect cartilage by inhibiting inducible nitric oxide synthase (iNOS) and NO which yields subsequent suppression of IL-1β, TNF-α. Furthermore, the growth factors PDGF-AA and TGFβ3 have been studied extensively for their ability to yield therapeutic outcomes, both in combination and independently of MSC therapeutic delivery. More specifically, PDGF is believed to support tissue regeneration and anti-inflammatory properties in clinical patients and can serve as a critical component of platelet rich plasma (PRP) therapeutics being delivered clinically to treat OA. Furthermore, in pre-clinical studies PDGF overexpressing MSCs were shown to exert anti-fibrotic, anti-inflammatory, and pro-chondrogenic capacities in a canine model of OA, through both the reduction of pro-inflammatory factors and MMP-13. Furthermore, the TGF-β superfamily of growth factors have received increasing interests due to their anabolic effects in articular cartilage and their roles in subchondral bone remodeling and osteophyte development during OA. While the role of each of these specific cytokines have been implicated in MSC therapeutic action in OA, this profile of secreted factors is unique.

To take a more holistic approach to identifying cellular attributes that relate to therapeutic efficacy of hMSCs in OA, RNA-Seq was utilized to quantify gene expression of all hMSC donors. While RNA-Seq demonstrated the formation of unique gene expression profiles GSVA was implemented to study the enrichment of gene expression pathways and demonstrated clear differences in more and less therapeutic hMSC gene expression pathways. More specifically, the GM-CSF, IL-4, and TGFβ3 gene expression pathways demonstrated increased enrichment in more therapeutic hMSCs relative to less therapeutic hMSCs, therefore further substantiating the outcomes obtained in the cytokine analysis study. However, while PDGF showed increased enrichment in the more therapeutic hMSC group, it was not significant and no gene enrichment pathway was available for the cytokine, GRO. While in these exploratory studies, OA related pathways (proteoglycan and GAG) were studied for differences in enrichment score none of these were found to be significant. However, in these exploratory studies a number of signaling pathways including the Akt, MAPK, and NF-κB signaling pathways were found to have significant differences in enrichment scores between more and less therapeutic hMSCs. Thus motivating all of these pathways to be screened for differences in intracellular signaling between more and less therapeutic hMSCs. These studies demonstrated that both the MAPK and Akt pathways yielded differences in signaling while no differences were identified in the NF-κB pathway. More specifically, more therapeutic hMSCs demonstrate increased p-Atf-2, p-JNK, p-mTOR, and p-PTEN in the MAPK pathway and decreased p-Akt phospho-protein expression in the Akt pathway. More in depth analysis of the phospho-protein signaling of hMSCs is provided in Example 3.

Outside of screening MSCs for their ability to adhere to plastic and express standard MSC phenotypic cell surface markers no further screening metrics are implemented for MSCs that are clinically administered. This absence of screening is likely a major contributor to the high variability that has been reported in clinical studies as it has been previously demonstrated that the standard criterion employed to characterize MSCs, including phenotypic characterization and adherence to plastic surfaces, defines a largely heterogenous population of cells. The findings presented in this Example may provide methods to better screen and more effectively treat patients with MSC therapeutics for OA.

### EXAMPLE 3: Identification of Human Mesenchymal Stromal Cell Intracellular Signaling Pathways Mediating Secretion of Cytokines That Yield Therapeutic Efficacy in Osteoarthritis

In the context of MSCs, the role of intracellular signaling in mediating the immune responsiveness of these cells has been studied extensively in the context of many disease states. While there are likely many intracellular signaling pathways mediating the immunomodulatory response of MSCs, the Akt and MAPK pathways have been implicated as major contributors in the immunomodulatory potential of these cells. Additionally, the NF-κB pathway has been implicated as a key mediator of MSC immunomodulation in an expanse of disease states. Furthermore, all four of these pathways have been shown to be upstream regulators of a large number of inflammatory mediators, including the immunomodulatory cytokines assessed in Example 2. However, the role of these phospho-protein signaling pathways in mediating hMSC responsiveness to the chronic inflammatory OA microenvironment remains an understudied area. Due to the central regulatory role these signaling pathways have in the paracrine signaling response of MSCs, the signaling phospho-proteins within these pathways provide viable therapeutic targets for mediating therapeutic MSC activity.

In this Example, the Akt and MAPK signaling pathways were analyzed due to the pivotal role these pathways play in MSC immunomodulation and their upstream regulation of the immunomodulatory factors quantified in Example 2. Furthermore, the Akt, MAPK, and NF-κB signaling pathways were screened in preliminary studies and the Akt and MAPK signaling pathways were found to be the only two to yield phospho-protein level differences between less therapeutic and more therapeutic hMSCs (as identified in Example 2). Phospho-proteins from these pathways were then targeted pharmacologically to identify phospho-signaling proteins that mediate the production of the therapeutic related cytokines identified in Example 2.

### Materials and Methods

hMSC Culture and Characterization

hMSCs derived from bone marrow were obtained from EPIC core facility at Emory University and RoosterBio (MSC-004; RoosterBio, Inc). EPIC hMSCs (2.1 and 2.2) were cultured in MSCB Mesenchymal Stem Cell Basal Medium (PT-3238; Lonza) supplemented with 10% heat-inactivated FBS (S11110H; Atlanta Biologicals), 1 mM L-glutamine (SH3003401; HyClone), and 100 μg/mL P/S (B21110; Atlanta Biologicals) and sub-cultured at 80% confluency. Rooster hMSCs were cultured in RoosterNourish-MSC (KT-001; RoosterBio, Inc) media supplemented with 2% RoosterBooster (SU-003; RoosterBio, Inc) and sub-cultured at 80% confluency. hMSC phenotypes for all donor hMSCs were confirmed by adipogenic, chondrogenic, and osteogenic differentiation. Flow cytometry was also used to characterize the hMSCs to confirm that all donor cells expressed characteristic MSC surface markers (CD73, CD90, CD105) and lacked hematopoietic markers (CD45, CD34, CD11b, CD79A, HLA-DR). Metadata is also included for all hMSC donors (Table 1).
In vitro OA Simulated Microenvironment

hMSCs were utilized in vitro. hMSCs were sub-cultured to 80% confluency in complete Lonza for hMSC donors 2.1 and 2.2 and Rooster medium for hMSC donors 2.3 and 2.4 in 12-well plates and cultured at 37° C., 5% CO₂. Cells were placed in monolayer culture in treatment medium (unconditioned or conditioned) in 12-well plates at 37° C., 5% CO₂. Each of the four hMSC donors were independently conditioned with unconditioned media (+CTRL) or 20 ng/ml IL-1β conditioned media IL-1β was used to model the OA inflammatory environment in the current study as it is a major pro-inflammatory modulator in OA. The IL-1β concentration weas selected based on prior experiments and preliminary data. For intracellular signaling quantification, condition media was added to hMSCs at 0 hr for a 5-, 15-, and 60-mins conditioning period in monolayer followed by cell lysate collection at 5-, 15-, and 60-mins, respectively. hMSCs were lysed using the Bio-Plex cell lysis kit (#171304011; Bio-Rad Laboratories) with the addition of one complete mini protease inhibitor tablet (11836153001; Roche Holding AG) and 20 μl of phenylmethylsulfonyl fluoride (PMSF; 10837091001; Roche Holding AG) per 5 ml of lysis buffer. Lysates were placed in microcentrifuge tubes and inverted at 4° C. for 10 min, then centrifuged at 4° C. for min at 13,000 rpm and supernatant was collected. For quantification of secreted cytokines, conditioned media was added to hMSCs at day 0 followed by a 24-hr conditioning period in monolayer culture with media collection at the study end point. Samples were stored at −80° C. until analysis was performed.
hMSC Intracellular Signaling Analysis

Cell lysates were thawed on ice and centrifuged at 4° C. for 10 min at 13,000 rpm. Protein concentrations were determined using a Pierce bicinchoninic acid assay (BCA) protein assay (23225; Thermo Fisher Scientific) and normalized with Milliplex MAP assay buffer (43-041; EMD Millipore Corporation) to 2 μg of protein/25 μl for both the MAPK and Akt pathway analysis as these dilutions fell within the linear range of the MAGPIX (MAGPIX-XPON4.1-CEIVD; EMD Millipore Corporation) system. MAPK and Akt pathway phospho-signaling proteins were quantified using the bead-based MAPK/SAPK signaling 10-plex (48-660MAG; EMD Millipore Corporation) and Akt/mTOR phosphoprotein 11-plex (48-611MAG; EMD Millipore Corporation) kits. Median fluorescent intensity values were read out using Luminex xPONENT software V4.3 in the MAGPIX system.
hMSC Cytokine Analysis

Cytokines were quantified for all donors with +CTRL (n=3/hMSC treatment condition) and +IL-1β (n=3/hMSC treatment condition), conditioned media, respectively. Loaded samples (2.03 μL) were determined to be within the linear range of detection of the MAGPIX (MAGPIX-XPON4.1-CEIVD; EMD Millipore Corporation) system. Cytokines were quantified using a bead based multiplex immunoassay, Luminex Cytokine/Chemokine 41 Plex Immunomodulatory Kit (HCYTMAG-60K-PX41; EMD Millipore Corporation). Median fluorescent intensity values were read out using Luminex xPONENT software V4.3 in the MAGPIX system. Background subtraction was performed on CTRL and IL-1β conditions using read out values from media only and 20 ng/mL conditioned media, respectively.
hMSC Treatment with Pharmacological Intervention

To assess the effects of pharmacological interventions on hMSC phospho-protein signaling, cells were treated in vitro 30 min prior to conditioning the cells with IL-1β. More therapeutic hMSCs (donors M1 and M2) were treated with p-p38 (SB203580; S1076; Selleckchem) and p-JNK (SP600125; S1460; Selleckchem) inhibitors independently and in combinations to perturb the MAPK signaling pathway. Analysis of the Akt signaling pathway implemented the use of a p-Akt activator (SC79; S7863; Selleckchem) in more therapeutic hMSCs. For less therapeutic hMSCs (donors L1 and L2) included the use of a p-JNK activator (Metformin; S1950; Selleckchem) in the MAPK signaling pathway and a p-insulin like growth factor 1 receptor (IGF1R; BMS-536924; S1012; Selleckchem), p-Akt (MK-2206; S1078; Selleckchem), and p-mTOR (Rapamycin; S1039; Selleckchem) inhibitors independently to analyze the Akt signaling pathway. Viability was confirmed (>90%) following administration of all therapeutic regimens when cell lysates and media were collected. Dosing studies were optimized for all therapeutic interventions employed (Table 4). To confirm pharmacological interventions were acting as expected on respective phospho-proteins, pharmacological interventions were added 30 mins prior to conditioning, followed by cell lysate collection at 15-mins, 60-mins, and 24 hours post conditioning (corresponding to media collection time point for cytokine analysis).

TABLE 3
Pharmacological interventions for
hMSC MAPK and Akt signaling.

| | | Signaling | Signaling | | |
|---|---|---|---|---|---|
| Drug | Activity | Pathway | Node | Concentration | |
| SB203580 | Inh | MAPK | p-p38 | 180 | nM |
| SP600125 | Inh | MAPK | p-JNK | 50 | nM |
| Metformin | Act | MAPK | p-JNK | 35 | mM |
| BMS-536924 | Inh | Akt | p-IGF1R | 10 | nM |
| MK-2206 | Inh | Akt | p-Akt | 2.5 | nM |
| SC79 | Act | Akt | p-Akt | 15 | nM |
| Rapamycin | Inh | Akt | p-mTOR | 100 | nM |

Partial Least Squares Discriminant Analysis

PLSDA was performed in MATLAB (Mathworks) using a function written by Cleiton Nunes (Mathworks File Exchange). This approach accounts for the multivariate nature of the data without overfitting. Prior to inputting the data into the algorithm, all data was z-scored [(observed-mean)/SD]. Intracellular signaling phospho-proteins and cytokine media read outs were used as the independent variables and the different hMSC donor's treatment conditions were used as the outcome variables. LVs in a multidimensional space (dimensionality varied by number of independent input variables) were defined and the two primary LVs were used for orthogonal rotation to best separate groups in the new plane defined by LV1 and LV2 (FIGS. 5a-5c).
Statistical Analysis

A post-hoc power analysis using α=0.05 and β=0.2, (power=0.8), using p-JNK and p-p38 levels and p-Akt and p-mTOR levels as the primary outcome measure for the MAPK and Akt signaling pathways, respectively, determined that a sample size of at least 3 cell wells was necessary to find statistical differences between treatment groups for the assessment of the MAPK and Akt signaling pathways. All data is presented as mean+SD. Significance for temporal traces for all phospho-protein quantification was determined with two-way ANOVA with condition and time implemented as independent variables. Statistical significance was set at p<0.05. All data was analyzed using the R stats, ggsignif, and ggpubr packages in R (The R Foundation).
Characterization of hMSCs

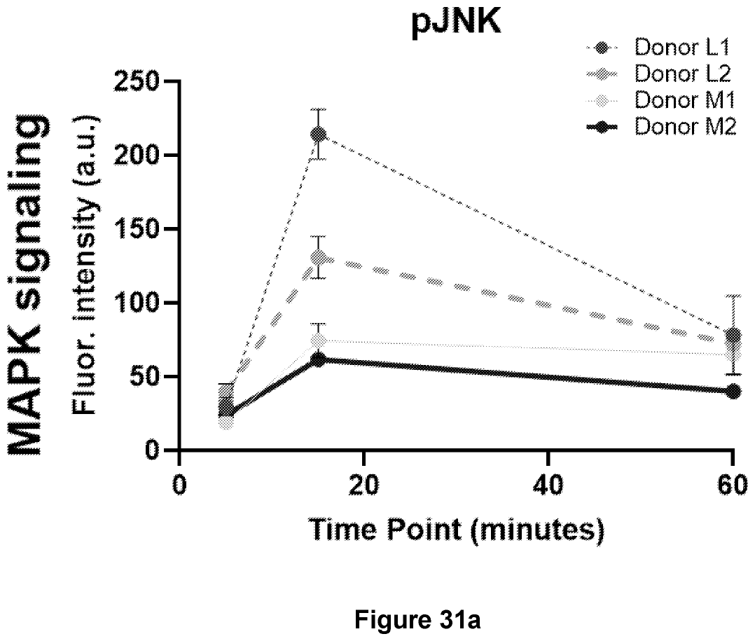

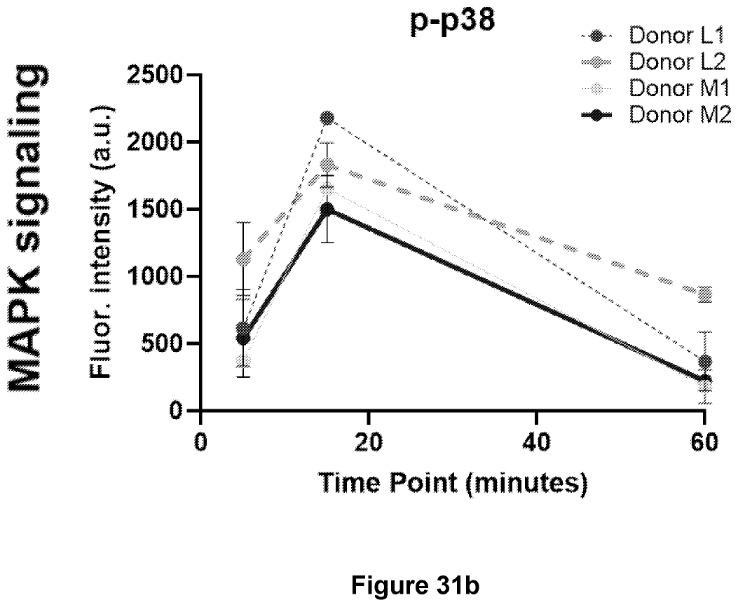

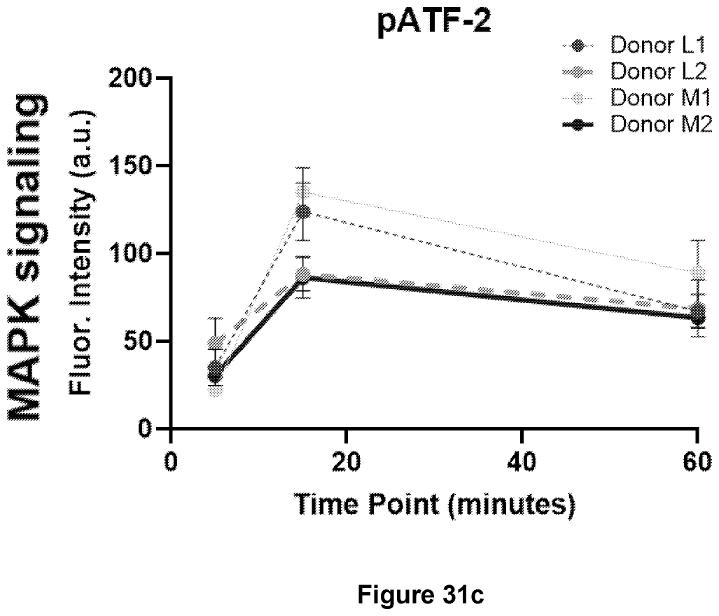

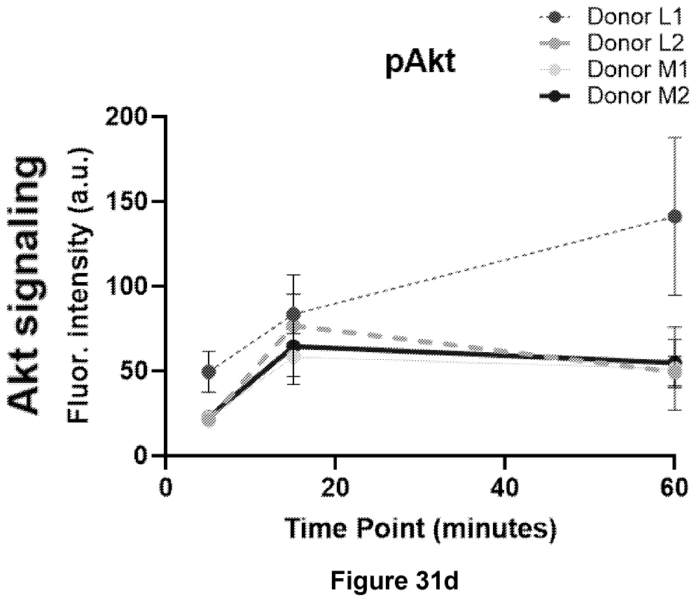

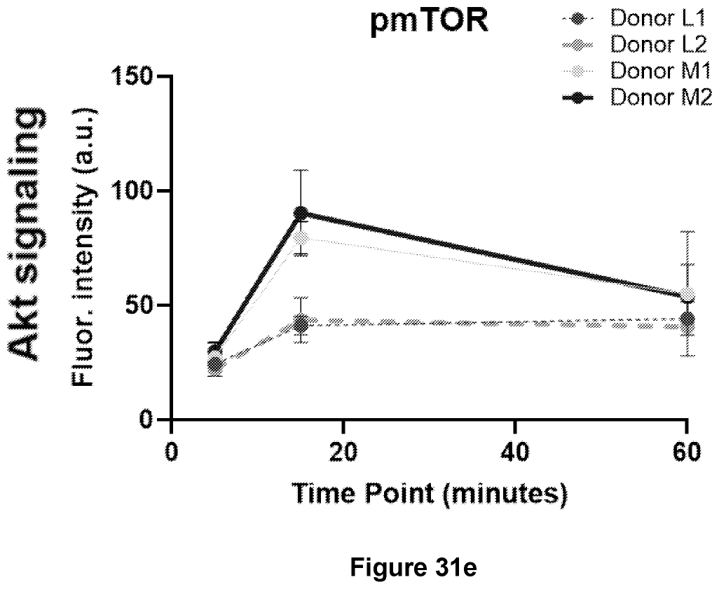

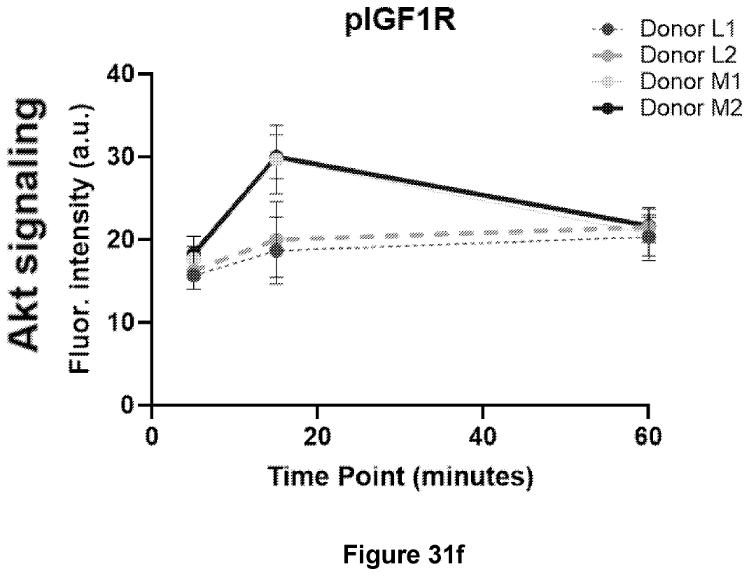

hMSC differentiation was confirmed for all four hMSC donors with immunofluorescent staining for type II collagen of paraffin-sectioned pellets, oil red O staining, and alizarin red S staining for chondrogenesis, adipogenesis, and osteogenesis, respectively. Additionally, all hMSC donors were confirmed to be positive for typical MSC markers, including CD73, CD90, and CD105, and negative for hematopoietic markers, including CD45, CD34, CD11b, CD79A, and HLA-DR.
hMSC MAPK and Akt Signaling Peak at 15 minutes post IL-1β Conditioning hMSC lysate was collected at 5, 15, and 60 mins post conditioning with IL-1β. To determine if temporal dynamics needed to be assessed or if a single time point could provide sufficient information to delineate differences in MAPK and/or Akt signaling in more therapeutic and less therapeutic hMSCs, temporal plots of MAPK and Akt signaling phospho-proteins were assessed, as illustrated in FIGS. 31a-31f. For all phospho-proteins quantified in the MAPK and Akt pathways, time showed a significant (p<0.0001) effect. Furthermore, in both the MAPK and Akt signaling pathways, the 15 min time point demonstrated a spike in signaling and thus motivated using the 15 min time point as the key time to assess differences between more therapeutic and less therapeutic hMSCs for the remaining intracellular signaling analysis (FIGS. 31a-31f).

As shown in FIGS. 31a-31c, p-JNK, p-p38, and p-Atf-2 phospho-protein signaling in the MAPK pathway yield signaling spikes in 15 mins, relative to the 5 and 60 min time points. Furthermore, p-Akt, p-mTOR, and P-IGF1R in the Akt pathway also yield spikes in signaling at 15 mins, relative to the 5 and 60 min time points, as shown in FIGS. 31d-31f. Time demonstrated a significant effect (p<0.0001) for all MAPK and Akt phospho-protein signaling assessed.
Inhibition of p-JNK phospho-protein Levels Reduced Secretion of GM-CSF, GRO, IL-4, and PDGF-AA In More Therapeutic hMSCs

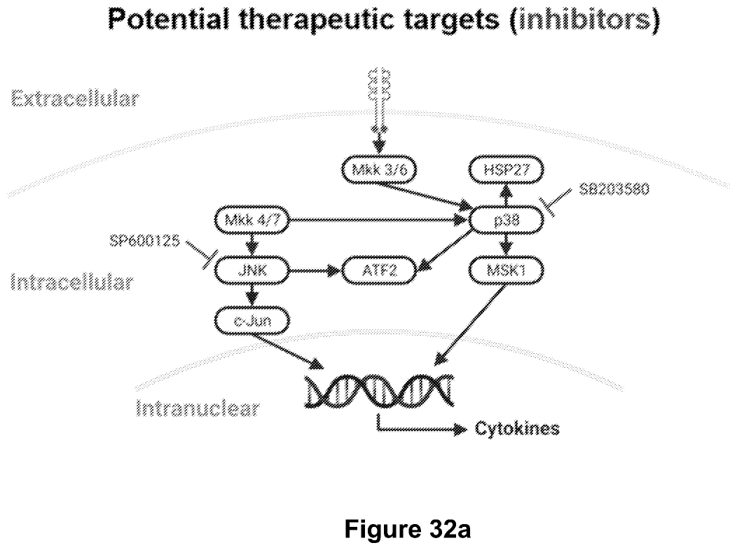

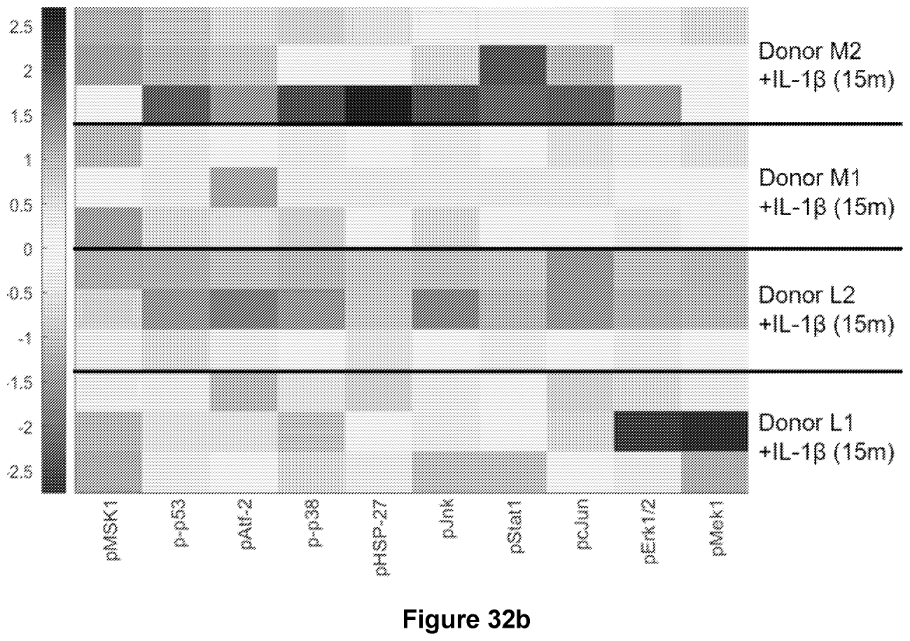

MAPK phospho-proteins were quantified at 5, 15, and 60 mins post IL-1β conditioning to assess for differences in phospho-protein signaling levels between more therapeutic and less therapeutic hMSCs, as shown in FIGS. 32a-32b. More therapeutic hMSCs at 15 mins (only time point assessed due to signaling spike observed temporally) demonstrated increased p-mitogen and stress activated protein kinase (MSK) 1, p-p53, p-Atf-2, p-p38, p-heat shock protein (HSP)-27, and p-JNK signaling relative to less therapeutic hMSCs (FIG. 32b). More specifically, the p-JNK and p-p38 phospho-proteins were targeted due to their central regulatory role they play in the MAPK pathway and due to the availability of well-established pharmacological interventions for these two phospho-proteins (FIG. 32a).

FIG. 32a illustrates signaling schematic of MAPK pathway (ERK pathway is not included) in hMSCs with inhibitor sites (labeled as SP600125 and SB203580) highlighted for the p-JNK and p-p38 phospho-protein signaling levels. FIG. 32b illustrates quantification of MAPK phospho-protein signaling levels in all cell lines demonstrated increased overall phospho-protein signaling levels in the more therapeutic hMSCs relative to the less therapeutic hMSCs; more specifically, more therapeutic hMSCs demonstrated increased p-JNK and p-p38 phospho-protein signaling levels relative to less therapeutic hMSCs.

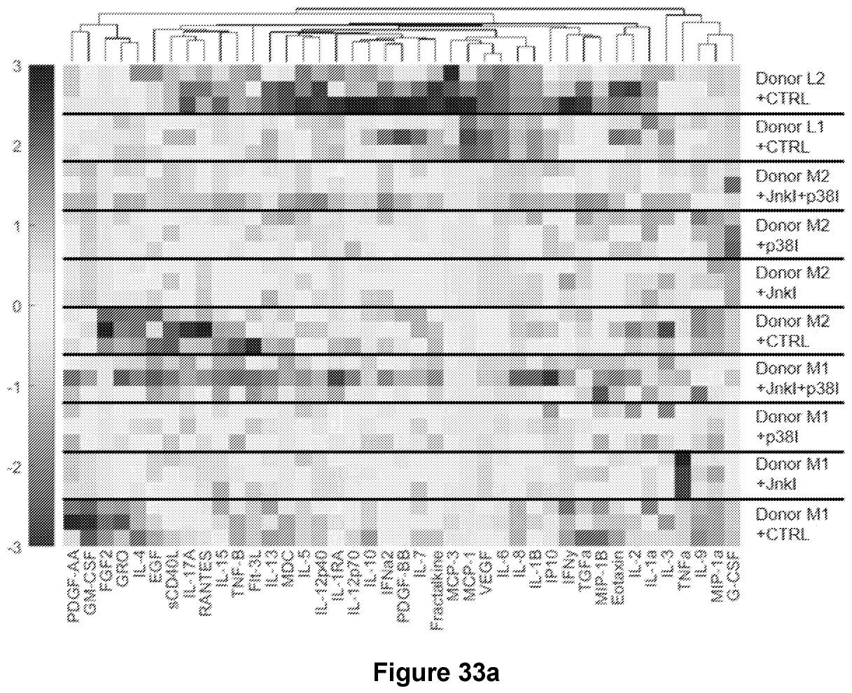

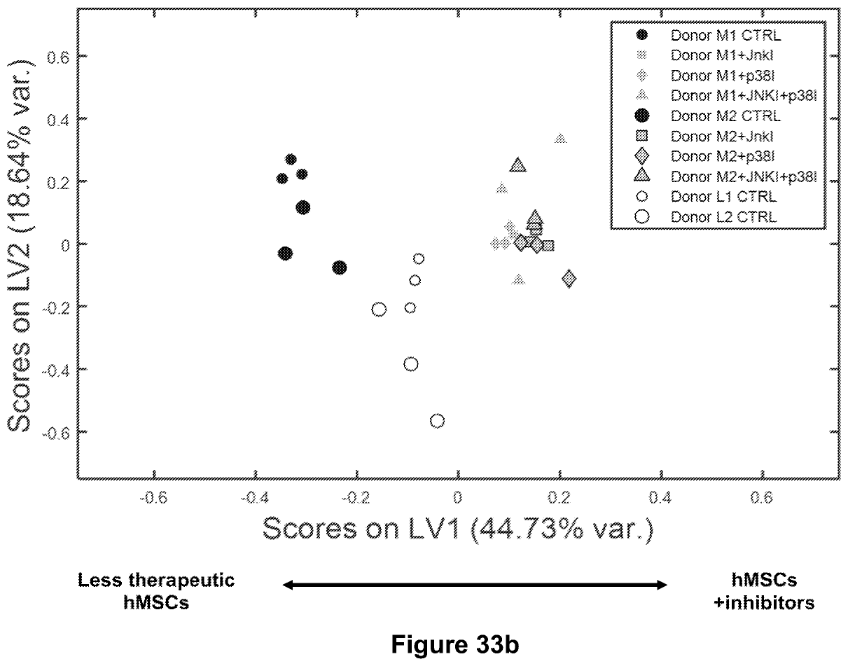

FIGS. 33a-33b illustrate paracrine signaling profiles of more therapeutic hMSCs (donors M1 and M2) treated with p-p38 (SB203580), p-JNK inhibitor (SP600125), and inhibitors in combination (p-p38+p-JNK). Inhibitors SP600125 and SB203580 were implemented to inhibit the p-JNK and p-p38 phospho-proteins, respectively, to assess their effects in mediating the paracrine response of more therapeutic hMSCs (FIG. 33a). While treatment of more therapeutic hMSCs with p-JNK, p-p38, and p-JNK+p-p38 inhibitors demonstrated clear shifts in the paracrine signaling profiles of donors M1 and M2, the paracrine signaling profiles yielded were not similar in profile to the paracrine signaling of less therapeutic hMSCs (donors L1 and L2; FIG. 33a). More specifically, treatment with p-JNK and p-p38 inhibitors yielded a rightward shift on LV1 relative to untreated less therapeutic hMSCs, in the PLSDA plot of the cytokine data; this was in the opposite direction of the more therapeutic hMSCs, which yielded a leftward shift on LV1, relative to the less therapeutic hMSCs (Donors L1 CTRL and L2 CTRL; FIG. 33b). While the profiles yielded for more therapeutic hMSCs did not align with the profiles of less therapeutic hMSCs, both the p-p38 and p-JNK inhibitors reduced cytokine secretion of the therapeutic related cytokines (GM-CSF, GRO, IL-4, PDGF-AA; FIG. 33a). Based on these findings the p-JNK inhibitor (SP600125) was implemented as an intervention to mediate hMSC cytokine secretion of the therapeutic related cytokines of more therapeutic hMSCs in OA.

FIG. 33a illustrates quantification of immunomodulatory cytokines for more therapeutic hMSCs, with different inhibitor conditions applied, show distinct shifts in overall cytokine signaling with addition of single and combinatorial inhibitor conditions. More specifically, treatment of more therapeutic hMSCs with all inhibitor conditions yielded decreased secretion of GM-CSF, GRO, IL-4, and PDGF-AA. As shown in FIG. 33b, PLSDA analysis identified a profile of cytokines along LV1 that identified a distinct separation between less therapeutic hMSCs to the left (Donors M1 CTRL and M2 CTRL) and more therapeutic hMSCs treated with inhibitors to the right (Donors M1+Jnkl, M1+p381, M1+JNKI+p381, M2+Jnkl, M2+p381, and M2+JNKI+p381), relative to more therapeutic hMSCs (Donors L1 CTRL and L2 CTRL). Variability accounted for in each LV is included on respective axes labels.
Activation of p-Akt phospho-protein Potentiated Nonspecific Cytokine Secretion In More Therapeutic hMSCs

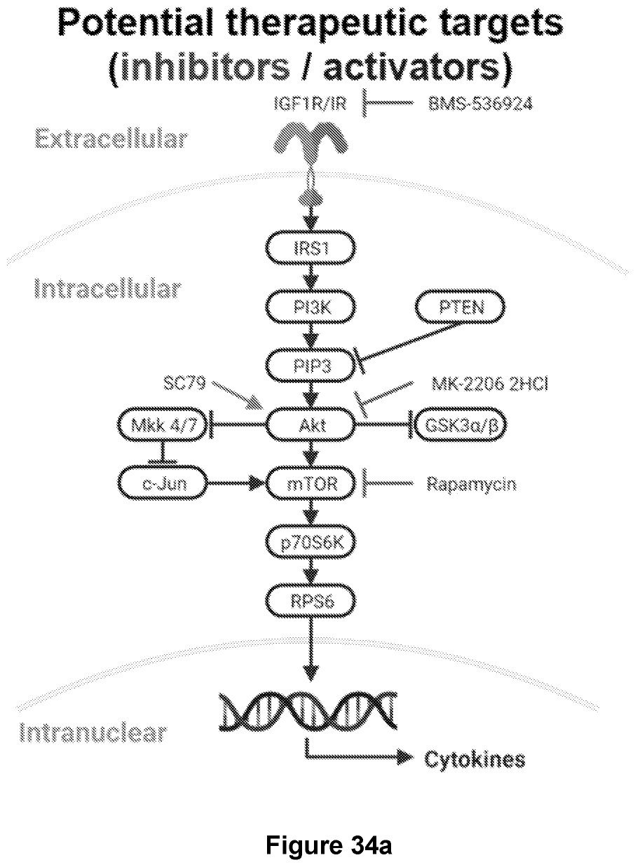

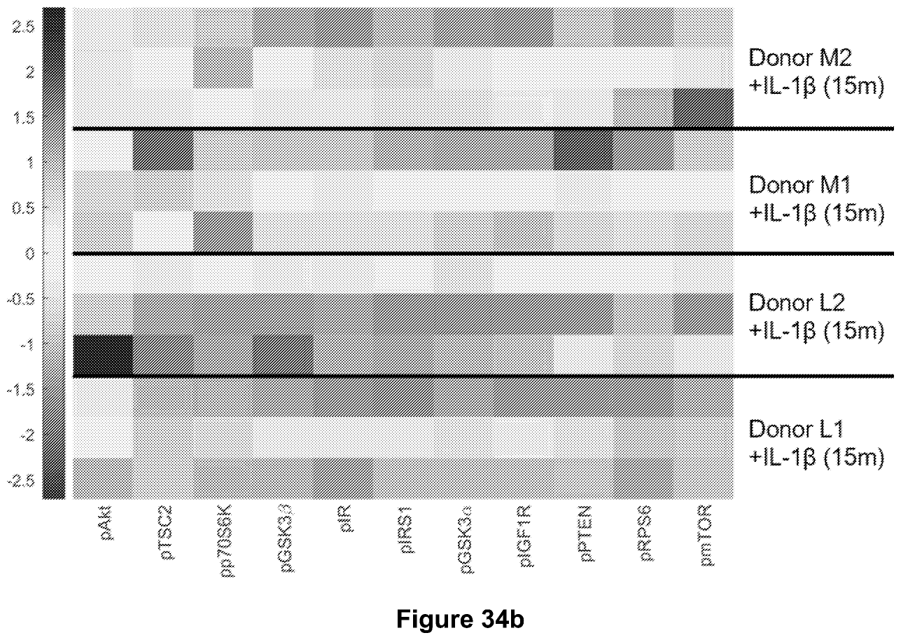

Akt phospho-proteins were quantified at 5, 15, and 60 mins post IL-1β conditioning to assess for differences in phospho-protein signaling levels between more therapeutic and less therapeutic hMSCs, as illustrated in FIGS. 34a-34b. More therapeutic hMSCs at 15 mins (only time point assessed due to signaling spike observed temporally) demonstrated increased p-tuberous sclerosis proteins (TSC) 2, p-p70S6K, p-GSK3β, p-insulin receptor (IR), p-insulin receptor substrate (IRS)₁, p-GSK3a, p-IGF1R, p-PTEN, p-ribosomal protein (RP) S6, and signaling relative to less therapeutic hMSCs (FIG. 34b). Furthermore, less therapeutic hMSCs demonstrated increased signaling for only the p-Akt phospho-protein, relative to more therapeutic hMSCs. More specifically, the p-IGF1R, p-mTOR and p-Akt phospho-proteins were targeted due to their central regulatory role they play in the Akt pathway and due to the availability of well-established pharmacological interventions for these two phospho-proteins (FIG. 34a).

FIG. 34a provides signaling schematic of Akt pathway in hMSCs with inhibitor (labeled as MK-2206 2HCl and Rapamycin) and activator (labeled as SC79) sites highlighted for the p-IGF1R, p-Akt, and p-mTOR phospho-proteins. FIG. 34b illustrates quantification of Akt phospho-protein signaling levels in all cell lines demonstrated increased signaling levels for all phospho-proteins, except Akt, in the more therapeutic hMSCs relative to the less therapeutic hMSCs; more specifically, more therapeutic hMSCs demonstrated increased p-IGF1R and p-mTOR phospho-protein signaling levels relative to less therapeutic hMSCs and less therapeutic hMSCs demonstrated increased p-Akt levels relative to more therapeutic hMSCs.

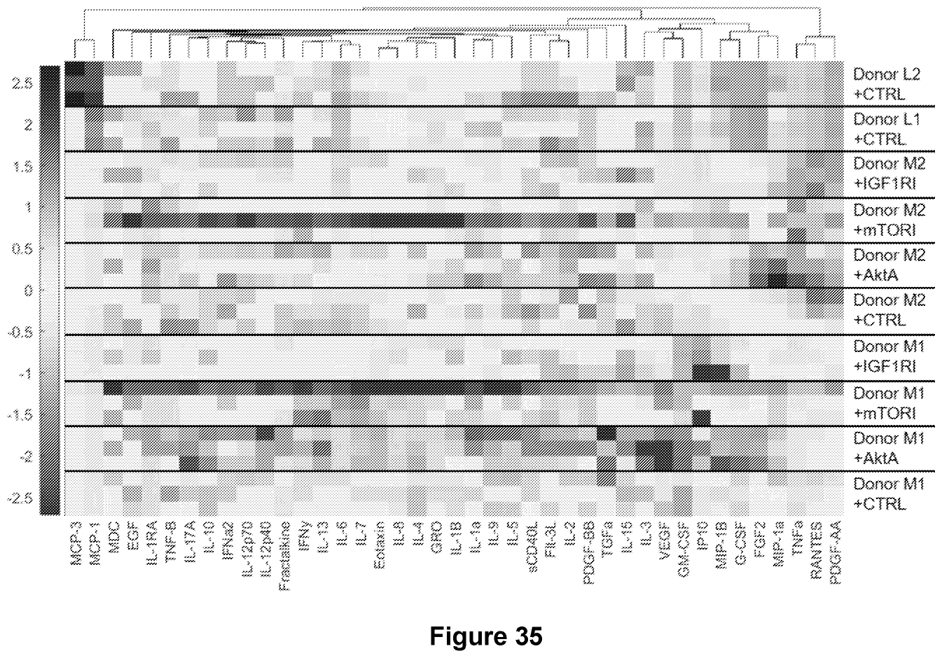

Inhibitors BMS-536924 and Rapamycin were implemented to inhibit the p-IGF1R and p-mTOR phospho-proteins, respectively, and an MK-2206 Akt activator were implemented to assess their effects in mediating the paracrine response of more therapeutic hMSCs, as illustrated in FIG. 35. While all therapeutic interventions yielded clear qualitative shifts on the paracrine signaling profiles of more therapeutic hMSCs (donors M1 and M2), treatment with the p-Akt activator and p-mTOR inhibitor demonstrated potentiation of the overall paracrine signaling response of more therapeutic hMSCs; while treatment with the p-IGF1R inhibitor yielded reduced overall paracrine signaling response (FIG. 35). However, treatment of both donors with the p-Akt activator yielded the greatest potentiation of paracrine signaling, relative to the CTRL conditions for both respective hMSC donors (FIG. 35). While these different intervention strategies had large effects on the paracrine signaling profiles of hMSC donors M1 and M2, there was no effect of these interventions on secreted levels of the therapeutic related cytokines GM-CSF, GRO, IL-4, and PDGF-AA (FIG. 35). Furthermore, none of the paracrine profiles yielded for any of the donor intervention conditions aligned with those of the less therapeutic hMSCs (donors L1 and L2). Based on these findings the p-Akt activator (SC79) was implemented as an intervention to mediate the hMSC cytokine secretion of nonspecific cytokines (cytokines that showed no relationship with therapeutic outcomes in OA) of more therapeutic hMSCs in OA.

Quantification of immunomodulatory cytokines for more therapeutic hMSCs, with different inhibitor conditions applied, show potentiated overall cytokine signaling with addition of an Akt activator and mTOR inhibitor. More specifically, treatment of more therapeutic hMSCs with an Akt activator potentiated overall cytokine signaling further than treatment with an mTOR or IGF1R inhibitor.

Inhibition of p-Akt phospho-protein In Akt Signaling Reduced Nonspecific Cytokine Secretion In Less Therapeutic hMSCs

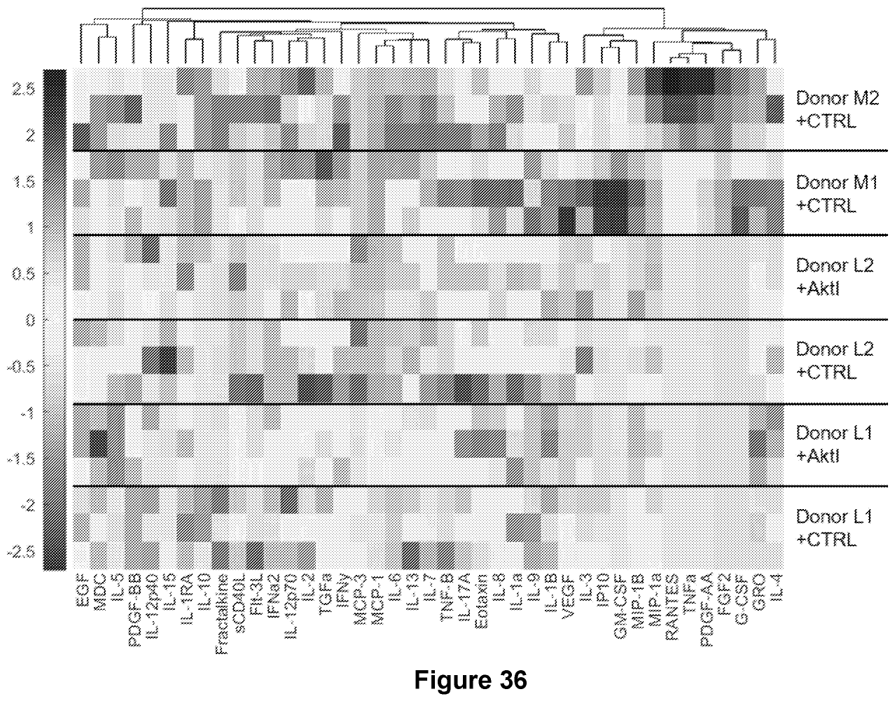

Inhibitor MK-2206 was implemented to inhibit the p-Akt phospho-protein to assess its effect in mediating the paracrine response of less therapeutic hMSCs, as illustrated in FIG. 36. Treatment of donors L1 and L2 with the p-Akt inhibitor yielded distinct reductions in nonspecific cytokines, relative to the respective CTRLs for each donor (FIG. 36). However, there was no effect of this inhibitor on secreted levels of the therapeutic related cytokines GM-CSF, GRO, IL-4, and PDGF-AA (FIG. 36). While this p-Akt inhibitor yielded distinct effects on the cytokine profiles of the less therapeutic hMSC donors, the resulting paracrine signaling profiles from treatment with intervention did not align with those yielded by more therapeutic hMSCs. Based on these findings the p-Akt inhibitor (MK-2206) was implemented as an intervention to mediate the hMSC cytokine secretion of nonspecific cytokines of less therapeutic hMSCs in OA.

FIG. 36 illustrates paracrine signaling profiles of less therapeutic hMSCs (donors L1 and L2) treated with an Akt inhibitor (MK-2206). Quantification of immunomodulatory cytokines for more therapeutic hMSCs, with different inhibitor conditions applied, show potentiated overall cytokine signaling with addition of all inhibitors implemented. More specifically, treatment of more therapeutic hMSCs with an Akt activator potentiated overall cytokine signaling further than treatment with an mTOR or IGF1R inhibitor.
Combination Therapy of SP600125 and SC79 Inhibits the p-JNK phospho-protein and Activates the p-Akt phospho-protein, Respectively, In More Therapeutic hMSCs

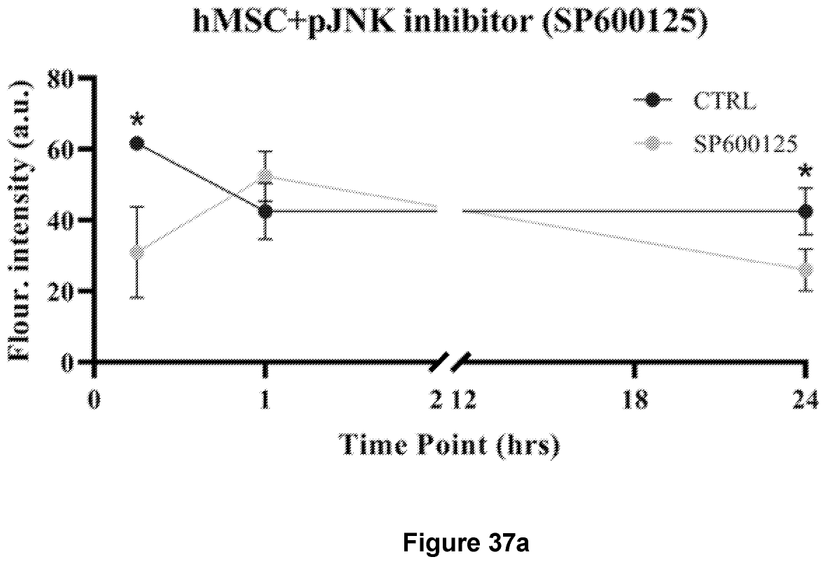

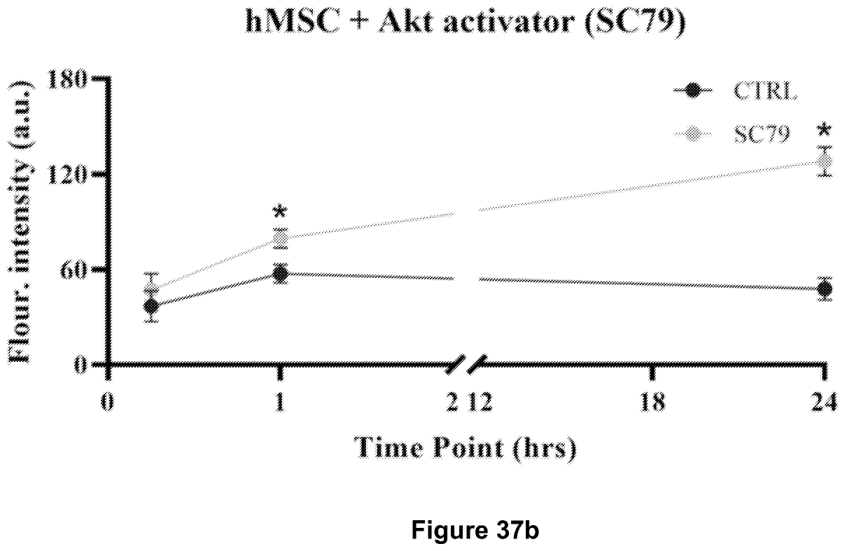

To confirm that the combinatorial pharmacological intervention strategy of SP600125 and SC79 inhibit p-JNK and activate p-Akt signaling, phospho-proteins in the MAPK and Akt signaling pathways were quantified in a single more therapeutic hMSC donor (M2), as illustrated in FIGS. 37a-37b. Treatment with a p-JNK inhibitor using SP600125 yielded a significant (p<0.05) decrease in p-JNK phospho-protein levels, relative to CTRL more therapeutic hMSCs (FIG. 37a). Furthermore, more therapeutic hMSCs treated with a p-Akt activator (SC79) yielded significantly (p<0.001) more p-Akt phospho-protein levels, relative to CTRL (FIG. 37b). These findings confirm the utility of SP600125 and SC79 combination therapy to inhibit p-JNK and activate p-Akt phospho-protein signaling.

As shown in FIG. 37a, p-JNK phospho-protein signaling in the MAPK pathway for hMSCs (donor M2) treated with SP600125 yielded a significant (p<0.05) decrease in overall p-JNK phospho-protein levels, relative to CTRL; significant decreases in p-JNK, with SP600125 treatment, were identified at 15 mins and 24 hours post conditioning. As shown in FIG. 37b, p-Akt phospho-protein signaling in the Akt pathway for hMSCs (donor M2) treated with SC79 yielded a significant (p<0.001) increase in overall p-Akt phospho-protein levels, relative to CTRL; significant increases in p-Akt, with SC79 treatment, were identified at 60 mins and 24 hours post conditioning. * represents significant differences (p<0.05) between inhibitor treated and CTRL groups at individual time points.
Combination Therapy of Metformin and MK-2206 Activates the p-JNK phospho-protein and Inhibits The p-Akt phospho-protein, Respectively, In Less Therapeutic hMSCs

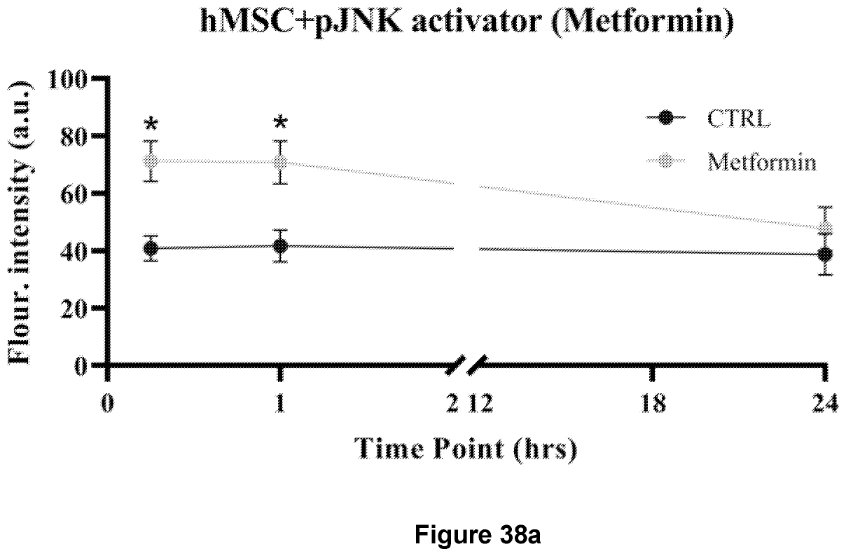

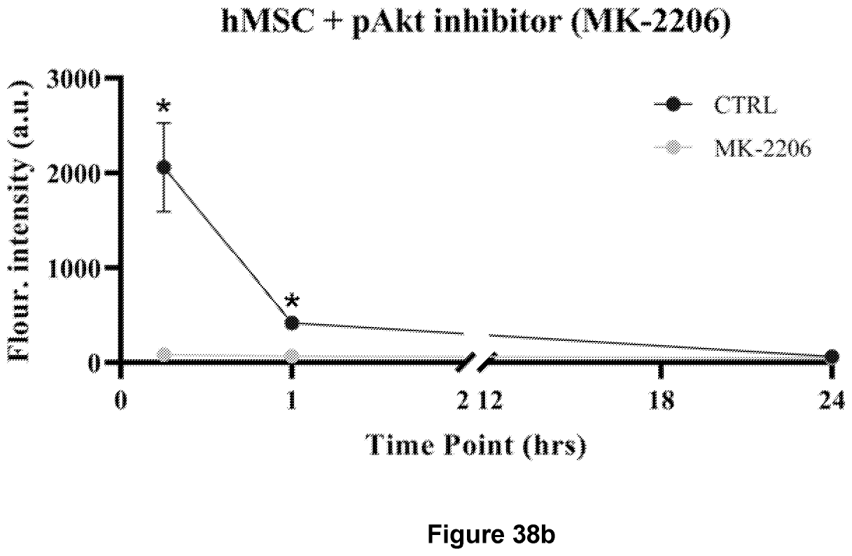

To confirm that the combinatorial pharmacological intervention strategy of Metformin and MK-2206 activate p-JNK and inhibit p-Akt signaling, phospho-proteins in the MAPK and Akt signaling pathways were quantified in a single less therapeutic hMSC donor (L2), as illustrated in FIGS. 38a-38b. Treatment with a p-JNK activator using Metformin yielded a significant (p<0.01) increase in p-JNK phospho-protein levels, relative to CTRL less therapeutic hMSCs (FIG. 38a). Furthermore, less therapeutic hMSCs treated with a p-Akt inhibitor (MK-2206) yielded significantly (p<0.01) less p-Akt phospho-protein levels, relative to CTRL (FIG. 38b). These findings confirm the utility of Metformin and MK-2206 combination therapy to activate p-JNK and inhibit p-Akt phospho-protein signaling.

As shown in FIG. 38a, p-JNK phospho-protein signaling in the MAPK pathway for hMSCs (donor L2) treated with Metformin yielded a significant (p<0.01) increase in overall p-JNK phospho-protein levels, relative to CTRL; significant increases in p-JNK, with Metformin treatment, were identified at 15 and 60 mins post conditioning. As shown in FIG. 38b, p-Akt phospho-protein signaling in the Akt pathway for hMSCs (donor L2) treated with MK-2206 yielded a significant (p<0.01) decrease in overall p-Akt phospho-protein levels, relative to CTRL; significant decreases in p-Akt, with MK-2206 treatment, were identified at 15 and 60 mins post conditioning. * represents significant differences (p<0.05) between inhibitor treated and CTRL groups at individual time points.
Combination Therapy of JNK Inhibitor and Akt Activator Shifts More Therapeutic hMSCs to a Less Therapeutic Paracrine Signaling Profile

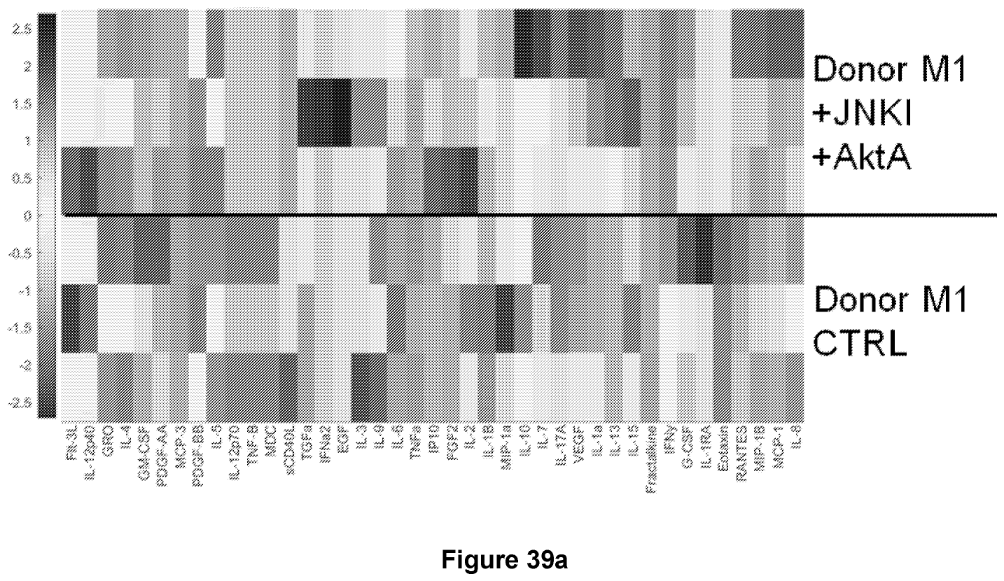

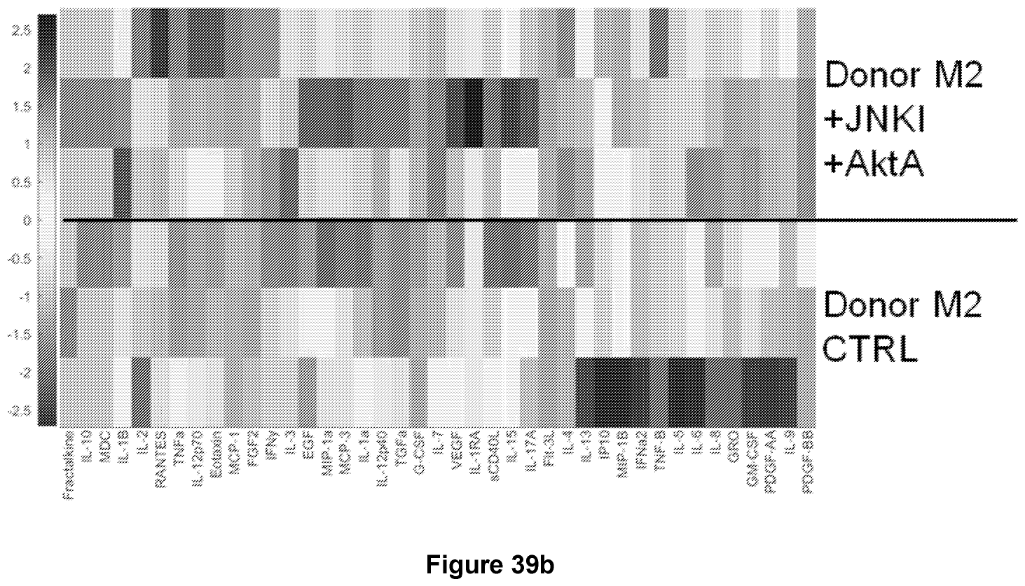

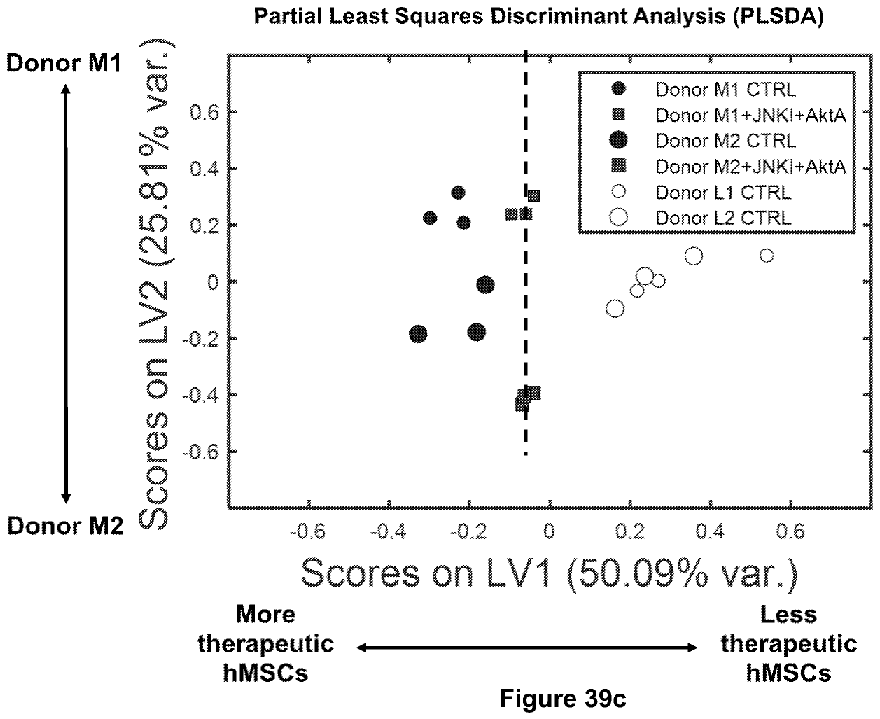

A p-JNK inhibitor (SP600125) and p-Akt activator (SC79) were administered in combination to shift the cytokine profile of more therapeutic hMSCs towards a less therapeutic paracrine signaling profile, as illustrated in FIGS. 39a-39c. This combination was based on the p-JNK inhibitor treated hMSCs (donor M1 and M2), alone, showing reduced secretion of the therapeutic related cytokines GM-CSF, GRO, IL-4, and PDGF-AA. Furthermore, the p-Akt activator treated hMSCs (donors M1 and M2) showed potentiated secretion of nonspecific cytokines in more therapeutic hMSCs. For more therapeutic hMSC donors treated with combination therapy, treated cells demonstrated a clear shift in paracrine signaling profiles, relative to the hMSC CTRLs for each respective donor (FIGS. 39a, 39b). More specifically, treated hMSCs demonstrated a less targeted cytokine profile (increase in the secretion of majority of cytokines assessed) and reduced secretion of the therapeutic related cytokines GM-CSF, GRO, IL-4, and PDGF-AA (FIGS. 39a, 39b). The intervention strategy implemented shifted the paracrine signaling profile of more therapeutic hMSCs to a less therapeutic hMSC cytokine profile (FIG. 39c). More specifically, treatment with a p-JNK inhibitor and p-Akt activator yielded a rightward shift on LV1, in the PLSDA plot of the cytokine data, towards less therapeutic hMSCs on the right (FIG. 39c). While each more therapeutic hMSC donors shifted to a less therapeutic phenotype (along LV1), each donor maintained their inherit unique paracrine signaling properties as clearly defined on LV2 (FIG. 39c). These findings indicate that the p-JNK phospho-protein signaling in the MAPK pathway mediates the secretion of therapeutic related immunomodulatory cytokines associated with hMSC therapeutic efficacy in OA treatment and that p-Akt phospho-protein signaling in the Akt pathway regulates secretion of cytokines associated with nontherapeutic outcomes in OA.

As shown in FIG. 39a, quantification of immunomodulatory cytokines for more therapeutic hMSC donor M1, with combination therapy applied, showed a less targeted (increase in overall cytokine secretion) paracrine profile relative to the donor M1 CTRL. More specifically, hMSCs (donor M1) treated with the Akt activator and JNK inhibitor showed reduced cytokine secretion of GM-CSF, GRO, IL-4, and PDGF-AA relative to the donor M1 CTRL. As shown in FIG. 39b, hMSC donor M2 yielded similar outcomes to donor M1 with less targeted cytokine secretion and reduced secretion of GM-CSF, GRO, IL-4, and PDGF-AA with treatment of an Akt activator and JNK inhibitor. As shown in FIG. 39c, PLSDA analysis identified a profile of cytokines along LV1 that identified a distinct separation between more therapeutic hMSCs to the left and less therapeutic hMSCs to the right. Addition of the combination therapy shifted the more therapeutic hMSCs towards a less therapeutic paracrine profile (rightward shift on LV1; Donors M1+JNKI+AktA and M2+JNKI+AktA). Furthermore, differences in donor paracrine signaling were maintained with interventions as indicated by separation along LV2. Variability accounted for in each LV is included on respective axes labels.

Combination Therapy of JNK Activator and Akt Inhibitor Shifts Less Therapeutic hMSCs To A More Therapeutic Paracrine Signaling Profile

A p-JNK activator (Metformin) and p-Akt inhibitor (MK-2206) were administered in combination to shift the cytokine profile of less therapeutic hMSCs towards a more therapeutic paracrine signaling profile, as illustrated in FIGS. 40a-40c. This combination was based on the p-JNK inhibitor treated hMSCs (donor M1 and M2), alone, showing reduced secretion of the therapeutic related cytokines GM-CSF, GRO, IL-4, and PDGF-AA. Thus, motivating the use of a p-JNK activator to potentiate the expression of these therapeutic defining cytokines in less therapeutic hMSCs. Furthermore, treatment of less related hMSCs with an p-Akt inhibitor showed reduced secretion of nonspecific cytokines in less therapeutic hMSCs. For both less therapeutic hMSC donors treated with combination therapy, treated cells demonstrated a clear shift in paracrine signaling profiles, relative to the hMSC CTRLs for each respective donor (FIGS. 40a, 40b). More specifically, treated hMSCs demonstrated a more targeted cytokine profile and increased secretion of the therapeutic related cytokines GM-CSF, GRO, IL-4, and PDGF-AA (FIGS. 40a, 40b). The intervention strategy implemented shifted the paracrine signaling profile of less therapeutic hMSCs to a more therapeutic hMSC cytokine profile (FIG. 40c). More specifically, treatment with a p-JNK activator and p-Akt inhibitor yielded a rightward shift on LV1, in the PLSDA plot of the cytokine data, towards more therapeutic hMSCs on the right (FIG. 40c).

While both less therapeutic hMSC donors shifted to a more therapeutic phenotype (along LV1), each donor maintained their inherent unique paracrine signaling properties as clearly defined on LV2 (FIG. 40c). These findings indicate that p-JNK phospho-protein signaling in the MAPK pathway and p-Akt phospho-protein signaling in the Akt pathway mediate the secretion of therapeutic immunomodulatory cytokines and can thus be targeted in hMSCs with less therapeutic potential to enhance their therapeutic potential.

As shown in FIG. 40a, quantification of immunomodulatory cytokines for less therapeutic hMSC donor L1, with combination therapy applied, showed a more targeted (decrease in overall cytokine secretion) paracrine profile relative to the donor L1 CTRL. More specifically, hMSCs (donor L1) treated with the Akt inhibitor and JNK activator showed potentiated cytokine secretion of GM-CSF, GRO, IL-4, and PDGF-AA relative to the donor L1 CTRL. As shown in FIG. 40b, hMSC donor L2 yielded similar outcomes to donor L1 with targeted cytokine secretion and reduced secretion of GM-CSF, GRO, and IL-4 (but not PDGF-AA) with treatment of an Akt inhibitor and JNK activator. As shown in FIG. 40c, PLSDA analysis identified a profile of cytokines along LV1 that identified a distinct separation between less therapeutic hMSCs to the left and more therapeutic hMSCs to the right. Addition of the combination therapy shifted the less therapeutic hMSCs towards a more therapeutic paracrine profile (rightward shift on LV1; Donors L1+JNKA+Akt1 and L2+JNKA+Akt1). Furthermore, differences in donor paracrine signaling were maintained with interventions as indicated by separation along LV2. Variability accounted for in each LV is included on respective axes labels.

### DISCUSSION

While there are likely many intracellular signaling pathways mediating the immunomodulatory response of MSCs, the Akt, MAPK, NF-κB pathways have been implicated as major contributors in the immunomodulatory potential of these cells and are upstream regulators of many critical immunomodulatory cytokines, including the majority of those assessed in Example 2. The data presented herein demonstrated that differences between less therapeutic and more therapeutic hMSCs in the Akt and MAPK signaling pathways, while no distinct differences were noted in the NF-κB signaling pathway. Thus, the phospho-protein signaling levels in both the MAPK and Akt pathway were quantified in all more therapeutic hMSC donors (M1 and M2) and less therapeutic hMSC(L1 and L2) donors in an OA simulated microenvironment (+IL-1β). These studies demonstrated that in more therapeutic hMSCs, MAPK and Akt signaling were potentiated, relative to less therapeutic hMSCs. More specifically, p-JNK and p-p38 phospho-protein levels in the MAPK pathway and p-Akt, p-IGF1R, and p-mTOR phospho-protein levels in the Akt pathway were potentiated in more therapeutic hMSCs.

Due to the central regulatory role these signaling pathways have in the paracrine signaling response of MSCs, the phospho-protein signals within these pathways provide viable therapeutic targets for mediating therapeutic MSC activity. To study the role of the MAPK pathway in hMSC paracrine signaling immunomodulatory cytokine profiles of hMSCs were quantified following pharmacological intervention with p-JNK and p-p38 inhibitors. While both interventions demonstrated clear mediation effects on hMSC paracrine signaling, more therapeutic hMSCs treated with a p-JNK inhibitor yielded reduced secretion of the cytokines GM-CSF, GRO, IL-4, and PDGF-AA, which were shown in Example 2 to relate to therapeutic outcomes in OA. A similar approach was implemented to the study the role of Akt signaling using a series of pharmacological interventions (both inhibitors and activators) for the p-IGF1R, p-Akt, and p-mTOR phospho-proteins. In these studies, all interventions demonstrated an ability to mediate the paracrine signaling response of hMSCs; of particular interest were the outcomes of less therapeutic hMSCs treated with a p-Akt inhibitor which yielded a more targeted paracrine signaling profile. However, even with this reduction in overall cytokine secretion, treatment of less therapeutic hMSCs with this p-Akt inhibitor did not yield increased secretion of the therapeutic related cytokines GM-CSF, GRO, IL-4, and PDGF-AA. The cumulative findings of these studies motivated implementation of a combination therapy using a p-JNK inhibitor and Akt activator combination in more therapeutic hMSCs and a p-JNK activator and Akt inhibitor in less therapeutic hMSCs.

To identify the cumulative signaling pathway dynamics mediating the paracrine signaling profile of more therapeutic hMSCs in OA, an intervention combination of a JNK inhibitor and Akt activator were used to treat hMSC donors M1 and M2. The hypothesis was that treatment of more therapeutic hMSCs with a combination of a JNK inhibitor and Akt activator would shift the cytokine secretion to a less therapeutic paracrine profile. In this study, treated more therapeutic hMSCs demonstrated a less targeted cytokine profile (increase in secretion of majority of cytokines assessed) and reduced secretion of the therapeutic related cytokines GM-CSF, GRO, IL-4, and PDGF-AA. With this intervention, treated cells demonstrated a clear shift towards the paracrine phenotype of less therapeutic hMSCs. These findings indicate that the p-JNK phospho-protein in the MAPK pathway mediates the secretion of therapeutic related immunomodulatory cytokines associated with hMSC therapeutic efficacy in OA treatment and the p-Akt phospho-protein in the Akt pathway regulates secretion of cytokines associated with nontherapeutic outcomes in OA. These findings motivated the study of pharmacological intervention strategies to take hMSCs with less therapeutic paracrine profiles and shift them towards a more therapeutic phenotype.

Treatment of less therapeutic hMSCs with a combination of a JNK activator and Akt inhibitor was shown to shift the cytokine secretion to a more therapeutic paracrine profile. Treatment of hMSCs with a less therapeutic paracrine signaling profile yielded a more therapeutic cytokine profile. More specifically, less therapeutic hMSCs demonstrated more targeted paracrine signaling and increased secretion of therapeutic related cytokines GM-CSF, GRO, IL-4, and PDGF-AA. Furthermore, with this intervention, treated cells demonstrated a clear shift towards the paracrine phenotype of more therapeutic hMSCs. This ability to mediate MSC paracrine signaling phenotypes using these therapeutic intervention strategies presents a promising approach to potentiate current MSC therapeutics used in the clinical space as allogeneic MSCs are already being used for treatment of OA in pre-clinical studies. While these studies have demonstrated therapeutic outcomes in OA patients, the scalability of these allogeneic MSC therapies remains a challenge due to the low number of MSCs that are collected in each isolation procedure, which leads to the need to pool MSCs from different donors together, therefore yielding a highly heterogenous population of cells, which presents many unique challenges as clearly demonstrated in this thesis. The data presented in this Example demonstrate that therapeutic interventions can be employed to mediate the intracellular signaling pathways of hMSCs to yield more therapeutic phenotypes (paracrine signaling specific) in OA.

Though Metformin has been studied previously in the context of MSC therapeutic for OA, the inhibition of Akt with MK-2206 in combination with Metformin remains a novel approach. Furthermore, MK-2206 has not been studied as an independent therapeutic intervention for MSCs as OA therapeutics. While this therapeutic remains not approved by the FDA, there are a number of ongoing clinical trials using this inhibitor to treat solid tumors in clinical patients. Thus, the pharmacological intervention proposed herein implemented clinically relevant therapeutics as interventions to mediate therapeutic hMSC paracrine signaling as OA therapeutics.

### EXAMPLE 4: Combination Therapy of Metformin and MK-2206 Potentiates Mesenchymal Stromal Cell Signaling Profile Associated with Therapeutic Efficacy for Osteoarthritis

MSC therapeutics for OA treatment have been widely studied, in both the pre-clinical and clinical environment. While pre-clinical studies have demonstrated improved cartilage repair with MSC treatment, effective translation into the clinic has been limited by numerous factors ranging from high variability and heterogeneity of MSCs to poor understanding of critical quality and potency attributes. Prior research has demonstrated that therapeutic potency varies in MSCs isolated from different human donors for treatment of other disease states; however, this donor heterogeneity remains understudied for MSCs as OA therapeutics.

Because hMSC function is thought to be mediated largely by immunomodulatory function exerted by secretion of paracrine signaling molecules, such as cytokines, paracrine signaling has recently been considered to be an essential quality attribute for hMSC potency for OA therapy. Based on this premise, the inventors decided to identify secreted paracrine factors related to therapeutic efficacy to gain insight into potential mechanisms responsible differences in paracrine factor secretion. Without wishing to be bound by theory, it is suggested that differences in certain central intracellular signaling pathways drive distinct paracrine secretion profiles, which may drive therapeutic outcomes.

### Materials and Methods

hMSC Culture and Characterization

hMSCs derived from bone marrow were obtained from Emory Personalized Immunotherapy Core (EPIC) at Emory University and RoosterBio (MSC-004; RoosterBio, Inc; Frederick, MD, USA). EPIC hMSCs were cultured in Mesenchymal Stem Cell Basal Medium (PT-3238; Lonza; Basel, Switzerland) supplemented with 10% heat-inactivated FBS (S11110H; Atlanta Biologicals; Oakwood, GA, USA), 1 mM L-glutamine (SH3003401; HyClone; Logan, UT, USA), and 100 μg/mL P/S (B21110; Atlanta Biologicals) and sub-cultured at 80% confluency. Rooster hMSCs were cultured in RoosterNourish-MSC (KT-001; RoosterBio, Inc) media supplemented with 2% RoosterBooster (SU-003; RoosterBio, Inc) and sub-cultured at 80% confluency. hMSC phenotypes for all donor hMSCs were confirmed by adipogenic, chondrogenic, and osteogenic differentiation. Flow cytometry was also used to characterize the hMSCs to confirm that all donor cells expressed characteristic MSC surface markers (CD73, CD90, CD105) and lacked hematopoietic markers (CD45, CD34, CD11b, CD79A, HLA-DR). Metadata was also collected for all hMSC donors, as provided below in Table 5.

TABLE 5
Metadata for hMSC donors used.

| Donor # | Source | Sex | Age | Tissue | Passage |
|---|---|---|---|---|---|
| 1 | EPIC | F | 5 | Bone Marrow | 4 |
| 2 | EPIC | M | 4-20 | Bone Marrow | 4 |
| 3 | RoosterBio | M | 18-30 | Bone Marrow | 4 |
| 4 | RoosterBio | F | 26 | Bone Marrow | 4 |

In vivo MMT Model of OA

Animal care and experiments were conducted in accordance with the institutional guidelines of the VAMC and experimental procedures were approved by the Atlanta VAMC IACUC (Protocol: V004-15). Weight-matched wild type male Lewis rats (strain code: 004; Charles River), weighing 300-350 g, were acclimatized for 1 week after they were received. A surgical instability animal model, MMT, was used to induce OA, as previously described. hMSC therapeutics were assessed for their ability to prevent further development of established OA using a six week time course, with therapeutics injected at three weeks (corresponding to OA phenotype presentation) followed by animal takedown three weeks later at the six-week end point. All MMT animals received 50 μL intra-articular injections using a 25-gauge needle. Animals were injected with 1) HBSS (MMT/Saline; n=8), 2) 5×10⁵ hMSC/knee of donor L1 [MMT/hMSC(Donor L1); n=8], 3) 5×10⁵ hMSC/knee of donor L2 [MMT/hMSC(Donor L2), 4) 5×10⁵ hMSC/knee; n=8] of donor M1 [MMT/hMSC(Donor M1); n=7], or 5) 5×10⁵ hMSC/knee of donor M2 [MMT/hMSC(Donor M2); n=8]. The cell dose (5×10⁵ cells/knee) used for injection was matched with the dosage optimized in a prior study. Sham animals were not injected post-surgery (n=8). Animals were euthanized at 6-weeks post-surgery via CO₂ asphyxiation. Left hindlimbs were dissected and fixed in 10% neutral buffered formalin. Muscle and connective tissues were removed from the hindlimbs. The femur was disarticulated from the tibia. Meniscus and residual soft tissue surrounding the medial tibial condyle were dissected and discarded.

microCT Quantitative Analysis of Articular Joint Parameters

Tibiae were immersed in 30% (diluted in PBS) hexabrix 320 contrast reagent (NDC 67684-5505-5; Guerbet) at 37° C. for 30 mins before being scanned. All samples were scanned using microCT through the use of a Scanco μCT 40 (Scanco Medical) using the following parameters: 45 kVp, 177 μA, 200 ms integration time, isotropic 16 μm voxel size, and about 27 min scan time. Scans were read out as 2D tomograms which were subsequently orthogonally transposed to yield 3D reconstructions for all scanned samples. All microCT parameters (articular cartilage, osteophyte, and subchondral bone) were evaluated as previously described. For cartilage parameters, thresholding of 110-435 mg hydroxyapatite per cubic cm (mg HA/cm³) was used to isolate the cartilage from the surrounding air and bone. Furthermore, for bone parameters, thresholds of 435-1200 mg HA/cm³ were implemented to isolate bone from the overlying cartilage.

### MATLAB Articular Cartilage Surface Roughness Analysis

Serial 2D images of the proximal tibiae were analyzed using a customized algorithm in MATLAB (Math Works) to quantify surface roughness, lesion volume, and full-thickness lesion area. Images were processed to generate a 3D surface of the articular cartilage surface. This 3D rendering was fitted along a 3D polynomial surface: fourth order along the ventral-dorsal axis and second order along the medial-lateral axis. The root mean square difference between the generated (actual) and polynomial fitted (predicted) surfaces was the measure of cartilage layer surface roughness. Lesion volume was calculated as the volume of root mean square difference between the generated fitted surface and the polynomial surface where >25% of total (predicted) cartilage thickness was exceeded. Full-thickness lesion area, also called exposed bone, was the sum of the area on the tibial condyle where no cartilage layer was present. Surface roughness, lesion volume and full-thickness lesion area were calculated for full and medial 1/3 region of the articular cartilage.

In vitro OA Simulated Microenvironment

hMSCs, matching donor with in vivo MMT model, were utilized in vitro. hMSCs were sub-cultured to 80% confluency in complete Lonza for hMSC donors L1 and L2 and Rooster medium for hMSC donors M1 and M2 in 12-well plates and cultured at 37° C., 5% CO₂. Each of the four hMSC donors were independently conditioned with 20 ng/ml IL-1β conditioned media (FHC05510; Promega; Madison, WI, USA; +IL-1β). IL-1β was used to model the OA inflammatory environment in the current study as it is a major pro-inflammatory modulator in OA. IL-1β concentrations were selected based on prior experiments and preliminary data. The immunomodulatory cytokine content of all SF samples was characterized using a bead based multiplex immunoassay, Luminex Cytokine/Chemokine 41 Plex Immunomodulatory Kit (HCYTMAG-60K-PX41; EMD Millipore Corporation; Burlington, MA, USA). Median fluorescent intensity values were read out using Luminex xPONENT software V4.3 in the MAGPIX system. Conditioned media was added to hMSCs at day 0 followed by a 24-hr conditioning period in monolayer culture with media collection (Luminex cytokine analysis) and cell lysate collection (RNA-Seq analysis) at the study end point. Samples were stored at −80° C. until analysis was performed.

hMSC Cytokine Analysis

Cytokines were quantified for all donors with IL-1β conditioning (n=3). Loaded samples (2.03 μL) were determined to be within the linear range of detection of the MAGPIX (MAGPIX-XPON4.1-CEIVD; EMD Millipore Corporation) system. Cytokines were quantified using a bead based multiplex immunoassay, Luminex Cytokine/Chemokine 41 Plex Immunomodulatory Kit (HCYTMAG-60K-PX41; EMD Millipore Corporation). Median fluorescent intensity values were read out using Luminex xPONENT software V4.3 in the MAGPIX system. Background subtraction was performed using read out values 20 ng/ml IL-1β conditioned media.

hMSC RNA-Seq Analysis

RNA transcripts were quantified for all four hMSC donors with +IL-1β conditioned media. RNA was isolated from hMSCs using the Qiagen RNeasy kit (217804; Qiagen; Hilden, Germany) according to the manufacturer's protocols. RNA samples were submitted to the Molecular Evolution Core at the Georgia Institute of Technology for sequencing. Quality control was run on all samples using a bioanalyzer to determine that the RNA Integrity Number (RIN) of the samples was greater than 7. A NEBNext Poly(A) mRNA Magnetic Isolation Module (E7490S; New England Biolab; Ipswich, MA, USA) and NEBNext Ultra II Directional RNA Library Prep Kit (E7760; New England Biolab) were used to generate libraries for sequencing. Quality control was run on all generated libraries using the bioanalyzer and the library was quantified using fluorometric methods. Sequencing was performed using the NovaSeq 6000 Sequencing System to obtain a sequencing depth of 30-40 million reads per sample. Samples were merged from four technical replicate lane. Transcripts obtained were aligned using DNAstar Array Star and Qseq and reads were mapped to the Homo sapiens (human) genome assembly GRCh38 (p14) from the genome reference database. For the read assignment, the threshold was set at 20 bp and 80% of the bases matching within each read. All duplicate reads were eliminated and genes with less than 20 total raw counts were removed. Counts were normalized using DESeq2 package in R (R).

### Gene Set Variation Analysis

To establish differences in each gene set, GSVA was used to identify enrichment of gene sets across all donors. GSVA is an improved gene set enrichment method that detects subtle variations of pathway activity over a sample population in an unsupervised manner. The GSVA was conducted using the Molecular Signatures Database C2 and C7 gene sets (MSigDB). Statistical differences in enrichment scores for each gene set between groups were computed by comparing the true differences in means against the differences computed by a random distribution obtained by permuting the gene labels 1000 times. FDR adjusted p-values were computed for detection of differences between donors with statistical significance set at FDR<0.25. GSVA was performed using the GSVA v1.36.1 in R (The R Foundation).

### Principal Component Analysis

PCA was conducted in R using the stats package v.3.6.2. Prior to inputting the data into the algorithm, all data was z-scored [(observed-mean)/SD]. An orthogonal rotation in the principal component (PC) 1-PC 2 plane was used to obtain new PC's that better separated treatment groups based on maximizing variance of the data set (covariance=0). Loadings plots were generated from this analysis and display the relative importance of input variables (cytokines) in contributing to the final composite values (scores) for each sample. RNA transcript read outs were used as the independent variable, while no dependent variable was set as PCA is an unsupervised approach.

### Partial Least Squares Discriminant Analysis

PLSDA was performed in MATLAB (Mathworks) using a function written by Cleiton Nunes (Mathworks File Exchange). This approach accounts for the multivariate nature of the data without overfitting. Prior to inputting the data into the algorithm, all data was z-scored [(observed−mean)/SD]. Secreted cytokines level read outs (from the pharmacological intervention studies) were used as the independent variables and the different hMSC donors were used as the outcome variables. LVs in a multidimensional space (dimensionality varied by number of independent input variables) were defined and the two primary LVs were used for orthogonal rotation to best separate groups in the new plane defined by LV1 and LV2.

### Partial Least Squares Regression

PLSR was performed in MATLAB (Mathworks) using the built in plsregress function. This approach accounts for the multivariate nature of the data without overfitting. Prior to inputting the data into the algorithm, all cytokines were z-scored [(observed-mean)/SD]. For determination of cytokines that relate to therapeutic outcomes of hMSCs in OA, cytokine secretion levels were used as the input with the output being microCT outcomes (articular cartilage, osteophyte, and subchondral bone analyses). LVs in a multidimensional space were defined and the two primary LVs were used for orthogonal rotation to best separate groups in the new plane defined by LV1 and LV2. Loadings plots were generated from this analysis and display the relative importance of input variables (cytokines) in contributing to the final composite values (scores) for each sample. Error bars on each cytokine (in the loadings plots) were computed by PLSR model regeneration using iterative (1000 iterations) LOOCV. PLSDA was performed in MATLAB (Mathworks) using a function written by Cleiton Nunes (Mathworks File Exchange).

### Statistical Analysis

All data is presented as mean±SD. Significance for all microCT parameters was determined with one-way ANOVA with post hoc Tukey honest test for articular cartilage and subchondral bone parameters. Bonferroni correction was used for post hoc analysis for the exposed bone and osteophyte parameters due to their nonparametric nature. To determine significant differences between different hMSC conditions (donor vs. donor, +CTRL vs. +drug, more therapeutic vs. less therapeutic) for individual cytokines and phospho-proteins, two tailed t-tests were used with Bonferroni correction to account for the independent analysis of multiple groups. To quantify the relationship between RNA gene expression pathways and articular cartilage microCT outcomes, least squares linear regression models were generated and Pearson's correlation coefficient, R, together with the p-value calculated from an F test (null hypothesis that the overall slope is zero) were reported. For all analysis, p <0.05 was considered statistically significant. All analyses were performed using GraphPad Prism 9 (GraphPad Software; La Jolla, CA, USA).

hMSCs from Different Donors Yield Variable Therapeutic Outcomes on Articular Cartilage

The inventors began the current study by asking if they could identify a panel of hMSC donors that exhibited a range of OA therapeutic outcomes. To do so, the inventors sourced four MSC donors from Emory Personalized Immunotherapy Core (EPIC) at Emory University and RoosterBio, and injected them for therapy in the rat MTT model of OA. Six weeks after surgery, the inventors collected tissues and conducted a detailed quantitative analysis of the articular cartilage changes in established OA using various morphological parameters for the total and medial 1/3 of the medial tibial condyle, as illustrated in FIGS. 41a-41p. For total articular cartilage volume, donors 3 and 4 showed reduced increases in articular cartilage volume relative to the saline control group; however, donors 1 and 2 showed no difference in volume compared with the saline (FIG. 41a). Similar outcomes were observed in the medial 1/3 region for articular cartilage volume (FIG. 41b). For articular cartilage thickness volume, while no differences were observed between MMT conditions for the total analyses in the medial 1/3 region, donors 3 and 4 showed reduced articular cartilage thickness relative to saline (FIGS. 41c, 41d). For articular cartilage attenuation, which is inversely related to articular cartilage proteoglycan content, no notable differences were observed between hMSC donors as all MMT conditions showed significantly higher attenuation in both the total and medial 1/3 analyses (FIGS. 41e, 41f). For total articular cartilage surface roughness, donors 3 and 4 showed attenuated articular cartilage surface roughness relative to saline; however, donors 1 and 2 showed no difference in surface roughness with the MMT/Saline disease control (FIG. 41g). Similar outcomes were observed in the medial 1/3 region for articular cartilage surface roughness (FIG. 41h). For articular cartilage lesion volume, donors 3 and 4 showed reduced lesion volume relative to the other donors as they showed no significant increase relative to the sham, while donors 1 and 2 did; furthermore, donors 3 and 4 showed significantly less lesion volume relative to donors 1 and 2 (FIG. 41i). No differences were observed between any MMT conditions for the exposed bone area (FIG. 41j). The data is presented as mean±/−SD; n=7 for MMT/hMSC(2.3); and n=8 for all other groups. * represents significant differences (p<0.05) between individual MMT groups and sham. Horizontal black bars indicate significance (p<0.05) between individual MMT groups.

Overall, these metrics reveal that donors 3 and 4 yielded more therapeutic outcomes, relative to donors 1 and 2, as they yielded reduced increases in articular cartilage swelling (volume and thickness), fibrillation development (surface roughness), and development of lesions (lesion volume). Representative microCT and histological images are provided for all groups assessed (FIGS. 41k-41p).

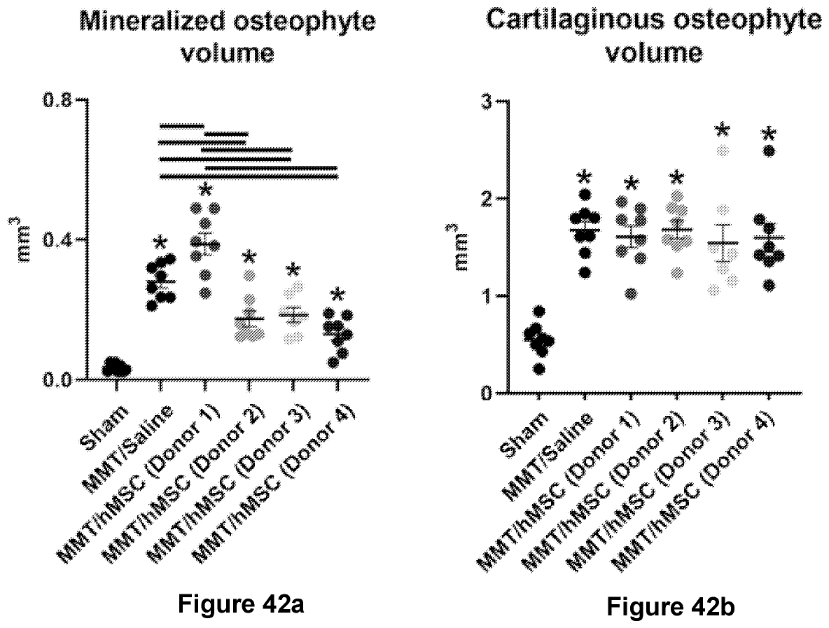

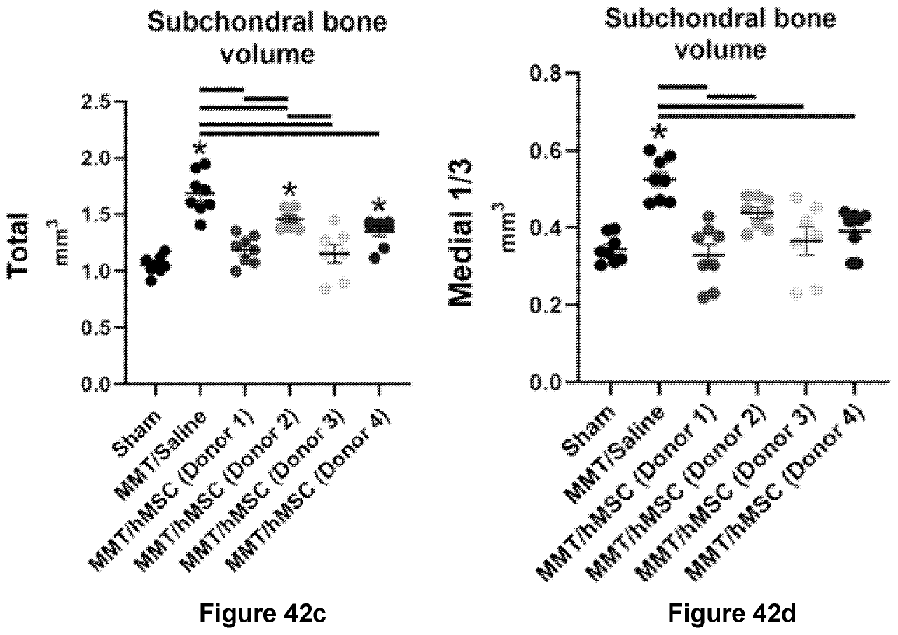

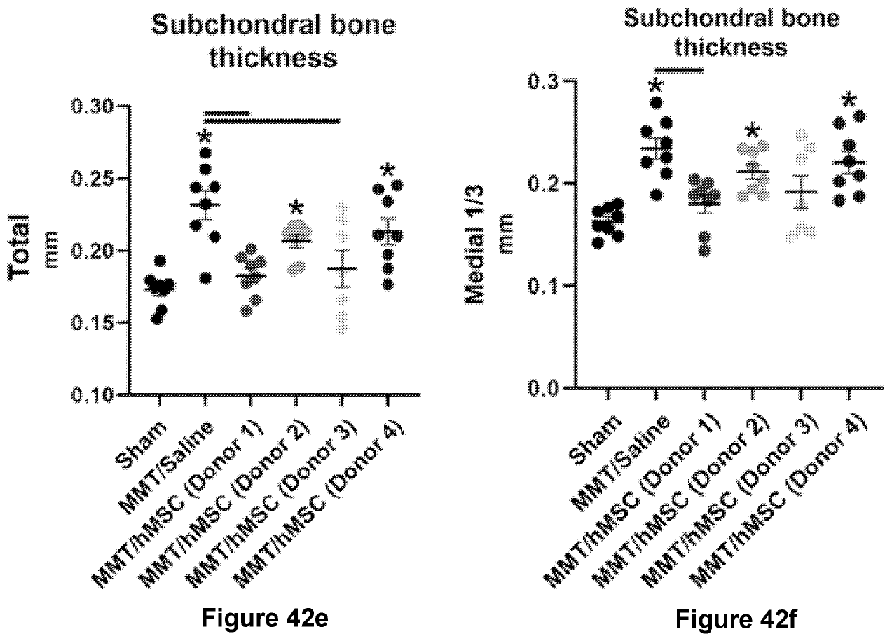

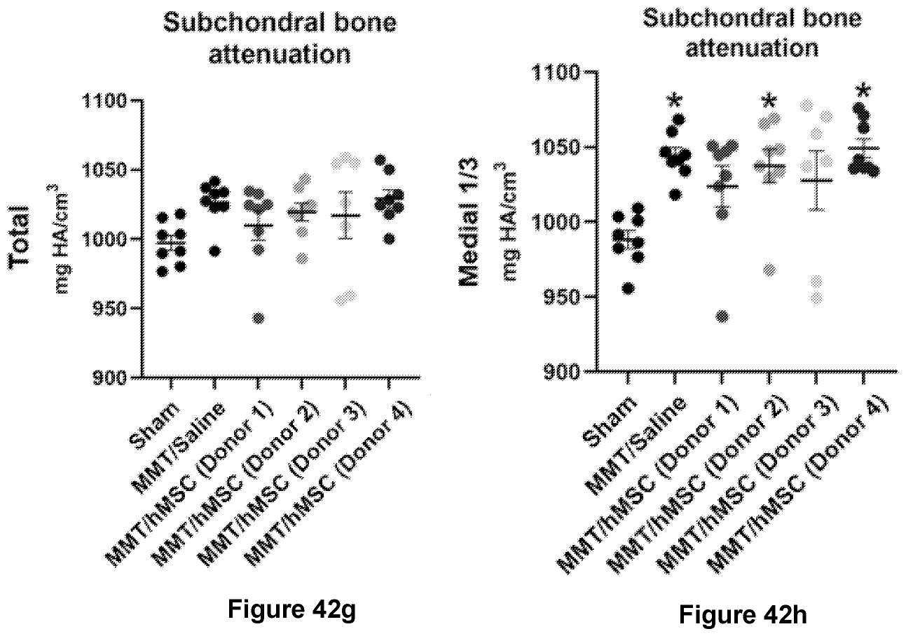

Quantitative analysis, using microCT, of osteophyte formations and subchondral bone remodeling was also performed for all samples. FIGS. 42a-42h illustrate the effect of hMSC donor heterogeneity on osteophyte and subchondral bone therapeutic outcomes in the total and medial 1/3 of the medial articular cartilage in 6-week MMT joints. As shown in FIG. 42a, mineralized osteophyte volumes were significantly increased in all MMT groups relative to sham; MMT/hMSC(2.1) showed significantly greater osteophyte volumes than MMT/Saline; MMT/hMSC(2.2), MMT/hMSC(2.3), and MMT/hMSC(2.4) showed reduced osteophyte volumes relative to MMT/Saline and MMT/hMSC(2.1). As shown in FIG. 42b, for cartilaginous osteophyte volumes, all MMT groups showed significantly greater volumes than sham; no differences were noted between MMT groups. As shown in FIG. 42c, for total subchondral bone volume MMT/Saline, MMT/hMSC(2.2), and MMT/hMSC(2.4) groups showed significantly higher volume than sham; all MMT/hMSC donor groups showed significantly reduced volume relative to sham. As shown in FIG. 42d, in the medial 1/3 region the MMT/Saline group was the only group to show significantly reduced volume relative to sham; all hMSC groups except MMT/hMSC(2.2) showed significantly reduced volume relative to MMT/Saline. As shown in FIG. 42e, for subchondral bone thickness the MMT/Saline, MMT/hMSC(2.2), and MMT/hMSC(2.4) showed significant increases in thickness relative to sham; MMT/hMSC(2.1) and MMT/hMSC(2.3) groups showed reduced thickness relative to MMT/Saline. As shown in FIG. 42f, in the medial 1/3 region results were similar to the total region analysis except no significant difference was noted between MMT/Saline and MMT/hMSC(2.3). As shown in FIG. 42g, for subchondral bone attenuation of the total tibial plateau no significant differences were noted. As shown in FIG. 42h, in the medial 1.3 region, the MMT/Saline, MMT/hMSC(2.2), and MMT/hMSC (2.4) showed significantly higher attenuation values than sham. This data is presented as mean±/−SD; n=7 for MMT/hMSC(2.3) and n=8 for all other groups. * represents significant differences (p<0.05) between individual MMT groups and sham. Horizontal black bars indicate significance (p<0.05) between individual MMT groups.

hMSC Secretion of GM-CSF, GRO, IL-4, and PDGF-AA, Relate to OA Therapeutic Outcomes

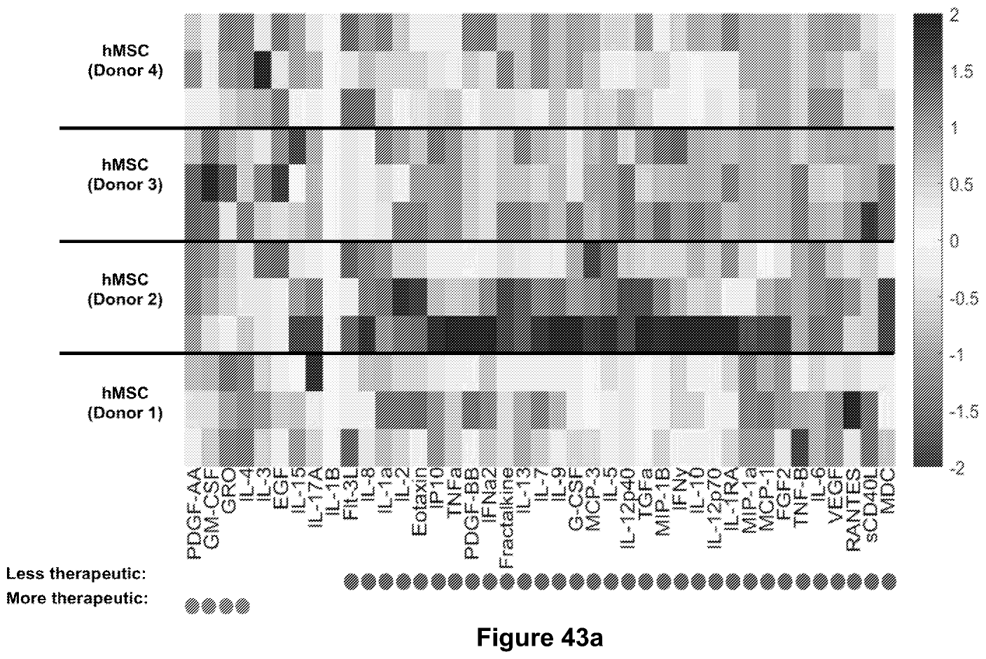

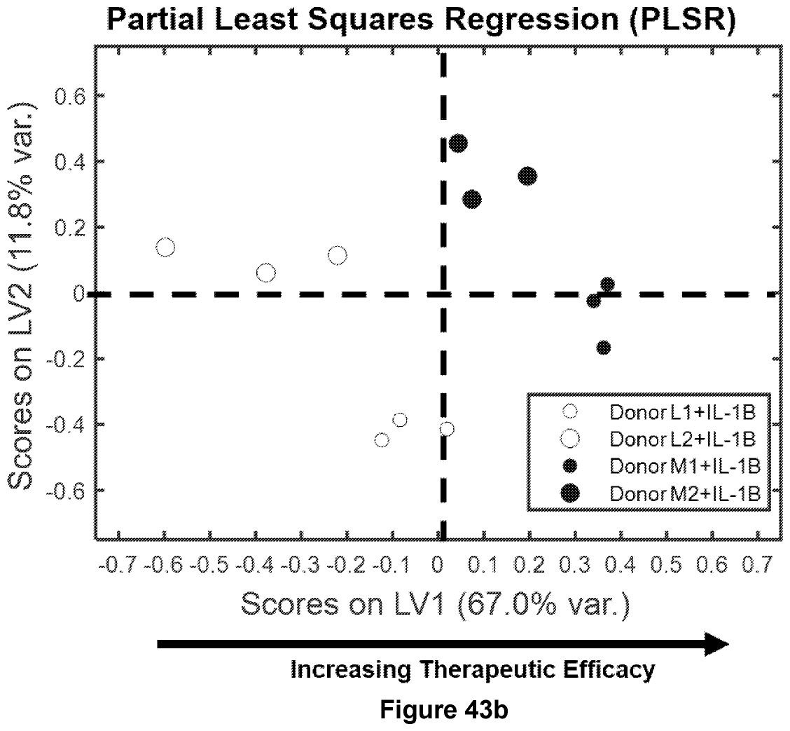

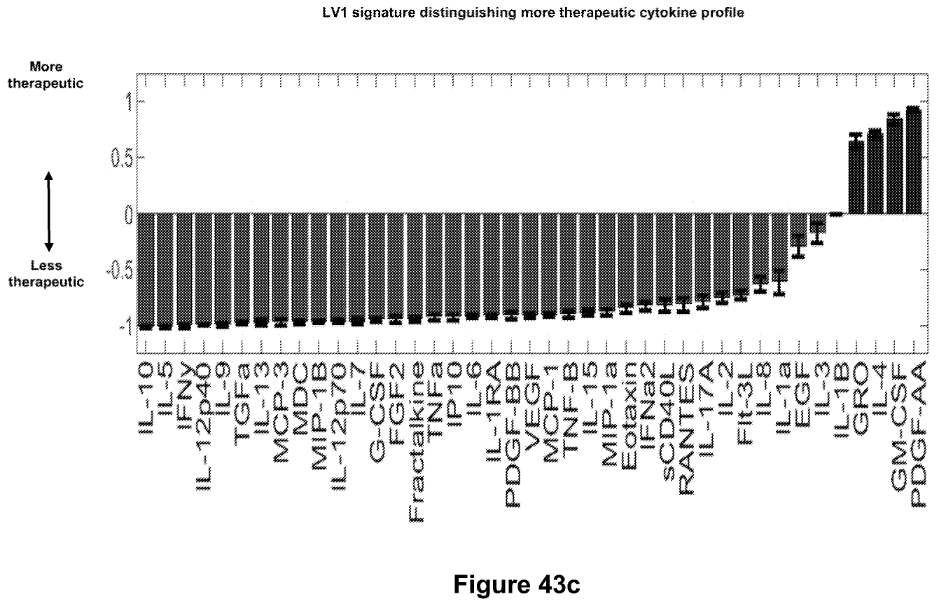

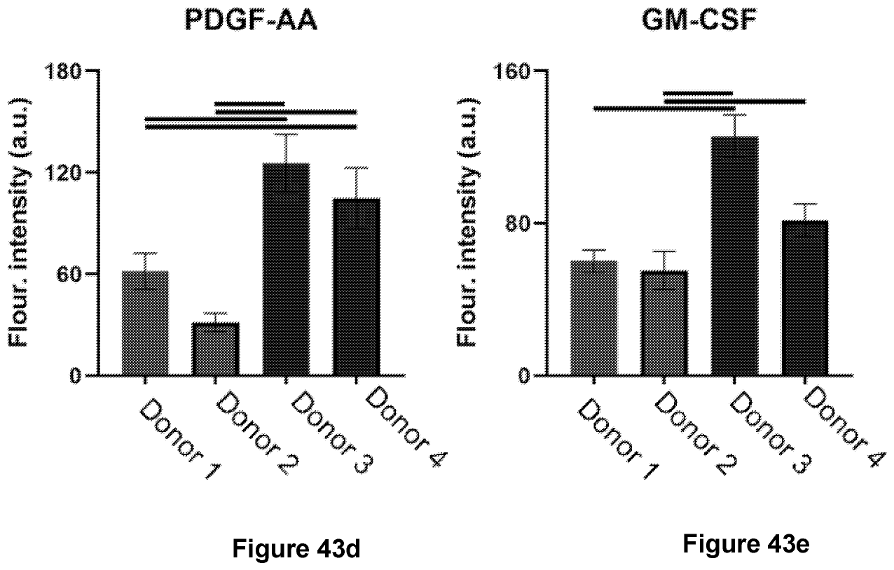

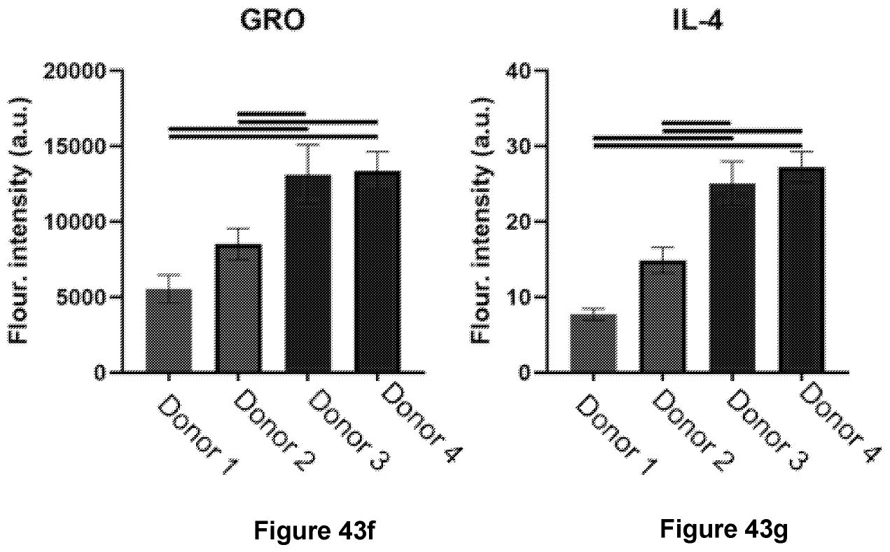

To assess differences in hMSC paracrine signaling between more therapeutic (donors 3 and 4) and less therapeutic donors (donors 1 and 2, as identified in FIG. 41a-41j), the inventors next used a multiplexed immunoassay to quantify a panel of 41 cytokines and growth factors secreted into the medium by each donor during 24 hr in culture. FIGS. 43a-43g illustrate paracrine signaling response of hMSCs in an IL-1β OA simulated microenvironment. FIG. 43a illustrates a multiplexed immunoassay analysis of 41 cytokines (columns; z-scored) secreted from hMSCs in IL-1β conditioned media. Donors L1 and L2 demonstrated potentiated overall cytokine secretion levels relative to donors M1 and M2, which yielded a more targeted paracrine profile. As shown in FIG. 43b, PLSR analysis (input: in vitro cytokine data; output: microCT data) identified a profile of cytokines along LV1 that identified a distinct separation between less therapeutic and more therapeutic hMSCs, as determined by microCT. Variability accounted for in each LV is included on respective axes labels. FIG. 43c provides a loadings plot demonstrating relative contribution of cytokines to PLSR scores obtained show that the cytokines GM-CSF, GRO, IL-4, and PDGF-AA contribute to separating out more therapeutic hMSC donors while all other cytokines assessed contribute more to less therapeutic hMSCs. As shown in FIGS. 43d-43g, GM-CSF, GRO, IL-4, and PDGF-AA were assessed independently and demonstrated significantly higher secretion levels in more therapeutic hMSC donors (M1 and M2) relative to less therapeutic donors (L1 and L2). Interestingly, the more therapeutic hMSCs (donors 3-4) secreted elevated levels of just a few factors, while the less therapeutic hMSCs (donors 1-2) secreted elevated levels of the majority of the panel. The inventors next used PLSR to identify a relationship between secretion level and microCT-computed therapeutic outcomes (FIG. 43b). The analysis identified a latent variable (LV1), which separated more therapeutic donors (3 and 4) to the right and less therapeutic donors (1 and 2) to the left. LV1 consisted of a profile of cytokines associated with more therapeutic (positive) or less therapeutic (negative) cells (FIG. 43c). Univariate analysis of the top four factors associated with more therapeutic hMSCs reveals significant upregulation of granulocyte macrophage colony stimulating factor (GM-CSF), chemokine ligand 1 (GRO), interleukin (IL)-4, and platelet derived growth factor (PDGF)-AA, in more therapeutic compared to less therapeutic hMSCs (FIGS. 43d-43g). Thus, these data show that there are significant differences in paracrine signaling between hMSC with more or less in vivo therapeutic efficacy.

More Therapeutic hMSCs Demonstrate Unique Gene Expression Pathway Profiles Relative To Less Therapeutic hMSCs

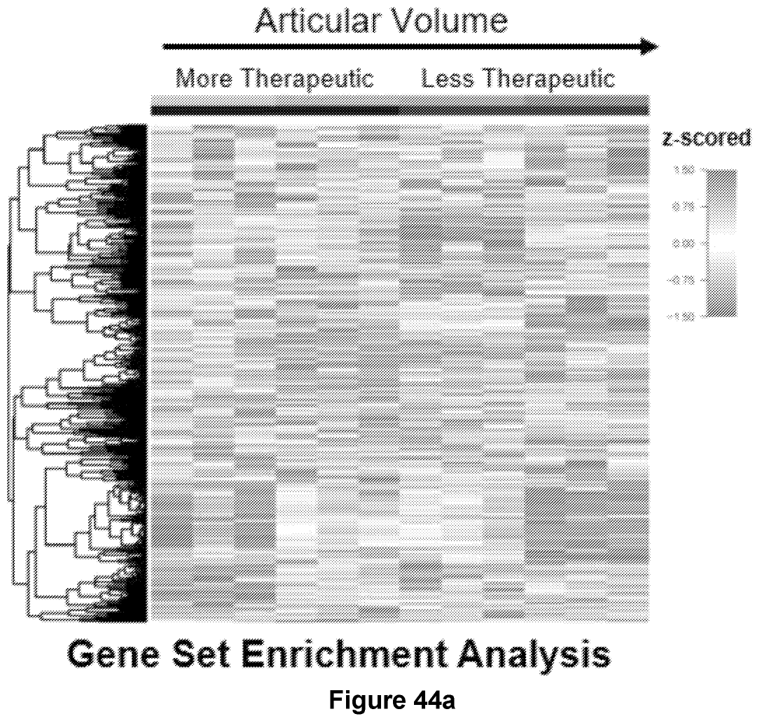

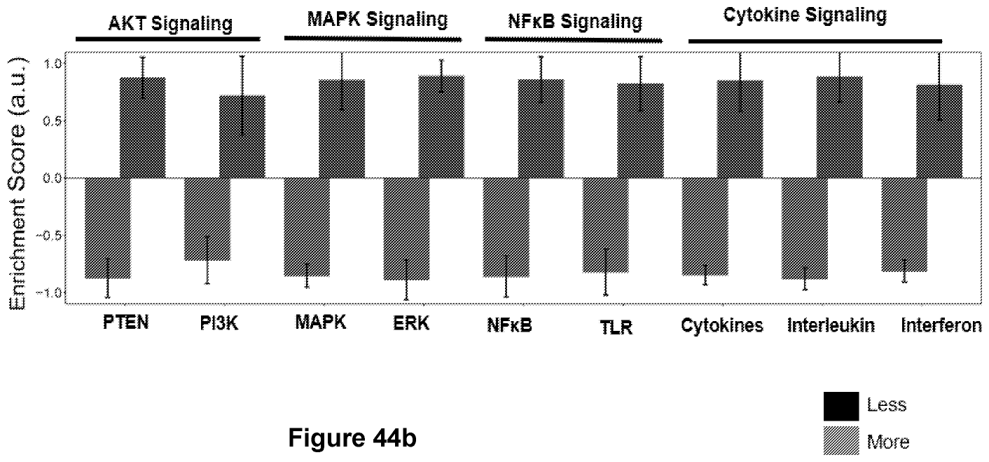

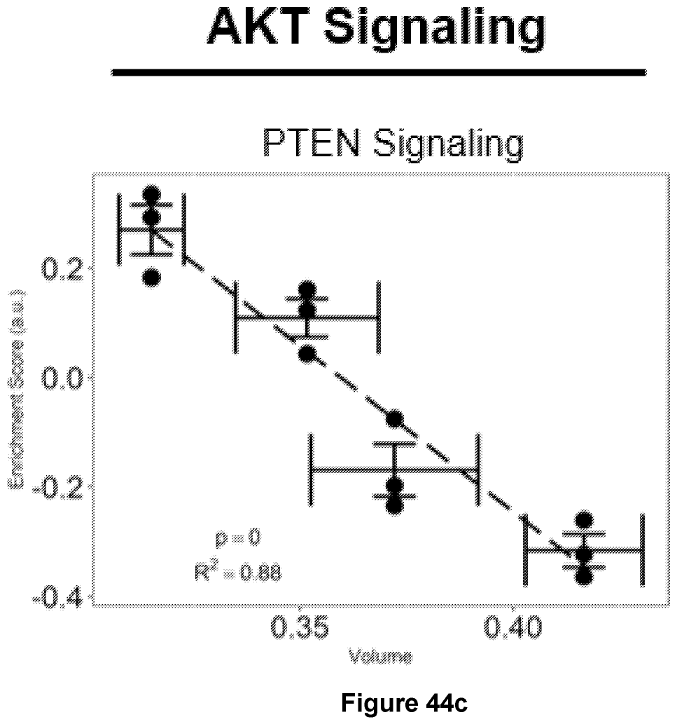

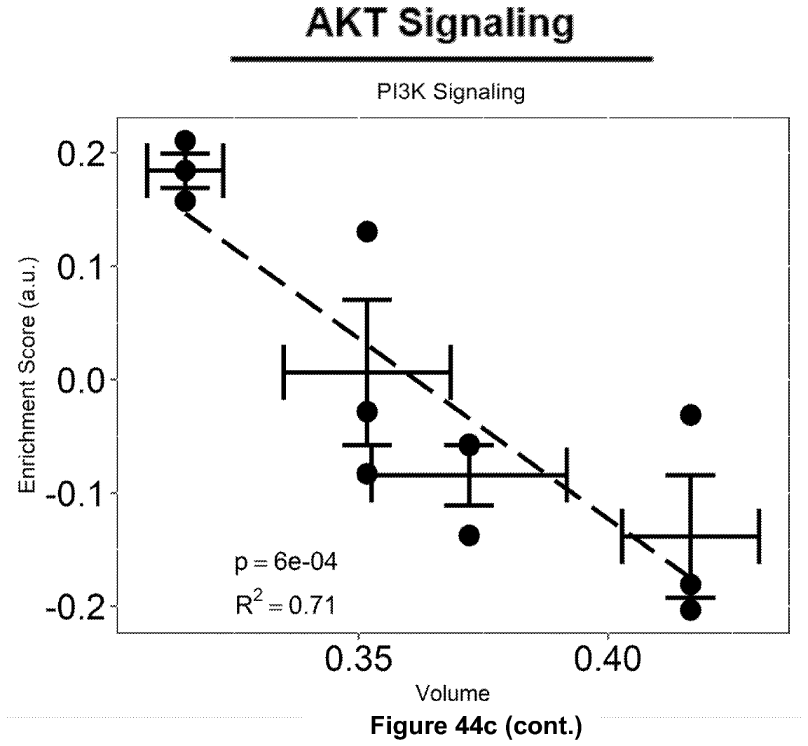

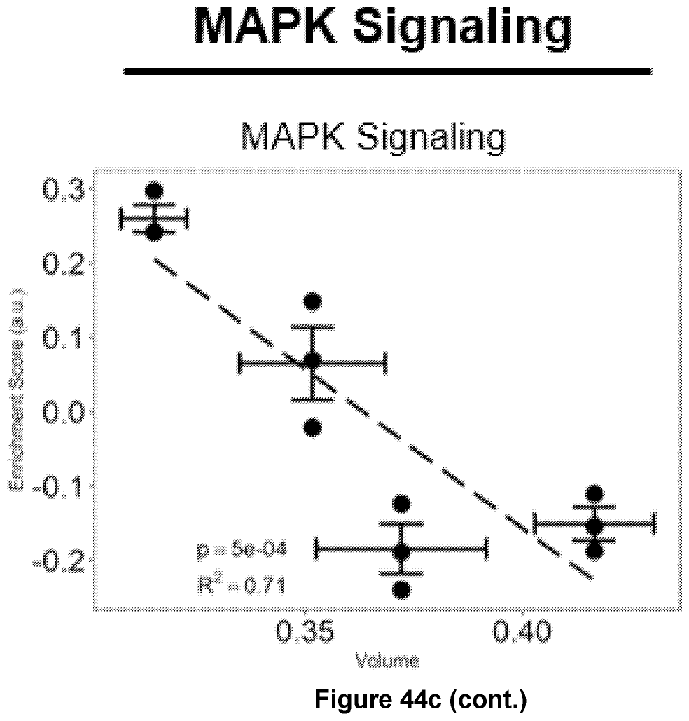

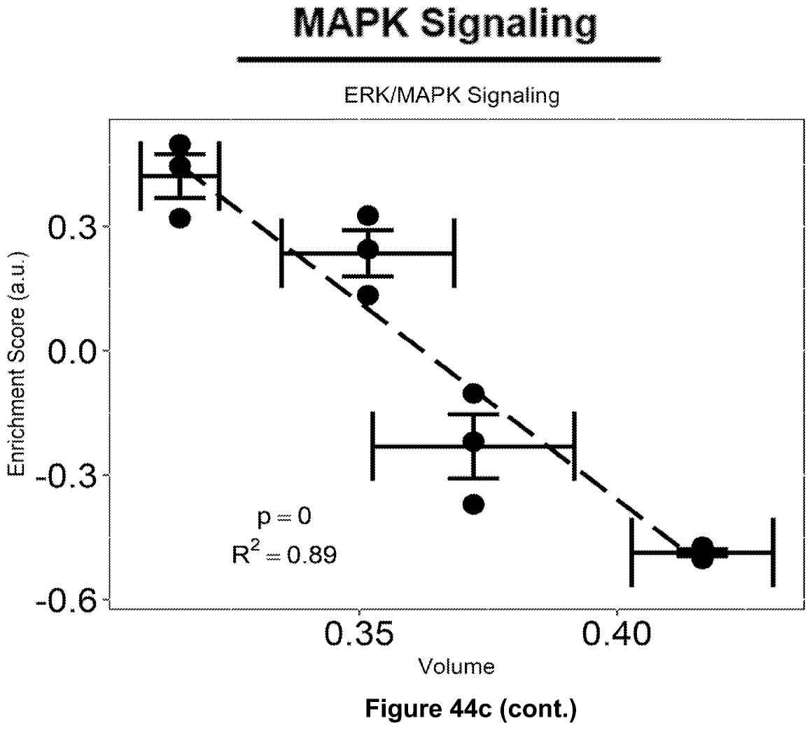

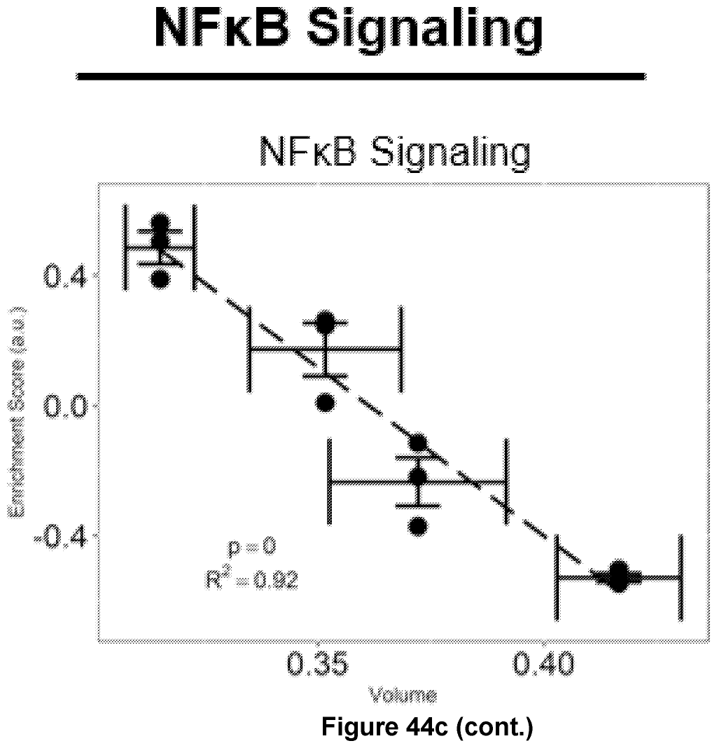

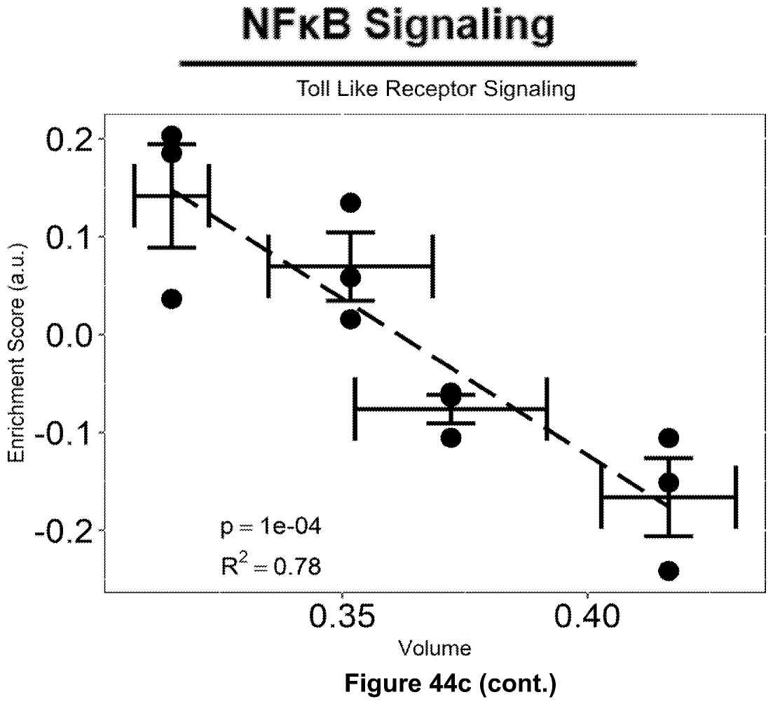

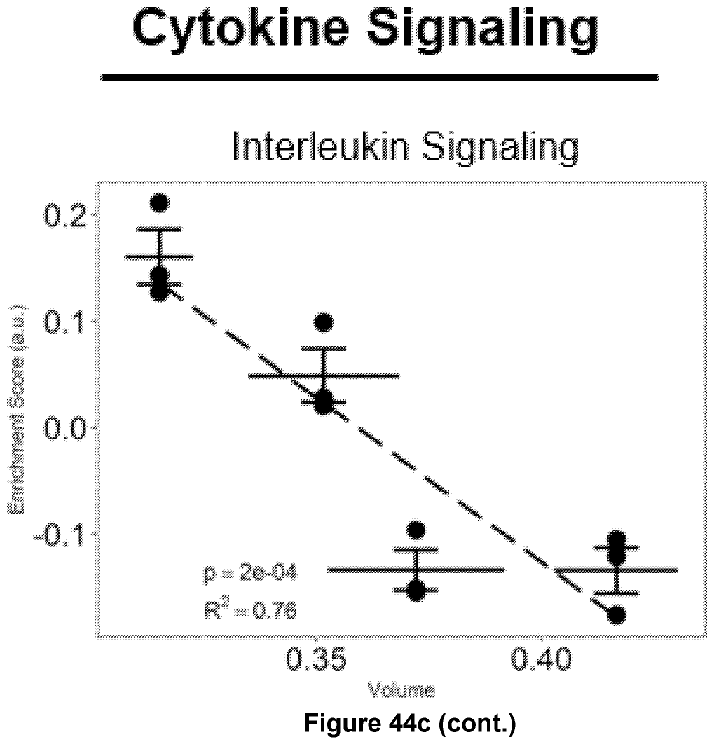

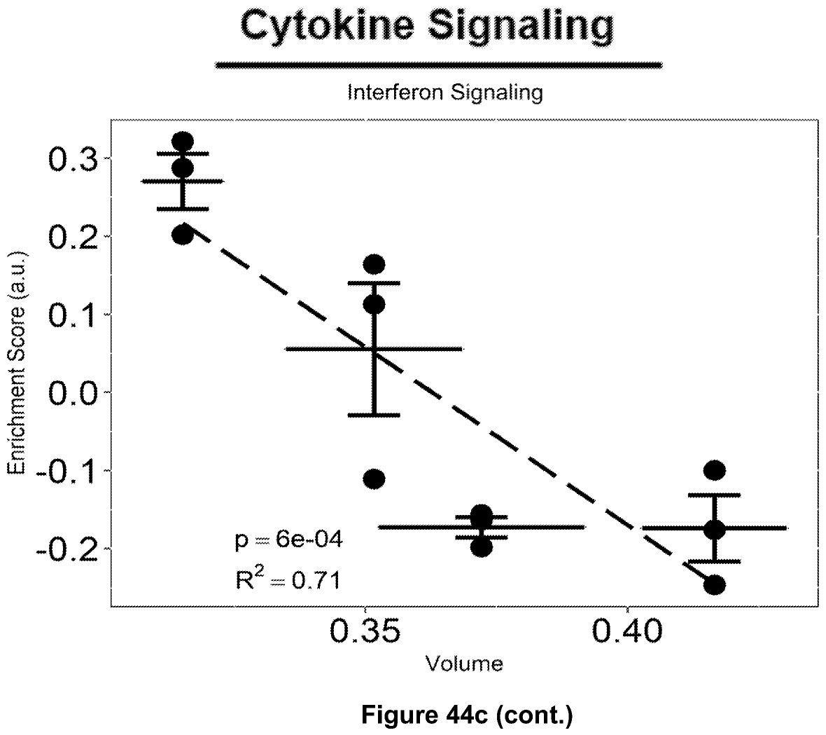

To holistically identify cellular attributes that relate to therapeutic efficacy, the inventors next used RNA-Seq to quantify gene expression from each hMSC donor after 24 hr of culture, then applied GSVA to identify genes that were enriched in 2,198 canonical pathways (C2 gene sets, Molecular Signatures Database, Broad). FIGS. 44a-44c illustrate unique gene set expression profiles of more therapeutic hMSCs and less therapeutic hMSCs following IL-1β conditioning in OA simulated microenvironment. As shown in FIG. 44a, quantification of 24,475 genes using RNA-Seq yielded two groupings with group 1 demonstrating unique gene expression of more therapeutic hMSCs and group 2 demonstrating the gene expression of less therapeutic hMSCs. FIG. 44b shows volcano plots of differentially expressed genes in less therapeutic hMSCs and more therapeutic hMSCs. As shown in FIG. 44c, these unique gene expression profiles were further demonstrated quantitatively using PCA which clearly showed that less therapeutic hMSCs (to the left) and more therapeutic hMSCs (to the right) separate along the PC1 axis. FIG. 44d provides a loadings plot demonstrating relative contribution of genes to PCA scores obtained demonstrate the top 50 genes in the loadings, corresponding to those that were most related to more therapeutic hMSCs, and the bottom 50 genes in the loadings, corresponding to those that were most related to less therapeutic hMSCS Hierarchical clustering of the GSVA-analyzed data identified a cluster of gene sets associated with more therapeutic cells and another cluster associated with less therapeutic cells (FIG. 44a, Table 5). Because of the importance of intracellular signaling pathways in regulating cytokine and growth factor expression, the inventors focused their attention on gene sets associated with canonical phosphoprotein signaling and cytokines. Importantly, the inventors found that gene sets for MAPK and PI3K/Akt pathways, as well cytokine signaling were enriched in more therapeutic hMSCs (FIG. 44b). The inventors additionally regressed these pathways against cartilage volume, finding that they each significantly inversely correlated (FIG. 44c). These data emphasize that these pathways are differentially regulated between more and less therapeutic hMSC groups.

Inhibition of p-JNK phospho-protein Levels Reduced Secretion of GM-CSF, GRO, IL-4, and PDGF-AA In More Therapeutic hMSCs

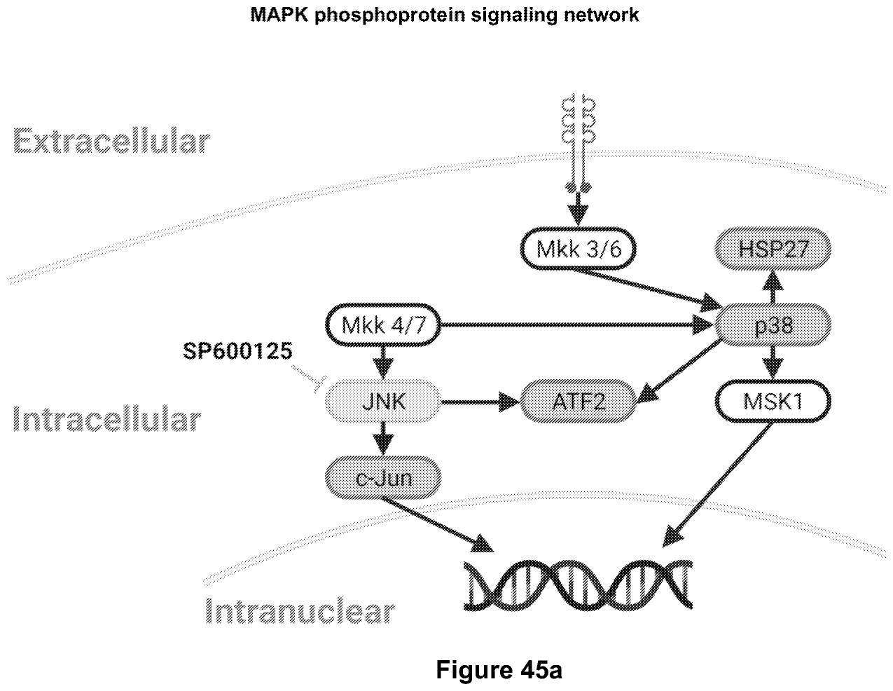

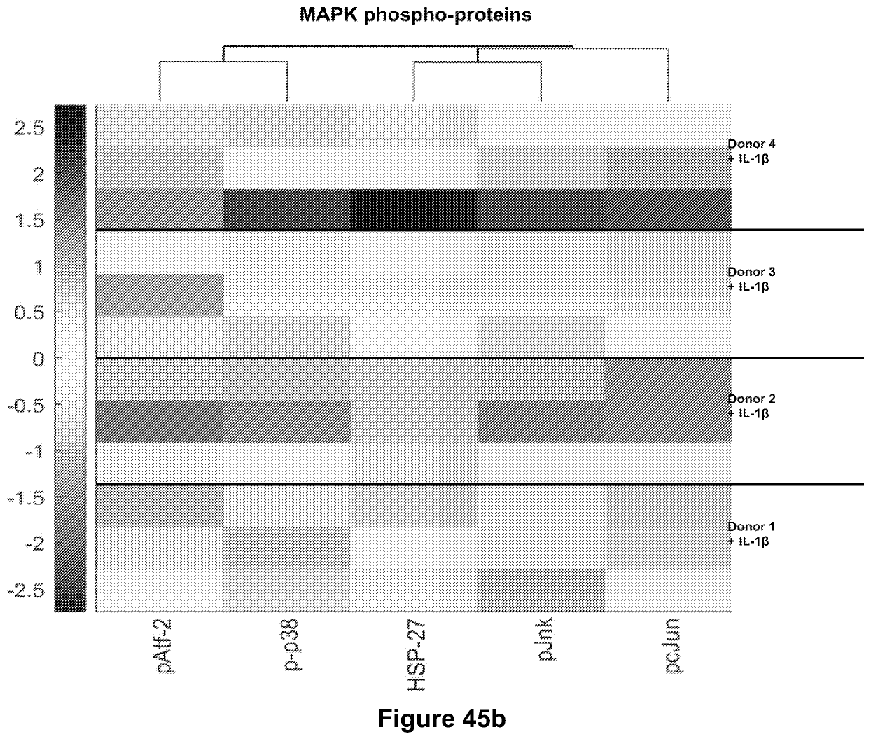

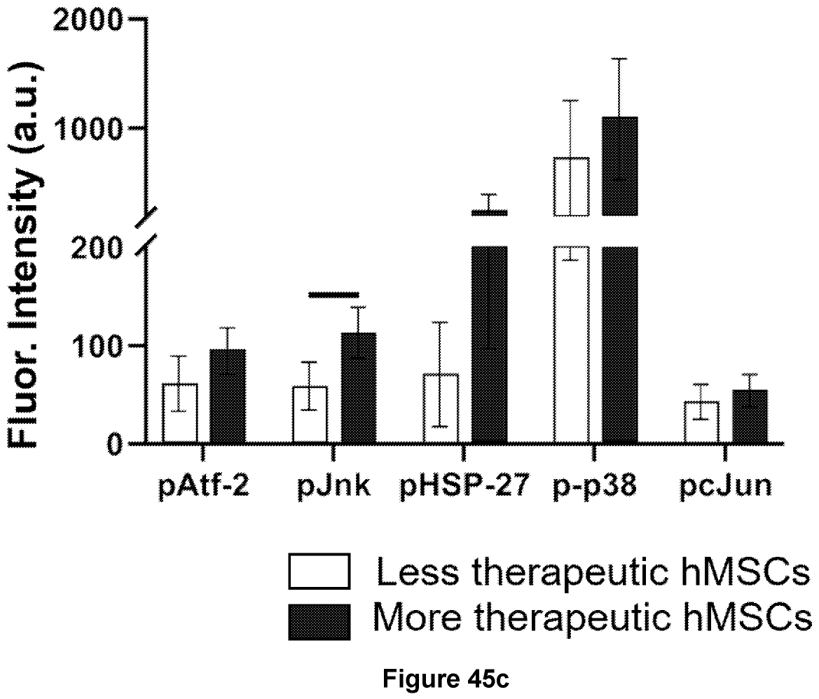

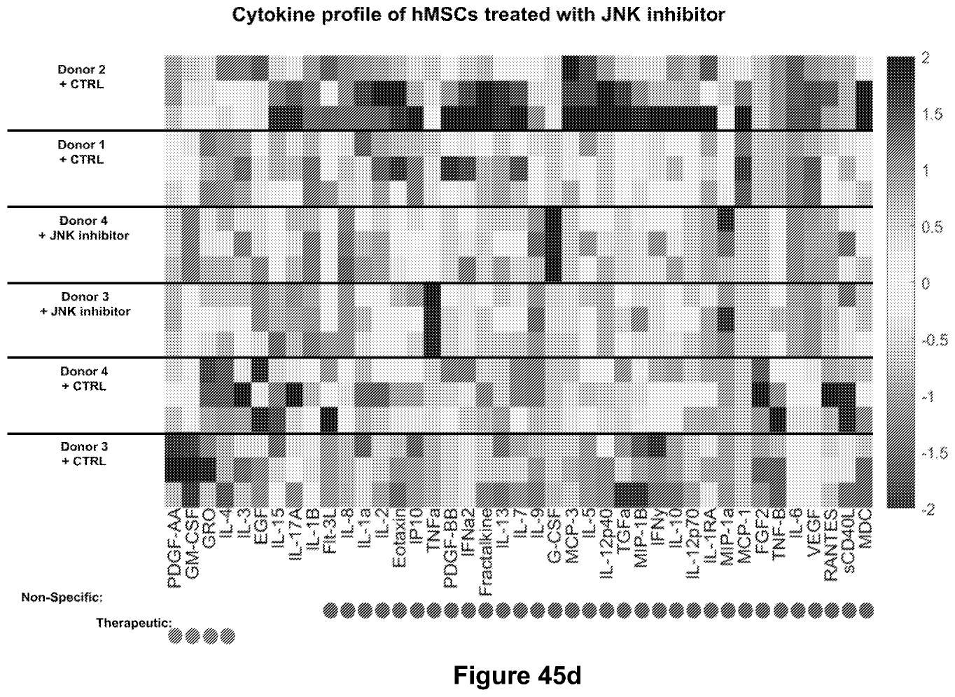

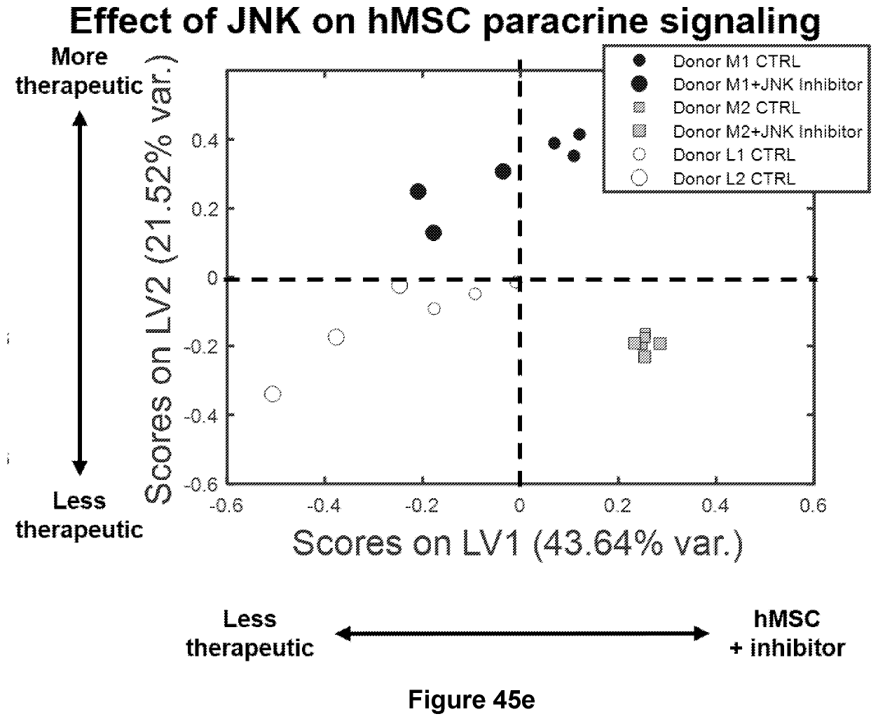

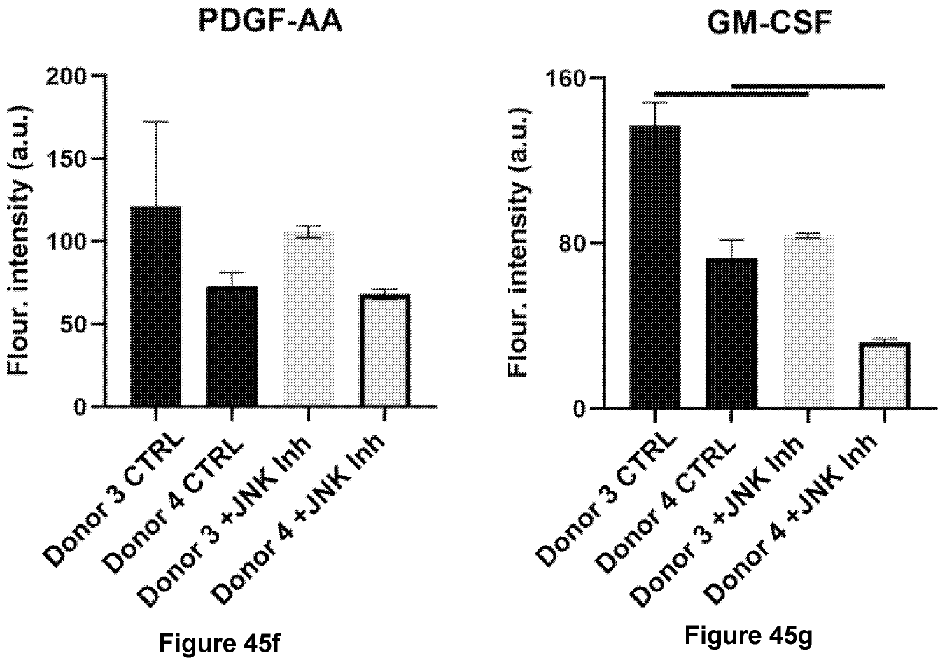

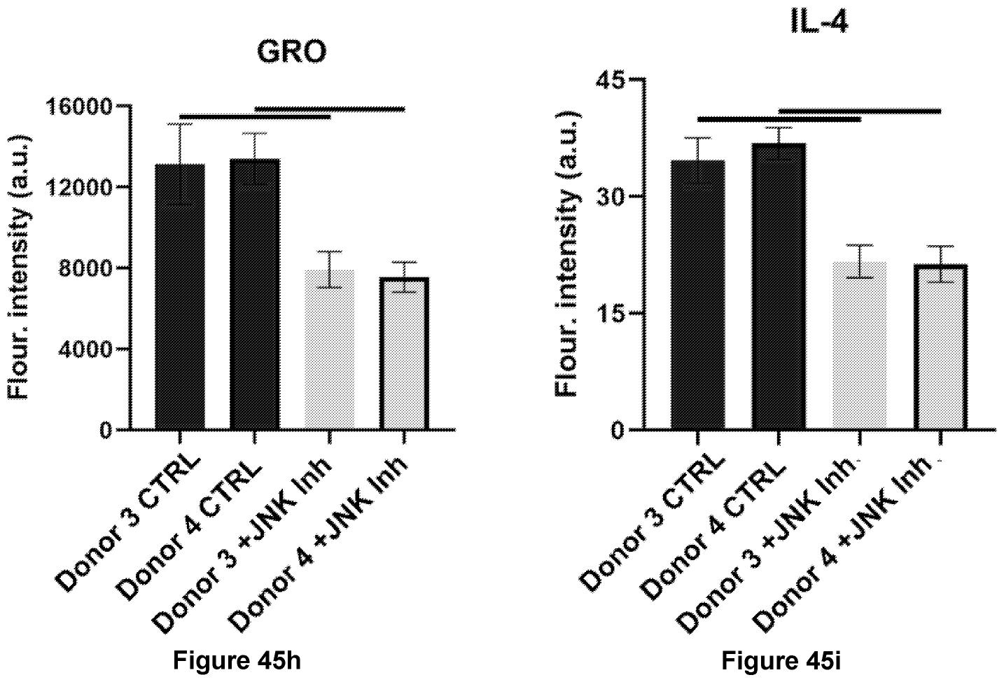

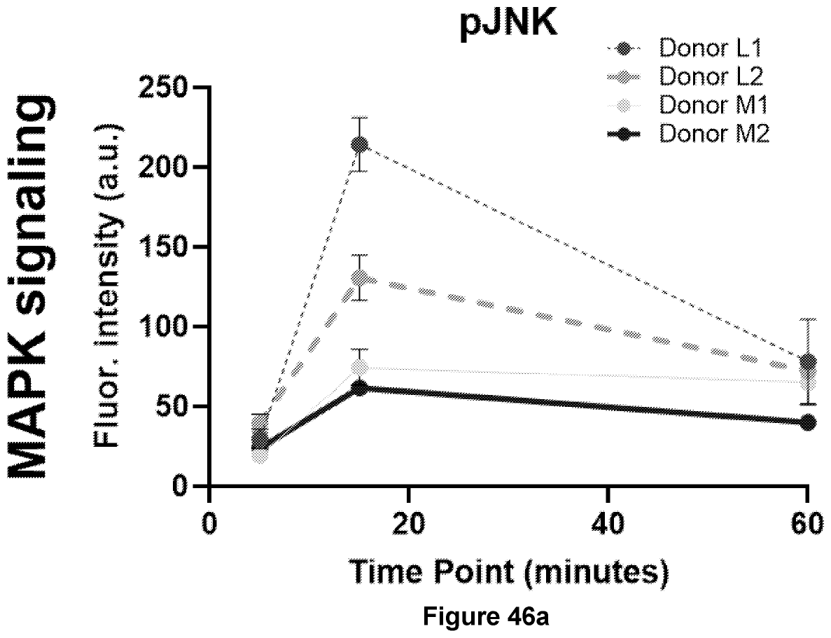

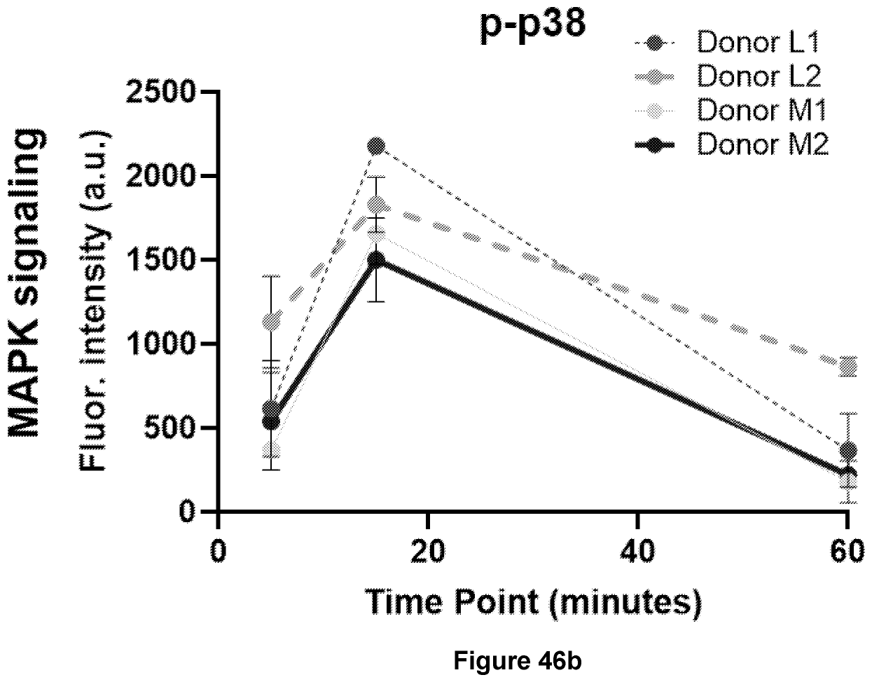

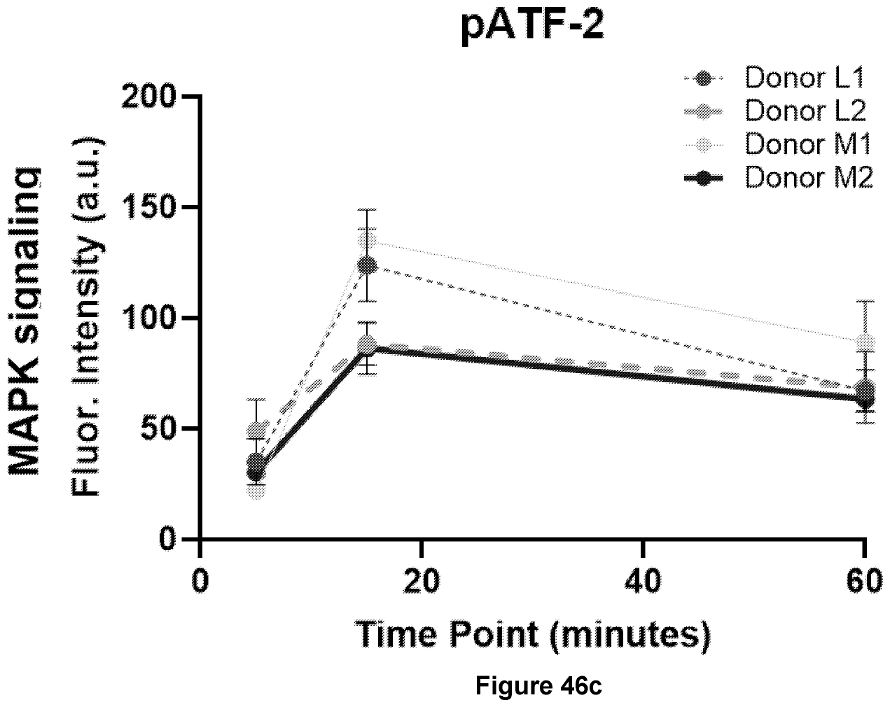

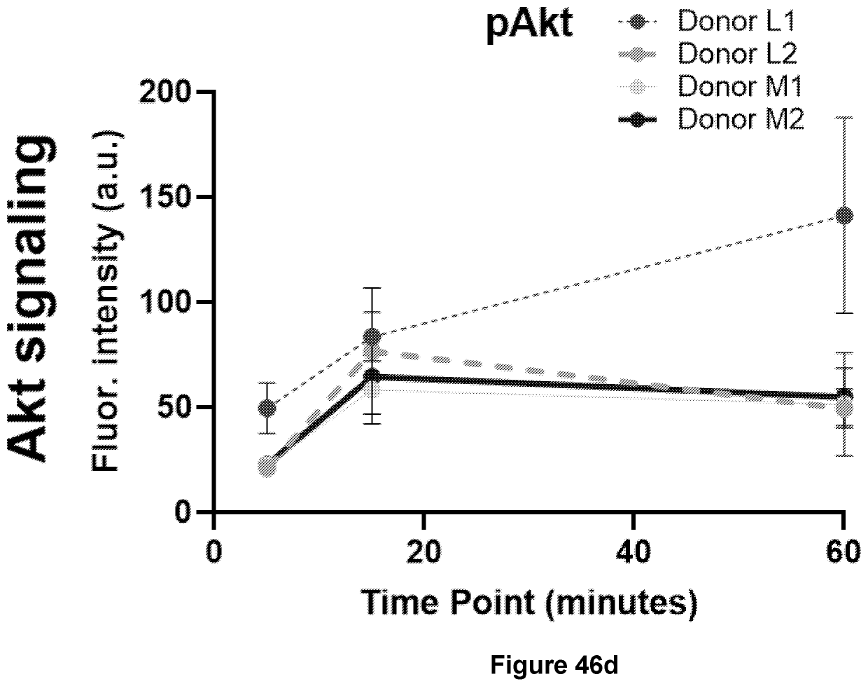

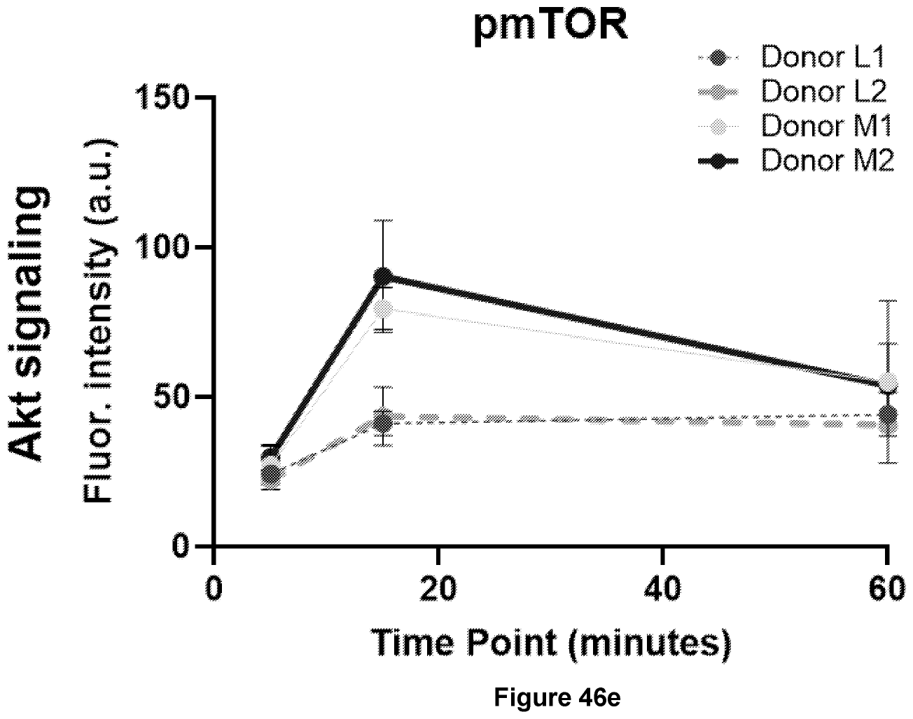

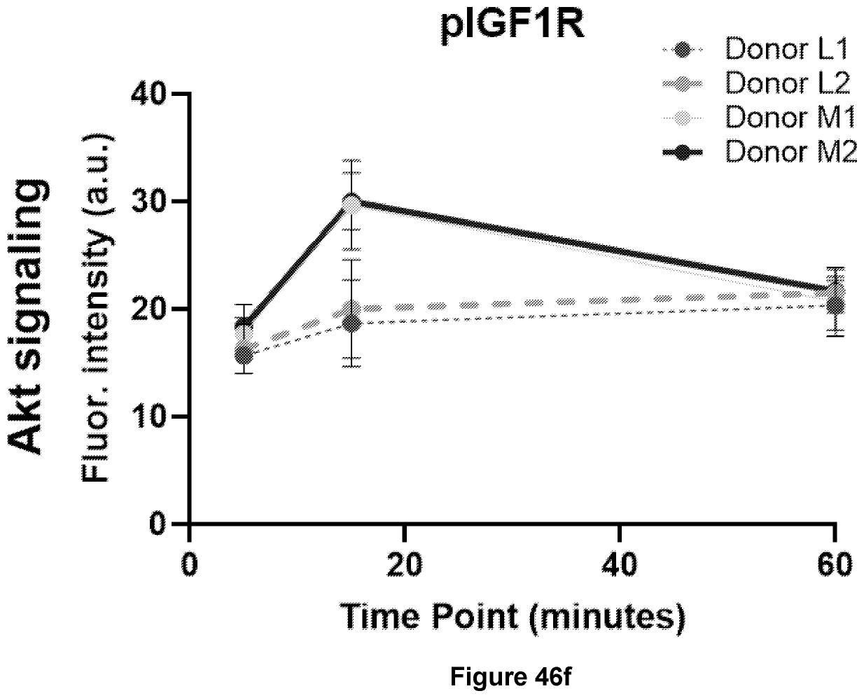

Having found that intracellular signaling pathways are differentially regulated based on RNA-seq analysis, the inventors next used Luminex multiplexed immunoassays to quantify MAPK pathway signaling at 5, 15, and 60 mins post IL-1β conditioning, as represented in FIGS. 45a-45i and FIGS. 46a-46f.

FIGS. 45a-45i illustrate paracrine signaling profiles of more therapeutic hMSCs (donors M1 and M2) treated with a p-JNK inhibitor (SP600125). FIG. 45a illustrates quantification of 6,287 gene expression pathways screened using GSVA yielded three distinct groupings, with group 1 demonstrating unique gene expression pathways of more therapeutic hMSCs, group 2 categorizing the gene expression pathways which had no relation to therapy, as they were conserved between more and les therapeutic hMSCs, and group 3 demonstrating the gene expression pathways of less therapeutic hMSCs. As shown in FIG. 45c, less therapeutic hMSCs demonstrate increased hematopoietic phenotype pathway expression while more therapeutic hMSCs demonstrated more similar phenotypes to isolated MSCs and a more anti-inflammatory phenotype. As shown in FIG. 45c, more therapeutic hMSCs demonstrated significant increased enrichment of GM-CSF and GCSF, IL-4, and TGFβ pathway signaling. As shown in FIG. 45d, less therapeutic hMSCs yielded increased enrichment of the Akt and NF-κB signaling pathways while more therapeutic hMSCs yielded increased enrichment of the MAPK signaling pathway. Horizontal bars indicate significance (p<0.25) between more and less therapeutic hMSCs enrichment scores. As shown in FIGS. 45e-45n, more therapeutic microCT outcomes were significantly correlated to MAPK signaling (FIGS. 45g, 45h), proteoglycan synthesis (FIG. 45i), TGFβ signaling (FIG. 45j), and FGF signaling (FIG. 45k); less therapeutic microCT outcomes were significantly correlated with Akt signaling (FIGS. 45e, 45f), IFNγ expression (FIG. 45l), TNF expression (FIG. 45m), and IL-6 expression (FIG. 45n). Pearson's correlation coefficient, R, together with the p-value calculated from an F test (null hypothesis that the overall slope is zero) were included for all regression analyses.

Pathway signaling peaked at 15 min, revealing increased p-activating transcription factor (Atf)-2, p-p38, p-heat shock protein (HSP)-27, and p-c-Jun N-terminal kinase (JNK) signaling relative to less therapeutic hMSCs (FIG. 45b). Having identified significant differences in JNK phosphorylation at this time point (FIG. 45c), the inventors next suppressed JNK signaling in more therapeutic hMSCs (donors 3-4) using the small molecule inhibitor SP600125 to determine the effect of JNK signaling on paracrine factor secretion. Doing broadly increased cytokine expression (FIG. 45d) and shifted the paracrine secretion profile of the more therapeutic cells toward a less therapeutic profile (FIG. 45e). Importantly, SP600125 also attenuated expression of cytokines/growth factors associated with more therapeutic hMSCs (FIGS. 43a-43g, 45f-45i). Collectively, these data suggest that JNK signaling may mediate therapeutic efficacy of more therapeutic hMSCs.

FIGS. 46a-46f illustrate temporal analysis of target phospho-protein signals in the MAPK and Akt signaling pathways. As shown in FIGS. 46a-46c, p-JNK, p-p38, and p-Atf-2 phospho-protein signaling in the MAPK pathway yield signaling spikes in 15 mins, relative to the 5 and 60 min time points. Furthermore, p-Akt, p-mTOR, and P-IGF1R in the Akt pathway also yield spikes in signaling at 15 mins, relative to the 5 and 60 min time points, as shown in FIGS. 46d-46f. Time demonstrated a significant effect (p<0.0001) for all MAPK and Akt phospho-protein signaling assessed.

Activation of p-Akt phospho-protein Potentiated Nonspecific Cytokine Secretion In More Therapeutic hMSCs

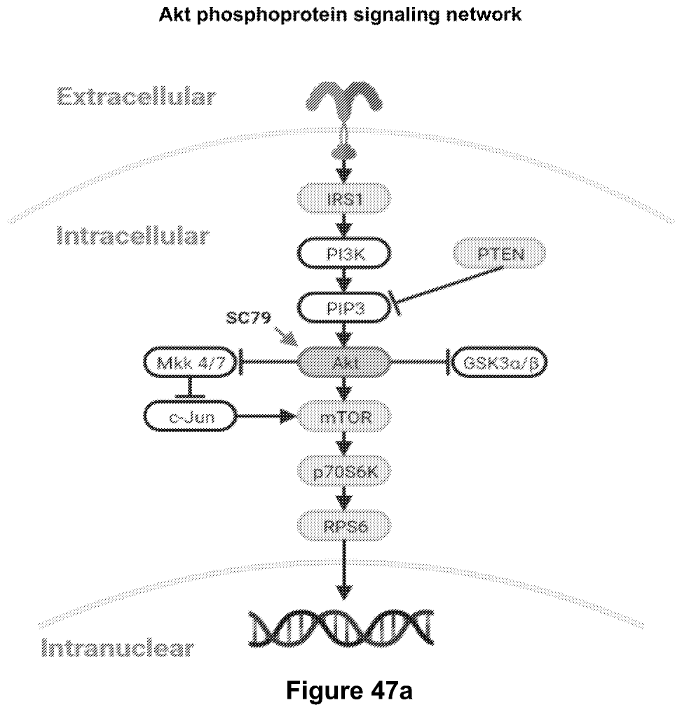

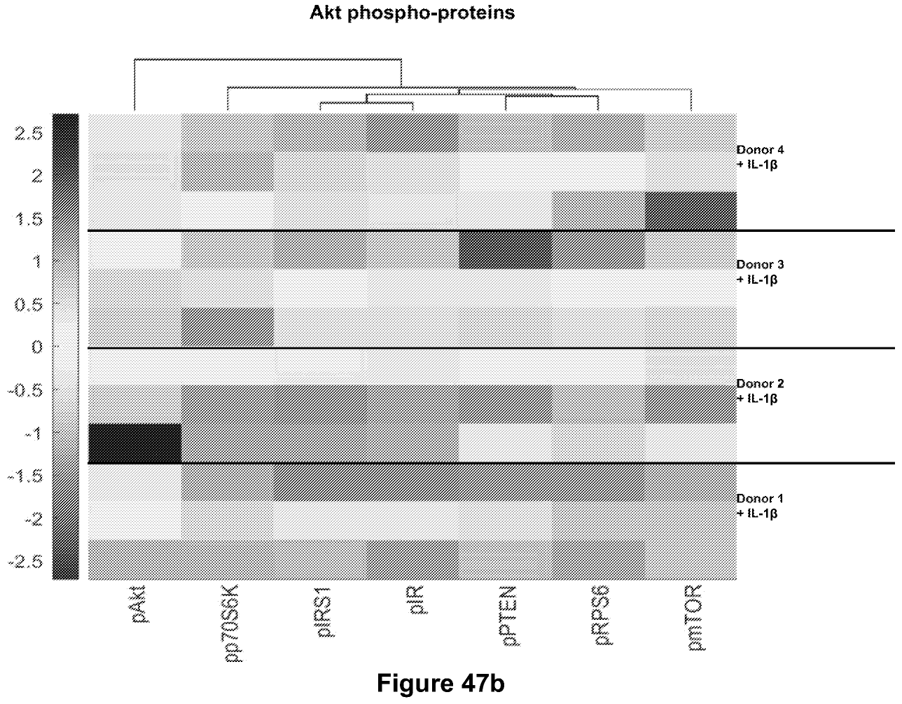

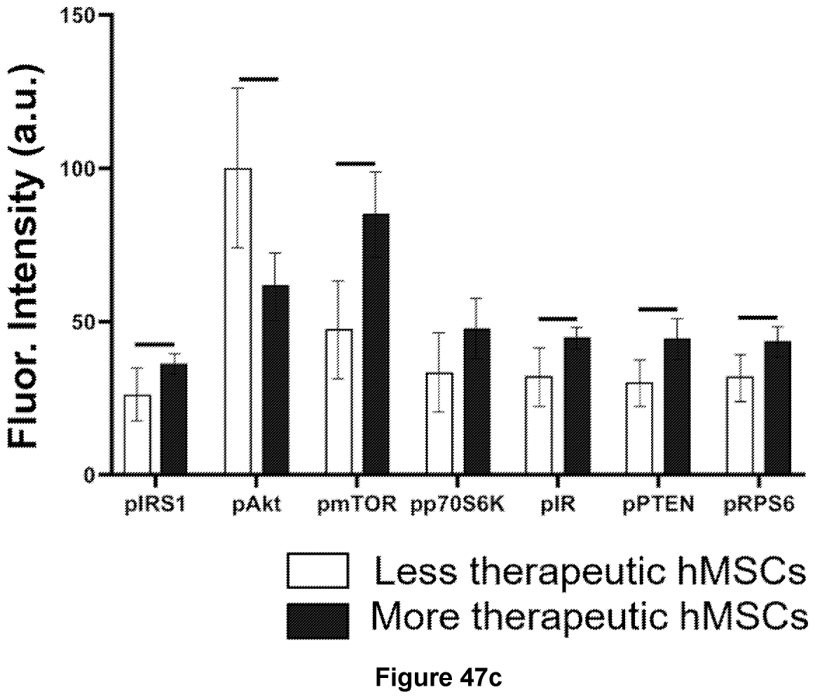

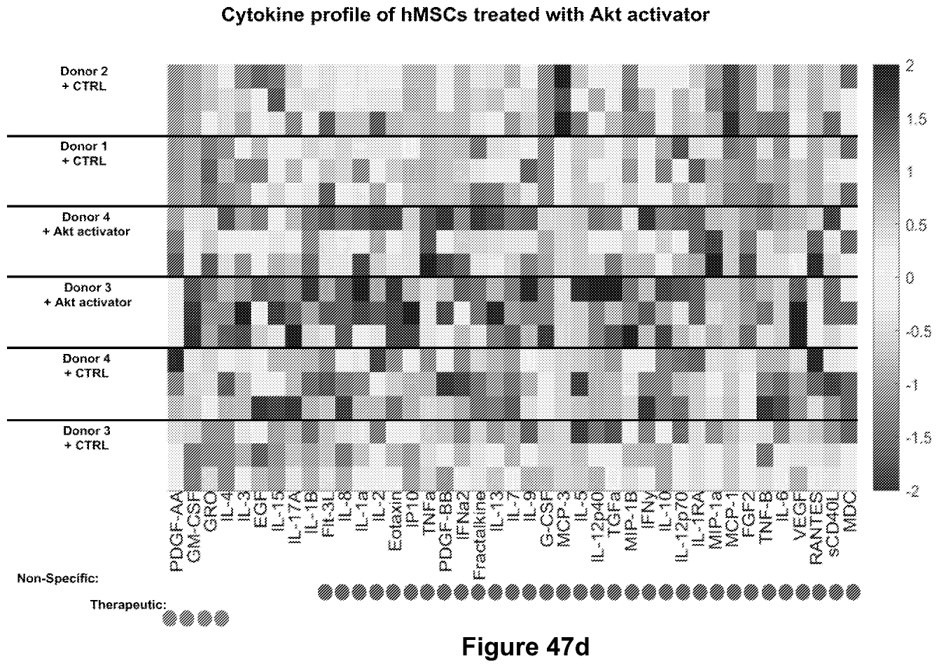

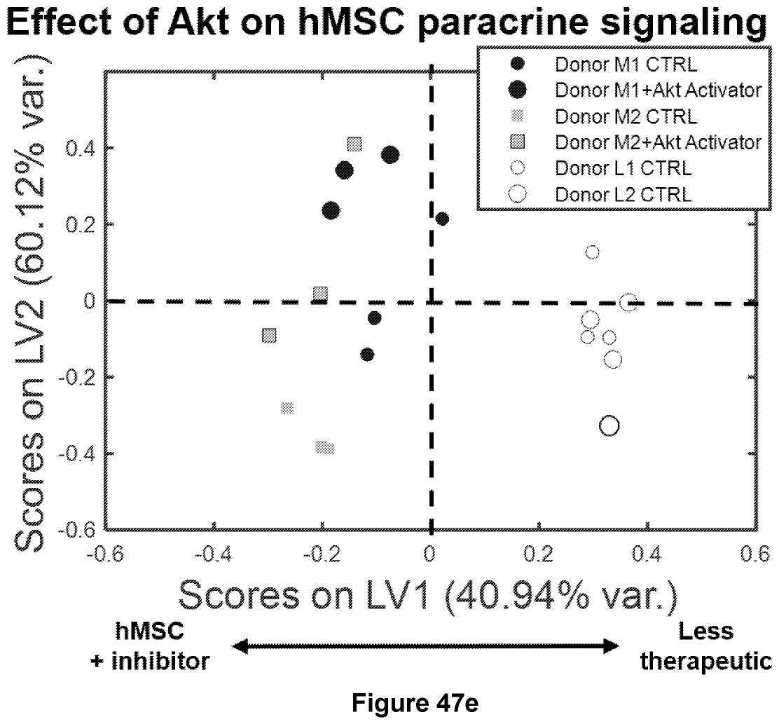

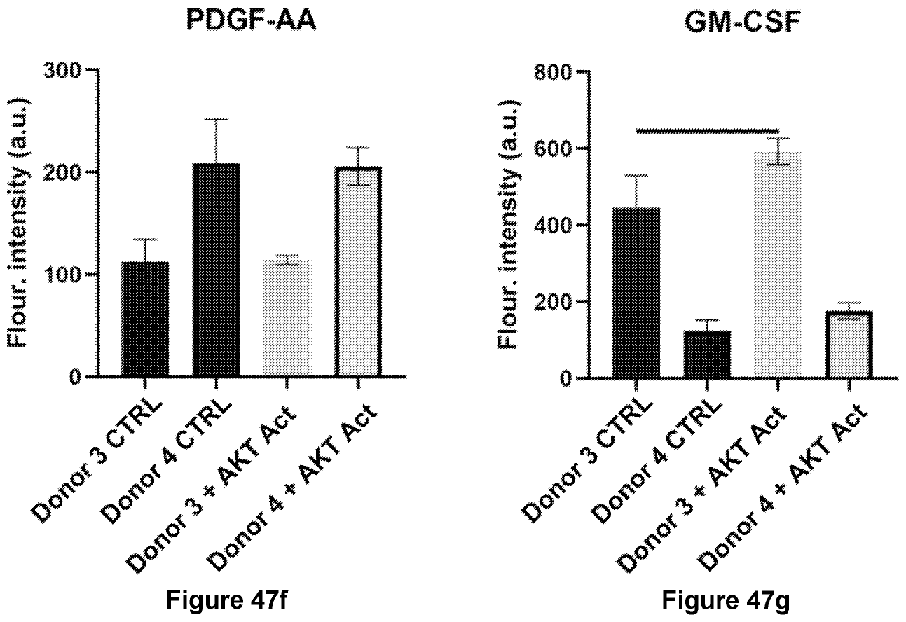

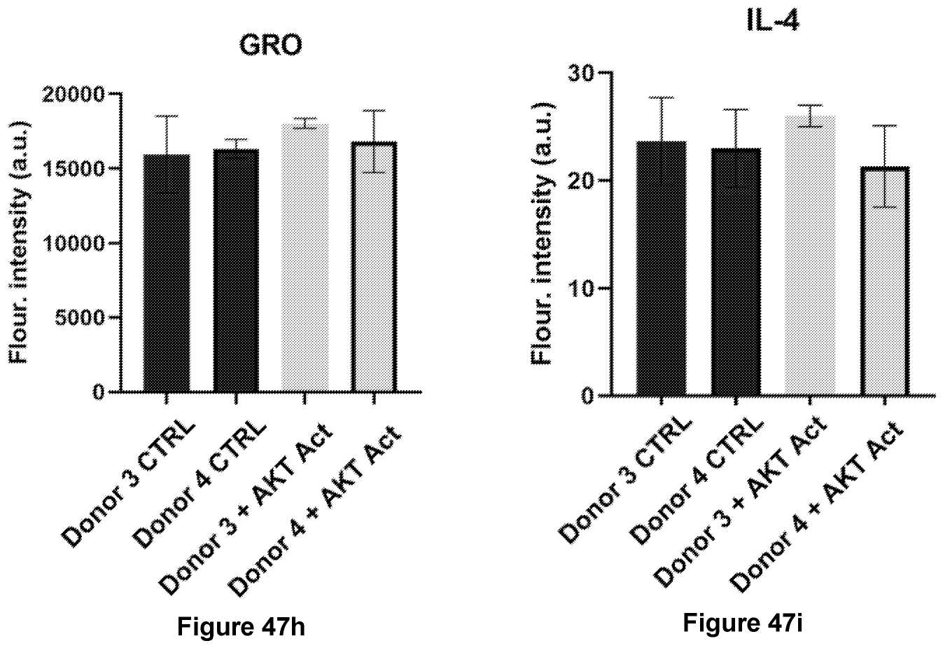

FIGS. 47a-47i illustrate paracrine signaling profiles of more therapeutic hMSCs (donors M1 and M2) treated with an Akt activator (SC79). Since the RNA-seq analysis also implicated the PI3K/Akt pathway signaling as being enriched in more therapeutic cells, and the essential role of this pathway in mediation of cytokine expression, the inventors used Luminex analysis to quantify PI3K/Akt at the 15 min time point, as provided in FIG. 47a, which illustrates a signaling schematic of the MAPK pathway (ERK pathway is not included) in hMSCs with the inhibitor site (labeled IRS1, PTEN, mTOR, p70S6K, RPS6) highlighted for the p-JNK phospho-protein signaling node.

FIG. 47b illustrates how quantification of MAPK phospho-protein signaling levels in all cell lines demonstrated increased overall phospho-protein signaling levels in the more therapeutic hMSCs relative to the less therapeutic hMSCs. More specifically, more therapeutic hMSCs demonstrated increased p-JNK, p-HSP-27, and p-ATF-2 phospho-protein signaling levels relative to less therapeutic hMSCs. More therapeutic hMSCs also demonstrated increased p-p70S6K, p-insulin receptor (IR), p-insulin receptor substrate (IRS) 1, p-PTEN, p-ribosomal protein (RP) S6, and p mammalian target of rapamycin (mTOR) signaling relative to less therapeutic hMSCs.

As shown in FIG. 47c, the p-JNK node was the only phospho-protein signaling node to show significantly different expression with more therapeutic hMSCs demonstrating increased levels. That is, less therapeutic hMSCs demonstrated elevated phosphorylation-Akt, relative to more therapeutic hMSCs.

While most of these phospho-proteins demonstrated significant differences between more therapeutic and less therapeutic hMSCs, the inventors next asked if Akt might be a central signaling node in control of the therapeutic paracrine profile. To test this, the inventors used a small molecule Akt activator (SC79) to determine if Akt activation would transform the more therapeutic cells to secrete a paracrine signature associated with less therapeutic hMSCs. Akt activation yielded a clear qualitative shift in the paracrine signaling profiles of more therapeutic hMSCs (donors 3 and 4), as shown in FIG. 47d, which illustrates how quantification of immunomodulatory cytokines for more therapeutic hMSCs, with a p-JNK inhibitor applied, shows distinct shifts in overall cytokine signaling with addition of the inhibitor. More specifically, treatment of more therapeutic hMSCs with SP600125 yielded decreased secretion of GM-CSF, GRO, IL-4, and PDGF-AA.

As shown in FIG. 47e, using PLSDA to interpret these changes revealed that the p-Akt activator (Donors M2 CTRL and M2+Akt Activator) yielded a leftward shift on LV1 relative to untreated more therapeutic hMSCs (Donors M1 CTRL and M1+Akt Activator), in the PLSDA plot of the cytokine data; however, this was in a different direction of the less therapeutic hMSCs (Donors L1 CTRL and L2 CTRL), which yielded a rightward shift on LV1, relative to the more therapeutic hMSCs (Donors M1 CTRL and M1+Akt Activator).

However, the pharmacological intervention strategy administered did not have an effect on the therapeutic related cytokines identified in the cytokine analysis. As shown in FIGS. 47f-47i, treatment with a p-JNK inhibitor yielded significantly less GM-CSF, GRO, and IL-4 secretion in more therapeutic hMSCs, relative to the more therapeutic hMSC control. Together, these data suggest that Akt signaling mediates expression of the cytokines associated with less therapeutic cells.

Combination Therapy of JNK Inhibitor and Akt Activator Shifts More Therapeutic hMSCs To A Less Therapeutic Paracrine Signaling Profile

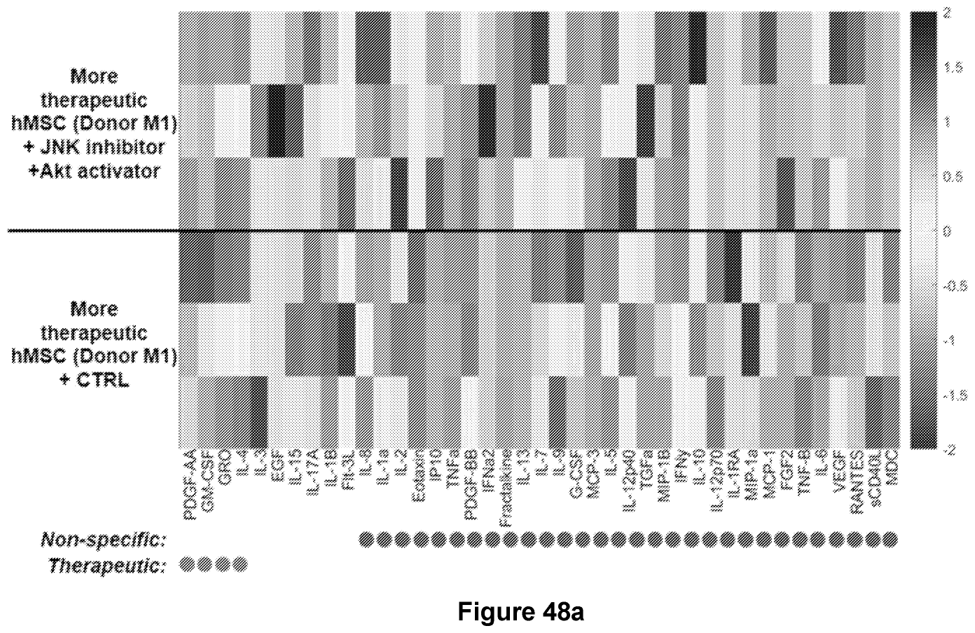

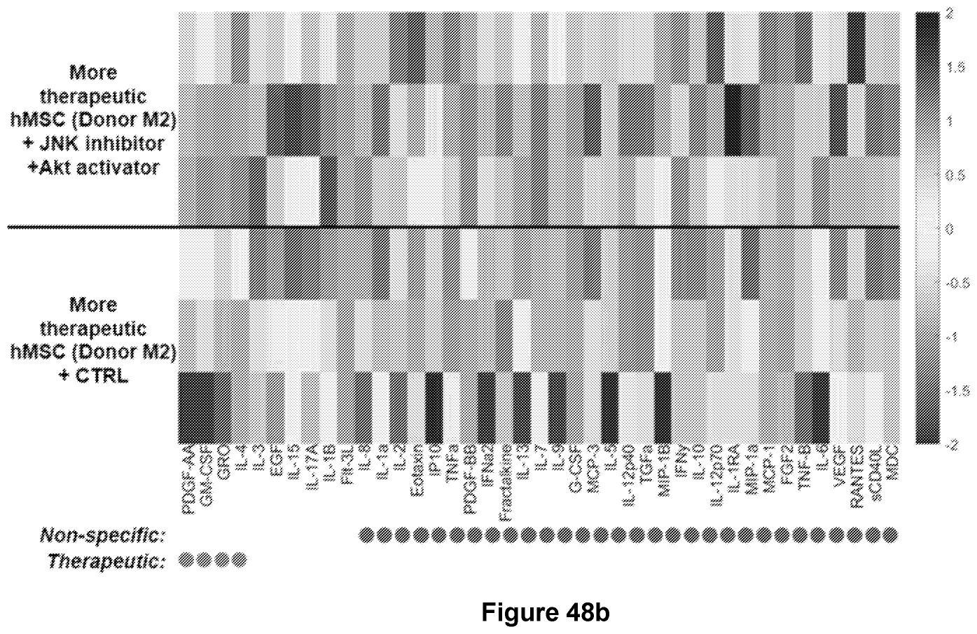

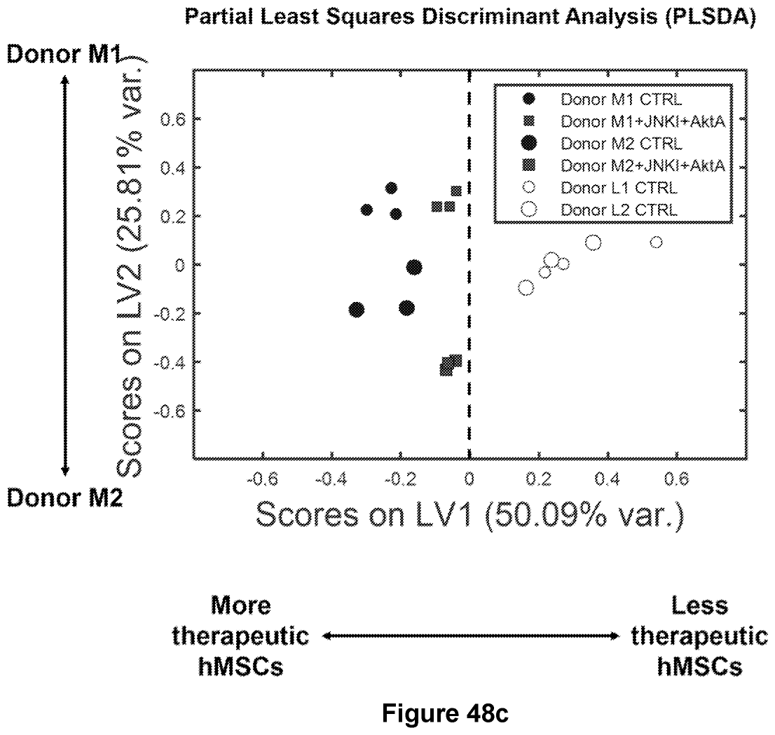

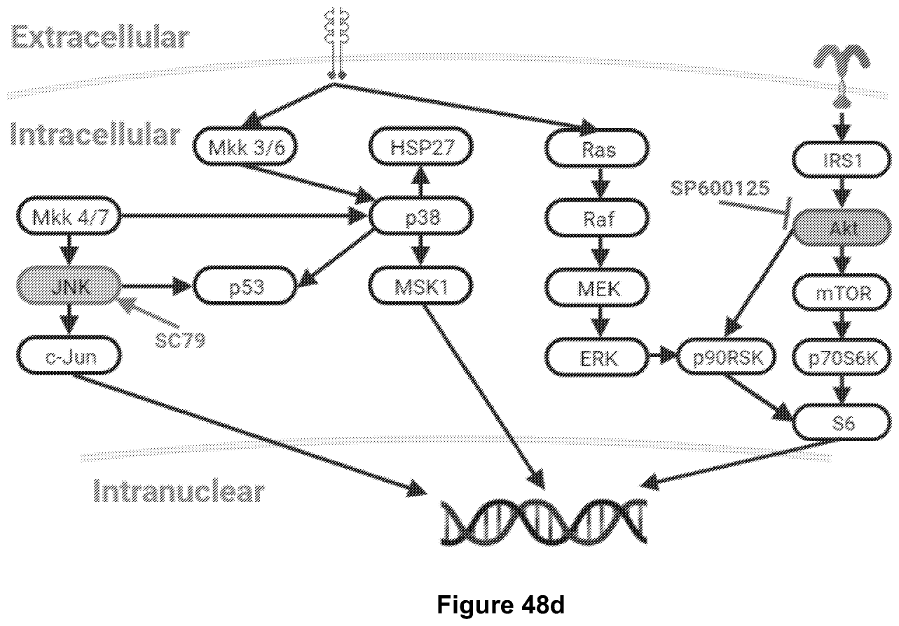

FIGS. 48a-48d illustrate paracrine signaling profiles of more therapeutic hMSCs (donors M1 and M2) treated with a JNK inhibitor (SP600125) and p-Akt activator (SC79). A p-JNK inhibitor (SP600125) and p-Akt activator (SC79) were administered in combination to shift the cytokine profile of more therapeutic hMSCs towards a less therapeutic paracrine signaling profile, as shown in FIG. 48d, which illustrates how quantification of immunomodulatory cytokines for more therapeutic hMSCs, with a p-Akt activator applied, shows distinct shifts in overall cytokine signaling with addition of the activator as overall cytokine levels were potentiated. This combination was based on the p-JNK inhibitor treated hMSCs (donor 3 and 4), alone, showing reduced secretion of the therapeutic related cytokines GM-CSF, GRO, IL-4, and PDGF-AA. Furthermore, the p-Akt activator treated hMSCs (donors 3 and 4) showed potentiated secretion of nonspecific cytokines in more therapeutic hMSCs.

For more therapeutic hMSC donors treated with combination therapy, treated cells demonstrated a clear shift in paracrine signaling profiles, relative to the hMSC CTRLs for each respective donor, as shown in FIGS. 48a and 48b. More specifically, treated hMSCs demonstrated a less targeted cytokine profile (increase in the secretion of majority of cytokines assessed) and reduced secretion of the therapeutic related cytokines GM-CSF, GRO, IL-4, and PDGF-AA (FIGS. 48a, 48b). FIG. 48a provides a signaling schematic of the Akt pathway in hMSCs with the activator site highlighted for the p-Akt phospho-protein signaling node, while FIG. 48b shows how quantification of Akt phospho-protein signaling levels in both more therapeutic cell lines demonstrated increased signaling levels for all phospho-proteins except p-Akt, relative to the less therapeutic hMSCs.

As shown in FIG. 48c, the intervention strategy implemented shifted the paracrine signaling profile of more therapeutic hMSCs to a less therapeutic hMSC cytokine profile. More specifically, treatment with a p-JNK inhibitor and p-Akt activator (Donors L1+JNK Inh+Akt Act) yielded a rightward shift on LV1, in the PLSDA plot of the cytokine data, towards less therapeutic hMSCs on the right (FIG. 48c). While each more therapeutic hMSC donor shifted to a less therapeutic phenotype (along LV1), each donor maintained their inherit unique paracrine signaling properties as clearly defined on LV2 (FIG. 48c). These findings indicate that the p-JNK phospho-protein signaling in the MAPK pathway mediates the secretion of therapeutic related immunomodulatory cytokines associated with hMSC therapeutic efficacy in OA treatment and that p-Akt phospho-protein signaling in the Akt pathway regulates secretion of cytokines associated with nontherapeutic outcomes in OA.

Combined Activation of JNK and Inhibition of Akt Inhibitor Shifts Less Therapeutic hMSCs To A More Therapeutic Paracrine Signaling Profile

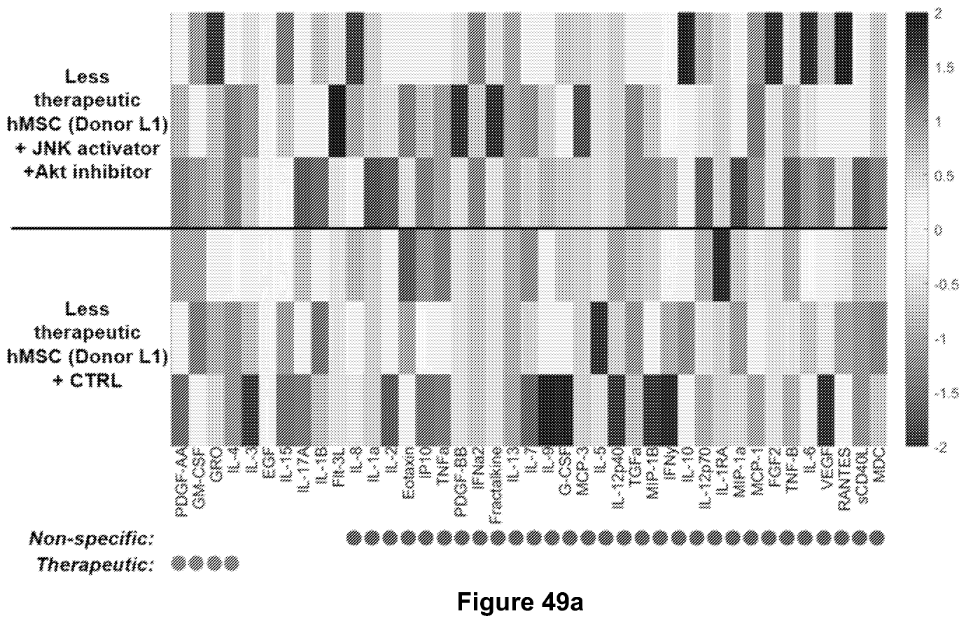

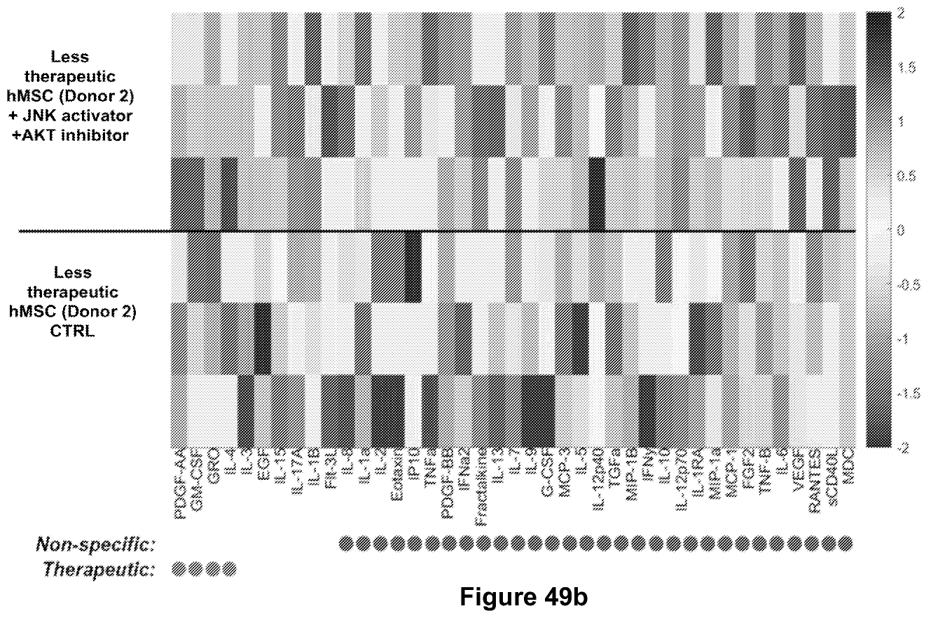

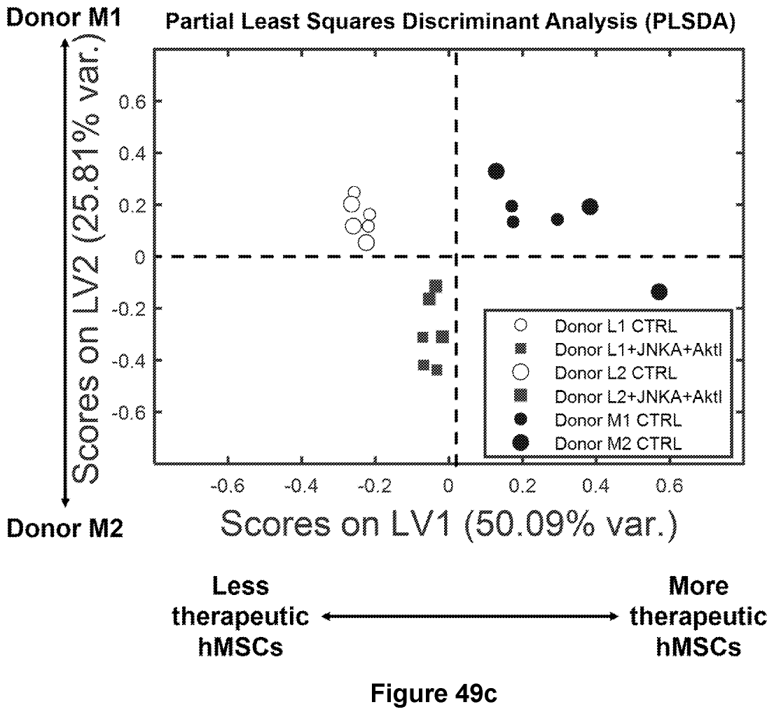

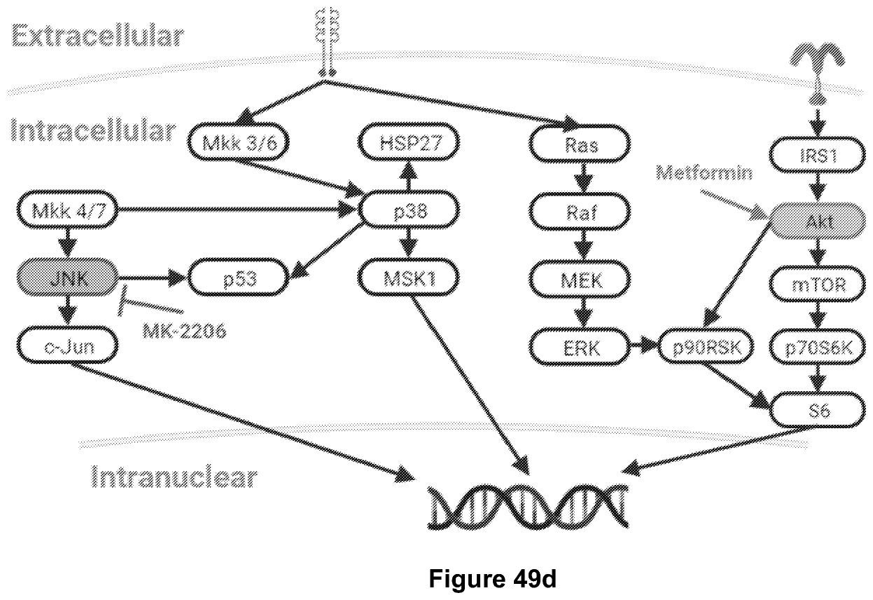

Having found that JNK appears to be in control of more therapeutic paracrine factors, while Akt appears to be in control of less therapeutic factors, the inventors next tested if combined treatment of less therapeutic cells (donors 1-2) with a JNK activator and Akt inhibitor would shift the cytokine profile of less therapeutic hMSCs towards a more therapeutic paracrine signaling profile, as shown in FIGS. 49a-49d, which illustrate paracrine signaling profiles of more therapeutic hMSCs (donors M1 and M2) treated with combination therapy of an Akt activator (SC79) and JNK inhibitor (SP600125).

To test this, the inventors co-treated less therapeutic cells with the JNK activator Metformin and the p-Akt inhibitor MK-2206, as shown in FIGS. 49a-49b. FIG. 49a illustrates quantification of immunomodulatory cytokines for more therapeutic hMSC donor M1, with combination therapy applied, showed a less targeted (increase in overall cytokine secretion) paracrine profile relative to the donor M1 CTRL. More specifically, hMSCs (donor M1) treated with the Akt activator and JNK inhibitor showed reduced cytokine secretion of GM-CSF, GRO, IL-4, and PDGF-AA relative to the donor M1 CTRL. FIG. 49b shows hMSC donor M2 yielded similar outcomes to donor M1 with less targeted cytokine secretion and reduced secretion of GM-CSF, GRO, IL-4, and PDGF-AA with treatment of an Akt activator and JNK inhibitor. For both donors 1 and 2, the combination treatment yielded targeted up-regulation of therapeutic-related cytokines/growth factors GM-CSF, GRO, IL-4, and PDGF-AA. Indeed, a PLSDA analysis demonstrated that combined treatment yielded a rightward shift of less therapeutic cells on LV1, shifting less therapeutic cells toward a paracrine secretary profile more consistent with more therapeutic hMSCs. Together, these data indicate that p-JNK phospho-protein signaling in the MAPK pathway and p-Akt phospho-protein signaling in the Akt pathway mediate the secretion of therapeutic immunomodulatory paracrine factors and thus represent important targets in control of hMSC therapeutic efficacy.

As shown in FIG. 49c, PLSDA analysis identified a profile of cytokines along LV1 that identified a distinct separation between more therapeutic hMSCs to the left and less therapeutic hMSCs to the right. Addition of the combination therapy shifted the more therapeutic hMSCs towards a less therapeutic paracrine profile (rightward shift on LV1; Donors L1+JNK Act+Akt Inh). Furthermore, differences in donor paracrine signaling were maintained with interventions as indicated by separation along LV2. Variability accounted for in each LV is included on respective axes labels. Finally, FIG. 49d illustrates a signaling schematic of the MAPK and Akt pathways in hMSCs with the specific nodes targeted in this combination therapy highlighted.

### DISCUSSION

While MSCs have shown promise as a treatment for OA, effective clinical translation has been limited by numerous factors including high variability and heterogeneity of MSCs and poor understanding of critical quality and potency attributes. These factors are of particular importance when considering therapeutic administration of these cellular therapeutics as the current standard of care in most clinical trials dictate MSCs be administered autologously to OA patients. Thus, participants in these trials are receiving highly heterogenous cellular therapeutic products, as prior work has demonstrated that therapeutic potency varies in MSCs isolated from different human donors. More specifically, prior research has demonstrated that MSCs isolated from different human donors exhibit differences in paracrine signaling activity, which is of particular importance as it has been demonstrated in prior studies that these paracrine signaling mechanisms serve as the major mechanism of action of MSCs in OA. However, while donor heterogeneity remains a pressing challenge facing the translation of MSC therapeutics from the pre-clinical to clinical environments; the majority of pre-clinical studies seeking to develop MSC therapeutics for OA utilize only a single donor. Thus, the objective of this Example was to assess donor heterogeneity to identify key secreted cytokines, RNA transcripts, and intracellular phospho-signaling proteins of hMSCs that relate to the therapeutic efficacy of hMSCs in OA.

To identify hMSC cellular attributes that relate to therapeutic outcomes in OA, donor heterogeneity was leveraged and four unique hMSC donors were used. All hMSCs implemented were from young and healthy patients, isolated from their bone marrow, and expanded to passage 4 to constrain for differences other than that of the human donor they were isolated from. An in vivo preclinical established model of OA (6-week MMT) was used to assess therapeutic efficacy as it serves as a more clinically relevant model, as patients present to the clinic following the presentation of disease manifestations after OA has developed. Quantitative microCT analysis of articular cartilage showed that donors 2.3 and 2.4 yielded more therapeutic outcomes, relative to hMSC donors 2.1 and 2.2, as they yielded reduced increases in articular cartilage swelling (volume and thickness), fibrillation development (surface roughness), and development of lesions (lesion volume).

To identify hMSC cellular attributes in vitro that relate to in vivo OA therapeutic outcomes, the microenvironment these cells are exposed to following their delivery in vivo was simulated in vitro. Inflammation has been well characterized as playing a key role in OA pathogenesis and thus in the current study an OA simulated microenvironment was engineered to quantify cellular attributes of hMSCs. To recapitulate the in vivo microenvironment (knee joint space following intra-articular injection) in vitro hMSCs were conditioned with IL-1β as it serves as a major pro-inflammatory modulator in OA. Relating in vitro hMSC cytokine secretion with in vivo therapeutic outcomes demonstrated that more therapeutic hMSCs yielded increased secretion of the chemokines (GM-CSF and GRO), cytokines (IL-4) and growth factors (PDGF-AA). For all these factors identified, there is a large body of prior research studying the role of these cytokines in MSCs in the context of OA, permitting this cytokine secretion to be contextualized. GM-CSF has been shown to enhance the mobilization of MSCs from the bone marrow and while this has not been directly linked to enhanced therapeutic efficacy in OA, increased MSC secretion of GM-CSF has demonstrated potentiated therapeutic efficacy of articular cartilage repair induced by microfracture. Furthermore, while the chemokine GRO (CXCL1) has been shown to initiate cartilage degradation and potentiate inflammation in the joints these resulting cellular actions make intuitive sense with the well-defined role GRO plays in monocyte and neutrophil trafficking to the site of injury. However, this proinflammatory event induced by hMSC secretion of GRO does not necessarily point to increased catabolismin the joint space as there may be important distinctions to be drawn between the chronic nature of the OA inflammatory environment and the acute response induced by the hMSC secretome. To resolve chronic inflammation, an acute event is needed to bring in immune cells and activate different inflammatory cascades to resolve and induce a pro-regenerative response. This mechanism is common in other chronic inflammatory and wound healing environments where acute inflammatory events are necessary to resolve chronic inflammation and transition to pro-regenerative immune responses to regulate inflammation. Thus, GRO-induced trafficking of monocytes and neutrophils may be contributing to this acute event (short duration inflammatory event) rather than potentiating the chronic inflammatory state of OA with more sustained inflammation. In addition to secretion of chemotactic factors, more therapeutic hMSCs demonstrated potentiated secretion of the potent anti-inflammatory cytokine IL-4. IL-4 has demonstrated chondroprotective effects in pre-clinical models of OA, where MSC spheroids transduced with IL-4 yielded better cartilage protection and pain relief, relative to naïve MSCs in vivo. These outcomes can be partly explained by further studies that have demonstrated IL-4 can protect cartilage by inhibiting inducible nitric oxide synthase (iNOS) and NO which yields subsequent suppression of IL-1β and TNF-α. Furthermore, the growth factor PDGF-AA has been studied extensively for its ability to yield therapeutic outcomes, both in combination and independently of MSC therapeutic delivery. More specifically, PDGF is believed to support tissue regeneration and anti-inflammatory properties in clinical patients and serves as a critical component of platelet rich plasma (PRP) therapeutics being delivered clinically to treat OA. Furthermore, in pre-clinical studies PDGF overexpressing MSCs were shown to exert anti-fibrotic, anti-inflammatory, and pro-chondrogenic capacities in a canine model of OA, through both the reduction of pro-inflammatory factors and MMP-13. While the role of each of these specific cytokines have been implicated in MSC therapeutic action in OA, this profile of secreted factors is unique and provides potential for development of a novel combinatorial therapeutic, accounting for all hMSC cytokines identified.

To take a more holistic approach to identifying cellular attributes that relate to therapeutic efficacy of hMSCs in OA, RNA-Seq was utilized to quantify gene expression of all hMSC donors. The genes EBF transcription factor 2 (EBF2), salt inducible kinase 2 (SIK2), and pro-platelet basic protein (PPBP) were identified as genes showing increased expression in more therapeutic hMSCs. These three genes have been identified in prior studies as key gene targets in pro-regenerative MSCs yielding therapeutic effects in OA and other associated musculoskeletal conditions. While RNA-Seq demonstrated the formation of unique gene expression profiles for both less therapeutic hMSCs and more therapeutic hMSCs, GSVA was implemented to study the enrichment of gene expression pathways. GSVA demonstrated clear differences in more and less therapeutic hMSC gene expression pathways. More specifically, more therapeutic hMSCs showed a less hematopoietic phenotype and a more anti-inflammatory phenotype. Furthermore, the GM-CSF and IL-4 gene expression pathways demonstrated increased enrichment in more therapeutic hMSCs relative to less therapeutic hMSCs, therefore further substantiating the outcomes obtained in the cytokine analysis study. However, while PDGF showed increased enrichment in the more therapeutic hMSC group, it was not significant and no gene enrichment pathway was available for the cytokine, GRO. Exploratory studies further implicated a number of signaling pathways including the Akt, MAPK, and NF-κB signaling pathways that were found to have significant differences in enrichment scores between more and less therapeutic hMSCs. The relevance of these signaling pathways was further supported in comparing gene pathway enrichment scores with therapeutic outcomes from microCT which further implicated the important of the MAPK and Akt pathways. Thus, motivating all of these pathways to be screened for differences in intracellular signaling between more and less therapeutic hMSCs.

Intracellular signaling is a key mediator of cytokine secretion as it permits cells to respond to their local environment and extracellular signals, through a series of signaling cascades which elicit a subsequent cellular response, including the secretion of immunomodulatory cytokines. While there are likely many intracellular signaling pathways mediating the immunomodulatory response of MSCs, the Akt, MAPK, NF-κB pathways were implicated as major contributors in the therapeutic efficacy of these cells. Furthermore, these pathways have been shown in in prior studies to be upstream regulators of many critical immunomodulatory cytokines, including the majority of those assessed in the immunomodulatory panel used for cytokine analysis. To study the role these pathways play in yielding therapeutic outcomes in hMSCs, all three pathways were screened in preliminary studies to identify those pathways which demonstrated distinct differences in phospho-protein signaling between less therapeutic hMSCs and more therapeutic hMSCs. These studies demonstrated that differences existed between less therapeutic and more therapeutic hMSCs in the Akt and MAPK signaling pathways; however, no distinct differences were noted in the NF-κB signaling pathway. Thus, the phospho-protein signaling levels in both the MAPK and Akt pathway were quantified in both more therapeutic hMSC donors (M1 and M2) and both less therapeutic hMSC donors (L1 and L2) in an OA simulated microenvironment (+IL-1β). These studies demonstrated that in more therapeutic hMSCs, MAPK and Akt signaling were potentiated, relative to less therapeutic hMSCs. More specifically, p-JNK phospho-protein levels in the MAPK pathway showed increased levels and the p-Akt showed decreased levels in more therapeutic hMSCs, relative to less therapeutic hMSCs. These findings are consistent with previous literature which have demonstrated the role that increased MAPK and Akt signaling in MSCs yield therapeutic outcomes in arthritic conditions. However, in these prior studies much the focus of the role of MAPK and Akt signaling has centered around their role in MSC proliferation, differentiation, apoptosis and senescence, and exosome production and not on the role these signaling pathways play in mediating the secretion of immunomodulatory cytokines.

Due to the central regulatory role these signaling pathways have in the paracrine signaling response of MSCs, the phospho-protein signals within these pathways provide viable therapeutic targets for mediating therapeutic MSC activity. To study the role of the MAPK pathway in hMSC paracrine signaling, immunomodulatory cytokine profiles of hMSCs were quantified following pharmacological intervention with a p-JNK inhibitor. This intervention demonstrated clear effects on hMSC paracrine signaling including more therapeutic hMSCs yielding reduced secretion of the cytokines GM-CSF, GRO, IL-4, and PDGF-AA (all therapeutic related cytokines). A similar approach was implemented to the study the role of Akt signaling using a p-Akt activator. While the p-Akt activator treatment increased secretion of non-specific cytokines, no reduced secretion of the therapeutic related cytokines were identified. The cumulative findings of these studies motivated implementation of a combination therapy using a p-JNK inhibitor and Akt activator combination in more therapeutic hMSCs and a p-JNK activator and Akt inhibitor in less therapeutic hMSCs.

To identify the cumulative signaling pathway dynamics mediating the paracrine signaling profile of more therapeutic hMSCs in OA, an intervention combination of a JNK inhibitor and Akt activator were used to treat hMSC donors M1 and M2. The hypothesis of the study was that treatment of more therapeutic hMSCs with a combination of a JNK inhibitor and Akt activator would shift the cytokine secretion to a less therapeutic paracrine profile. In this study, treated more therapeutic hMSCs demonstrated a less targeted cytokine profile (increase in secretion of majority of cytokines assessed) and reduced secretion of the therapeutic related cytokines GM-CSF, GRO, IL-4, and PDGF-AA. With this intervention, treated cells demonstrated a clear shift towards the paracrine phenotype of less therapeutic hMSCs. Furthermore, it was confirmed that both intervention strategies, SP600125 and SC79, effectively decreased p-JNK signaling and increased p-Akt signaling relative to untreated hMSCs. These findings indicate that the p-JNK phospho-protein in the MAPK pathway mediates the secretion of therapeutic related immunomodulatory cytokines associated with hMSC therapeutic efficacy in OA treatment and the p-Akt phospho-protein in the Akt pathway regulates secretion of cytokines associated with nontherapeutic outcomes in OA. These findings motivated the study of pharmacological intervention strategies to take hMSCs with less therapeutic paracrine profiles and shift them towards a more therapeutic phenotype.

It was suggested that treatment of less therapeutic hMSCs with a combination of a JNK activator and Akt inhibitor could shift the cytokine secretion to a more therapeutic paracrine profile. Treatment of hMSCs with a less therapeutic paracrine signaling profile yielded a more therapeutic cytokine profile. More specifically, less therapeutic hMSCs demonstrated more targeted paracrine signaling and increased secretion of therapeutic related cytokines GM-CSF, GRO, IL-4, and PDGF-AA. Furthermore, with this intervention, treated cells demonstrated a clear shift towards the paracrine phenotype of more therapeutic hMSCs. It was also confirmed that both intervention strategies, Metformin and MK-2206, effectively increased p-JNK signaling and decreased p-Akt signaling relative to untreated hMSCs. This ability to mediate MSC paracrine signaling phenotypes using these therapeutic intervention strategies presents a promising approach to potentiate current MSC therapeutics used in the clinical space. While Metformin has been studied previously in the context of MSC therapeutic for OA, the inhibition of Akt with MK-2206 in combination with Metformin remains a novel approach. Furthermore, MK-2206 has not been studied as an independent therapeutic intervention for MSCs as OA therapeutics. While this therapeutic is currently not FDA approved, there are a number of ongoing clinical trials using this inhibitor to treat solid tumors in clinical patients. Thus, the pharmacological intervention proposed herein implemented clinically relevant therapeutics as interventions to mediate therapeutic hMSC paracrine signaling as OA therapeutics.

Disparities in the administration of MSC therapeutics for OA treatment, with clinical trials yielding mixed therapeutic findings, in the clinic has motivated the adaptation of screening metrics and mediation strategies to yield more consistent therapeutic outcomes following treatment delivery. Outside of screening MSCs for their ability to adhere to plastic and express standard MSC phenotypic cell surface markers, no further screening metrics are currently implemented for MSCs that are clinically administered. This absence of screening is likely a major contributor to the high variability that has been reported in clinical studies. To address this knowledge gap in this study, cellular attributes that relate to therapeutic outcomes of hMSCs in OA were identified. More specifically, the secreted cytokines, RNA transcripts, and intracellular signaling phospho-proteins were identified (due to their role in paracrine signaling) that relate to therapeutic outcomes in a pre-clinical model of OA. Pharmacological intervention strategies were also devised to mediate the production of the therapeutic cytokine identified. The data presented herein shows a potential therapeutic strategy for potentiating hMSC treatment of OA.

### LIST OF EMBODIMENTS

The following non-exhaustive list of embodiments is contemplated herein.

Item 1. A method of distinguishing highly therapeutic mesenchymal stromal cells (MSCs) from less therapeutic MSCs for use in treating osteoarthritis in a subject in need thereof, the method comprising:
isolating MSCs from a biological sample;
incubating the MSCs with interleukin 1 beta (IL-1β), IL-6, interferon gamma (IFN-γ), tumor necrosis factor alpha (TNF-alpha), and combinations thereof;
measuring
   (i) levels of cytokines, chemokines, and/or growth factors secreted by the MSCs and/or
   (ii) measuring proteomic, phospho-proteomic, or transcription profiles of genes in mitogen-activated protein kinase (MAPK) and phosphatidylinositol 3-kinase/protein kinase B (PI3K/Akt) pathways; and
distinguishing highly therapeutic MSCs from less therapeutic MSCs by one or more steps of:
   (i) classifying the MSCs as being highly therapeutic if there is greater secretion of cytokines, chemokines, and/or growth factors associated with an increased phospho-c-Jun N-terminal kinase (p-JNK) level relative to secretion from a standard control MSC, or if the secretion of the cytokines, chemokines, and/or growth factors associated with the increased p-JNK level is substantially similar to secretion from a standard control MSC that has been previously identified as being highly therapeutic;
   (ii) classifying the MSCs as being less therapeutic if there is equal or lower secretion of cytokines, chemokines, and/or growth factors associated with the increased p-JNK level relative to secretion from a standard control MSC, or if the secretion of the cytokines, chemokines, and/or growth factors associated with the increased p-JNK level is substantially similar to secretion from a standard control MSC that has been previously identified as being less therapeutic; and/or
   (iii) classifying the MSCs as being highly therapeutic if levels of proteins, phospho-proteins, and/or expression of genes in the MAPK pathway are increased relative to levels of the same proteins, phospho-proteins, and/or genes in the MAPK pathway from a standard control MSC or substantially similar to levels of the same proteins, phospho-proteins, and/or genes in the MAPK pathway from a standard control MSC that has been previously identified as being highly therapeutic, and/or classifying the MSCs as being less therapeutic if levels of proteins, phospho-proteins, or expression of genes in the PI3K/Akt pathway are increased relative to levels of proteins, phospho-proteins, and/or genes in the PI3K/Akt pathway from a standard control MSC that has been previously identified as being highly therapeutic or are substantially similar to levels of proteins, phospho-proteins, and/or genes in the PI3K/Akt pathway from a standard control MSC that has been previously identified as being less therapeutic.

2. The method of item 1, wherein the biological source is bone marrow aspirate or bone marrow aspirate concentrate (BMAC); a lipoaspirate; a micronized lipoaspirate stromal vascular fraction; or tissue isolated from a placenta or umbilical cord.

3. The method of items 1 or 2, wherein the cytokines, chemokines, and/or growth factors associated with the increased p-JNK level comprise granulocyte macrophage colony stimulating factor (GM-CSF), chemokine ligand 1 (GRO), interleukin-4 (IL-4), and/or platelet derived growth factor (PDGF)-AA.

4. The method of any of items 1-3, wherein the proteins, phospho-proteins, and/or genes in the MAPK pathway associated with highly therapeutic MSCs comprise cJun, JNK, heat shock protein (HSP)-27, p38 MAP kinase, extracellular signal-regulated kinase (ERK), MAPK/ERK kinase (MEK), and/or activating transcription factor (Atf)-2.

5. The method of any of items 1-4, wherein the proteins, phospho-proteins, and/or genes in the PI3K/Akt pathway associated with less therapeutic MSCs comprise Akt, glycogen synthase kinase 3 (GSK3)-alpha, GSK3-beta, insulin like growth factor 1 receptor (IGF1R), insulin receptor (IR), insulin receptor substrate 1 (IRS1), mammalian target of rapamycin (mTor), ribosomal protein S6 kinase (p70S6k), phosphatase and tensin homologue (PTEN), ribosomal protein S6 (RPS6), and/or tuberous sclerosis complex 2 (TSC2).

6. A method of identifying and/or producing mesenchymal stromal cells as being therapeutically effective for treating osteoarthritis in a subject in need thereof, the method comprising:
incubating the MSCs with interleukin 1 beta (IL-1β), IL-6, interferon gamma (IFN-γ), tumor necrosis factor alpha (TNF-alpha), and combinations thereof;
measuring levels of cytokines, chemokines, and/or growth factors secreted by the MSCs;
optionally measuring transcription profiles of genes in mitogen-activated protein kinase (MAPK) and phosphatidylinositol 3-kinase/protein kinase B (PI3K/Akt) pathways; and
identifying the MSCs as being therapeutic if:
   (i) there is greater secretion of cytokines, chemokines, and/or growth factors associated with an increased phospho-c-Jun N-terminal kinase (p-JNK) level relative to secretion from a standard control MSC, or if the secretion of the cytokines, chemokines, and/or growth factors associated with the increased p-JNK level is substantially similar to secretion from a standard control MSC that has been previously identified as being therapeutic; and/or
   (ii) the levels of proteins, phospho-proteins, and/or expression of genes in the MAPK pathway are increased relative to levels of the same proteins, phospho-proteins, and/or genes in the MAPK pathway from a standard control MSC or substantially similar to levels of the same proteins, phospho-proteins, and/or genes in the MAPK pathway from a standard control MSC that has been previously identified as being highly therapeutic; and optionally propagating therapeutic MSCs.

7. The method of item 6, wherein the biological source is bone marrow aspirate or bone marrow aspirate concentrate (BMAC); a lipoaspirate; a micronized lipoaspirate stromal vascular fraction; or tissue isolated from a placenta or umbilical cord.

8. The method of items 6 or 7, wherein the cytokines, chemokines, and/or growth factors associated with increased p-JNK levels comprise granulocyte macrophage colony stimulating factor (GM-CSF), chemokine ligand 1 (GRO), interleukin-4 (IL-4), and/or platelet derived growth factor (PDGF)-AA.

9. The method of any of items 6-8, wherein the proteins, phospho-proteins, and/or genes in the MAPK pathway associated with therapeutic MSCs comprise cJun, JNK, heat shock protein (HSP)-27, p38 MAP kinase, extracellular signal-regulated kinase (ERK), MAPK/ERK kinase (MEK), and/or activating transcription factor (Atf)-2.

10. The method of claim any of items 6-9, wherein the step of propagating the therapeutic MSCs comprises tissue culture.

11. A method of altering the cytokine, chemokine, and/or growth factor expression profile of a plurality of mesenchymal stromal cells (MSCs) for use in treating osteoarthritis in a subject in need thereof, the method comprising:
isolating MSCs from a biological sample;
incubating the MSCs with interleukin 1 beta (IL-1β), IL-6, interferon gamma (IFN-γ), tumor necrosis factor alpha (TNF-alpha), and combinations thereof;
measuring
   (i) levels of cytokines, chemokines, and/or growth factors secreted by the MSCs and/or
   (ii) measuring proteomic, phospho-proteomic, or transcription profiles of genes in mitogen-activated protein kinase (MAPK) and phosphatidylinositol 3-kinase/protein kinase B (PI3K/Akt) pathways; and
classifying the MSCs as being highly therapeutic or less therapeutic by one or more steps of:
   (i) classifying the MSCs as being highly therapeutic if there is greater secretion of cytokines, chemokines, and/or growth factors associated with an increased phospho-c-Jun N-terminal kinase (p-JNK) level relative to secretion from a standard control MSC, or if the secretion of the cytokines, chemokines, and/or growth factors associated with the increased p-JNK level is substantially similar to secretion from a standard control MSC that has been previously identified as being highly therapeutic; and/or
   (ii) classifying the MSCs as being less therapeutic if there is equal or lower secretion of cytokines, chemokines, and/or growth factors associated with the increased p-JNK level relative to secretion from a standard control MSC, or if the secretion of the cytokines, chemokines, and/or growth factors associated with the increased p-JNK level is substantially similar to secretion from a standard control MSC that has been previously identified as being less therapeutic; and/or
   (iii) classifying the MSCs as being highly therapeutic if levels of proteins, phospho-proteins, and/or expression of genes in the MAPK pathway are increased relative to levels of the same proteins, phospho-proteins, and/or genes in the MAPK pathway from a standard control MSC or substantially similar to levels of the same proteins, phospho-proteins, and/or genes in the MAPK pathway from a standard control MSC that has been previously identified as being highly therapeutic, and/or classifying the MSCs as being less therapeutic if levels of proteins, phospho-proteins, and/or expression of genes in the PI3K/Akt pathway are increased relative to levels of the same proteins, phospho-proteins, and/or genes in the PI3K/Akt pathway from a standard control MSC or substantially similar to levels of the same proteins, phospho-proteins, and/or genes in the PI3K/Akt pathway from a standard control MSC that has been previously identified as being less therapeutic; and
treating the less therapeutic MSCs with an activator of the MAPK pathway and/or an inhibitor of the PI3K/Akt pathway, optionally prior to or simultaneously with administration of the less therapeutic MSCs to the subject in need thereof; and/or
treating the highly therapeutic MSCs with an activator of the MAPK pathway and/or an inhibitor of the PI3K/Akt pathway, optionally prior to or simultaneously with administration of the less therapeutic MSCs to the subject in need thereof.

12. The method of item 11, wherein the biological source is bone marrow aspirate or bone marrow aspirate concentrate (BMAC); a lipoaspirate; a micronized lipoaspirate stromal vascular fraction; or tissue isolated from a placenta or umbilical cord.

13. The method of items 11 or 12, wherein the cytokines, chemokines, and/or growth factors associated with increased p-JNK levels comprise granulocyte macrophage colony stimulating factor (GM-CSF), chemokine ligand 1 (GRO), interleukin-4 (IL-4), and/or platelet derived growth factor (PDGF)-AA.

14. The method of any of items 11-13, wherein the proteins, phospho-proteins, and/or genes in the MAPK pathway associated with highly therapeutic MSCs comprise cJun, JNK, heat shock protein (HSP)-27, p38 MAP kinase, extracellular signal-regulated kinase (ERK), MAPK/ERK kinase (MEK), and/or activating transcription factor (Atf)-2.

15. The method of any of items 11-14, wherein the proteins, phospho-proteins, or genes in the PI3K/Akt pathway associated with less therapeutic MSCs comprise Akt, glycogen synthase kinase 3 (GSK3)-alpha, GSK3-beta, insulin like growth factor 1 receptor (IGF1R), insulin receptor (IR), insulin receptor substrate 1 (IRS1), mammalian target of rapamycin (mTor), ribosomal protein S6 kinase (p70S6k), phosphatase and tensin homologue (PTEN), ribosomal protein S6 (RPS6), and/or tuberous sclerosis complex 2 (TSC2).

16. The method of any of items 11-15, wherein the activator of the MAPK pathway comprises a JNK activator.

17. The method of item 16, wherein the JNK activator comprises metformin, Sodium phenylbutyrate, AEBSF hydrocholoride, Azaspiracid-1, Scriptaid, MT-21, Anisomycin, Angiotensin II, and combinations thereof.

18. The method of any of items 11-16, wherein the inhibitor of the PI3K/Akt pathway comprises a phosphorylated Akt (p-Akt) inhibitor.

19. The method of item 18, wherein the p-Akt inhibitor comprises MK-2206, Miltefosine, magnolia extract NSC 293100, NSC 154020, KRX-0401, and combinations thereof.

20. A method of treating osteoarthritis in a subject in need thereof, the method comprising:
isolating MSCs from a biological sample;
incubating the MSCs with interleukin 1 beta (IL-1β), IL-6, interferon gamma (IFN-γ), tumor necrosis factor alpha (TNF-alpha), and combinations thereof;
measuring
   (i) levels of cytokines, chemokines, and/or growth factors secreted by the MSCs and/or
   (ii) measuring proteomic, phospho-proteomic, or transcription profiles of genes in mitogen-activated protein kinase (MAPK) and phosphatidylinositol 3-kinase/protein kinase B (PI3K/Akt) pathways; and
distinguishing highly therapeutic MSCs from less therapeutic MSCs by one or more steps of:
   (i) classifying the MSCs as being highly therapeutic if there is greater secretion of cytokines, chemokines, and/or growth factors associated with an increased phospho-c-Jun N-terminal kinase (p-JNK) level relative to secretion from a standard control MSC, or if the secretion of the cytokines, chemokines, and/or growth factors associated with the increased p-JNK level is substantially similar to secretion from a standard control MSC that has been previously identified as being highly therapeutic; and/or
   (ii) classifying the MSCs as being less therapeutic if there is equal or lower secretion of cytokines, chemokines, and/or growth factors associated with the increased p-JNK level relative to secretion from a standard control MSC, or if the secretion of the cytokines, chemokines, and/or growth factors associated with the increased p-JNK level is substantially similar to secretion from a standard control MSC that has been previously identified as being less therapeutic; and/or
   (iii) classifying the MSCs as being highly therapeutic if levels of proteins, phospho-proteins, and/or expression of genes in the MAPK pathway are increased relative to levels of the same proteins, phospho-proteins, and/or genes in the MAPK pathway from a standard control MSC or substantially similar to levels of the same proteins, phospho-proteins, and/or genes in the MAPK pathway from a standard control MSC that has been previously identified as being highly therapeutic, and/or classifying the MSCs as being less therapeutic if levels of proteins, phospho-proteins, and/or expression of genes in the PI3K/Akt pathway are increased relative to levels of the same proteins, phospho-proteins, and/or genes in the PI3K/Akt pathway from a standard control MSC or substantially similar to levels of the same proteins, phospho-proteins, and/or genes in the PI3K/Akt pathway from a standard control MSC that has been previously identified as being less therapeutic; and
      wherein:
the highly therapeutic MSCs are administered to the subject in need thereof or are optionally treated with an activator of the MAPK pathway and/or an inhibitor of the PI3K/Akt pathway prior to or simultaneously with administration of the highly therapeutic MSCs to the subject in need thereof;
the less therapeutic MSCs are treated with an activator of the MAPK pathway and/or an inhibitor of the PI3K/Akt pathway prior to or simultaneously with administration of the less therapeutic MSCs to the subject in need thereof.

21. The method of item 20, wherein the biological source is bone marrow aspirate or bone marrow aspirate concentrate (BMAC); a lipoaspirate; a micronized lipoaspirate stromal vascular fraction; or tissue isolated from a placenta or umbilical cord.

22. The method of items 20 or 21, wherein the cytokines, chemokines, and/or growth factors associated with increased p-JNK levels comprise granulocyte macrophage colony stimulating factor (GM-CSF), chemokine ligand 1 (GRO), interleukin-4 (IL-4), and/or platelet derived growth factor (PDGF)-AA.

23. The method of any of items 20-22, wherein the genes in the MAPK pathway associated with highly therapeutic MSCs comprise cJun, JNK, heat shock protein (HSP)-27, p38 MAP kinase, extracellular signal-regulated kinase (ERK), MAPK/ERK kinase (MEK), and/or activating transcription factor (Atf)-2.

24. The method of claim any of items 20-23, wherein the proteins, phospho-proteins, or genes in the PI3K/Akt pathway associated with less therapeutic MSCs comprise Akt, glycogen synthase kinase 3 (GSK3)-alpha, GSK3-beta, insulin like growth factor 1 receptor (IGF1R), insulin receptor (IR), insulin receptor substrate 1 (IRS1), mammalian target of rapamycin (mTor), ribosomal protein S6 kinase (p70S6k), phosphatase and tensin homologue (PTEN), ribosomal protein S6 (RPS6), and/or tuberous sclerosis complex 2 (TSC2).

25. The method of any of items 20-24, wherein the activator of the MAPK pathway comprises a JNK activator.

26. The method of item 25, wherein the JNK activator comprises metformin, Sodium phenylbutyrate, AEBSF hydrochloride, Azaspiracid-1, Scriptaid, MT-21, Anisomycin, Angiotensin II, and combinations thereof.

27. The method of any of items 20-25, wherein the inhibitor of the PI3K/Akt pathway comprises a phosphorylated Akt (p-Akt) inhibitor.

28. The method of item 27, wherein the p-Akt inhibitor comprises MK-2206, Miltefosine, magnolia extract NSC 293100, NSC 154020, KRX-0401, and combinations thereof.

29. The method of any of items 20-28, wherein the administration comprises intra-articular injection of the MSCs into a joint of the subject, the joint having osteoarthritis.

30. The method of any of items 20-29, wherein the subject is a mammal.

31. The method of claim 30, wherein the subject is a human, a horse, a cat, or a dog.

While several possible embodiments are disclosed above, embodiments of the present invention are not so limited. These exemplary embodiments are not intended to be exhaustive or to unnecessarily limit the scope of the invention, but instead were chosen and described in order to explain the principles of the present invention so that others skilled in the art may practice the invention. Indeed, various modifications of the invention in addition to those described herein will become apparent to those skilled in the art from the foregoing description. Such modifications are intended to fall within the scope of the appended claims.

1. A method comprising:
isolating mesenchymal stromal cells (MSCs) from a biological sample;
incubating the MSCs with interleukin 1 beta (IL-1β), IL-6, interferon gamma (IFN-γ), tumor necrosis factor alpha (TNF-alpha), and combinations thereof;
measuring at least one of:
levels of cytokines, chemokines, and/or growth factors secreted by the MSCs; or
proteomic, phospho-proteomic, or transcription profiles of genes in mitogen-activated protein kinase (MAPK) and phosphatidylinositol 3-kinase/protein kinase B (PI3K/Akt) pathways;
classifying at least one of:
the MSCs as being highly therapeutic if there is greater secretion of cytokines, chemokines, and/or growth factors associated with an increased phospho-c-Jun N-terminal kinase (p-JNK) level relative to secretion from a standard control MSC, or if the secretion of the cytokines, chemokines, and/or growth factors associated with the increased p-JNK level is substantially similar to secretion from a standard control MSC that has been previously identified as being highly therapeutic;
the MSCs as being less therapeutic if there is equal or lower secretion of cytokines, chemokines, and/or growth factors associated with the increased p-JNK level relative to secretion from a standard control MSC, or if the secretion of the cytokines, chemokines, and/or growth factors associated with the increased p-JNK level is substantially similar to secretion from a standard control MSC that has been previously identified as being less therapeutic;
the MSCs as being highly therapeutic if levels of proteins, phospho-proteins, and/or expression of genes in the MAPK pathway are increased relative to levels of the same proteins, phospho-proteins, and/or genes in the MAPK pathway from a standard control MSC or substantially similar to levels of the same proteins, phospho-proteins, and/or genes in the MAPK pathway from a standard control MSC that has been previously identified as being highly therapeutic; or
the MSCs as being less therapeutic if levels of proteins, phospho-proteins, and/or expression of genes in the PI3/Akt pathway are increased relative to levels of proteins, phospho-proteins, and/or genes in the PI3/Akt pathway from a standard control MSC that has been previously identified as being highly therapeutic or are substantially similar to levels of proteins, phospho-proteins, and/or genes in the PI3/Akt pathway from a standard control MSC that has been previously identified as being less therapeutic.

2. The method of claim 1, wherein the classifying distinguishes highly therapeutic MSCs from less therapeutic MSCs.

3. The method of claim 1, wherein at least one of:
the biological source is bone marrow aspirate or bone marrow aspirate concentrate (BMAC); a lipoaspirate; a micronized lipoaspirate stromal vascular fraction; or tissue isolated from a placenta or umbilical cord;
the cytokines, chemokines, and/or growth factors associated with the increased p-JNK level comprise granulocyte macrophage colony stimulating factor (GM-CSF), chemokine ligand 1 (GRO), interleukin-4 (IL-4), and/or platelet derived growth factor (PDGF)-AA;
the proteins, phospho-proteins, and/or genes in the MAPK pathway associated with highly therapeutic MSCs comprise cJun, JNK, heat shock protein (HSP)-27, p38 MAP kinase, extracellular signal-regulated kinase (ERK), MAPK/ERK kinase (MEK), and/or activating transcription factor (Atf)-2; or
the proteins, phospho-proteins, and/or genes in the PI3K/Akt pathway associated with less therapeutic MSCs comprise Akt, glycogen synthase kinase 3 (GSK3)-alpha, GSK3-beta, insulin like growth factor 1 receptor (IGF1R), insulin receptor (IR), insulin receptor substrate 1 (IRS1), mammalian target of rapamycin (mTor), ribosomal protein S6 kinase (p70S6k), phosphatase and tensin homologue (PTEN), ribosomal protein S6 (RPS6), and/or tuberous sclerosis complex 2 (TSC2).

4.-5. (canceled)

6. A method of identifying and/or producing mesenchymal stromal cells as being therapeutically effective for treating osteoarthritis in a subject in need thereof, the method comprising:
incubating the MSCs with interleukin 1 beta (IL-1β), IL-6, interferon gamma (IFN-γ), tumor necrosis factor alpha (TNF-alpha), and combinations thereof;
measuring levels of cytokines, chemokines, and/or growth factors secreted by the MSCs;
optionally measuring transcription profiles of genes in mitogen-activated protein kinase (MAPK) and phosphatidylinositol 3-kinase/protein kinase B (PI3K/Akt) pathways; and
identifying the MSCs as being therapeutic; and
optionally propagating therapeutic MSCs;
wherein the MSCs are identified as being therapeutic if at least one of:
there is greater secretion of cytokines, chemokines, and/or growth factors associated with an increased phospho-c-Jun N-terminal kinase (p-JNK) level relative to secretion from a standard control MSC, or if the secretion of the cytokines, chemokines, and/or growth factors associated with the increased p-JNK level is substantially similar to secretion from a standard control MSC that has been previously identified as being therapeutic; or
the levels of proteins, phospho-proteins, and/or expression of genes in the MAPK pathway are increased relative to levels of the same proteins, phospho-proteins, and/or genes in the MAPK pathway from a standard control MSC or substantially similar to levels of the same proteins, phospho-proteins, and/or genes in the MAPK pathway from a standard control MSC that has been previously identified as being highly therapeutic.

7. The method of claim 6, wherein at least one of:
the biological source is bone marrow aspirate or bone marrow aspirate concentrate (BMAC); a lipoaspirate; a micronized lipoaspirate stromal vascular fraction; or tissue isolated from a placenta or umbilical cord;
the cytokines, chemokines, and/or growth factors associated with increased p-JNK levels comprise granulocyte macrophage colony stimulating factor (GM-CSF), chemokine ligand 1 (GRO), interleukin-4 (IL-4), and/or platelet derived growth factor (PDGF)-AA; or
the proteins, phospho-proteins, and/or genes in the MAPK pathway associated with therapeutic MSCs comprise cJun, JNK, heat shock protein (HSP)-27, p38 MAP kinase, extracellular signal-regulated kinase (ERK), MAPK/ERK kinase (MEK), and/or activating transcription factor (Atf)-2.

8.-9. (canceled)

10. The method of claim 6, wherein the step of propagating the therapeutic MSCs comprises tissue culture.

11. The method of claim 1 further comprising at least one of:
treating the less therapeutic MSCs with an activator of the MAPK pathway and/or an inhibitor of the PI3K/Akt pathway, optionally prior to or simultaneously with administration of the treated less therapeutic MSCs to a subject in need thereof; or
treating the highly therapeutic MSCs with an activator of the MAPK pathway and/or an inhibitor of the PI3K/Akt pathway, optionally prior to or simultaneously with administration of the treated highly therapeutic MSCs to a subject in need thereof.

12. The method of claim 11, wherein at least one of:
the biological source is bone marrow aspirate or bone marrow aspirate concentrate (BMAC); a lipoaspirate; a micronized lipoaspirate stromal vascular fraction; or tissue isolated from a placenta or umbilical cord;
the cytokines, chemokines, and/or growth factors associated with increased p-JNK levels comprise granulocyte macrophage colony stimulating factor (GM-CSF), chemokine ligand 1 (GRO), interleukin-4 (IL-4), and/or platelet derived growth factor (PDGF)-AA;
the proteins, phospho-proteins, and/or genes in the MAPK pathway associated with highly therapeutic MSCs comprise cJun, JNK, heat shock protein (HSP)-27, p38 MAP kinase, extracellular signal-regulated kinase (ERK), MAPK/ERK kinase (MEK), and/or activating transcription factor (Atf)-2; or
the proteins, phospho-proteins, or genes in the PI3K/Akt pathway associated with less therapeutic MSCs comprise Akt, glycogen synthase kinase 3 (GSK3)-alpha, GSK3-beta, insulin like growth factor 1 receptor (IGF1R), insulin receptor (IR), insulin receptor substrate 1 (IRS1), mammalian target of rapamycin (mTor), ribosomal protein S6 kinase (p70S6k), phosphatase and tensin homologue (PTEN), ribosomal protein S6 (RPS6), and/or tuberous sclerosis complex 2 (TSC2).

13.-15. (canceled)

16. The method of claim 11, wherein the activator of the MAPK pathway comprises a JNK activator.

17. The method of claim 16, wherein the JNK activator comprises metformin, Sodium phenylbutyrate, AEBSF hydrocholoride, Azaspiracid-1, Scriptaid, MT-21, Anisomycin, Angiotensin II, and combinations thereof.

18. The method of claim 11, wherein the inhibitor of the PI3K/Akt pathway comprises a phosphorylated Akt (p-Akt) inhibitor.

19. The method of claim 18, wherein the p-Akt inhibitor comprises MK-2206, Miltefosine, magnolia extract NSC 293100, NSC 154020, KRX-0401, and combinations thereof.

20. The method of claim 1 further comprising at least one of:
administering the highly therapeutic MSCs to a subject in need thereof;
treating the highly therapeutic MSCs with an activator of the MAPK pathway and/or an inhibitor of the PI3K/Akt pathway prior to or simultaneously with administration of the treated highly therapeutic MSCs to the subject in need thereof; or
treating the less therapeutic MSCs with an activator of the MAPK pathway and/or an inhibitor of the PI3K/Akt pathway prior to or simultaneously with administration of the treated less therapeutic MSCs to a subject in need thereof.

21. The method of claim 20, wherein at least one of:
the biological source is bone marrow aspirate or bone marrow aspirate concentrate (BMAC); a lipoaspirate; a micronized lipoaspirate stromal vascular fraction; or tissue isolated from a placenta or umbilical cord;
the cytokines, chemokines, and/or growth factors associated with increased p-JNK levels comprise granulocyte macrophage colony stimulating factor (GM-CSF), chemokine ligand 1 (GRO), interleukin-4 (IL-4), and/or platelet derived growth factor (PDGF)-AA;
the genes in the MAPK pathway associated with highly therapeutic MSCs comprise cJun, JNK, heat shock protein (HSP)-27, p38 MAP kinase, extracellular signal-regulated kinase (ERK), MAPK/ERK kinase (MEK), and/or activating transcription factor (Atf)-2; or
the proteins, phospho-proteins, or genes in the PI3K/Akt pathway associated with less therapeutic MSCs comprise Akt, glycogen synthase kinase 3 (GSK3)-alpha, GSK3-beta, insulin like growth factor 1 receptor (IGF1R), insulin receptor (IR), insulin receptor substrate 1 (IRS1), mammalian target of rapamycin (mTor), ribosomal protein S6 kinase (p70S6k), phosphatase and tensin homologue (PTEN), ribosomal protein S6 (RPS6), and/or tuberous sclerosis complex 2 (TSC2).

22.-24. (canceled)

25. The method of claim 20, wherein the activator of the MAPK pathway comprises a JNK activator.

26. The method of claim 25, wherein the JNK activator comprises metformin, Sodium phenylbutyrate, AEBSF hydrocholoride, Azaspiracid-1, Scriptaid, MT-21, Anisomycin, Angiotensin II, and combinations thereof.

27. The method of claim 20, wherein the inhibitor of the PI3K/Akt pathway comprises a phosphorylated Akt (p-Akt) inhibitor.

28. The method of claim 27, wherein the p-Akt inhibitor comprises MK-2206, Miltefosine, magnolia extract NSC 293100, NSC 154020, KRX-0401, and combinations thereof.

29. The method of claim 20, wherein the administration comprises intra-articular injection of the MSCs into a joint of the subject, the joint having osteoarthritis.

30. The method of claim 20, wherein the subject is a mammal.

31. The method of claim 30, wherein the subject is a human, a horse, a cat, or a dog.
